(19) United States
(12) Patent Application Publication
(10) Pub. No.: US 2026/0200910 A1
(43) Pub. Date: Jul. 16, 2026

(54) PYRAZOLO[1,5-A]PYRIDINE AND IMIDAZO[1,2-A]PYRIDINE DERIVATIVES AS FGFR3 INHIBITORS FOR THE TREATMENT OF CANCER

(71) Applicant: Eli Lilly and Company, Indianapolis, IN (US)

(72) Inventor: Timothy Scott KERCHER, Longmont, CO (US)

(21) Appl. No.: 19/103,299

(22) PCT Filed: Sep. 6, 2023
(86) PCT No.: PCT/US2023/073501

§ 371 Date: Feb. 12, 2025

Related U.S. Application Data
(60) Provisional application No. 63/404,304, filed on Sep. 7, 2022.

(51) Int. Cl. C07D 471/04; A61K 31/439; A61K 31/444; A61K 31/4545; A61K 31/5377; C07D 519/00

(52) U.S. Cl. CPC C07D 471/04; A61K 31/439; A61K 31/444; A61K 31/4545; A61K 31/5377; C07D 519/00

(57) ABSTRACT

The present invention provides compounds of the formula as fibroblast growth factor receptor 3 (FGFR3) inhibitors for the treatment of e.g. cancer, systemic sclerosis, fibrosis, pulmonary fibrosis, achondroplasia, thanatophoric dysplasia, severe achondroplasia with developmental delay and acanthosis nigricans (SADDAN), and muenke syndrome. Preferred compounds are e.g. pyrazolo[1,5-a]pyridine and imidazo[1,2-a]pyridine derivatives.

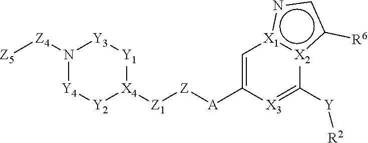

### BACKGROUND

Fibroblast growth factor (FGF) has been recognized as an important mediator of many physiological processes, such as morphogenesis during development, fibrosis, and angiogenesis. The fibroblast growth factor receptor (FGFR) family consists of five members four of which (FGFR 1-4) are glycoproteins composed of extracellular immunoglobulin (Ig)-like domains, a hydrophobic transmembrane region and a cytoplasmic part containing a tyrosine kinase domain. FGF binding leads to FGFR dimerization, followed by receptor autophosphorylation and activation of downstream signaling pathways. Receptor activation is sufficient for the recruitment and activation of specific downstream signaling partners that participate in the regulation of diverse processes such as cell growth, cell metabolism and cell survival. Thus, the FGF/FGFR signaling pathway has pleiotropic effects on many biological processes critical to tumor cell proliferation, migration, invasion, and angiogenesis.

It would be useful to develop new forms of FGFR3 inhibitors to treat cancer.

### SUMMARY

Provided herein are compounds of the formula:

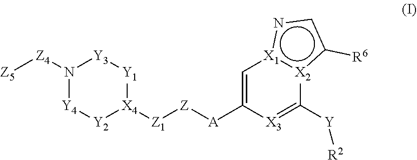

or a pharmaceutically acceptable salt thereof, wherein A, X₁, X₂, X₃, X₄, Y, Y₁, Y₂, Y₃, Y₄, Z, Z₁, Z₄, Z₅, R² and R⁶ are as defined herein.

Provided herein are compounds of the formula:

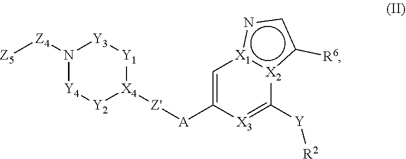

or a pharmaceutically acceptable salt thereof, wherein A, X₁, X₂, X₃, X₄, Y, Y₁, Y₂, Y₃, Y₄, Z′, Z₄, Z₅, R² and R⁶ are as defined herein.

Provided herein are compounds of the formula:

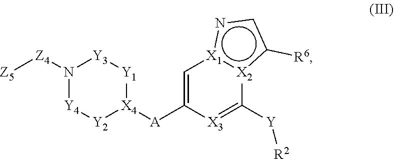

or a pharmaceutically acceptable salt thereof, wherein A, X₁, X₂, X₃, X₄, Y, Y₁, Y₂, Y₃, Y₄, Z₄, Z₅, R² and R⁶ are as defined herein.

Provided herein are compounds of the formula:

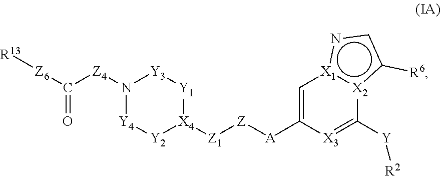

or a pharmaceutically acceptable salt thereof, wherein A, X₁, X₂, X₃, X₄, Y, Y₁, Y₂, Y₃, Y₄, Z, Z₁, Z₄, Z₆, R², R⁶ and R¹³ are as defined herein.

Provided herein are compounds of the formula:

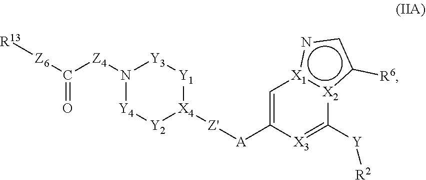

or a pharmaceutically acceptable salt thereof, wherein A, X₁, X₂, X₃, X₄, Y, Y₁, Y₂, Y₃, Y₄, Z′, Z₄, Z₆, R², R⁶ and R¹³ are as defined herein.

Provided herein are compounds of the formula:

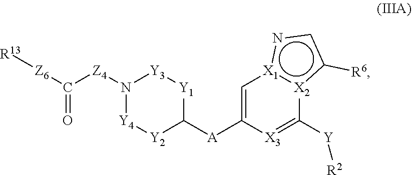

or a pharmaceutically acceptable salt thereof, wherein A, X₁, X₂, X₃, X₄, Y, Y₁, Y₂, Y₃, Y₄, Z₄, Z₆, R², R⁶ and R¹³ are as defined herein.

Provided herein are pharmaceutical compositions comprising a compound of formula (I), (II), (III), (IA), (IIA) or (IIIA), or a pharmaceutically acceptable salt thereof, with one or more pharmaceutically acceptable carriers, diluents, or excipients.

Provided herein are methods of using the compounds of formula (I), (II), (III), (IA), (IIA) or (IIIA), or a pharmaceutically acceptable salt thereof, and pharmaceutical compositions thereof, to treat proliferative disorders such as cancer, particularly to treat FGFR3-associated cancer. The methods include administering an effective amount of a compound of formula (I), (II), (III), (IA), (IIA) or (IIIA), or a pharmaceutically acceptable salt thereof, to a patient in need.

Provided herein, are compounds of formula (I), (II), (III), (IA), (IIA) or (IIIA), or a pharmaceutically acceptable salt thereof, for use in therapy. Further provided herein, are the compounds of formula (I), (II), (III), (IA), (IIA) or (IIIA), or a pharmaceutically acceptable salt thereof, for use in the treatment of cancer, particularly for use in the treatment of FGFR3-associated cancer. The use of compounds of formula (I), (II), (III), (IA), (IIA) or (IIIA), or a pharmaceutically acceptable salt thereof, in the manufacture of a medicament for treating cancer, particularly for use in the treatment of FGFR3-associated cancer, is also provided.

### DESCRIPTION

Provided herein are compounds believed to have clinical use for the treatment of cancer and particularly for the treatment of FGFR3-associated cancer.

Certain compounds provided herein have superior FGFR3 potency compared to certain previously known FGFR inhibitors. Certain compounds provided herein have superior selectivity for FGFR3 over FGFR1 compared to certain previously known FGFR inhibitors, reducing potential dose limiting toxicity caused by inhibition of FGFR1 (e.g. hyperphosphatemia).

The compounds provided herein are of formula (I):

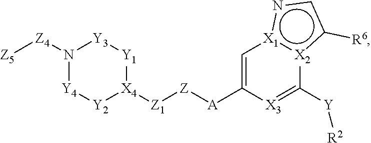

wherein
Z₅ is

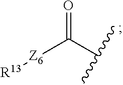

A is pyrazole, triazole, thiadiazole or oxadiazole, substituted with R¹ and R^{1A};
R¹ is hydrogen or C₁-C₃ alkyl;
R^{1A} is hydrogen, halo, CN, or C₁-C₃ alkyl optionally substituted with one or more substituents independently selected from halo, OH, and OCH₃;
X₁ and X₂ are independently selected from N and C, wherein when one of X₁ or X₂ is N the other is C;
X₃ is N or CH;
X₄ is N or C—R⁹;
Y is NH, O, S or a bond;
Y₁ is a bond, CHR⁷, CH₂—CHR⁷ or CHR⁷—CH₂, CF₂, CH₂—CF₂ or CF₂—CH₂;
Y₂ is a bond, CHR³, CH₂—CHR³ or CHR³—CH₂, CF₂, CH₂—CF₂ or CF₂—CH₂;
Y₃ is CR⁴R⁵ or CF₂;
Y₄ is CR³R⁴, or CF₂;
Y₅ is CR^{5A}R^{6A} or 3-6 membered cycloalkyl;
Z is a bond, CHR^{9A}, CR⁴R^{4A}, CR⁴R^{4A}—CH₂, CH₂—CR⁴R^{4A}, cyclobutyl, cyclopentyl, cyclohexyl, bicyclo(1.1.1)pentane, bicyclo(2.1.1)hexane, azetidine, pyrrolidine or piperidine;
Z₁ is a bond when Z is a bond, CR⁴R^{4A}, CR⁴R^{4A}—CH₂, CH₂—CR⁴R^{4A}, cyclobutyl, cyclopentyl, cyclohexyl, bicyclo(1.1.1)pentane, bicyclo(2.1.1)hexane, azetidine, pyrrolidine or piperidine, or Z₁ is CH₂ or CH₂—CH₂ when Z is CHR^{9A};
Z₂ is a bond, C(O), SO₂ or —NR⁴C(O);
Z₃ is a bond, C(O), SO₂ or —NR⁴C(O);
Z₄ is a bond, Y₅—NR¹⁵ or CH₂—Y₅—NR¹⁵ wherein the N of NR¹⁵ is connected to Z₅;
Z₆ is C═C or C≡C, wherein C═C is optionally substituted with R¹⁴;
R² is C₁-C₅ alkyl or R⁸, wherein C₁-C₅ alkyl is optionally substituted with one or more substituents independently selected from halo, OH, CN, oxo, —OC₁-C₄ alkyl, —OC₃-C₅ cycloalkyl, —Z₂—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN;
R³ is hydrogen, F, OH, OCH₃, C₁-C₃ alkyl, cyclopropyl, or one R³ is fused with R⁵ or R⁷ to form CH₂, CH₂—CH₂ or CH₂OCH₂;
R⁴ is hydrogen or C₁-C₃ alkyl;
R^{4A} is hydrogen, halo, OH, or C₁-C₃ alkyl;
R⁵ is hydrogen, F, OH, OCH₃, C₁-C₃ alkyl, cyclopropyl, or is fused with one R³ to form CH₂, CH₂—CH₂ or CH₂OCH₂;
R^{5A} is hydrogen or C₁-C₃ alkyl;
R⁶ is hydrogen, halo, C₁-C₅ alkyl, CN, 3-6 membered cycloalkyl, 4-6 membered heterocycloalkyl, 5-6 membered aryl or 5-6 membered heteroaryl, wherein 3-6 membered cycloalkyl, 4-6 membered heterocycloalkyl, 5-6 membered aryl and 5-6 membered heteroaryl are optionally substituted with one or more substituents independently selected from halo, methyl, halomethyl, OH or OCH₃ and wherein C₁-C₅ alkyl is optionally substituted with one or more substituents independently selected from halo, OH and OCH₃;
R^{6A} is hydrogen or C₁-C₃ alkyl;
R⁷ is hydrogen, F, OH, OCH₃, C₁-C₃ alkyl or is fused with one R³ to form CH₂, CH₂—CH₂ or CH₂OCH₂;
R⁸ is 3-6 membered cycloalkyl, 4-6 membered heterocycloalkyl, 5-6 membered aryl or 5-6 membered heteroaryl, optionally fused or substituted with R^{8A};
R^{8A} is 3-6 membered cycloalkyl, 4-6 membered heterocycloalkyl, 5-6 membered aryl or 5-6 membered heteroaryl;
R⁹ is hydrogen, C₁-C₃ alkyl, or is fused with R^{9A} to form CH₂ or CH₂—CH₂;
R¹⁰ is 3-6 membered cycloalkyl, 4-6 membered heterocycloalkyl, 5-6 membered aryl or 5-6 membered heteroaryl, optionally fused or substituted with R^{8A};
R₁₁ is C₁-C₄ alkyl, NH₂, NHC₁-C₃ alkyl, NHC₃-C₅ cycloalkyl or N(C₁-C₃ alkyl)₂, wherein C₁-C₄ alkyl, C₁-C₃ alkyl and C₃-C₅ cycloalkyl are optionally substituted with one or more substituents independently selected from halo, OH, OCH₃, methylamine, N,N-dimethylamine and CN;
R¹² is C₁-C₄ alkyl, C₃-C₅ cycloalkyl, NH₂, NHC₁-C₃ alkyl, NHC₃-C₅ cycloalkyl or N(C₁-C₃ alkyl)₂, wherein C₁-C₄ alky, C₁-C₃ alkyl and C₃-C₅ cycloalkyl are optionally substituted with one or more substituents independently selected from halo, OH, OCH₃, methylamine, N,N-dimethylamine and CN;
R¹³ is hydrogen, C₁-C₅ alkyl or R¹⁷, wherein C₁-C₅ alkyl is optionally substituted with one or more substituents independently selected from halo, OH, CN, —OC₁-C₃ alkyl, NH₂, NHC₁-C₃ alkyl or N(C₁-C₃ alkyl)₂, R¹⁷, NR¹⁶R¹⁷ and —OR¹⁷, wherein C₁-C₃ alkyl is optionally substituted with one or more substituents independently selected from halo, OH, OCH₃, and CN;
R¹⁴ is F, CF₃ or CN;
R¹⁵ is hydrogen or C₁-C₃ alkyl;
R¹⁶ is hydrogen or C₁-C₃ alkyl
R¹⁷ is 3-6 membered cycloalkyl, 4-6 membered heterocycloalkyl, 5-6 membered aryl, 5-6 membered heteroaryl or 7-12 membered spiroheteroalkyl having 1-2 ring nitrogen atoms, wherein 3-6 membered cycloalkyl, 4-6 membered heterocycloalkyl, 5-6 membered aryl and 5-6 membered heteroaryl are optionally fused or substituted with R^{17A};
R^{17A} is 3-6 membered cycloalkyl, 4-6 membered heterocycloalkyl, 5-6 membered aryl or 5-6 membered heteroaryl; and
R⁸, R¹⁰, R¹⁷, R^{8A} and R^{17A} are optionally substituted with one or more substituents independently selected from halo, OH, CN, —OC₁-C₄ alkyl, —OC₃-C₅ cycloalkyl and —Z₃—R¹² wherein C₁-C₄ alkyl and C₃-C₅ cycloalkyl are optionally substituted with one or more substituents independently selected from halo, OH, OCH₃, methylamine, N,N-dimethylamine and CN;
   or a pharmaceutically acceptable salt thereof.

In formula (II) and (IIA), A, X₁, X₂, X₃, Y, Y₁, Y₂, Y₃, Y₄, Z₄, Z₅, Z₆, R², R⁶ and R¹³ are as defined above for formula (I); and
X₄ is N or C—R⁹, wherein R⁹ is hydrogen or C₁-C₃ alkyl;
Z′ is a bond, CR⁴R^{4A}, CR⁴R^{4A}—CH₂, CH₂—CR⁴R^{4A}, cyclobutyl, cyclopentyl, cyclohexyl, bicyclo(1.1.1)pentane, bicyclo(2.1.1)hexane, azetidine, pyrrolidine or piperidine.

In formula (III) and (IIIA),
A, X₁, X₂, X₃, Y, Y₁, Y₂, Y₃, Y₄, Z₄, Z₆, R², R⁶ and R¹³ are as defined above for formula (I); and
X₄ is N or C—R⁹, wherein R⁹ is hydrogen or C₁-C₃ alkyl.

In the compounds of formula (I), (II), (III), (IA), (IIA) or (IIIA), X₁ can be C, and X₂ can be N; or X₁ can be N, and X₂ can be C.

In the compounds of formula (I), (II), (III), (IA), (IIA) or (IIIA), X₁ can be C, and X₂ can be N, forming:

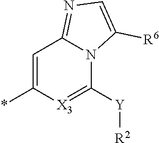

wherein * indicates the connection point to A in formula (I), (II), (III), (IA), (IIA) or (IIIA).

In the compounds of formula (I), (II), (III), (IA), (IIA) or (IIIA), X₁ can be N, and X₂ can be C, forming:

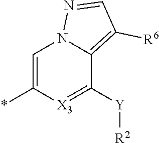

wherein * indicates the connection point to A in formula (I), (II), (III), (IA), (IIA) or (IIIA).

In the compounds of formula (I), (II), (III), (IA), (IIA) or (IIIA), X₁ can be C, X₂ can be N, and X₃ can be CH, forming:

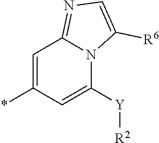

wherein * indicates the connection point to A in formula (I), (II), (III), (IA), (IIA) or (IIIA).

In the compounds of formula (I), (II), (III), (IA), (IIA) or (IIIA), X₁ can be N, X₂ can be C, and X₃ can be CH, forming:

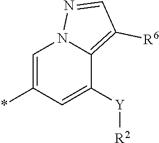

wherein * indicates the connection point to A in formula (I), (II), (III), (IA), (IIA) or (IIIA).

The specific chemical naming conventions used herein are intended to be familiar to one of skill in the chemical arts. Some terms are defined specifically for additional clarity.

As used herein, the term “alkyl” refers to a hydrocarbon chain that may be a straight chain or branched chain, containing the indicated number of carbon atoms. For example, the term “C₁-C₅ alkyl” as used herein refers to saturated linear or branched-chain monovalent hydrocarbon radicals of one, two, three, four or five carbon atoms. Examples of C₁-C₅ alkyl include, but are not limited to, methyl, ethyl, 1-propyl, isopropyl, 1-butyl, isobutyl, sec-butyl, tert-butyl, 2-methyl-2-propyl, pentyl and neopentyl. Examples of C₁-C₄ alkyl include, but are not limited to, methyl, ethyl, 1-propyl, isopropyl, 1-butyl, isobutyl, sec-butyl, tert-butyl and 2-methyl-2-propyl. Examples of C₁-C₃ alkyl include, but are not limited to, methyl, ethyl, 1-propyl or isopropyl.

As used herein, the term “cycloalkyl” means a saturated cyclic hydrocarbon group containing the indicated number of carbon atoms. For example, the term “3-6 membered cycloalkyl” as used herein refers to a saturated cyclic hydrocarbon group having three, four, five or six carbon atoms. Examples of 3-6 membered cycloalkyl include, cyclopropyl, cyclobutyl, cyclopentyl and cyclohexyl. Examples of 3-5 membered cycloalkyl include, cyclopropyl, cyclobutyl and cyclopentyl.

As used herein, the term “heterocycloalkyl” means a saturated cyclic group containing the indicated number of atoms selected from C(O)₀₋₁, N, O and S(O)₀₋₂. For example, the term “5-6 membered heterocycloalkyl” as used herein refers to a saturated cyclic ring system having five or six ring atoms, one, two or three of which are selected from N, O and S(O)₀₋₂, the remainder being C(O)₀₋₁. Examples of 4-6 membered heterocycloalkyl groups include, but are not limited to, azetidinyl, pyrrolidinyl, piperidinyl, piperazinyl, pyrrolidinyl, pyrrolidin-2-onyl, dioxanyl, morpholinyl, oxetanyl, tetrahydrofuranyl, tetrahydrothiophenyl, tetrahydropyranyl, oxazolidinyl, isothiazolidinyl oxozolid-2-onyl and isothiazolid-2-onyl. Examples of 5-6 membered heterocycloalkyl groups include, but are not limited to, piperidinyl, piperazinyl, pyrrolidinyl, pyrrolidin-2-onyl, dioxanyl, morpholinyl, tetrahydrofuranyl, tetrahydrothiophenyl, tetrahydropyranyl, oxazolidinyl, isothiazolidinyl oxozolid-2-onyl and isothiazolid-2-onyl.

As used herein, the term “aryl” refers to an aromatic cyclic hydrocarbon group having the indicated number of carbon atoms. For example, the term “5-6 membered aryl” as used herein refers to an aromatic cyclic hydrocarbon group having five or six carbon atoms. Examples of 5-6 membered aryls include cyclopentadienyl and phenyl.

As used herein, the term “heteroaryl” refers to an aromatic cyclic group having the indicated number of atoms selected from C, N, O and S. For example, the term “5-6 membered heteroaryl” as used herein refers to an aromatic cyclic group having five or six ring atoms, one, two or three of which are selected from N, O and S, the remainder being C. Examples of 5-6 membered heteroaryls include, but are not limited to, pyridinyl, pyrazinyl, pyrimidinyl, pyridazinyl, furanyl, pyrrolyl, thiophenyl, imidazolyl, pyrazolyl, triazolyl, oxazolyl, isoxazolyl, thiazolyl, isothiazolyl, oxadiazolyl and thiadiazolyl. Examples of 6 membered heteroaryls include, but are not limited to, pyridinyl, pyrazinyl, pyrimidinyl and pyridazinyl.

As used herein the term “halogen” or “halo” refers to F (fluoro), Cl (chloro), Br (bromo) and I (iodo).

As used herein the term “halomethyl” refers to —CH₃, in which one or more hydrogen atoms is/are replaced with an independently selected halo.

As used herein the term “oxo” refers to the substitution of CH₂ with O to form C(O).

As used herein the term spiroheteroalkyl means a saturated spirocyclic group containing the indicated number of atoms selected from C, N, O and S. For example, the term “7-12 membered spiroheteroalkyl having 1-2 ring nitrogen atoms” as used herein refers to a saturated spirocyclic ring system having seven, eight, nine, ten, eleven or twelve atoms, two of which are selected from N, the remainder being C. Examples of 7-12 membered spiroheteroalkyl having 1-2 ring nitrogen atoms include, but are not limited to, 2,6-diazaspiro[3.3]heptanyl, 2,7-diazaspiro[3.4]octanyl and 2,7-diazaspiro[3.5]nonanyl.

As used herein the term “N(C₁-C₃ alkyl)₂” allows the independent selection of each C₁-C₃ alkyl substituent, for example, N may be substituted by methyl and ethyl.

As used herein the substituent —NR⁴C(O) is connected to R² through N.

In the compounds of formula (I), (II), (III), (IA), (IIA) or (IIIA), A can be pyrazole, 1,2,3 triazole, 1,2,4 triazole, 1,2,3 thiadiazole, 1,2,4 thiadiazole, 1,2,5 thiadiazole, 1,3,4 thiadiazole, 1,2,3 oxadiazole, 1,2,4 oxadiazole, 1,2,5 oxadiazole or 1,3,4 oxadiazole, substituted with R¹ and R^{1A}.

In the compounds of formula (I), (II), (III), (IA), (IIA) or (IIIA), A can be pyrazole, 1,2,3 triazole or 1,2,4 triazole, substituted with R¹ and R^{1A}.

In the compounds of formula (I), (II), (III), (IA), (IIA) or (IIIA), A can be pyrazole, 1,2,3 triazole or 1,2,4 triazole, substituted with R¹ and R^{1A}, wherein R^{1A} is hydrogen and R¹ is C₁-C₃ alkyl.

In the compounds of formula (I), (II), (III), (IA), (IIA) or (IIIA), A can be pyrazole, 1,2,3 triazole or 1,2,4 triazole, substituted with R¹ and R^{1A}, wherein R^{1A} is hydrogen and R¹ is CH₃.

In the compounds of formula (I), II), (III), (IA) or (IIA), A can be:

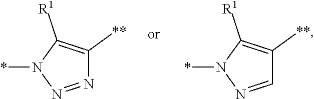

wherein * indicates the connection point to Z, Z′ or X₄ and ** indicates the other connection point from A in formula (I), (II), (III), (IA) or (IIA); and R¹ can be C₁-C₃ alkyl;

In the compounds of formula (IIIA), A can be:

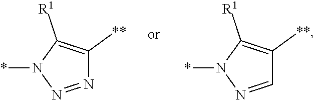

wherein * indicates the connection point to the substituent comprising Y₁ and ** indicates the other connection point from A in formula (IIIA); and R¹ can be C₁-C₃ alkyl;

In the compounds of formula (I), Z can be CHR^{9A}, cyclobutyl, azetidine, pyrrolidine or piperidine.

In the compounds of formula (I) or (IA), Z can be a bond,

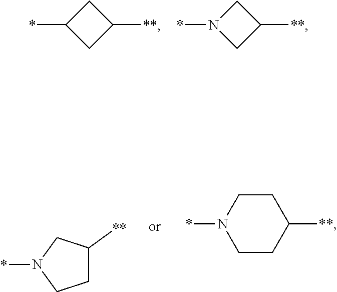

wherein * indicates the connection point to Z₁ and ** indicates the connection point to A in formula (I) or (IA).

In the compounds of formula (I) or (IA), Z can be a bond,

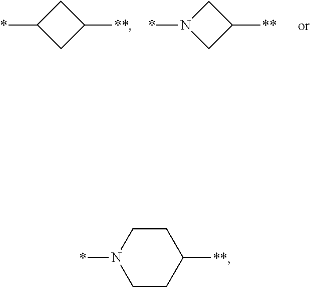

wherein * indicates the connection point to Z₁ and ** indicates the connection point to A in formula (I) or (IA).

In the compounds of formula (I) or (IA), Z can be CHR^{9A}, Z₁ can be selected from CH₂ or CH₂—CH₂, and R⁹ can be fused with R^{9A} to form CH₂ or CH₂—CH₂.

In the compounds of formula (I) or (IA), Z can be CHR^{9A}, Z₁ can be CH₂, and R⁹ can be fused with R^{9A} to form CH₂ or CH₂—CH₂.

In the compounds of formula (I) or (IA), Z can be CHR^{9A}, Z₁ can be CH₂—CH₂, and R⁹ can be fused with R^{9A} to form CH₂ or CH₂—CH₂.

In the compounds of formula (I) or (IA), Z can be CHR^{9A}, Z₁ can be selected from CH₂ or CH₂—CH₂, and R⁹ can be fused with R^{9A} to form CH₂.

In the compounds of formula (I) or (IA), Z can be CHR^{9A}, Z₁ can be selected from CH₂ or CH₂—CH₂, and R⁹ can be fused with R^{9A} to form CH₂—CH₂.

In the compounds of formula (I) or (IA), Z can be CHR^{9A}, Z₁ can be CH₂, and R⁹ can be fused with R^{9A} to form CH₂.

In the compounds of formula (I) or (IA), Z can be CHR^{9A}, Z₁ can be CH₂—CH₂, and R⁹ can be fused with R^{9A} to form CH₂—CH₂.

In the compounds of formula (II) or (IIA), Z′ can be:

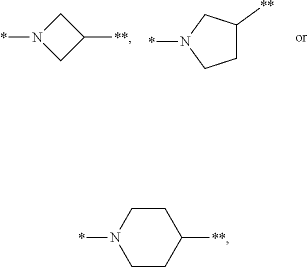

wherein ** indicates the connection point to A and * indicates the other connection point from Z′ in formula (II) or (IIA).

In the compounds of formula (II) or (IIA), Z′ can be:

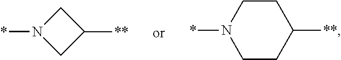

wherein ** indicates the connection point to A and * indicates the other connection point from Z′ in formula (II) or (IIA).

In the compounds of formula (I) or (IA), Z can be a bond.

In the compounds of formula (II) or (IIA), Z′ can be a bond.

In the compounds of formula (I), (II), (IA) or (IIA), Z₁ can be a bond.

In the compounds of formula (I), (II), (III), (IA), (IIA) or (IIIA), Y can be NH or O.

In the compounds of formula (I), (II), (III), (IA), (IIA) or (IIIA), Y can be O.

In the compounds of formula (I), (II), (III), (IA), (IIA) or (IIIA), Y₁ can be a bond, CHR⁷, CH₂—CHR⁷ or CHR⁷—CH₂, wherein R⁷ is selected from hydrogen, F, OH and CH₃; and Y₂ can a bond, CHR³, CH₂—CHR³ or CHR³—CH₂, wherein R³ is selected from hydrogen, F, OH and CH₃.

In the compounds of formula (I), (II), (III), (IA), (IIA) or (IIIA), Y₁ can be a bond or CHR⁷, wherein R⁷ is hydrogen, F, OH or CH₃; and Y₂ can a bond or CHR³, wherein R³ is hydrogen, F, OH or CH₃.

In the compounds of formula (I), (II), (III), (IA) or (IIA), Y₁ can be a bond, CHR⁷, CH₂—CHR⁷ or CHR⁷—CH₂, wherein R⁷ is hydrogen, F, OH or CH₃; and Y₂ can a bond, CHR³, CH₂—CHR³ or CHR³—CH₂, wherein R³ is hydrogen, F, OH or CH₃, forming:

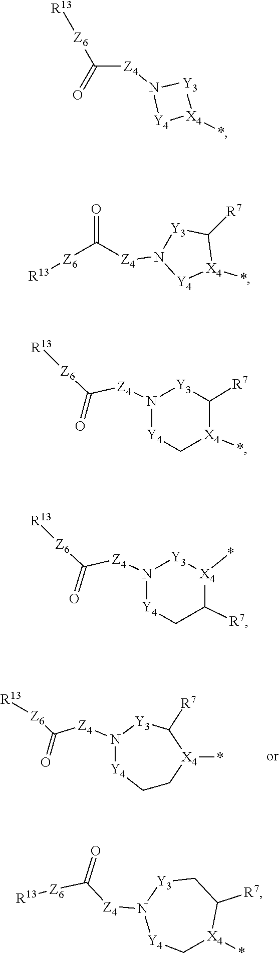

wherein * indicates the connection point to Z₁, in formula (I) or (IA); Z′ in formula (II) or (IIA); or A in formula (III).

In the compounds of formula (I), (II), (III), (IA) or (IIA), Y₁ can be a bond or CHR⁷, wherein R⁷ is hydrogen, F, OH or CH₃; and Y₂ can a bond or CHR³, wherein R³ is hydrogen, F, OH or CH₃, forming:

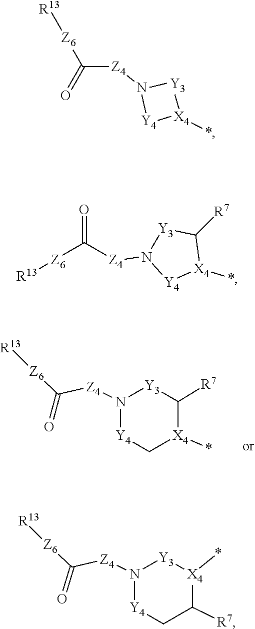

wherein * indicates the connection point to Z₁ in formula (I) or (IA); Z′ in formula (II) or (IIA); or A in formula (III).

In the compounds of formula (I), (II), (III), (IA), (IIA) or (IIIA), Y₁ can be a bond, CHR⁷, CH₂—CHR⁷ or CHR⁷—CH₂, wherein R⁷ is hydrogen, F, OH or CH₃; and Y₂ can a bond, CHR³, CH₂—CHR³ or CHR³—CH₂, wherein R³ is hydrogen, F, OH or CH₃, forming:

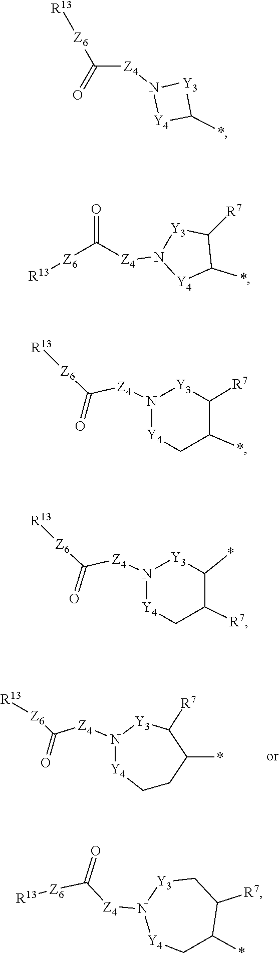

wherein * indicates the connection point to Z₁ in formula (I) or (IA); Z′ in formula (II) or (IIA); or A in formula (III) or (IIIA).
(IIIA).

In the compounds of formula (I), (II), (III), (IA), (IIA) or (IIIA), Y₁ can be a bond or CHR⁷, wherein R⁷ is hydrogen, F, OH or CH₃; and Y₂ can a bond or CHR³, wherein R³ is hydrogen, F, OH or CH₃, forming:

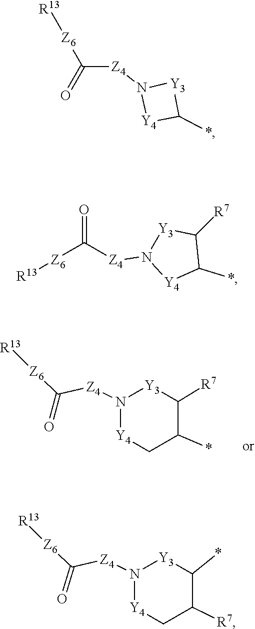

wherein * indicates the connection point to Z₁ in formula (I) or (IA); Z′ in formula (II) or (IIA); or A in formula (III) or (IIIA).

In the compounds of formula (I), (II), (III), (IA), (IIA) or (IIIA), R^{1A} can be hydrogen or C₁-C₃ alkyl optionally substituted with one or more substituents independently selected from halo, OH and OCH₃.

In the compounds of formula (I), (II), (III), (IA), (IIA) or (IIIA), R^{1A} can be hydrogen or CH₃.

In the compounds of formula (I), (II), (III), (IA), (IIA) or (IIIA), R^{1A} can be hydrogen.

In the compounds of formula (I), (II), (III), (IA), (IIA) or (IIIA), R¹ can be methyl, ethyl or propyl.

In the compounds of formula (I), (II), (III), (IA), (IIA) or (IIIA), R¹ can be methyl.

In the compounds of formula (I), (II), (III), (IA), (IIA) or (IIIA), R² can be C₁-C₃ alkyl optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH3, methylamine, N,N-dimethylamine and CN.

In the compounds of formula (I), (II), (III), (IA), (IIA) or (IIIA), R² can be C₁-C₃ alkyl optionally substituted with one or more substituents independently selected from F, OH, CN, oxo, —OCH₃, —OC₃ cycloalkyl and R¹⁰.

In the compounds of formula (I), (II), (III), (IA), (IIA) or (IIIA), R² can be:

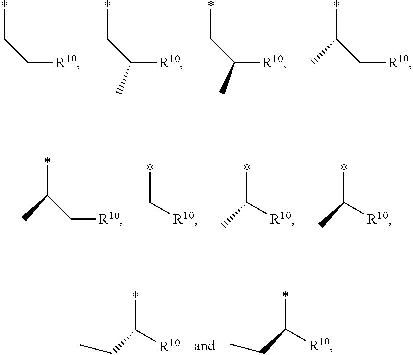

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I), (II), (III), (IA), (IIA) or (IIIA).

In the compounds of formula (I), (II), (III), (IA), (IIA) or (IIIA), R² can be:

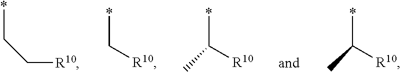

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I), (II), (III), (IA), (IIA) or (IIIA).

In the compounds of formula (I), (II), (III), (IA), (IIA) or (IIIA), R² can be:

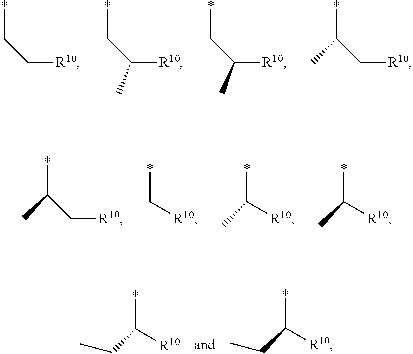

optionally substituted with one, two, three or four substituents independently selected from F, OH, CN, oxo, —OCH₃, —OC₃ cycloalkyl and R¹⁰, wherein * indicates the connection point to Y in formula (I), (II), (III), (IA), (IIA) or (IIIA).

In the compounds of formula (I), (II), (III), (IA), (IIA) or (IIIA), R² can be:

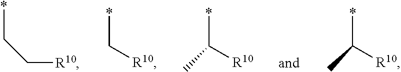

optionally substituted with one, two, three or four substituents independently selected from F, OH, CN, oxo, —OCH₃ and —OC₃ cycloalkyl and R¹⁰, wherein * indicates the connection point to Y in formula (I), (II), (III), (IA), (IIA) or (IIIA).

In the compounds of formula (I), (II), (III), (IA), (IIA) or (IIIA), Y₁ can be a bond, CHR⁷, CH₂—CHR⁷ or CHR⁷—CH₂, wherein R⁷ can be selected from hydrogen, F, OH and CH₃.

In the compounds of formula (I), (II), (III), (IA), (IIA) or (IIIA), Y₂ can be a bond, CHR³, CH₂—CHR³ or CHR³—CH₂, wherein R³ can be selected from hydrogen, F, OH and CH₃.

In the compounds of formula (I), (II), (III), (IA), (IIA) or (IIIA), Y₃ can be CR⁴R⁵ or CF₂, wherein R⁴ is hydrogen or CH₃ and R⁵ is hydrogen, F, OH or CH₃; and Y₄ is CR³R⁴ or CF₂ wherein R⁴ is hydrogen or CH₃, and R³ is hydrogen, F, OH or CH₃.

In the compounds of formula (I), (II), (III), (IA), (IIA) or (IIIA), Y₃ can be CR⁴R⁵, wherein R⁴ is hydrogen and R⁵ is fused with one R³ to form CH₂, CH₂—CH₂ or CH₂OCH₂; and Y₄ is CR³R⁴ wherein R⁴ is hydrogen, and R³ is fused with R⁵ to form CH₂, CH₂—CH₂ or CH₂OCH₂.

In the compounds of formula (I), (II), (III), (IA) or (IIA), Y₃ can be CR⁴R⁵, wherein R⁴ is hydrogen and R⁵ is fused with one R³ to form CH₂, CH₂—CH₂ or CH₂OCH₂; and Y₄ is CR³R⁴ wherein R⁴ is hydrogen, and R³ is fused with R⁵ to form CH₂, CH₂—CH₂ or CH₂OCH₂, forming:

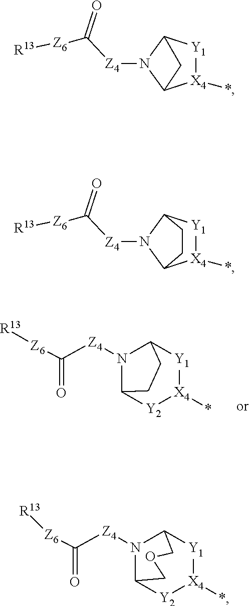

wherein * indicates the connection point to Z₁ in formula (I) or (IA); Z′ in formula (II) or (IIA); or A in formula (III).

In the compounds of formula (IIIA), Y₃ can be CR⁴R⁵, wherein R⁴ is hydrogen and R⁵ is fused with one R³ to form CH₂, CH₂—CH₂ or CH₂OCH₂; and Y₄ is CR³R⁴ wherein R⁴ is hydrogen, and R³ is fused with R⁵ to form CH₂, CH₂—CH₂ or CH₂OCH₂, forming:

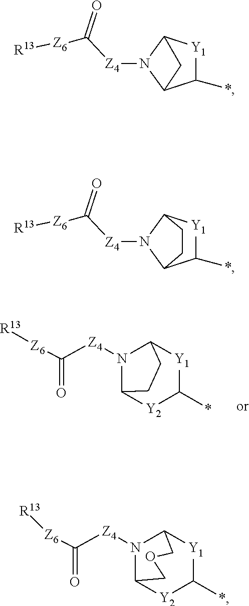

wherein * indicates the connection point to Z₁ in formula (I) or (IA); Z′ in formula (II) or (IIA); or A in formula (III) or (IIIA).

In the compounds of formula (I), (II), (III), (IA) or (IIA), X₄ can be N or C—R⁹ wherein R⁹ is hydrogen or CH₃.

In the compounds of formula (I) or (IA), X₄ can be C—R⁹ wherein R⁹ is fused with R^{9A} to form CH₂ or CH₂—CH₂; and Z₁ is CH₂ or CH₂—CH₂.

In the compounds of formula (I), (II), (III), (IA) or (IIA), X₄ can be N or CH.

In the compounds of formula (I), (II), (III), (IA), (IIA) or (IIIA), R⁶ can be CN, F, Cl, CH₃, CF₃ or cyclopropyl.

In the compounds of formula (I), (II), (III), (IA), (IIA) or (IIIA), R⁶ can be CN, F, Cl or CF₃.

In the compounds of formula (I), (II), (III), (IA), (IIA) or (IIIA), R⁶ can be CN or C₁.

In the compounds of formula (I), (II), (III), (IA), (IIA) or (IIIA), R⁶ can be CN.

In the compounds of formula (I), (II), (III), (IA), (IIA) or (IIIA), R⁶ can be Cl.

In the compounds of formula (I), (II), (III), (IA), (IIA) or (IIIA), R⁸ can be 5-6 membered cycloalkyl, 5-6 membered heterocycloalkyl, 5-6 membered aryl or 5-6 membered heteroaryl, optionally fused or substituted with R^{8A}.

In the compounds of formula (I), (II), (III), (IA), (IIA) or (IIIA), R⁸ can be 5-6 membered cycloalkyl or 5-6 membered heterocycloalkyl, optionally fused with R^{8A}.

In the compounds of formula (I), (II), (III), (IA), (IIA) or (IIIA), R⁸ can be cyclopentyl, cyclohexyl, piperidinyl, pyrrolidinyl, tetrahydrofuranyl, tetrahydropyranyl, phenyl or pyridinyl, optionally fused with R^{8A}.

In the compounds of formula (I), (II), (III), (IA), (IIA) or (IIIA), R⁸ can be cyclopentyl, cyclohexyl, piperidinyl, pyrrolidinyl, tetrahydrofuranyl or tetrahydropyranyl, fused with R^{8A}.

In the compounds of formula (I), (II), (III), (IA), (IIA) or (IIIA), R⁸ can be cyclopentyl, cyclohexyl, piperidinyl, pyrrolidinyl, tetrahydrofuranyl or tetrahydropyranyl, fused with R^{8A}, wherein R^{8A} can be phenyl or 6 membered heteroaryl.

In the compounds of formula (I), (II), (III), (IA) or (IIA), R⁹ can be hydrogen.

In the compounds of formula (I), (II), (III), (IA), (IIA) or (IIIA), R¹⁰ can be 3-6 membered cycloalkyl, 5-6 membered heterocycloalkyl, 5-6 membered aryl or 5-6 membered heteroaryl, optionally fused with R^{8A}.

In the compounds of formula (I), (II), (III), (IA), (IIA) or (IIIA), R¹⁰ can be 3-6 membered cycloalkyl, 5-6 membered heterocycloalkyl, phenyl or 5-6 membered heteroaryl, optionally fused or substituted with R^{8A}.

In the compounds of formula (I), (II), (III), (IA), (IIA) or (IIIA), R¹⁰ can be cyclopropyl, cyclobutyl, cyclopentyl, cyclohexyl, piperidinyl, piperazinyl, pyrrolidinyl, pyrrolidin-2-onyl, dioxanyl, morpholinyl, tetrahydrofuranyl, tetrahydrothiophenyl, tetrahydropyranyl, oxazolidinyl, isothiazolidinyl, oxozolid-2-onyl, isothiazolid-2-onyl, phenyl, pyridinyl, pyrazinyl, pyrimidinyl, pyridazinyl, furanyl, pyrrolyl, thiophenyl, imidazolyl, pyrazolyl, triazolyl, oxazolyl, isoxazolyl, thiazolyl, isothiazolyl, oxadiazolyl or thiadiazolyl, optionally fused or substituted with R^{8A}.

In the compounds of formula (I), (II), (III), (IA), (IIA) or (IIIA), R¹⁰ can be cyclopropyl, cyclobutyl, cyclopentyl, cyclohexyl, tetrahydrofuranyl, tetrahydropyranyl, phenyl, pyridinyl, pyrazinyl, pyrimidinyl, pyridazinyl, pyrazolyl, triazolyl, oxazolyl, isoxazolyl, thiazolyl, isothiazolyl, oxadiazolyl or thiadiazolyl, optionally fused or substituted with R^{8A}.

In the compounds of formula (I), (II), (III), (IA), (IIA) or (IIIA), R¹⁰ can be cyclopropyl, cyclobutyl, cyclopentyl, cyclohexyl, tetrahydrofuranyl, tetrahydropyranyl, phenyl, pyridinyl, pyrazinyl, pyrimidinyl, pyridazinyl, oxazolyl, isoxazolyl, thiazolyl, isothiazolyl, oxadiazolyl or thiadiazolyl, optionally fused or substituted with R^{8A}. In the compounds of formula (I), (II), (III), (IA), (IIA) or (IIIA), R¹⁰ can be cyclopropyl, cyclobutyl, phenyl, pyridinyl, oxazolyl, isoxazolyl, thiazolyl or isothiazolyl, optionally fused or substituted with R^{8A}.

In the compounds of formula (I), (II), (III), (IA), (IIA) or (IIIA), R¹⁰ can be cyclopropyl, cyclobutyl, cyclopentyl, cyclohexyl, piperidinyl, piperazinyl, pyrrolidinyl, pyrrolidin-2-onyl, dioxanyl, morpholinyl, tetrahydrofuranyl, tetrahydrothiophenyl, tetrahydropyranyl, oxazolidinyl, isothiazolidinyl, oxozolid-2-onyl, isothiazolid-2-onyl, phenyl, pyridinyl, pyrazinyl, pyrimidinyl, pyridazinyl, furanyl, pyrrolyl, thiophenyl, imidazolyl, pyrazolyl, triazolyl, oxazolyl, isoxazolyl, thiazolyl, isothiazolyl, oxadiazolyl or thiadiazolyl.

In the compounds of formula (I), (II), (III), (IA), (IIA) or (IIIA), R¹⁰ can be cyclopropyl, cyclobutyl, cyclopentyl, cyclohexyl, tetrahydrofuranyl, tetrahydropyranyl, phenyl, pyridinyl, pyrazinyl, pyrimidinyl, pyridazinyl, pyrazolyl, triazolyl, oxazolyl, isoxazolyl, thiazolyl, isothiazolyl, oxadiazolyl or thiadiazolyl.

In the compounds of formula (I), (II), (III), (IA), (IIA) or (IIIA), R¹⁰ can be cyclopropyl, cyclobutyl, cyclopentyl, cyclohexyl, tetrahydrofuranyl, tetrahydropyranyl, phenyl, pyridinyl, pyrazinyl, pyrimidinyl, pyridazinyl, oxazolyl, isoxazolyl, thiazolyl, isothiazolyl, oxadiazolyl or thiadiazolyl.

In the compounds of formula (I), (II), (III), (IA), (IIA) or (IIIA), R¹⁰ can be cyclopropyl, cyclobutyl, phenyl, pyridinyl, oxazolyl, isoxazolyl, thiazolyl or isothiazolyl.

In the compounds of formula (I), (II), (III), (IA), (IIA) or (IIIA), R¹⁰ can be cyclopentyl, cyclohexyl, tetrahydrofuranyl, tetrahydropyranyl, phenyl, pyridinyl, pyrazinyl, pyrimidinyl, pyridazinyl, pyrazolyl, triazolyl, oxazolyl, isoxazolyl, thiazolyl, isothiazolyl, oxadiazolyl or thiadiazolyl, fused or substituted with R^{8A}.

In the compounds of formula (I), (II), (III), (IA), (IIA) or (IIIA), R¹⁰ can be cyclopentyl, cyclohexyl, piperidinyl, pyrrolidinyl, tetrahydrofuranyl, tetrahydropyranyl, phenyl or pyridinyl, fused or substituted with R^{8A}.

In the compounds of formula (I), (II), (III), (IA), (IIA) or (IIIA), R¹⁰ can be cyclopentyl, cyclohexyl, piperidinyl, pyrrolidinyl, tetrahydrofuranyl, tetrahydropyranyl, phenyl or pyridinyl, fused or substituted with R^{8A}.

In the compounds of formula (I), (II), (III), (IA), (IIA) or (IIIA), R¹⁰ can be cyclopentyl, cyclohexyl, piperidinyl, pyrrolidinyl, tetrahydrofuranyl, tetrahydropyranyl, phenyl or pyridinyl, fused or substituted with R^{8A}.

In the compounds of formula (I), (II), (III), (IA), (IIA) or (IIIA), R¹⁰ can be cyclopentyl, cyclohexyl, phenyl or pyridinyl, fused or substituted with R^{8A}.

In the compounds of formula (I), (II), (III), (IA), (IIA) or (IIIA), R¹⁰ can be phenyl or pyridinyl, fused with R^{8A} wherein R^{8A} can be 5-6 membered heterocycloalkyl or 5-6 membered heteroaryl.

In the compounds of formula (I), (II), (III), (IA), (IIA) or (IIIA), R¹⁰ can be phenyl or pyridinyl, fused with R^{8A} wherein R^{8A} can be pyrrolidinyl, pyrrolidin-2-onyl, dioxanyl, tetrahydrofuranyl, tetrahydrothiophenyl, tetrahydropyranyl, oxazolidinyl, isothiazolidinyl, oxozolid-2-onyl, isothiazolid-2-onyl furanyl, pyrrolyl, thiophenyl, imidazolyl, pyrazolyl, triazolyl, oxazolyl, isoxazolyl, thiazolyl, isothiazolyl, oxadiazolyl or thiadiazolyl.

In the compounds of formula (I), (II), (III), (IA), (IIA) or (IIIA), R¹⁰ can be phenyl or pyridinyl, fused with R^{8A} wherein R^{8A} can be tetrahydrofuranyl, tetrahydrothiophenyl, tetrahydropyranyl, oxazolidinyl, isothiazolidinyl, oxozolid-2-onyl, isothiazolid-2-onyl, furanyl, pyrrolyl, thiophenyl, imidazolyl, pyrazolyl, triazolyl, oxazolyl, isoxazolyl, thiazolyl, isothiazolyl, oxadiazolyl or thiadiazolyl.

In the compounds of formula (I), (II), (III), (IA), (IIA) or (IIIA), R¹⁰ can be phenyl or pyridinyl, fused with R^{8A} wherein R^{8A} can be tetrahydrofuranyl, tetrahydrothiophenyl, tetrahydropyranyl, oxazolidinyl, isothiazolidinyl, oxozolid-2-only or isothiazolid-2-onyl.

In the compounds of formula (I), (II), (III), (IA), (IIA) or (IIIA), Z₆ can be C═C optionally substituted with R¹⁴.

In the compounds of formula (I), (II), (III), (IA), (IIA) or (IIIA), Z₆ can be C═C substituted with R¹⁴.

In the compounds of formula (I), (II), (III), (IA), (IIA) or (IIIA), Z₆ can be C═C substituted with R¹⁴, wherein R¹⁴ is F or CN.

In the compounds of formula (I), (II), (III), (IA), (IIA) or (IIIA), Z₆ can be C═C.

In the compounds of formula (I), (II), (III), (IA), (IIA) or (IIIA), R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (I), (II), (III), (IA), (IIA) or (IIIA), R¹³ can be hydrogen or C₁-C₃ alkyl, wherein C₁-C₃ alkyl is optionally substituted with one or more substituents independently selected from halo, OH and CN.

In the compounds of formula (I), (II), (III), (IA), (IIA) or (IIIA), Z₆ can be C≡C, and R¹³ can be hydrogen or C₁-C₅ alkyl, wherein C₁-C₅ alkyl is optionally substituted with one or more substituents independently selected from halo, OH and CN.

In the compounds of formula (I), (II), (III), (IA), (IIA) or (IIIA), Z₄ can be a bond.

In the compounds of formula (I), (II), (III), (IA), (IIA) or (IIIA), Z₆ can be C═C optionally substituted with R¹⁴, and Z₄ can be a bond.

In the compounds of formula (I), (II), (III), (IA), (IIA) or (IIIA), Z₆ can be C═C substituted with R¹⁴, and Z₄ can be a bond.

In the compounds of formula (I), (II), (III), (IA), (IIA) or (IIIA), Z₆ can be C═C substituted with R¹⁴, wherein R¹⁴ is F or CN, and Z₄ can be a bond.

In the compounds of formula (I), (II), (III), (IA), (IIA) or (IIIA), Z₆ can be C═C, and Z₄ can be a bond.

In the compounds of formula (I), (II), (III), (IA), (IIA) or (IIIA), Z₆ can be C═C optionally substituted with R¹⁴, Z₄ can be a bond, and R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (I), (II), (III), (IA), (IIA) or (IIIA), Z₆ can be C═C substituted with R¹⁴, Z₄ can be a bond, and R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (I), (II), (III), (IA), (IIA) or (IIIA), Z₆ can be C═C optionally substituted with R¹⁴, Z₄ can be a bond, and R¹³ can be hydrogen or C₁-C₃ alkyl, wherein C₁-C₃ alkyl is optionally substituted with one or more substituents independently selected from halo, OH and CN.

In the compounds of formula (I), (II), (III), (IA), (IIA) or (IIIA), Z₆ can be C═C substituted with R¹⁴, Z₄ can be a bond, and R¹³ can be hydrogen or C₁-C₃ alkyl, wherein C₁-C₃ alkyl is optionally substituted with one or more substituents independently selected from halo, OH and CN.

In the compounds of formula (I), (II), (III), (IA), (IIA) or (IIIA), Z₆ can be C═C substituted with R¹⁴, wherein R¹⁴ is F or CN, Z₄ can be a bond, and R¹³ can be hydrogen or C₁-C₃ alkyl, wherein C₁-C₃ alkyl is optionally substituted with one or more substituents independently selected from halo, OH and CN.

In the compounds of formula (I), (II), (III), (IA), (IIA) or (IIIA), Z₆ can be C═C, Z₄ can be a bond, and R¹³ can be hydrogen or C₁-C₃ alkyl, wherein C₁-C₃ alkyl is optionally substituted with one or more substituents independently selected from halo, OH and CN.

In the compounds of formula (I), (II), (III), (IA), (IIA) or (IIIA), Z₆ can be C≡C, R¹³ can be hydrogen or C₁-C₅ alkyl, wherein C₁-C₅ alkyl is optionally substituted with one or more substituents independently selected from halo, OH and CN, and Z₄ can be a bond.

In the compounds of formula (I) or (IA), where both Z and Z₁ are a bond, together they form a single bond.

In the compounds of formula (I) or (IA), Z can be CHR^{9A}, Z₁ can be CH₂, X₄ can be C—R⁹, and R⁹ can be fused with R^{9A} to form CH₂, forming:

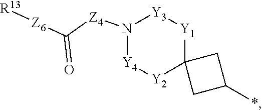

wherein * indicates the connection point to A.

In the compounds of formula (I), (II), (III), (IA) or (IIA), R⁵ can be fused with one R³ to form CH₂—CH₂, for example forming:

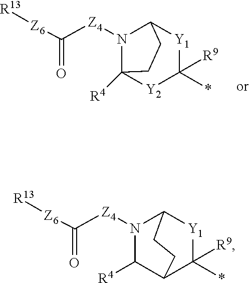

wherein * indicates the connection point to Z₁ in formula (I) or (IA); or Z′ in formula (II) or (IIA); or A in formula (III).

In the compounds of formula (I), (II), (III), (IA), (IIA) or (IIIA), R⁵ can be fused with one R³ to form CH₂—CH₂, for example forming:

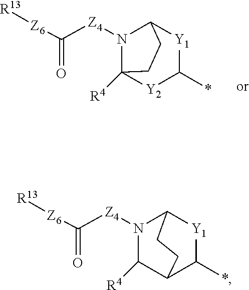

wherein * indicates the connection point to Z₁ in formula (I) or (IA); or Z′ in formula (II) or (IIA); or A in formula (III) or (IIIA).

In the compounds of formula (I), (II), (III), (IA), (IIA) or (IIIA), R⁸ can be cyclopentyl, fused with R^{8A}, wherein R^{8A} can be pyridinyl, for example forming:

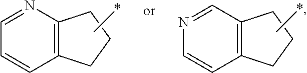

wherein * indicates the connection point to Y.

In the compounds of formula (I), (II), (III), (IA), (IIA) or (IIIA), A can be pyrazole, 1,2,3 triazole or 1,2,4 triazole, substituted with R¹ and R^{1A}; and Y can be NH or O.

In the compounds of formula (I), (II), (III), (IA), (IIA) or (IIIA), A can be pyrazole, 1,2,3 triazole or 1,2,4 triazole, substituted with R¹ and R^{1A}, wherein R^{1A} is hydrogen and R¹ is C₁-C₃ alkyl; and Y can be NH or O.

In the compounds of formula (I), (II), (III), (IA) or (IIA), A can be:

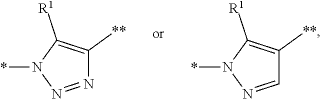

wherein * indicates the connection point to Z, Z′ or Z₂ and ** indicates the other connection point from A in formula (I), (II), (III), (IA) or (IIA); R¹ can be C₁-C₃ alkyl; and Y can be NH or O.

In the compounds of formula (IIIA), A can be:

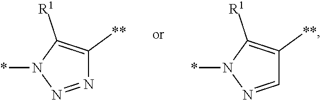

wherein * indicates the connection point to the substituent comprising Y₁ and ** indicates the other connection point from A in formula (IIIA); R¹ can be C₁-C₃ alkyl; and Y can be NH or O.

In the compounds of formula (I), (II), (III), (IA), (IIA) or (IIIA), A can be pyrazole, 1,2,3 triazole or 1,2,4 triazole, substituted with R¹ and R^{1A}; and Y can be O.

In the compounds of formula (I), (II), (III), (IA), (IIA) or (IIIA), A can be pyrazole, 1,2,3 triazole or 1,2,4 triazole, substituted with R¹ and R^{1A}, wherein R^{1A} is hydrogen and R¹ is C₁-C₃ alkyl; and Y can be O.

In the compounds of formula (I), (II), (III), (IA) or (IIA), A can be:

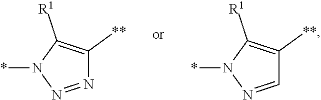

wherein * indicates the connection point to Z, Z′ or Z₂ and ** indicates the other connection point from A in formula (I), (II), (III), (IA) or (IIA); R¹ can be C₁-C₃ alkyl; and Y can be O.

In the compounds of formula (IIIA), A can be:

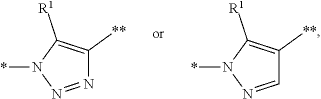

wherein * indicates the connection point to the substituent comprising Y₁ and ** indicates the other connection point from A in formula (IIIA); R¹ can be C₁-C₃ alkyl; and Y can be O.

In the compounds of formula (I), (II), (III), (IA), (IIA) or (IIIA), A can be pyrazole, 1,2,3 triazole or 1,2,4 triazole, substituted with R¹ and R^{1A}; and R⁶ can be CN, F, Cl, CH₃, CF₃ or cyclopropyl.

In the compounds of formula (I), (II), (III), (IA), (IIA) or (IIIA), A can be pyrazole, 1,2,3 triazole or 1,2,4 triazole, substituted with R¹ and R^{1A}, wherein R^{1A} is hydrogen and R¹ is C₁-C₃ alkyl; and R⁶ can be CN, F, Cl or CF₃.

In the compounds of formula (I), (II), (III), (IA) or (IIA), A can be:

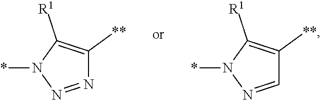

wherein * indicates the connection point to Z, Z′ or Z₂ and ** indicates the other connection point from A in formula (I), (II), (III), (IA) or (IIA); and R⁶ can be CN, F, Cl, CH₃, CF₃ or cyclopropyl.

In the compounds of formula (IIIA), A can be:

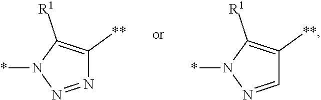

wherein * indicates the connection point to the substituent comprising Y₁ and ** indicates the other connection point from A in formula (IIIA) and R⁶ can be CN, F, Cl, CH₃, CF₃ or cyclopropyl.

In the compounds of formula (I), (II), (III), (IA) or (IIA), A can be:

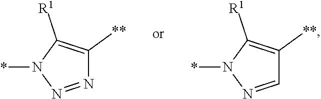

wherein * indicates the connection point to Z, Z′ or Z₂ and ** indicates the other connection point from A in formula (I), (II), (III), (IA) or (IIA); and R⁶ can be CN, F, Cl or CF₃.

In the compounds of formula (IIIA), A can be:

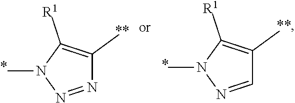

wherein * indicates the connection point to the substituent comprising Y₁ and ** indicates the other connection point from A in formula (IIIA) and R⁶ can be CN, F, Cl, CH₃ or CF₃.

In the compounds of formula (I), (II), (III), (IA) or (IIA), A can be:

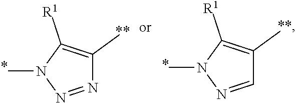

wherein * indicates the connection point to Z, Z′ or Z₂ and ** indicates the other connection point from A in formula (I), (II), (III), (IA) or (IIA); R¹ can be C₁-C₃ alkyl; and R⁶ can be CN or Cl.

In the compounds of formula (IIIA), A can be:

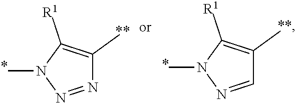

wherein * indicates the connection point to the substituent comprising Y₁ and ** indicates the other connection point from A in formula (IIIA); R¹ can be C₁-C₃ alkyl; and R⁶ can be CN or Cl.

In the compounds of formula (I), (II), (III), (IA), (IIA) or (IIIA), A can be pyrazole, 1,2,3 triazole or 1,2,4 triazole, substituted with R¹ and R^{1A}; R⁶ can be CN, F, Cl, CH₃, CF₃ or cyclopropyl; and Y can be NH or O.

In the compounds of formula (I), (II), (III), (IA), (IIA) or (IIIA), A can be pyrazole, 1,2,3 triazole or 1,2,4 triazole, substituted with R¹ and R^{1A}, wherein R^{1A} is hydrogen and R¹ is C₁-C₃ alkyl; R⁶ can be CN, F, Cl or CF₃; and Y can be NH or O.

In the compounds of formula (I), (II), (III), (IA) or (IIA), A can be:

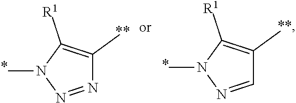

wherein * indicates the connection point to Z, Z′ or Z₂ and ** indicates the other connection point from A in formula (I), (II), (III), (IA) or (IIA); R⁶ can be CN, F, Cl, CH₃, CF₃ or cyclopropyl; and Y can be NH or O.

In the compounds of formula (IIIA), A can be:

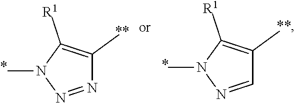

wherein * indicates the connection point to the substituent comprising Y₁ and ** indicates the other connection point from A in formula (IIIA); R⁶ can be CN, F, Cl, CH₃, CF₃ or cyclopropyl; and Y can be NH or O.

In the compounds of formula (I), (II), (III), (IA) or (IIA), A can be:

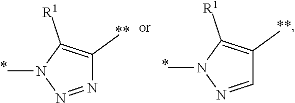

wherein * indicates the connection point to Z, Z′ or Z₂ and ** indicates the other connection point from A in formula (I), (II), (III), (IA) or (IIA); R⁶ can be CN, F, Cl or CF₃; and Y can be NH or O.

In the compounds of formula (IIIA), A can be:

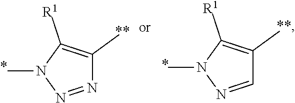

wherein * indicates the connection point to the substituent comprising Y₁ and ** indicates the other connection point from A in formula (IIIA); R⁶ can be CN, F, Cl or CF₃; and Y can be NH or O.

In the compounds of formula (I), (II), (III), (IA), (IIA) or (IIIA), A can be pyrazole, 1,2,3 triazole or 1,2,4 triazole, substituted with R¹ and R^{1A}; R⁶ can be CN, F, Cl, CH₃, CF₃ or cyclopropyl; and Y can be O.

In the compounds of formula (I), (II), (III), (IA), (IIA) or (IIIA), A can be pyrazole, 1,2,3 triazole or 1,2,4 triazole, substituted with R¹ and R^{1A}, wherein R^{1A} is hydrogen and R¹ is C₁-C₃ alkyl; R⁶ can be CN, F, Cl or CF₃; and Y can be O.

In the compounds of formula (I), (II), (III), (IA) or (IIA), A can be:

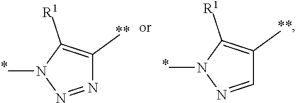

wherein * indicates the connection point to Z, Z′ or Z₂ and ** indicates the other connection point from A in formula (I), (II), (III), (IA) or (IIA); R⁶ can be CN, F, Cl, CH₃, CF₃ or cyclopropyl; and Y can be O.

In the compounds of formula (IIIA), A can be:

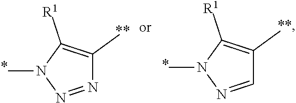

wherein * indicates the connection point to the substituent comprising Y₁ and ** indicates the other connection point from A in formula (IIIA); R⁶ can be CN, F, Cl, CH₃, CF₃ or cyclopropyl; and Y can be O.

In the compounds of formula (I), (II), (III), (IA) or (IIA), A can be:

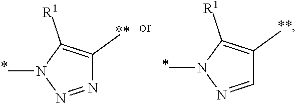

wherein * indicates the connection point to Z, Z′ or Z₂ and ** indicates the other connection point from A in formula (I), (II), (III), (IA) or (IIA); R⁶ can be CN, F, Cl or CF₃; and Y can be O.

In the compounds of formula (IIIA), A can be:

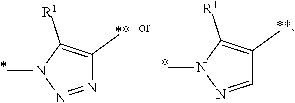

wherein * indicates the connection point to the substituent comprising Y₁ and ** indicates the other connection point from A in formula (IIIA); R⁶ can be CN, F, Cl or CF₃; and Y can be O.

In the compounds of formula (I), (II), (III), (IA) or (IIA), A can be:

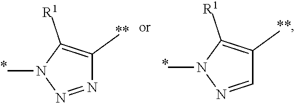

wherein * indicates the connection point to Z, Z′ or Z₂ and ** indicates the other connection point from A in formula (I), (II), (III), (IA) or (IIA); R¹ can be C₁-C₃ alkyl; R⁶ can be CN or Cl; and Y can be O.

In the compounds of formula (IIIA), A can be:

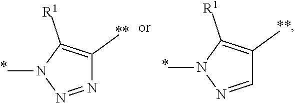

wherein * indicates the connection point to the substituent comprising Y₁ and ** indicates the other connection point from A in formula (IIIA); R⁶ can be CN, F, Cl or CF₃; and Y can be O.

In the compounds of formula (I), (II), (III), (IA), (IIA) or (IIIA), A can be pyrazole, 1,2,3 triazole or 1,2,4 triazole, substituted with R¹ and R^{1A}; R⁶ can be CN, F, Cl, CH₃, CF₃ or cyclopropyl; Y can be NH or O; and R² can be C₁-C₃ alkyl optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN.

In the compounds of formula (I), (II), (III), (IA), (IIA) or (IIIA), A can be pyrazole, 1,2,3 triazole or 1,2,4 triazole, substituted with R¹ and R^{1A}; R⁶ can be CN, F, Cl, CH₃, CF₃ or cyclopropyl; Y can be NH or O; and R² can be C₁-C₄ alkyl optionally substituted with one or more substituents independently selected from F, OH, CN, oxo, —OCH₃, —OC₃ cycloalkyl and R¹⁰.

In the compounds of formula (I), (II), (III), (IA), (IIA) or (IIIA), A can be pyrazole, 1,2,3 triazole or 1,2,4 triazole, substituted with R¹ and R^{1A}; R⁶ can be CN, F, Cl or CF₃; Y can be NH or O; and R² can be:

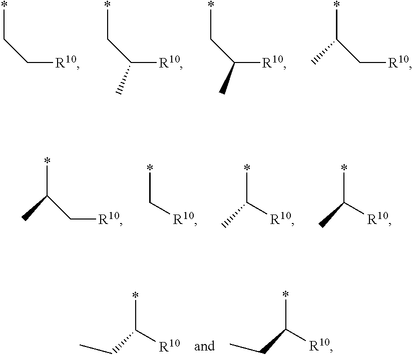

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I), (II), (III), (IA), (IIA) or (IIIA).

In the compounds of formula (I), (II), (III), (IA), (IIA) or (IIIA), A can be pyrazole, 1,2,3 triazole or 1,2,4 triazole, substituted with R¹ and R^{1A}; R⁶ can CN, F, Cl or CF₃; Y can be NH or O; and R² can be:

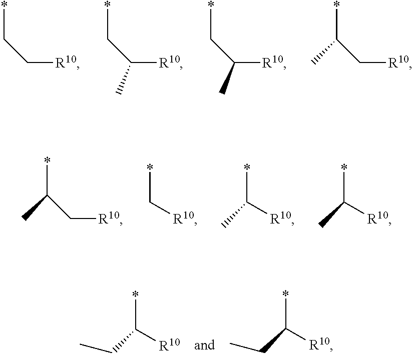

optionally substituted with one, two, three or four substituents independently selected from F, OH, CN, oxo, —OCH₃, —OC₃ cycloalkyl and R¹⁰, wherein * indicates the connection point to Y in formula (I), (II), (III), (IA), (IIA) or (IIIA).

In the compounds of formula (I), (II), (III), (IA), (IIA) or (IIIA), A can be pyrazole, 1,2,3 triazole or 1,2,4 triazole, substituted with R¹ and R^{1A}; R⁶ can be CN, F, Cl or CF₃; Y can be NH or O; and R² can be:

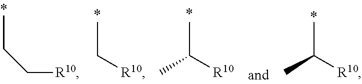

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I), (II), (III), (IA), (IIA) or (IIIA).

In the compounds of formula (I), A can be pyrazole, 1,2,3 triazole or 1,2,4 triazole, substituted with R¹ and R^{1A}; R⁶ can CN, F, Cl or CF₃; Y can be NH or O; and R² can be:

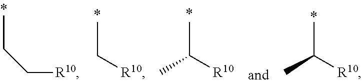

optionally substituted with one, two, three or four substituents independently selected from F, OH, CN, oxo, —OCH₃, —OC₃ cycloalkyl and R¹⁰, wherein * indicates the connection point to Y in formula (I), (II), (III), (IA), (IIA) or (IIIA).

In the compounds of formula (I), (II), (III), (IA) or (IIA), A can be:

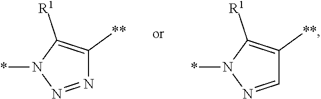

wherein * indicates the connection point to Z, Z′ or Z₂ and ** indicates the other connection point from A in formula (I), (II), (III), (IA) or (IIA); R⁶ can be CN, F, Cl, CH₃, CF₃ or cyclopropyl; Y can be NH or O; and R² can be C₁-C₃ alkyl optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN.

In the compounds of formula (I), (II), (III), (IA) or (IIA), A can be:

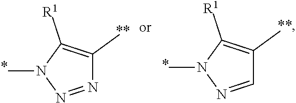

wherein * indicates the connection point to Z, Z′ or Z₂ and ** indicates the other connection point from A in formula (I), (II), (III), (IA) or (IIA); R⁶ can be CN, F, Cl, CH₃, CF₃ or cyclopropyl; Y can be NH or O; and R² can be C₁-C₄ alkyl optionally substituted with one or more substituents independently selected from F, OH, CN, oxo, —OCH₃, —OC₃ cycloalkyl and R¹⁰.

In the compounds of formula (I), (II), (III), (IA) or (IIA), A can be:

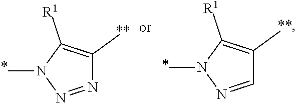

wherein * indicates the connection point to Z, Z′ or Z₂ and ** indicates the other connection point from A in formula (I), (II), (III), (IA) or (IIA); R⁶ can be CN, F, Cl or CF₃; Y can be NH or O; and R² can be:

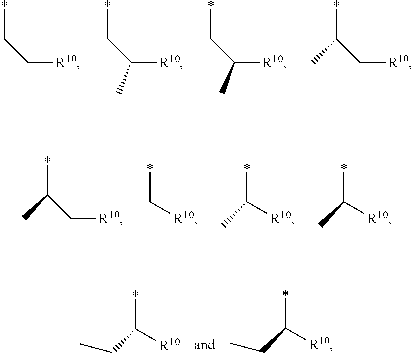

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I), (II), (III), (IA) or (IIA).

In the compounds of formula (I), (II), (III), (IA) or (IIA), A can be:

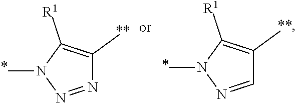

wherein * indicates the connection point to Z, Z′ or Z₂ and ** indicates the other connection point from A in formula (I), (II), (III), (IA) or (IIA); R⁶ can CN, F, Cl or CF₃; Y can be NH or O; and R² can be:

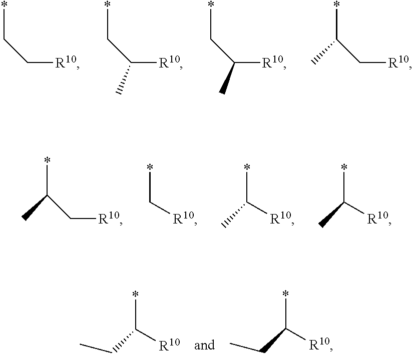

optionally substituted with one, two, three or four substituents independently selected from F, OH, CN, oxo, —OCH₃, —OC₃ cycloalkyl and R¹⁰, wherein * indicates the connection point to Y in formula (I), (II), (III), (IA) or (IIA).

In the compounds of formula (I), (II), (III), (IA) or (IIA), A can be:

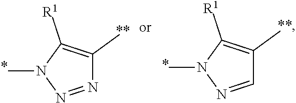

wherein * indicates the connection point to Z, Z′ or Z₂ and ** indicates the other connection point from A in formula (I), (II), (III), (IA) or (IIA); R⁶ can be CN, F, Cl or CF₃; Y can be NH or O; and R² can be:

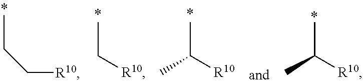

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I), (II), (III), (IA) or (IIA).

In the compounds of formula (I), (II), (III), (IA) or (IIA), A can be:

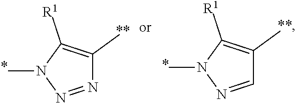

wherein * indicates the connection point to Z, Z′ or Z₂ and ** indicates the other connection point from A in formula (I), (II), (III), (IA) or (IIA); R⁶ can CN, F, Cl or CF₃; Y can be NH or O; and R² can be:

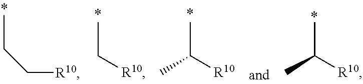

optionally substituted with one, two, three or four substituents independently selected from F, OH, CN, oxo, —OCH₃, —OC₃ cycloalkyl and R¹⁰, wherein * indicates the connection point to Y in formula (I), (II), (III), (IA) or (IIA).

In the compounds of formula (IIIA), A can be:

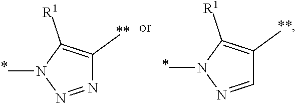

wherein * indicates the connection point to the substituent comprising Y₁ and ** indicates the other connection point from A in formula (IIIA); R⁶ can CN, F, Cl or CF₃; Y can be NH or O; and R² can be:

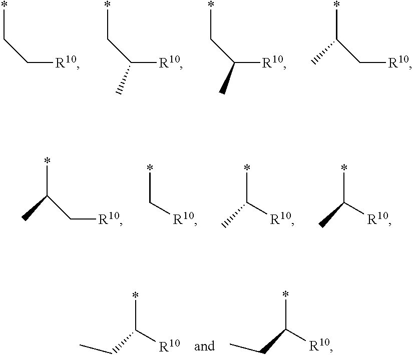

optionally substituted with one, two, three or four substituents independently selected from F, OH, CN, oxo, —OCH₃, —OC₃ cycloalkyl and R¹⁰, wherein * indicates the connection point to Y in formula (IIIA).

In the compounds of formula (IIIA), A can be:

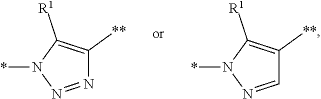

wherein * indicates the connection point to the substituent comprising Y₁ and ** indicates the other connection point from A in formula (IIIA); R⁶ can be CN, F, Cl or CF₃; Y can be NH or O; and R² can be:

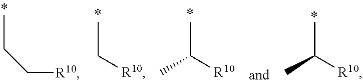

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (IIIA).

In the compounds of formula (IIIA), A can be:

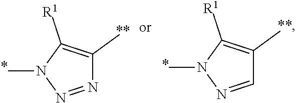

wherein * indicates the connection point to the substituent comprising Y₁ and ** indicates the other connection point from A in formula (IIIA); R⁶ can CN, F, Cl or CF₃; Y can be NH or O; and R² can be:

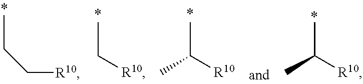

optionally substituted with one, two, three or four substituents independently selected from F, OH, CN, oxo, —OCH₃, —OC₃ cycloalkyl and R¹⁰, wherein * indicates the connection point to Y in formula (IIIA).

In the compounds of formula (I), (II), (III), (IA) or (IIA), A can be:

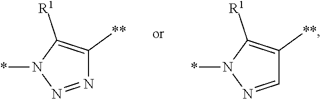

wherein * indicates the connection point to Z, Z′ or Z₂ and ** indicates the other connection point from A in formula (I), (II), (III), (IA) or (IIA); R⁶ can be CN, F, Cl, CH₃, CF₃ or cyclopropyl; Y can be NH or O; and R² can be C₁-C₃ alkyl optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN; Z₆ can be C═C optionally substituted with R¹⁴, Z₄ can be a bond, and R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (I), (II), (III), (IA) or (IIA), A can be:

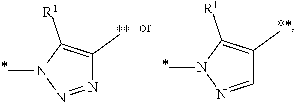

wherein * indicates the connection point to Z, Z′ or Z₂ and ** indicates the other connection point from A in formula (I), (II), (III), (IA) or (IIA); R⁶ can be CN, F, Cl, CH₃, CF₃ or cyclopropyl; Y can be NH or O; and R² can be C₁-C₄ alkyl optionally substituted with one or more substituents independently selected from F, OH, CN, oxo, —OCH₃, —OC₃ cycloalkyl and R¹⁰; Z₆ can be C═C optionally substituted with R¹⁴, Z₄ can be a bond, and R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (I), (II), (III), (IA) or (IIA), A can be:

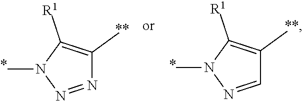

wherein * indicates the connection point to Z, Z′ or Z₂ and ** indicates the other connection point from A in formula (I), (II), (III), (IA) or (IIA); R⁶ can be CN, F, Cl or CF₃; Y can be NH or O; and R² can be:

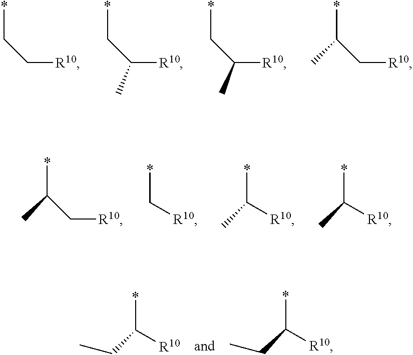

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I), (II), (III), (IA) or (IIA); Z₆ can be C═C optionally substituted with R¹⁴, Z₄ can be a bond, and R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (I), (II), (III), (IA) or (IIA), A can be:

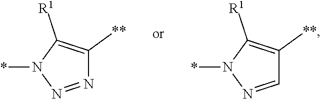

wherein * indicates the connection point to Z, Z′ or Z₂ and ** indicates the other connection point from A in formula (I), (II), (III), (IA) or (IIA); R⁶ can CN, F, Cl or CF₃; Y can be NH or O; and R² can be:

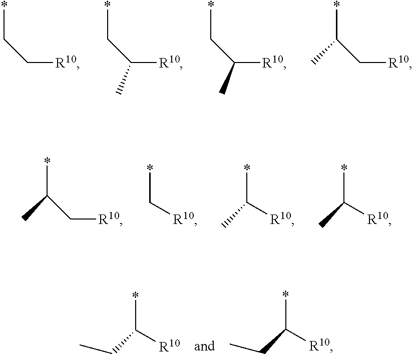

optionally substituted with one, two, three or four substituents independently selected from F, OH, CN, oxo, —OCH₃, —OC₃ cycloalkyl and R¹⁰, wherein * indicates the connection point to Y in formula (I), (II), (III), (IA) or (IIA); Z₆ can be C═C optionally substituted with R¹⁴, Z₄ can be a bond, and R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (I), (II), (III), (IA) or (IIA), A can be:

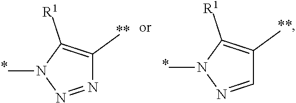

wherein * indicates the connection point to Z, Z′ or Z₂ and ** indicates the other connection point from A in formula (I), (II), (III), (IA) or (IIA); R⁶ can be CN, F, Cl or CF₃; Y can be NH or O; and R² can be:

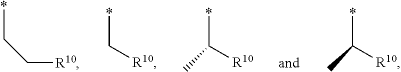

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I), (II), (III), (IA) or (IIA); Z₆ can be C═C optionally substituted with R¹⁴, Z₄ can be a bond, and R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (I), (II), (III), (IA) or (IIA), A can be:

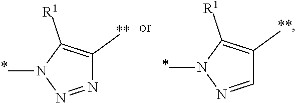

wherein * indicates the connection point to Z, Z′ or Z₂ and ** indicates the other connection point from A in formula (I), (II), (III), (IA) or (IIA); R⁶ can CN, F, Cl or CF₃; Y can be NH or O; and R² can be:

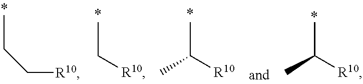

optionally substituted with one, two, three or four substituents independently selected from F, OH, CN, oxo, —OCH₃, —OC₃ cycloalkyl and R¹⁰, wherein * indicates the connection point to Y in formula (I), (II), (III), (IA) or (IIA); Z₆ can be C═C optionally substituted with R¹⁴, Z₄ can be a bond, and R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (IIIA), A can be:

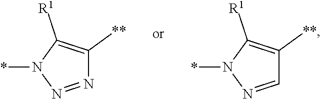

wherein * indicates the connection point to the substituent comprising Y₁ and ** indicates the other connection point from A in formula (IIIA); R⁶ can CN, F, Cl or CF₃; Y can be NH or O; and R² can be:

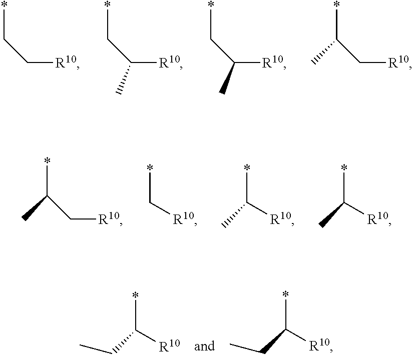

optionally substituted with one, two, three or four substituents independently selected from F, OH, CN, oxo, —OCH₃, —OC₃ cycloalkyl and R¹⁰, wherein * indicates the connection point to Y in formula (IIIA); Z₆ can be C═C optionally substituted with R¹⁴, Z₄ can be a bond, and R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (IIIA), A can be:

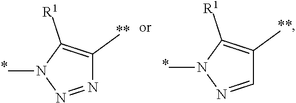

wherein * indicates the connection point to the substituent comprising Y₁ and ** indicates the other connection point from A in formula (IIIA); R⁶ can be CN, F, Cl or CF₃; Y can be NH or O; and R² can be:

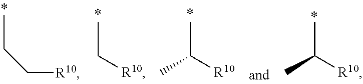

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (IIIA); Z₆ can be C═C optionally substituted with R¹⁴, Z₄ can be a bond, and R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (IIIA), A can be:

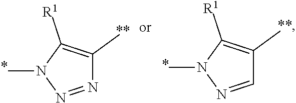

wherein * indicates the connection point to the substituent comprising Y₁ and ** indicates the other connection point from A in formula (IIIA); R⁶ can CN, F, Cl or CF₃; Y can be NH or O; and R² can be:

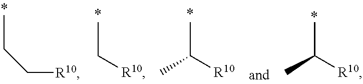

optionally substituted with one, two, three or four substituents independently selected from F, OH, CN, oxo, —OCH₃, —OC₃ cycloalkyl and R¹⁰, wherein * indicates the connection point to Y in formula (IIIA); Z₆ can be C═C optionally substituted with R¹⁴, Z₄ can be a bond, and R¹³ can be hydrogen, C₁-C₅ alkyl or R¹⁷, wherein C₁-C₅ alkyl is optionally substituted with one or more substituents independently selected from halo, OH, CN and R¹⁷.

In the compounds of formula (I), (II), (III), (IA) or (IIA), A can be:

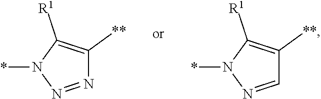

wherein * indicates the connection point to Z, Z′ or Z₂ and ** indicates the other connection point from A in formula (I), (II), (III), (IA) or (IIA); R⁶ can be CN, F, Cl, CH₃, CF₃ or cyclopropyl; Y can be NH or O; and R² can be C₁-C₃ alkyl optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN; Z₆ can be C≡C, and R¹³ can be hydrogen or C₁-C₅ alkyl, wherein C₁-C₅ alkyl is optionally substituted with one or more substituents independently selected from halo, OH and CN.

In the compounds of formula (I), (II), (III), (IA) or (IIA), A can be:

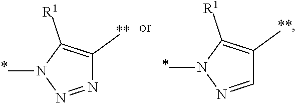

wherein * indicates the connection point to Z, Z′ or Z₂ and ** indicates the other connection point from A in formula (I), (II), (III), (IA) or (IIA); R⁶ can be CN, F, Cl, CH₃, CF₃ or cyclopropyl; Y can be NH or O; and R² can be C₁-C₄ alkyl optionally substituted with one or more substituents independently selected from F, OH, CN, oxo, —OCH₃, —OC₃ cycloalkyl and R¹⁰; Z₆ can be C≡C, and R¹³ can be hydrogen or C₁-C₅ alkyl, wherein C₁-C₅ alkyl is optionally substituted with one or more substituents independently selected from halo, OH and CN.

In the compounds of formula (I), (II), (III), (IA) or (IIA), A can be:

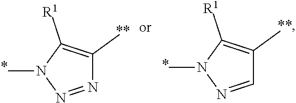

wherein * indicates the connection point to Z, Z′ or Z₂ and ** indicates the other connection point from A in formula (I), (II), (III), (IA) or (IIA); R⁶ can be CN, F, Cl or CF₃; Y can be NH or O; and R² can be:

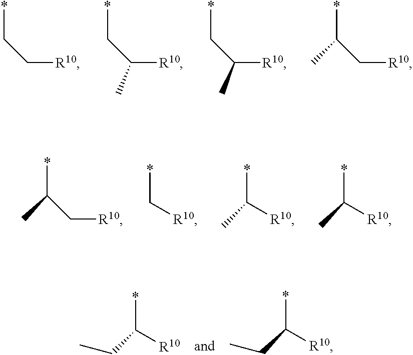

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I), (II), (III), (IA) or (IIA); Z₆ can be C≡C, and R¹³ can be hydrogen or C₁-C₅ alkyl, wherein C₁-C₅ alkyl is optionally substituted with one or more substituents independently selected from halo, OH and CN.

In the compounds of formula (I), (II), (III), (IA) or (IIA), A can be:

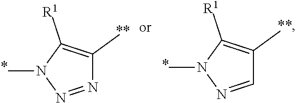

wherein * indicates the connection point to Z, Z′ or Z₂ and ** indicates the other connection point from A in formula (I), (II), (III), (IA) or (IIA); R⁶ can CN, F, Cl or CF₃; Y can be NH or O; and R² can be:

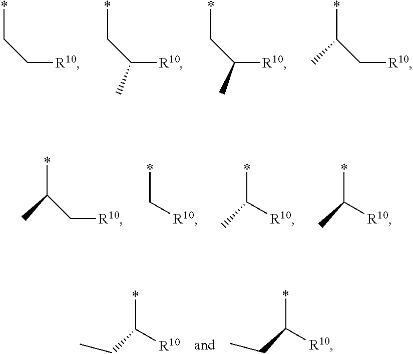

optionally substituted with one, two, three or four substituents independently selected from F, OH, CN, oxo, —OCH₃, —OC₃ cycloalkyl and R¹⁰, wherein * indicates the connection point to Y in formula (I), (II), (III), (IA) or (IIA); Z₆ can be C≡C, and R¹³ can be hydrogen or C₁-C₅ alkyl, wherein C₁-C₅ alkyl is optionally substituted with one or more substituents independently selected from halo, OH and CN.

In the compounds of formula (I), (II), (III), (IA) or (IIA), A can be:

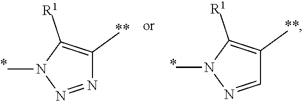

wherein * indicates the connection point to Z, Z′ or Z₂ and ** indicates the other connection point from A in formula (I), (II), (III), (IA) or (IIA); R⁶ can be CN, F, Cl or CF₃; Y can be NH or O; and R² can be:

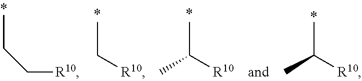

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I), (II), (III), (IA) or (IIA); Z₆ can be C≡C, and R¹³ can be hydrogen or C₁-C₅ alkyl, wherein C₁-C₅ alkyl is optionally substituted with one or more substituents independently selected from halo, OH and CN.

In the compounds of formula (I), (II), (III), (IA) or (IIA), A can be:

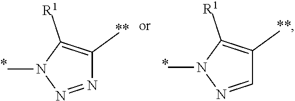

wherein * indicates the connection point to Z, Z′ or Z₂ and ** indicates the other connection point from A in formula (I), (II), (III), (IA) or (IIA); R⁶ can CN, F, Cl or CF₃; Y can be NH or O; and R² can be:

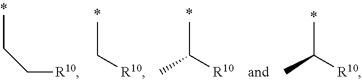

optionally substituted with one, two, three or four substituents independently selected from F, OH, CN, oxo, —OCH₃, —OC₃ cycloalkyl and R¹⁰, wherein * indicates the connection point to Y in formula (I), (II), (III), (IA) or (IIA); Z₆ can be C≡C, and R¹³ can be hydrogen or C₁-C₅ alkyl, wherein C₁-C₅ alkyl is optionally substituted with one or more substituents independently selected from halo, OH and CN.

In the compounds of formula (IIIA), A can be:

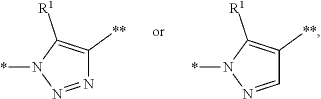

wherein * indicates the connection point to the substituent comprising Y₁ and ** indicates the other connection point from A in formula (IIIA); R⁶ can CN, F, Cl or CF₃; Y can be NH or O; and R² can be:

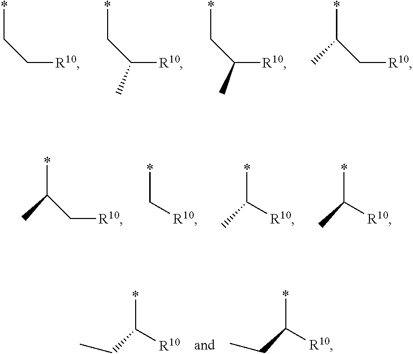

optionally substituted with one, two, three or four substituents independently selected from F, OH, CN, oxo, —OCH₃, —OC₃ cycloalkyl and R¹⁰, wherein * indicates the connection point to Y in formula (IIIA); Z₆ can be C≡C, and R¹³ can be hydrogen or C₁-C₅ alkyl, wherein C₁-C₅ alkyl is optionally substituted with one or more substituents independently selected from halo, OH and CN.

In the compounds of formula (IIIA), A can be:

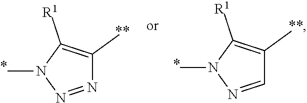

wherein * indicates the connection point to the substituent comprising Y₁ and ** indicates the other connection point from A in formula (IIIA); R⁶ can be CN, F, Cl or CF₃; Y can be NH or O; and R² can be:

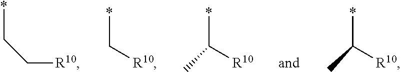

optionally substituted with one, two, three or four substituents independently selected from halo, OH, CN, oxo, —OC₁-C₄ alkyl, —OC₃-C₅ cycloalkyl, —Z₃—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (IIIA); Z₆ can be C≡C, and R¹³ can be hydrogen or C₁-C₅ alkyl, wherein C₁-C₅ alkyl is optionally substituted with one or more substituents independently selected from halo, OH and CN.

In the compounds of formula (IIIA), A can be:

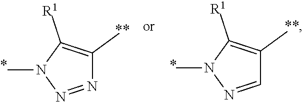

wherein * indicates the connection point to the substituent comprising Y₁ and ** indicates the other connection point from A in formula (IIIA); R⁶ can CN, F, Cl or CF₃; Y can be NH or O; and R² can be:

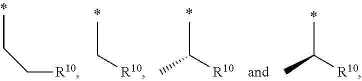

optionally substituted with one, two, three or four substituents independently selected from F, OH, CN, oxo, —OCH₃, —OC₃ cycloalkyl and R¹⁰, wherein * indicates the connection point to Y in formula (IIIA); Z₆ can be C≡C, and R¹³ can be hydrogen or C₁-C₅ alkyl, wherein C₁-C₅ alkyl is optionally substituted with one or more substituents independently selected from halo, OH and CN.

In one embodiment, the compounds of Formula (I) are selected from the group consisting of:

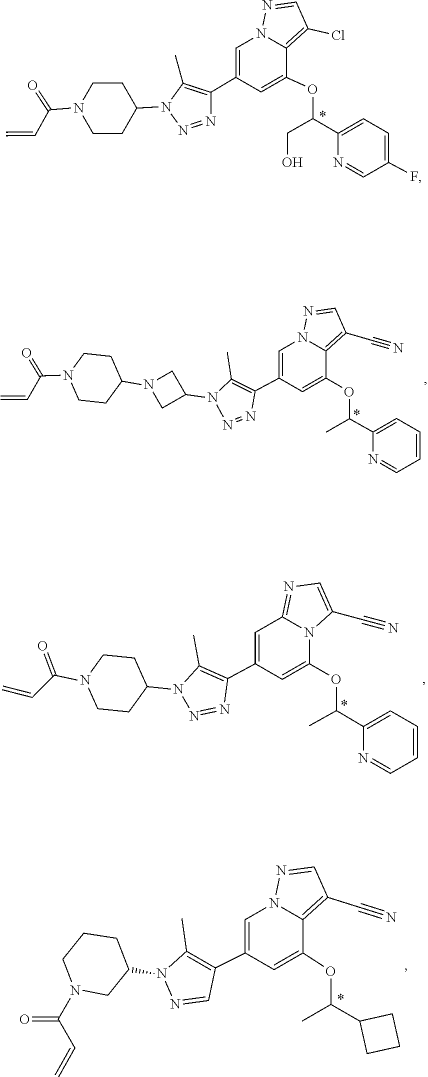

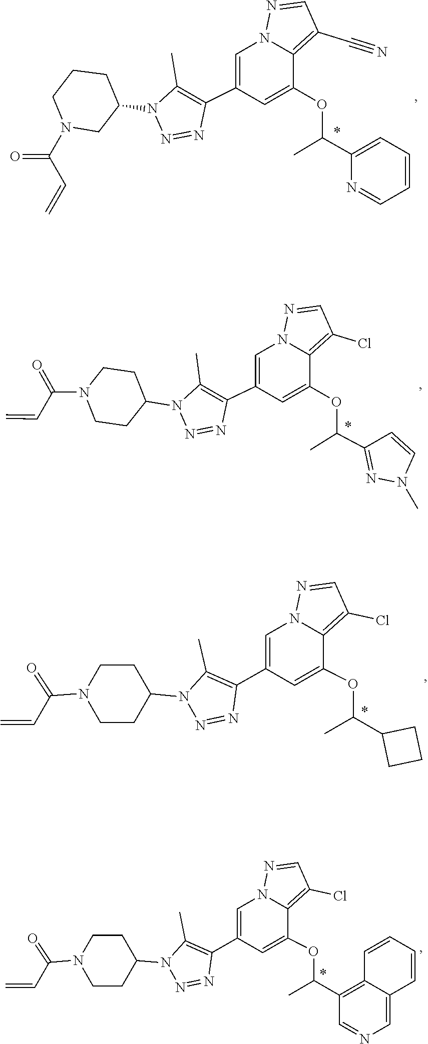

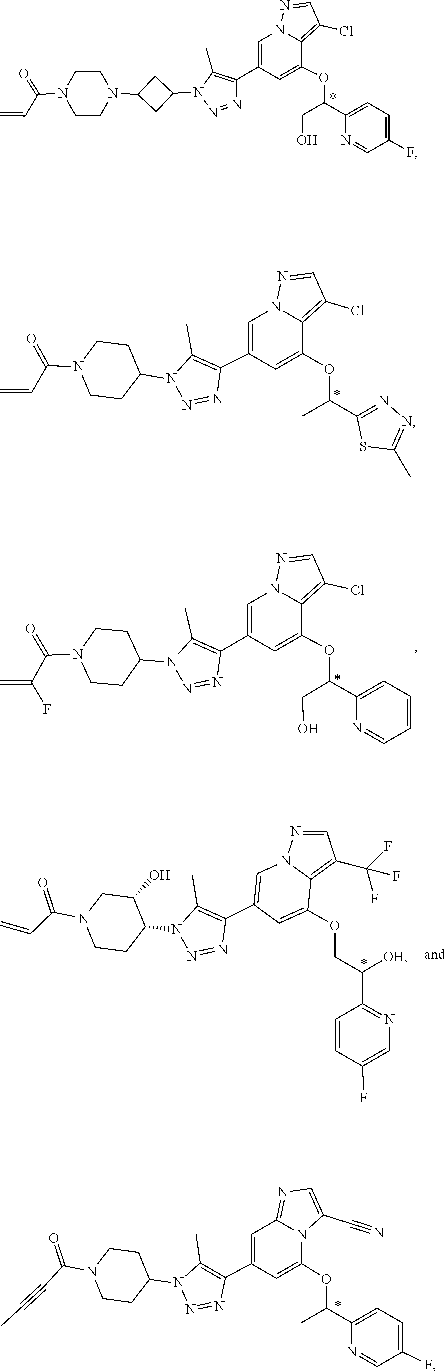

or a pharmaceutically acceptable salt thereof,
wherein the bond at the * position is as represented,

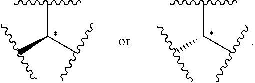

For example, for the compound of formula:

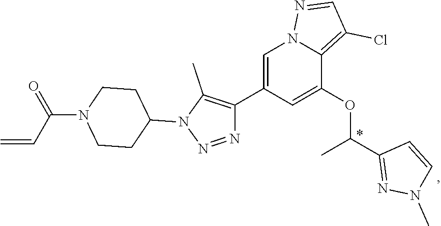 [SMILES: C=CC(=O)N1CCC(n2nnc(-c3cc(OC(C)c4ccn(C)n4)c4c(Cl)cnn4c3)c2C)CC1]

where the bond at the * position is as represented,

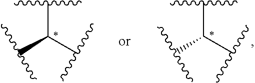

forms the compounds:

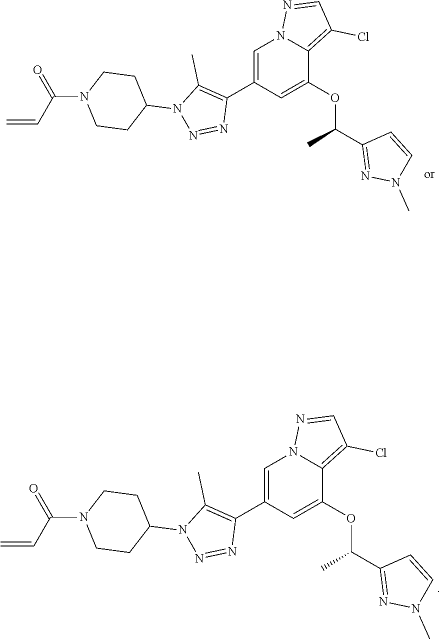 [SMILES: C=CC(=O)N1CCC(n2nnc(-c3cc(O[C@H](C)c4ccn(C)n4)c4c(Cl)cnn4c3)c2C)CC1 ; C=CC(=O)N1CCC(n2nnc(-c3cc(O[C@@H](C)c4ccn(C)n4)c4c(Cl)cnn4c3)c2C)CC1]

In a further embodiment, the compounds of Formula (I) are selected from the group consisting of:

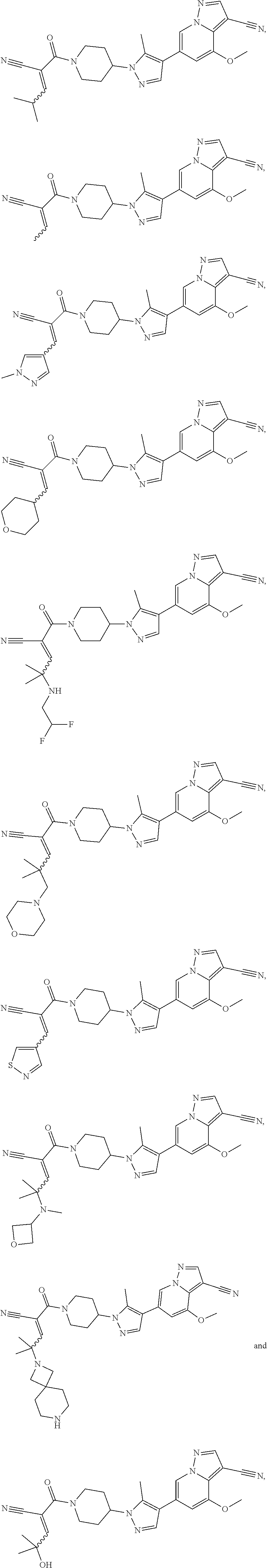

or a pharmaceutically acceptable salt thereof, where the bond is represented as [symbol] , indicates the E or Z isomer.

For example, for the compound of formula:

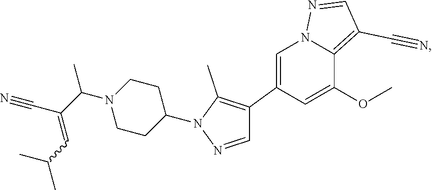 [SMILES: COc1cc(-c2cnn(C3CCN(C(C)C(C#N)=CC(C)C)CC3)c2C)cn2ncc(C#N)c12]

where the bond is represented as [symbol] , forms the compounds:

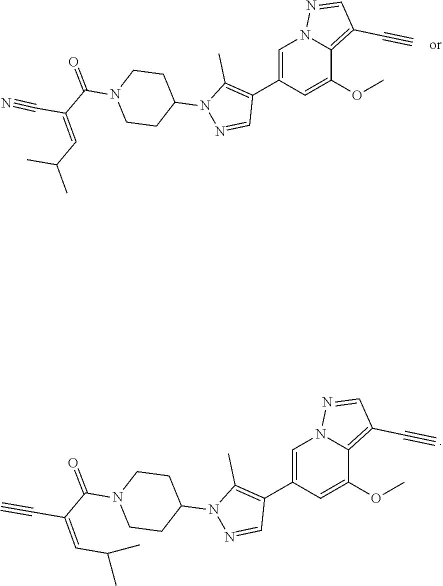

In a further embodiment, the compounds of Formula (I) are selected from the group consisting of:

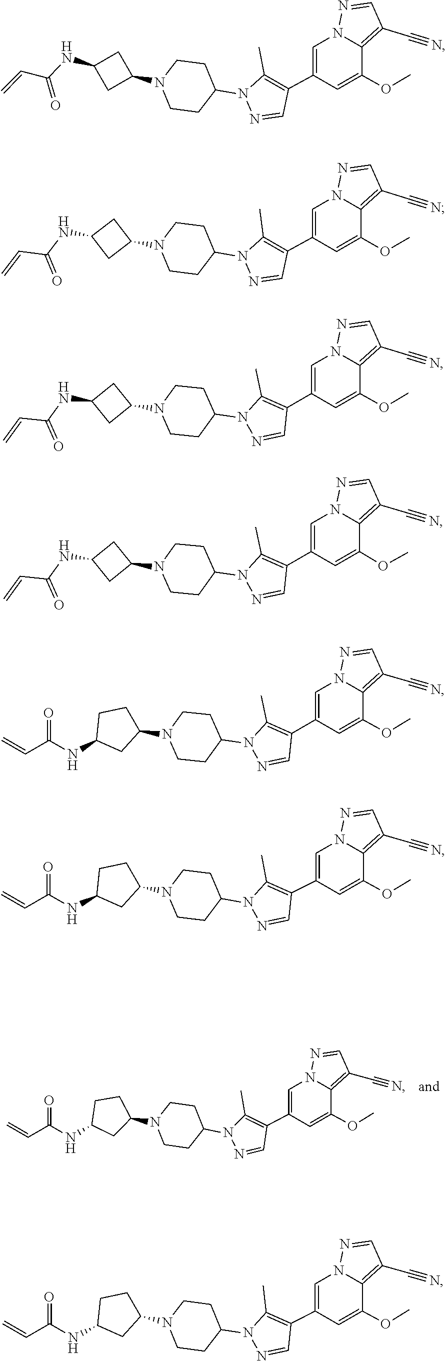

or a pharmaceutically acceptable salt thereof.

In a further embodiment, the compounds of Formula (I) are selected from the group consisting of:

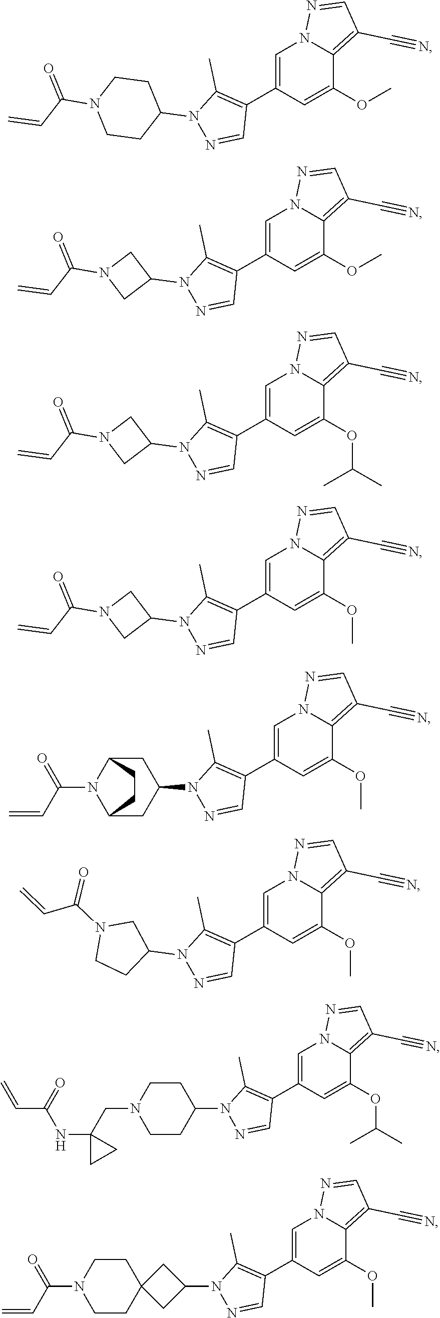

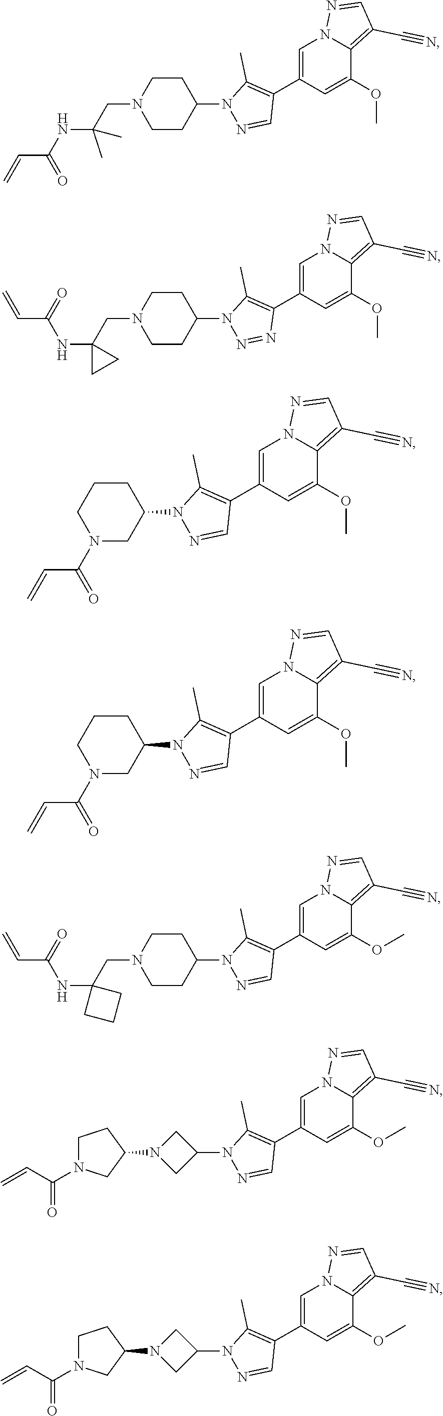

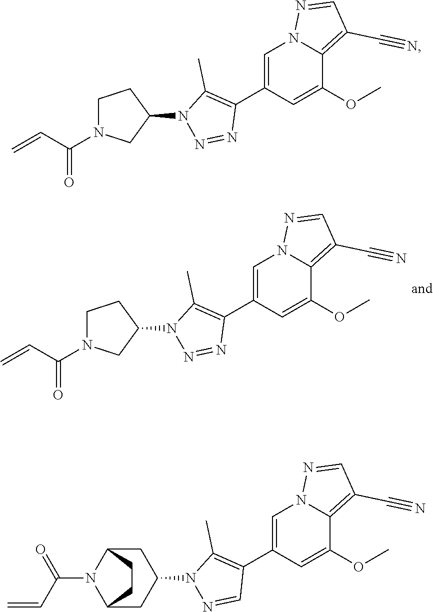 [SMILES: C=CC(=O)N1CC[C@@H](n2nnc(-c3cc(OC)c4c(C#N)cnn4c3)c2C)C1 ; C=CC(=O)N1CC[C@H](n2nnc(-c3cc(OC)c4c(C#N)cnn4c3)c2C)C1 ; C=CC(=O)N1[C@@H]2CC[C@H]1C[C@H](n1ncc(-c3cc(OC)c4c(C#N)cnn4c3)c1C)C2]

or a pharmaceutically acceptable salt thereof.

In a further embodiment, the compounds of Formula (I) are selected from the group consisting of:

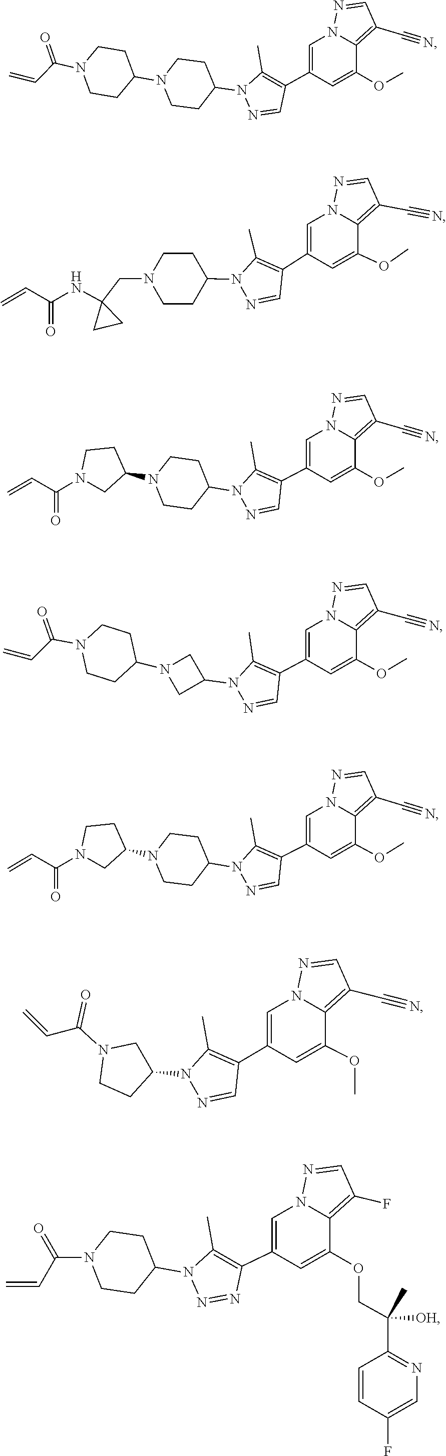

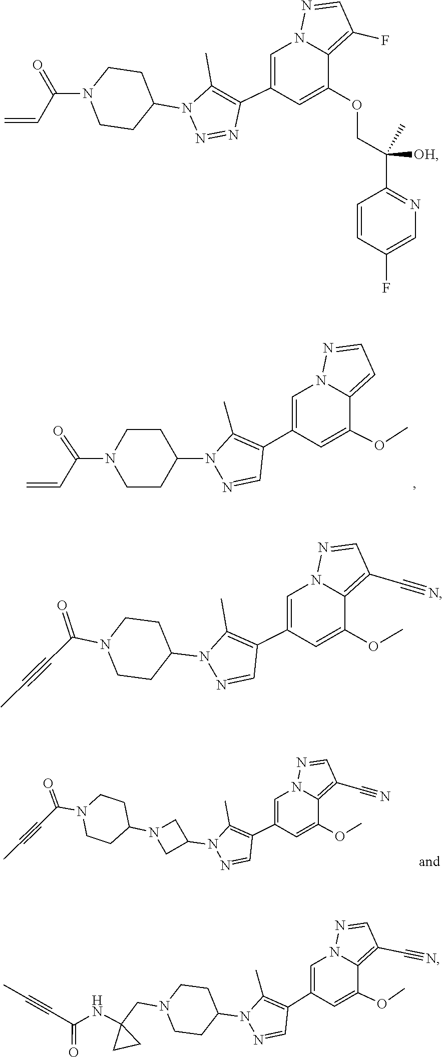

or a pharmaceutically acceptable salt thereof.

The compounds of formula (I), (II), (III), (IA), (IIA) or (IIIA), provided herein, or a pharmaceutically acceptable salt thereof, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, ethyl refers to both C₂H₅ or C₂H₅ where from 1 to 5 hydrogens are replaced by deuterium, such as in C₂DₓH₅₋ₓ.

The compounds of formula (I), (II), (III), (IA), (IIA) or (IIIA) provided herein may form pharmaceutically acceptable salts. The Examples provided herein may form pharmaceutically acceptable salts. Such pharmaceutically acceptable salts are intended to be included. Pharmaceutically acceptable salts and common methodology for preparing them are well known in the art (see, e.g., P. Stahl, et al. Handbook of Pharmaceutical Salts: Properties, Selection and Use, 2^{nd} Revised Edition (Wiley-VCH, 2011); S. M. Berge, et al., “Pharmaceutical Salts,” Journal of Pharmaceutical Sciences, Vol. 66, No. 1, January 1977).

The compounds of formula (I), (II), (III), (IA), (IIA) or (IIIA) provided herein, or a pharmaceutically acceptable salt thereof, can be mixed with one or more pharmaceutically acceptable carriers, diluents, or excipients. More particularly, the compounds of formula (I), (II), (III), (IA), (IIA) or (IIIA) provided herein, or a pharmaceutically acceptable salt thereof, can be formulated as pharmaceutical compositions. Such pharmaceutical compositions and processes for preparing the same are well known in the art (see, e.g., Remington: The Science and Practice of Pharmacy (A. Gennaro, et al., eds., 21st ed., Mack Publishing Co., 2005)).

The compounds of formula (I), (II), (III), (IA), (IIA) or (IIIA) provided herein, or a pharmaceutically acceptable salt thereof, and their pharmaceutical compositions can be administered by a variety of routes. Such routes of administration include oral and intravenous.

The compounds of formula (I), (II), (III), (IA), (IIA) or (IIIA) provided herein, or a pharmaceutically acceptable salt thereof, can be combined with one or more other therapeutic agents.

The compounds of formula (I), (II), (III), (IA), (IIA) or (IIIA) provided herein, or a pharmaceutically acceptable salt thereof, can be a component in a pharmaceutical composition for the treatment of cancer with one or more pharmaceutically acceptable carriers, diluents, or excipients, and optionally with one or more additional therapeutic agents.

The compounds of formula (I), (II), (III), (IA), (IIA) or (IIIA) provided herein, or a pharmaceutically acceptable salt thereof, can be a component in a pharmaceutical composition for the treatment of cancer with one or more pharmaceutically acceptable carriers, diluents, or excipients, and optionally with one or more additional therapeutic agents.

The compounds of formula (I), (II), (III), (IA), (IIA) or (IIIA) provided herein, or a pharmaceutically acceptable salt thereof, can be combined with one or more other therapeutic agents for simultaneous, separate or sequential administration.

The compounds of formula (I), (II), (III), (IA), (IIA) or (IIIA) provided herein, or a pharmaceutically acceptable salt thereof, and their pharmaceutical compositions can be used in the methods described herein.

The compounds of formula (I), (II), (III), (IA), (IIA) or (IIIA) provided herein, or a pharmaceutically acceptable salt thereof, are generally effective over a wide dosage range. For example, dosages per day normally fall within the range of about 0.5 to about 100 mg/kg of body weight. In some instances, dosage levels below the lower limit of the aforesaid range may be more than adequate, while in other cases still larger doses may be employed without causing any harmful side effect, and therefore the above dosage range is not intended to limit the scope of the invention in any way. It will be understood that the amount of the compound actually administered will be determined by a physician, in the light of the relevant circumstances, including the condition to be treated, the chosen route of administration, the actual compound or compounds administered, the age, weight, and response of the individual patient, and the severity of the patient's symptoms.

Certain compounds of formula (I), (II), (III), (IA), (IIA) or (IIIA), or a pharmaceutically acceptable salt thereof, selectively target FGFR3. For example, certain compounds of formula (I), (II), (III), (IA), (IIA) or (IIIA), or a pharmaceutically acceptable salt thereof, selectively target FGFR3 over another FGFR. For example, certain compounds of formula (I), (II), (III), (IA), (IIA) or (IIIA), or a pharmaceutically acceptable salt thereof, selectively target FGFR3 over FGFR1. For example, certain compounds of formula (I), (II), (III), (IA), (IIA) or (IIIA), or a pharmaceutically acceptable salt thereof, are at least about 3 fold (e.g. at least about 4-, 5-, 6-, 7-, 8-, 9-, 10-, 15-, 20-, 30-, 40-, 50-fold, or more) more selective for FGFR3 than for FGFR1.

As used herein, the term “selectivity” of a compound refers to the compound having more potent activity at the first target than the second target. A fold selectivity can be calculated by any method known in the art. For example, a fold selectivity can be calculated by dividing the IC₅₀ value of a compound for the second target (e.g., FGFR1) by the IC₅₀ value of the same compound for the first target (e.g., FGFR3). An IC₅₀ value can be determined by any method known in the art. For example, an IC₅₀ value can be determined as described in the assays below.

As used herein, the term “cancer” refers to or describes the physiological condition in patients that is typically characterized by unregulated cell proliferation. Included in this definition are benign and malignant cancers, primary and metastatic cancers.

As used herein, the term “FGFR3-associated cancer” refers to cancers having a dysregulation of the FGFR3 gene, the FGFR3 kinase protein, or expression or activity, or level of any of the same. Non-limiting examples of FGFR3-associated cancer include but are not limited to breast cancer (e.g. invasive ductal cancer, invasive lobular cancer), lung cancer (e.g. non-small-cell lung cancer, lung adenocarcinoma, squamous cell lung cancer and small-cell lung cancer), urothelial cancer, bladder cancer (e.g. urothelial bladder cancer, non-muscle invasive bladder cancer, high risk non-muscle invasive bladder cancer, intermediate risk non-muscle invasive bladder cancer, Bacillus Calmette-Guerin (BCG)-unresponsive non-muscle invasive bladder cancer, Bacillus Calmette-Guerin (BCG) recurrent non-muscle invasive bladder cancer, muscle invasive bladder cancer), upper tract cancer (e.g. urothelial upper tract cancer), urethral cancer, gastric cancer, pancreatic cancer, prostate cancer, colorectal cancer, multiple myeloma, liver cancer, melanoma (e.g. cutaneous melanoma), head and neck cancer (e.g. oral cancer), thyroid cancer, renal cancer (e.g. renal pelvis cancer), glioblastoma, endometrial cancer, cervical cancer, ovarian cancer, and testicular cancer.

As used herein, the term “treating” (or “treatment”) refers to restraining, slowing, stopping, or reversing the progression or severity of an existing symptom, condition or disorder.

As used herein, the term “patient” refers to a mammal, particularly a human.

Provided herein, are compounds of formula (I), (II), (III), (IA), (IIA) or (IIIA), or a pharmaceutically acceptable salt thereof, for use in therapy.

Provided herein, are compounds of formula (I), (II), (III), (IA), (IIA) or (IIIA), or a pharmaceutically acceptable salt thereof, for use in the treatment of cancer.

Provided herein, are compounds of formula (I), (II), (III), (IA), (IIA) or (IIIA), or a pharmaceutically acceptable salt thereof, for use in the treatment of a FGFR3-associated cancer, wherein the FGFR3-associated cancer is non-muscle invasive bladder cancer.

Provided herein, are compounds of formula (I), (II), (III), (IA), (IIA) or (IIIA), or a pharmaceutically acceptable salt thereof, for use in the treatment of a FGFR3-associated cancer, wherein the FGFR3-associated cancer is intermediate risk non-muscle invasive bladder cancer.

Provided herein, are compounds of formula (I), (II), (III), (IA), (IIA) or (IIIA), or a pharmaceutically acceptable salt thereof, for use in the treatment of a FGFR3-associated cancer, wherein the FGFR3-associated cancer is Bacillus Calmette-Guerin (BCG)-unresponsive non-muscle invasive bladder cancer or Bacillus Calmette-Guerin (BCG) recurrent non-muscle invasive bladder cancer.

Provided herein, are compounds of formula (I), (II), (III), (IA), (IIA) or (IIIA), or a pharmaceutically acceptable salt thereof, for use in the treatment of a FGFR3-associated cancer, wherein the FGFR3-associated cancer is high risk non-muscle invasive bladder cancer.

Provided herein, are the use of compounds of formula (I), (II), (III), (IA), (IIA) or (IIIA), or a pharmaceutically acceptable salt thereof, in the manufacture of a medicament for treating a FGFR3-associated cancer.

Provided herein, are the use of compounds of formula (I), (II), (III), (IA), (IIA) or (IIIA), or a pharmaceutically acceptable salt thereof, in the manufacture of a medicament for treating a FGFR3-associated cancer, wherein the FGFR3-associated cancer is non-muscle invasive bladder cancer.

Provided herein, are the use of compounds of formula (I), (II), (III), (IA), (IIA) or (IIIA), or a pharmaceutically acceptable salt thereof, in the manufacture of a medicament for treating a FGFR3-associated cancer, wherein the FGFR3-associated cancer is intermediate risk non-muscle invasive bladder cancer.

Provided herein, are the use of compounds of formula (I), (II), (III), (IA), (IIA) or (IIIA), or a pharmaceutically acceptable salt thereof, in the manufacture of a medicament for treating a FGFR3-associated cancer, wherein the FGFR3-associated cancer is Bacillus Calmette-Guerin (BCG)-unresponsive non-muscle invasive bladder cancer or Bacillus Calmette-Guerin (BCG) recurrent non-muscle invasive bladder cancer.

Provided herein, are the use of compounds of formula (I), (II), (III), (IA), (IIA) or (IIIA), or a pharmaceutically acceptable salt thereof, in the manufacture of a medicament for treating a FGFR3-associated cancer, wherein the FGFR3-associated cancer is high risk non-muscle invasive bladder cancer.

Provided herein are methods of treating cancer, comprising administering to a patient in need of such treatment an effective amount of the compounds of formula (I), (II), (III), (IA), (IIA) or (IIIA), or a pharmaceutically acceptable salt thereof.

Provided in the methods and uses herein, the cancer is selected from the group consisting of stomach cancer, hepatobiliary cancer, cancer of unknown primary, gallbladder cancer (e.g. gallbladder adenocarcinoma), bile duct cancer (e.g. intrahepatic bile duct cancer, extrahepatic bile duct cancer), sarcoma, esophagogastric cancer (e.g. gastroesophageal junction adenocarcinoma, gastric remnant adenocarcinoma), esophageal cancer (e.g. esophageal squamous cell cancer, esophageal adenocarcinoma), glioma (e.g. astrocytoma, oligodendroglioma, ependymoma), Non-Hodgkin Lymphoma (e.g. B-cell Non-Hodgkin Lymphoma), gastrointestinal stromal tumor, breast cancer (e.g. invasive ductal cancer, invasive lobular cancer), lung cancer (e.g. non-small-cell lung cancer, lung adenocarcinoma, squamous cell lung cancer and small-cell lung cancer), urothelial cancer, bladder cancer (e.g. urothelial bladder cancer, non-muscle invasive bladder cancer, high risk non-muscle invasive bladder cancer, intermediate risk non-muscle invasive bladder cancer, Bacillus Calmette-Guerin (BCG)-unresponsive non-muscle invasive bladder cancer, Bacillus Calmette-Guerin (BCG) recurrent non-muscle invasive bladder cancer, muscle invasive bladder cancer), gastric cancer (e.g. gastric adenocarcinoma), pancreatic cancer (e.g. pancreatic adenocarcinoma), prostate cancer (e.g. prostate adenocarcinoma), colorectal cancer (e.g. colorectal adenocarcinoma, colon adenocarcinoma,), multiple myeloma, liver cancer (e.g. hepatocellular cancer, fibrolamellar hepatocellular cancer), skin cancer (e.g. squamous cell skin cancer), melanoma (e.g. cutaneous melanoma), head and neck cancer (e.g. head and neck squamous cell cancer, hypopharyngeal cancer, laryngeal cancer, lip and oral cavity cancer, salivary gland cancer), glioblastoma, endometrial cancer (e.g. endometrial endometrioid adenocarcinoma), cervical cancer and ovarian cancer (e.g. epithelial ovarian cancer). Particularly, the cancer is selected from the group consisting of stomach cancer, hepatobiliary cancer, cancer of unknown primary, gallbladder cancer (e.g. gallbladder adenocarcinoma), bile duct cancer (e.g. intrahepatic bile duct cancer, extrahepatic bile duct cancer), esophagogastric cancer (e.g. gastroesophageal junction adenocarcinoma, gastric remnant adenocarcinoma), esophageal cancer (e.g. esophageal squamous cell cancer, esophageal adenocarcinoma), glioma (e.g. astrocytoma, oligodendroglioma, ependymoma), breast cancer (e.g. invasive ductal cancer, invasive lobular cancer), lung cancer (e.g. non-small-cell lung cancer, lung adenocarcinoma, squamous cell lung cancer and small-cell lung cancer), gastric cancer (e.g. gastric adenocarcinoma), pancreatic cancer (e.g. pancreatic adenocarcinoma), colorectal cancer (e.g. colorectal adenocarcinoma, colon adenocarcinoma,), liver cancer (e.g. hepatocellular cancer, fibrolamellar hepatocellular cancer), skin cancer (e.g. squamous cell skin cancer), melanoma (e.g. cutaneous melanoma), head and neck cancer (e.g. head and neck squamous cell cancer, hypopharyngeal cancer, laryngeal cancer, lip and oral cavity cancer, salivary gland cancer), glioblastoma, endometrial cancer (e.g. endometrial endometrioid adenocarcinoma) and ovarian cancer (e.g. epithelial ovarian cancer). More particularly, the cancer is selected from the group consisting of hepatobiliary cancer, cancer of unknown primary, gallbladder cancer (e.g. gallbladder adenocarcinoma), bile duct cancer (e.g. intrahepatic bile duct cancer, extrahepatic bile duct cancer), breast cancer (e.g. invasive ductal cancer, invasive lobular cancer), liver cancer (e.g. hepatocellular cancer, fibrolamellar hepatocellular cancer), skin cancer (e.g. squamous cell skin cancer), melanoma (e.g. cutaneous melanoma) and endometrial cancer (e.g. endometrial endometrioid adenocarcinoma). Most particularly, the cancer is selected from the group consisting of hepatobiliary cancer, gallbladder cancer (e.g. gallbladder adenocarcinoma), bile duct cancer (e.g. intrahepatic bile duct cancer, extrahepatic bile duct cancer), breast cancer (e.g. invasive ductal cancer, invasive lobular cancer), liver cancer (e.g. hepatocellular cancer, fibrolamellar hepatocellular cancer and endometrial cancer (e.g. endometrial endometrioid adenocarcinoma).

The terms “NMIBC” or “non-muscle invasive bladder cancer” mean bladder cancer staged as T0, Ta, T1, or CIS according to the Tumor, Node, Metastasis Classification (TNM). The term “T0” means the first stage of disease where there is no evidence of primary tumor according to the Tumor, Node, Metastasis Classification (TNM). The terms “Ta”, “T1”, “T2”, “T3”, and “T4” mean the size or extent of the primary tumor according to the Tumor, Node, Metastasis Classification (TNM).

The terms “intermediate risk non-muscle invasive bladder cancer”, “intermediate risk NMIBC”, or “IR NMIBC” mean multiple or recurrent low-grade Ta tumors. The following factors to be considered are number of tumors such as greater than one, size of tumors such as greater than 3 cm, timing such as recurrence within 1 year, frequency of recurrences such as greater than one recurrence per year, and previous treatment.

The terms “high risk non-muscle invasive bladder cancer”, “high risk NMIBC”, or “HR NMIBC” mean recurrent, bacillus Calmette-Guerin (BCG) unresponsive, high-grade, T1 or CIS tumors wherein recurrence can be after BCG therapy. The following factors to be considered are tumor grade, size of tumors such as greater than 3 cm, timing such as recurrence within 1 year, frequency of recurrences such as greater than one recurrence per year, and previous treatment.

The compounds provided herein can be prepared as illustrated in the preparations and examples below.

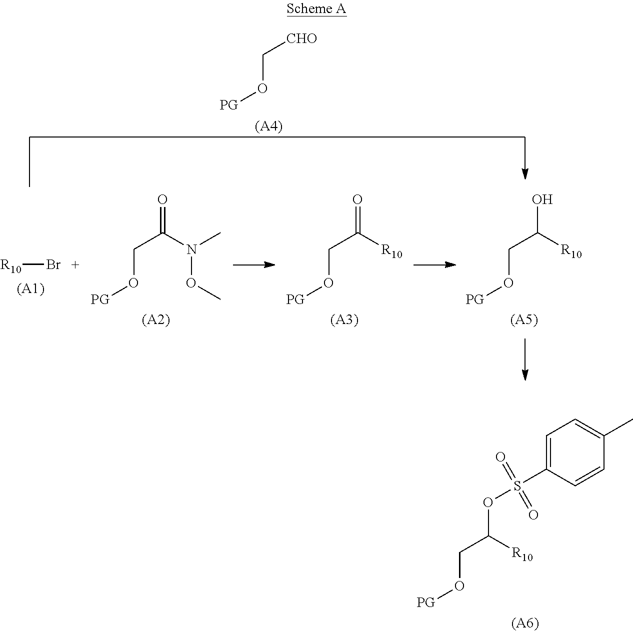

Scheme A depicts the two methods of preparation of (A5) that will be further elaborated to Formula 6. Scheme A also depicts the preparation of (A6) that will be further elaborated to Formula 1. In the first method for preparation of (A5), treatment of (A1), where R₁₀ is pyridyl, with i-PrMgCl followed by the addition of aldehyde (A4) may result in alcohol (A5). In the second method for preparation of (A5), treatment of (A1), where R₁₀ is pyridyl, with i-PrMgCl followed by the addition of 2-(benzyloxy)-N-methoxy-N-methylacetamide (A2) may afford ketone (A3). Reduction of ketone (A3) with sodium borohydride may afford alcohol (A5). In some instances, the enantiomers of alcohol (A5) may be separated by chiral chromatography. Scheme A also depicts the preparation of Tosylate (A6). Reaction of alcohol (A5) with 4-methylbenzenesulfonylchloride in the presence of a base may afford tosylate (A6). Compounds of alcohol (A5) or tosylate (A6) may be further elaborated to compounds of formula 1.

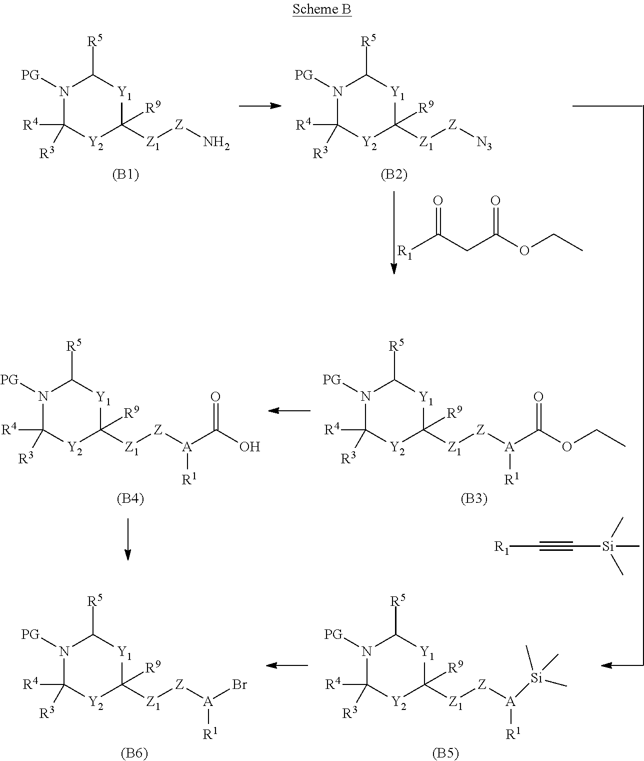

Scheme B depicts two methods for the preparation of (B6) which will be further elaborated to Formulas 1, 2, 3, 4, 9 and 10. In the first method, amine (B1) may be treated with 1H-imidazole-1-sulfonyl azide hydrochloride in the presence of a base and CuSO₄·H₂O to provide azide (B2). Reaction of (B2) with ethyl acetoacetate may afford ester (B3) which is then hydrolized to yield carboxylic acid (B4). Treatment of (B4) with bromine under basic conditions may provide bromide (B6). Alternatively, azide (B2) may be reacted with trimethyl(prop-1-yn-1-yl)silane to yield trimethylsilyl (B5) which is then treated with NBS in the presence of SiO₂ to afford (B6).

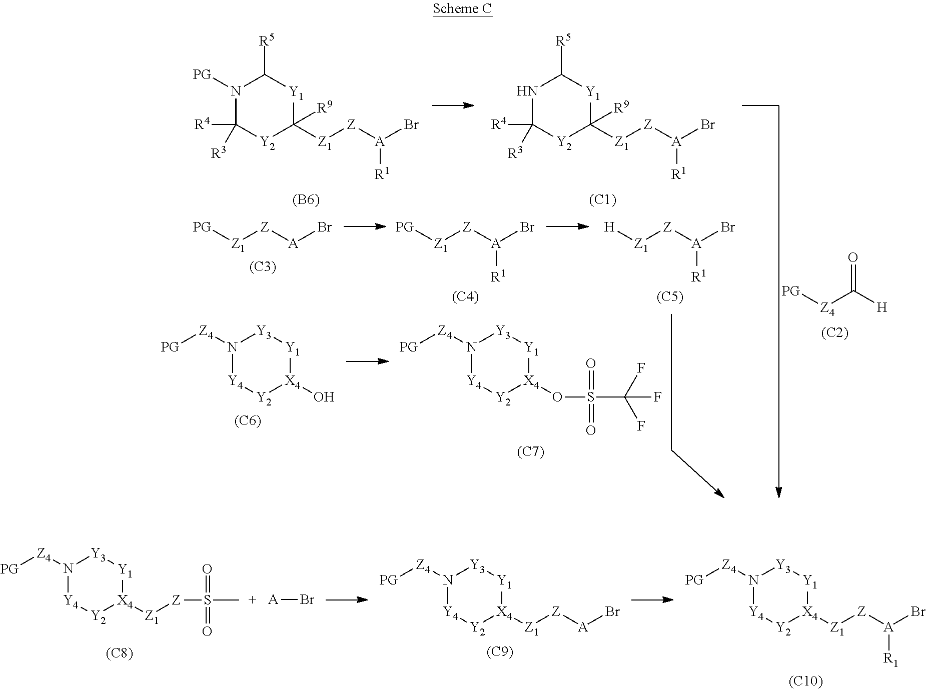

Scheme B depicts three methods for the preparation of (C10) which will be further elaborated to Formula 12. In the first method, deprotection of (B6) under acidic conditions may yield (C1) which is the subjected to reductive amination conditions with an appropriate aldehyde (C2) to afford (C10). In the second method, treatment of compound (C3) with LDA and an appropriate alkylating agent may afford compound (C4). This compound is then deprotected under acidic conditions to provide (C5). Alkylation of (C5) may be accomplished by reacting with triflate (C7) to form (C10). The triflate (C7) used in the alkylation of (C5) may be formed in-situ by reacting alcohol (C6) with trifluoromethanesulfonic anhydride. In the third method, reaction of ABr with an appropriate mesylate (C8) may provide (C9). Treatment of (C9) with LDA and an appropriate alkylating agent may afford (C10).

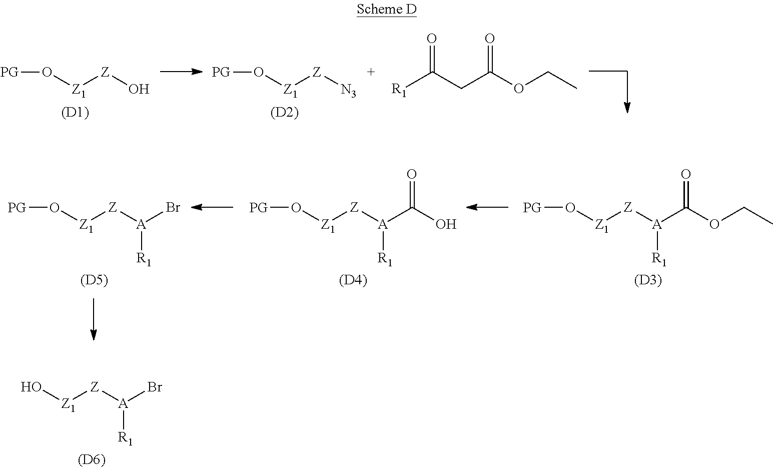

Scheme D depicts a method for the preparation of (D6) which will be further elaborated to Formula 6. Formation of azide (D2) may be accomplished by reacting the alcohol (D1) with PPh₃ and DEAD followed by DPPA. Reaction of azide (D2) with ethyl acetoacetate may yield ester (D3). Hydrolysis of (D3) under basic conditions may afford acid (D4) which then may be subjected to bromination under basic conditions to provide bromide (D5). Removal of the protecting group from (D5) in the presence of FeCl₃ may afford alcohol (D6).

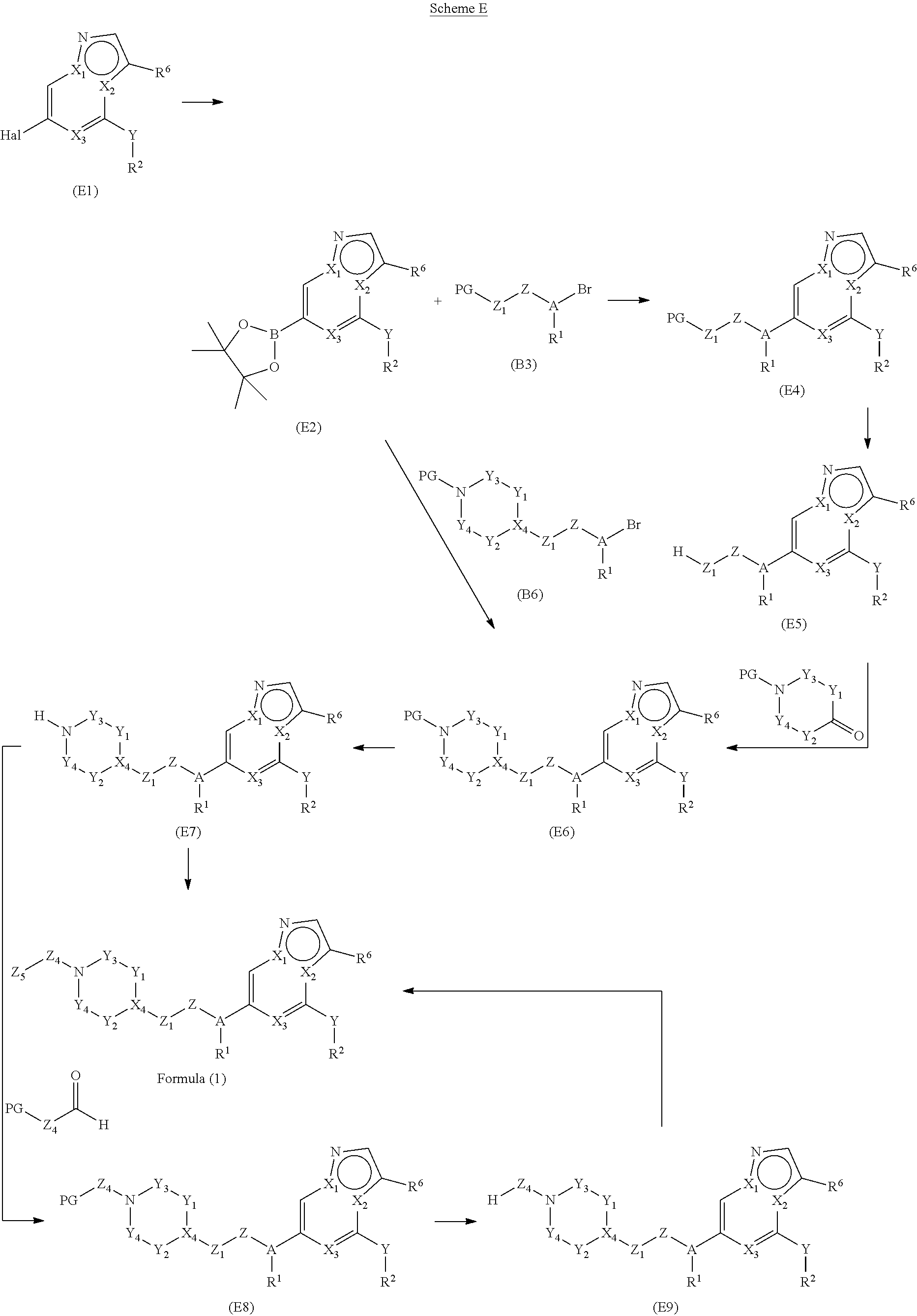

Scheme E depicts the preparation for compounds of Formula 1. Treatment of (E1) with bis(pinacolato)diboron under palladium catalyzed conditions may afford boronate ester (E2). From this intermediate two different methods may be used to arrive at compounds of (E6). In the first method, boronate ester (E2) may be reacted with the appropriate bromide (B6) under palladium catalyzed conditions to afford (E6). In the second method, boronate ester (E2) may be reacted with (B3) under palladium catalyzed conditions to provide (E4) which is then subjected to acidic conditions to remove the protecting group to afford amine (E5). Reaction of amine (E5) with the appropriate ketone under reductive amination conditions may provide (E6). Deprotection of (E6) under acidic conditions may yield amine (E7). From this intermediate, three methods may be used to arrive at compounds of Formula 1. In the first method, treatment of the amine (E7) with an appropriate alkenyl acid halide may provide compounds of Formula 1. In the second method compounds of Formula 1 may be synthesized by treatment of amine (E7) with an appropriate alkynyl carboxylic acid in the presence of a coupling reagent. In the third method, amine (E7) may be reacted with an appropriate aldehyde to afford (E8) which is then deprotected under acidic conditions to yield amine (E9). Treatment of the amine (E9) with an appropriate alkenyl acid halide may provide compounds of Formula 1. Alternatively, compounds of Formula 1 may be synthesized by treatment of amine (E9) with an appropriate alkynyl carboxylic acid in the presence of a coupling reagent.

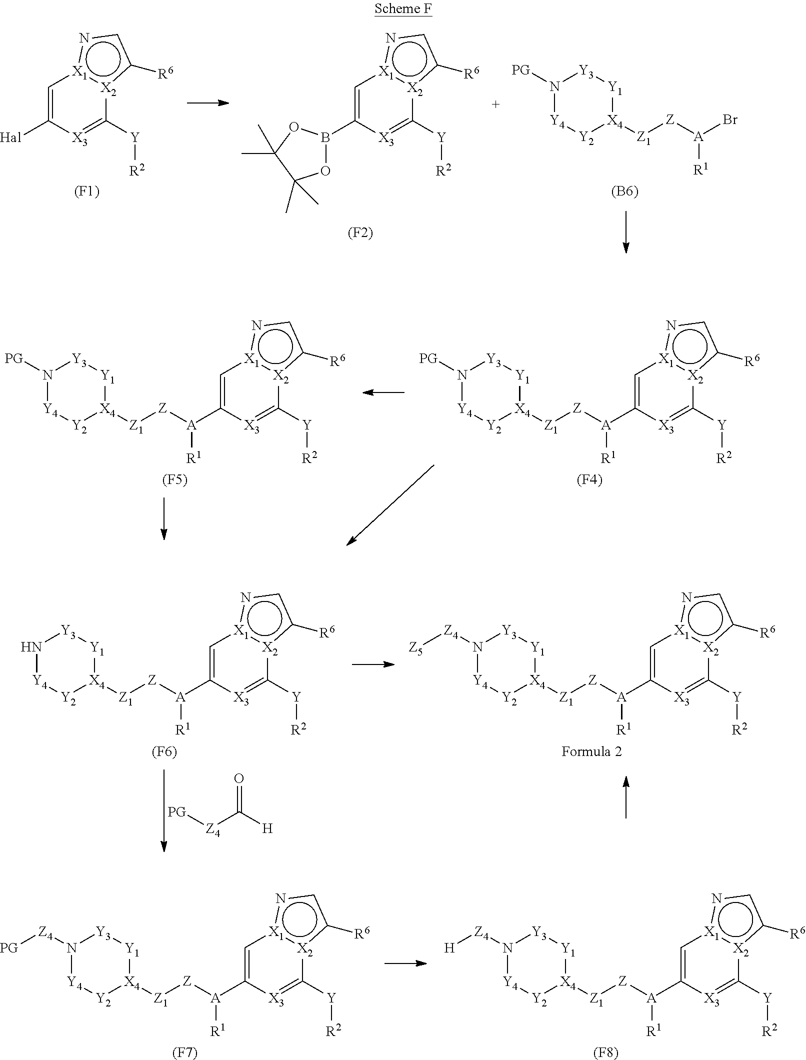

Scheme F depicts the preparation for compounds of Formula 2. Compound (F1), where R⁶=H, may be treated with bis(pinacolato)diboron under palladium catalyzed conditions to afford boronate ester (F2). Reaction of the boronate ester (F2) with the appropriate bromide (B6) under palladium catalyzed conditions may yield (F4). Halogenation of (F4) in the presence of NCS may afford (F5) which is then treated under acidic conditions to remove the protecting group that may provide compound (F6). In cases where R⁶=H, (F4) may be deprotected under acidic conditions to afford (F6). Subsequent treatment of the compound (F6) with the appropriate alkenyl acid halide may provide compounds of Formula 2. Alternatively, amine (F6) may be subjected to reductive amination conditions with an appropriate aldehyde to afford (F7) which may then be deprotected under acidic conditions to afford amine (F8). Subsequent treatment of the amine (F8) with the appropriate alkenyl acid halide may provide compounds of Formula 2. Compounds of Formula 2 may also be synthesized by treatment of amine (F8) with an appropriate alkynyl carboxylic acid in the presence of a coupling reagent.

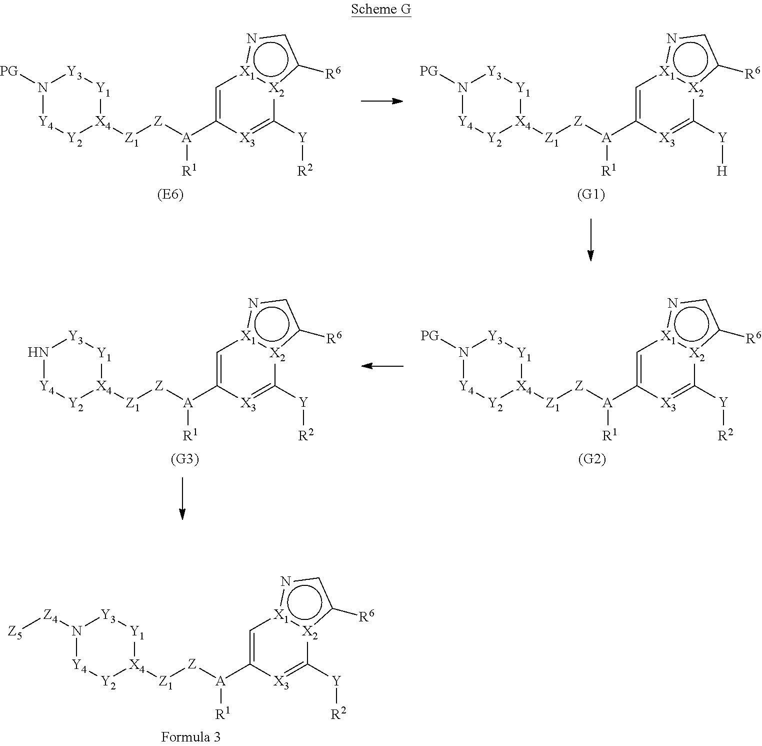

Scheme G depicts the preparation for compounds of Formula 3. Demethylation of the compound (E6) with NDM may afford demethylated (G1). Reaction of (G1) with the appropriate R²OH alcohol under Mitsunobu conditions may yield (G2). Alternatively, (G1) may be alkylated with tosylate (A6). Subsequent removal of the protecting group(s) may be accomplished under acidic conditions to afford amine (G3) which then would be treated with an appropriate alkenyl acid halide to provide compounds of Formula 3.

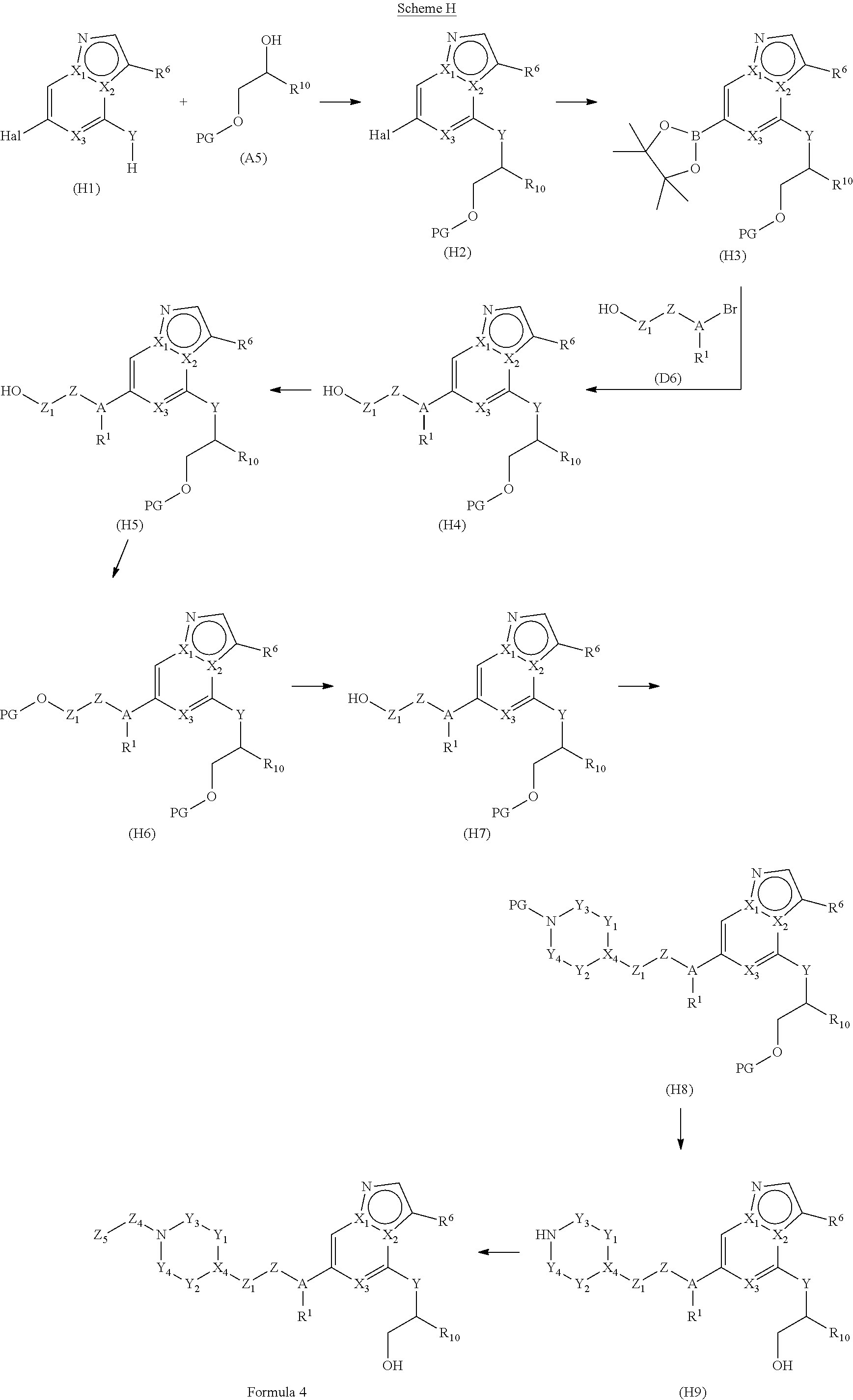

Scheme H depicts the preparation for compounds of Formula 4. Reaction of (A5) and (H1) under Mitsunobu conditions may afford compound (H2). Treatment of (H2) with bis(pinacolato)diboron under palladium catalyzed conditions may afford boronate ester (H3). Reaction of the boronate ester (H3) with (D4) under palladium catalyzed conditions may afford compound (H4). Treatment of (H4) in the presence of NCS may provide (H5). Reprotection of alcohol (H5) in the presence of TBDMSCl followed by chiral chromatography to individually isolate the enantiomers may afford (H6). Removal of the protecting group from (H6) may be accomplished by treatment under acid conditions to afford (H7). Compound (H7) may be treated with trifluoromethanesulfonic anhydride at −78° C. followed by reaction with tert-butyl-1-piperazinecarboxylate may afford (H8). Removal of the protecting groups by treatment with BCl₃ at −78° C. may provide (H9) which is then reacted with the appropriate alkenyl acid halide to afford compounds of Formula 4.

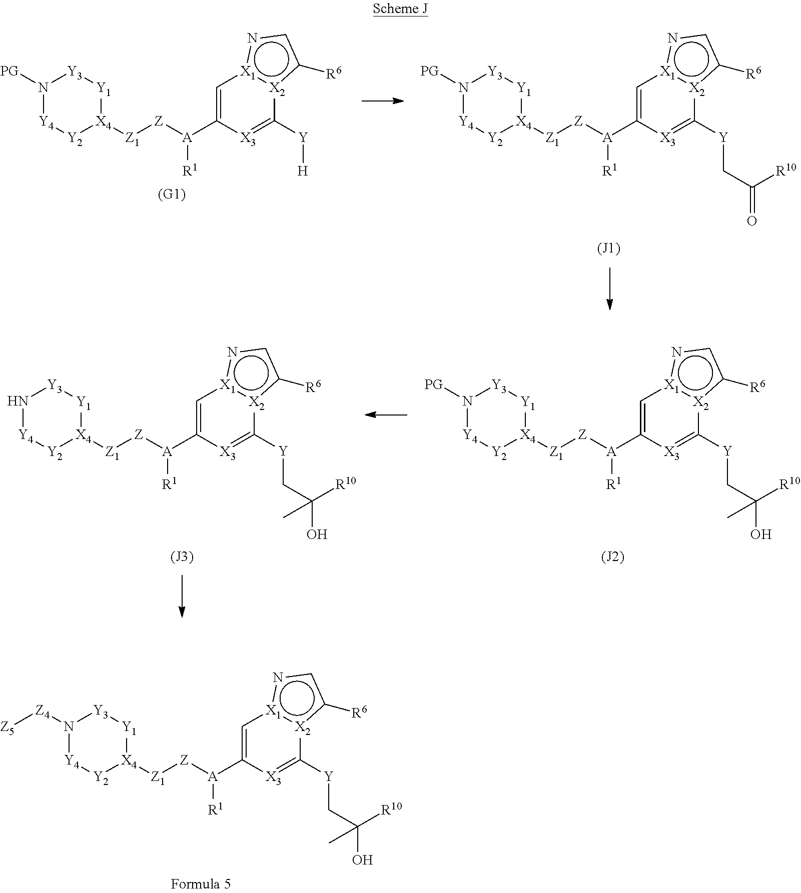

Scheme J depicts the preparation for compounds of Formula 5. Alkylation of (G1) with an appropriate alpha haloketone may provide (J1). Reaction of (J1) with the appropriate Grignard reagent may afford tertiary alcohol (J2). The tertiary alcohol is then treated under acidic conditions to provide the deprotected amine (J3). Subsequent treatment of the amine (J3) with the appropriate alkenyl acid halide may provide compounds of Formula 5.

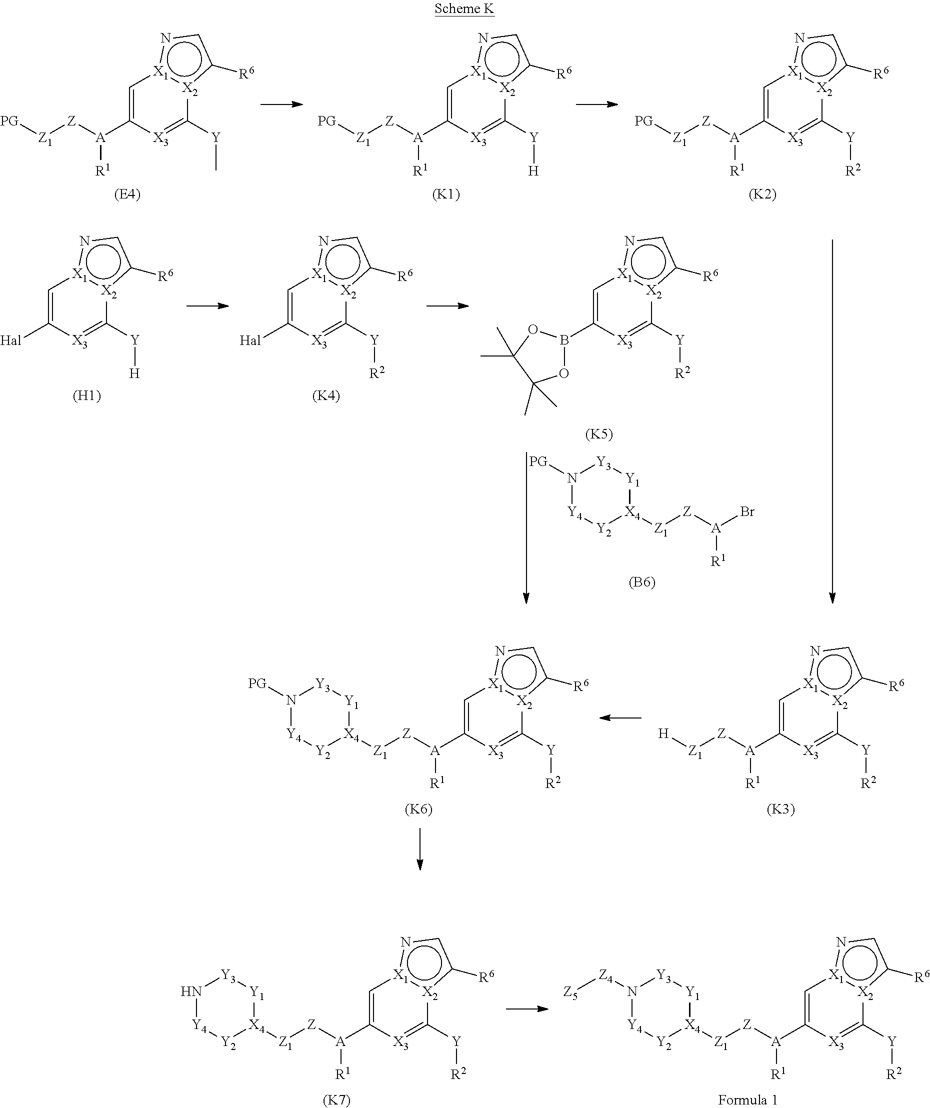

Scheme K depicts an alternative preparation for compounds of Formula 1. Two methods are described for the preparation of (K6). In the first method, (E4) is subjected to demethylation conditions in the presence of NDM which may provide (K1). Reaction of (K1) with an appropriate alcohol using Mitsunobu conditions to afford (K2). Removal of the protecting group under acidic conditions may yield amine (K3). Reaction of amine (K3) with the appropriate ketone under reductive amination conditions may afford (K6). In the second method, treatment of (H1) may be alkylated with the appropriate halide to afford (K4). Treatment of (K4) with bis(pinocolato)diboron under palladium catalyzed conditions may provide the boronic acid (K5) which is then reacted with (B6) under palladium catalyzed conditions to afford (K6). Deprotection of (K6) under acidic conditions may provide amine (K7) which is then reacted with the appropriate alkenyl acid halide to afford compounds of Formula 1.

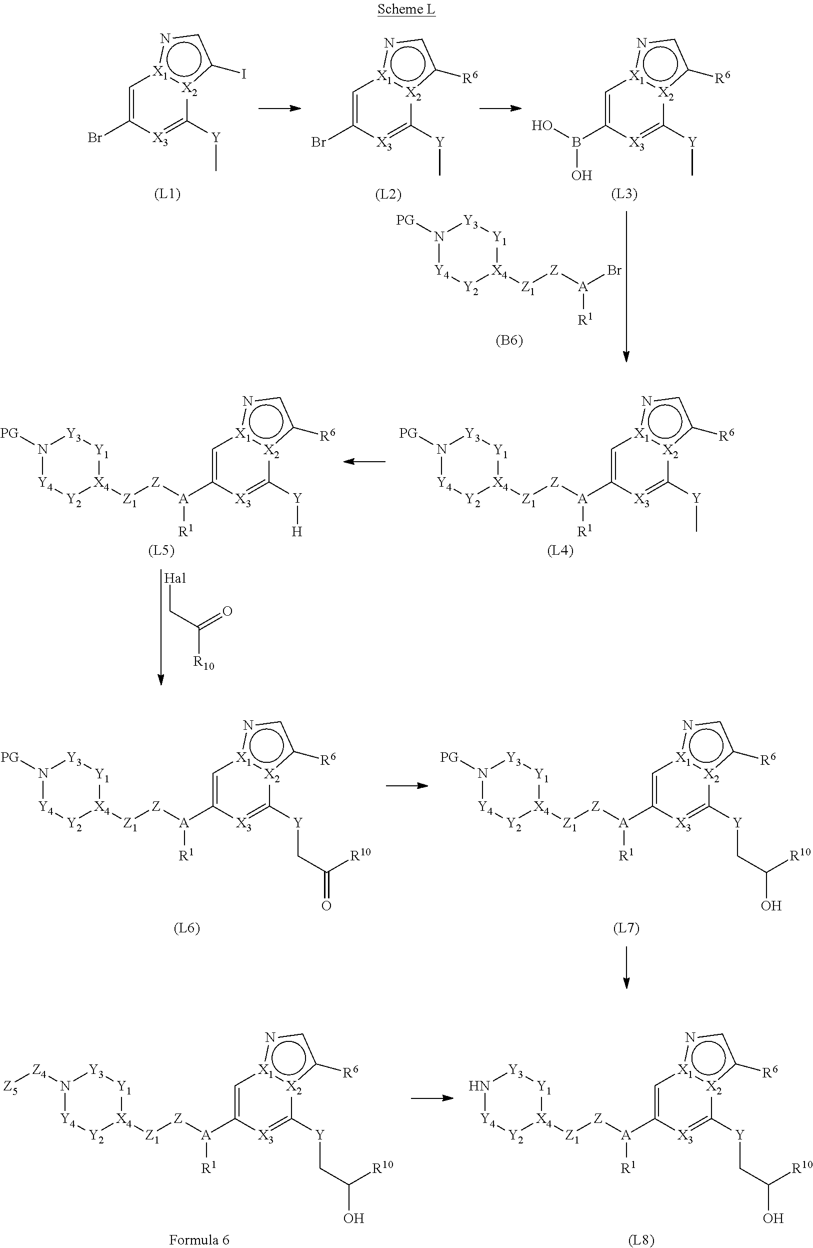

Scheme L depicts the preparation for compounds of Formula 6. Reaction of (L1) with methyl 2,2-difluoro-2-(fluorosulfonyl)acetate in the presence of CuI may provide (L2) where R⁶=F. Treatment of (L2) with bis(pinacolato)diboron under palladium catalyzed conditions may afford boronic acid (L3). Palladium catalyzed coupling with the appropriate bromide (B6) and boronic acid (L3) may yield (L4) which is then demethylated with NDM to afford (L5). Alkylation of (L5) with the appropriate alpha-haloketone may afford ketone (L6) which is then reduced with NaBH₄ to provide (L7). Removal of the protecting group under acidic conditions may yield (L8) which then is reacted with the appropriate acid halide to provide compounds of Formula 6.

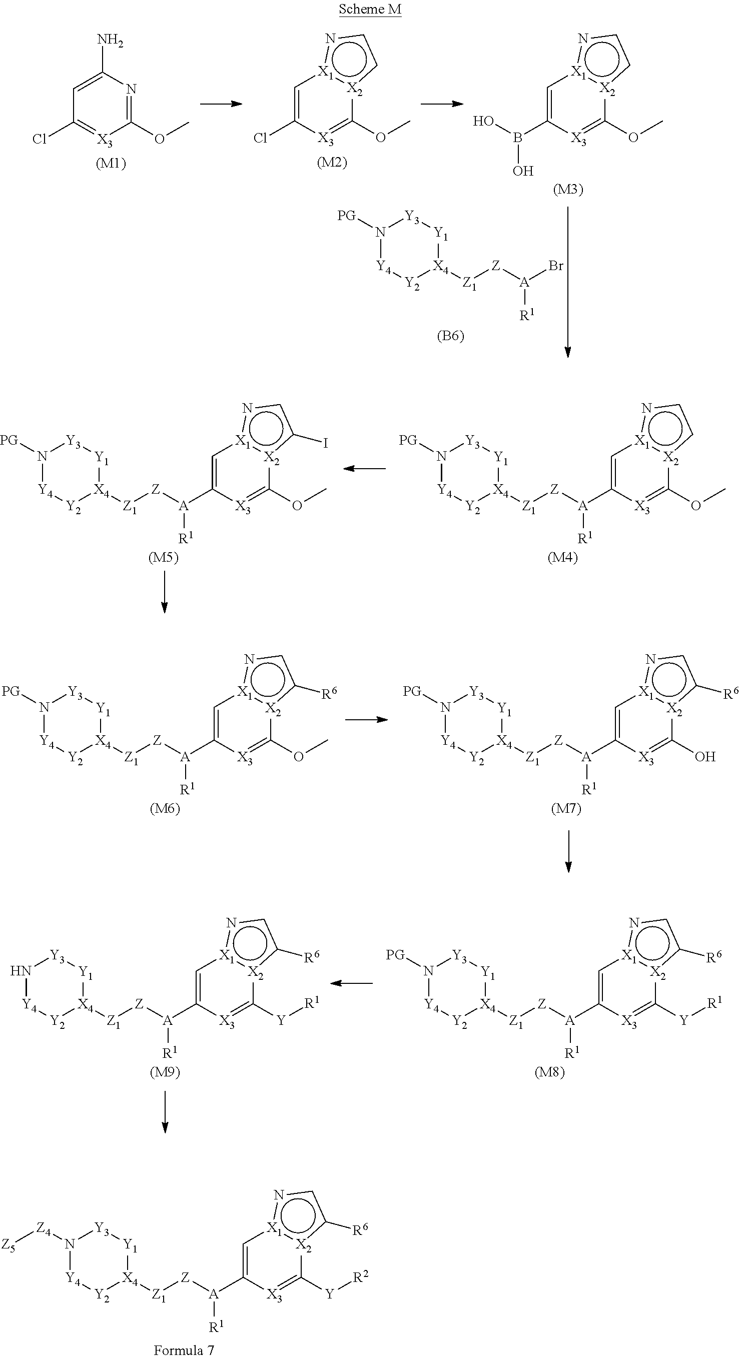

Scheme M depicts the preparation for compounds of Formula 7. Reaction of 4-chloro-6-methoxypyridin-2-amine (M1) with chloroacetaldehyde may afford the imidazo[1,2-a]pyridine (M2). Treatment of (M2) with bis(pinocolato)diboron under palladium catalyzed conditions may provide the boronic acid (M3) which is then reacted with (B6) under palladium catalyzed conditions to afford (M4). Iodination of (M4) in the presence of NIS may provide iodo (M5) which is then reacted with CuCN to afford cyano (M6). Demethylation of (M6) may be accomplished by reacting with NDM to provide (M7). Treatment of (M7) with the appropriate alcohol under Mitsunobu conditions may yield (M8). Removal of the protecting group may be accomplished by treatment under acidic conditions to afford (M9). Subsequent treatment of the amine (M9) with the appropriate alkenyl acid halide may provide compounds of Formula 7. Alternatively, treatment of (M9) with an appropriate alkynyl carboxylic acid in the presence of a coupling reagent may afford compounds of the Formula 7.

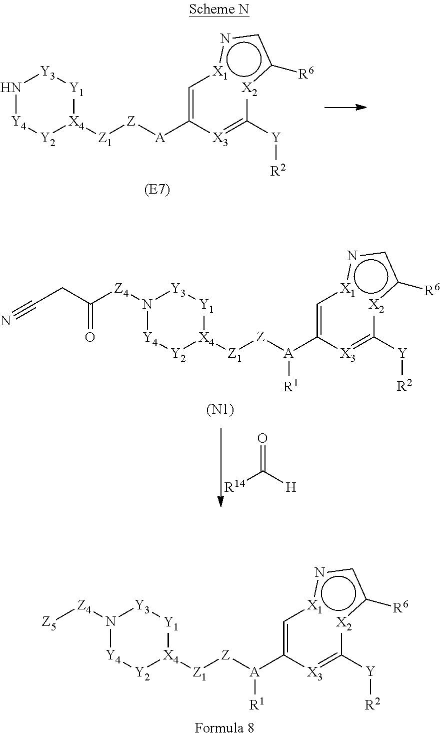

Scheme N depicts the preparation for compounds of Formula 8. Reaction of (E7) with 2-cyanoacetic acid using an appropriate coupling reagent may provide (N1). Compound (N1) is then condensed with an appropriate aldehyde to provide compounds of Formula 8.

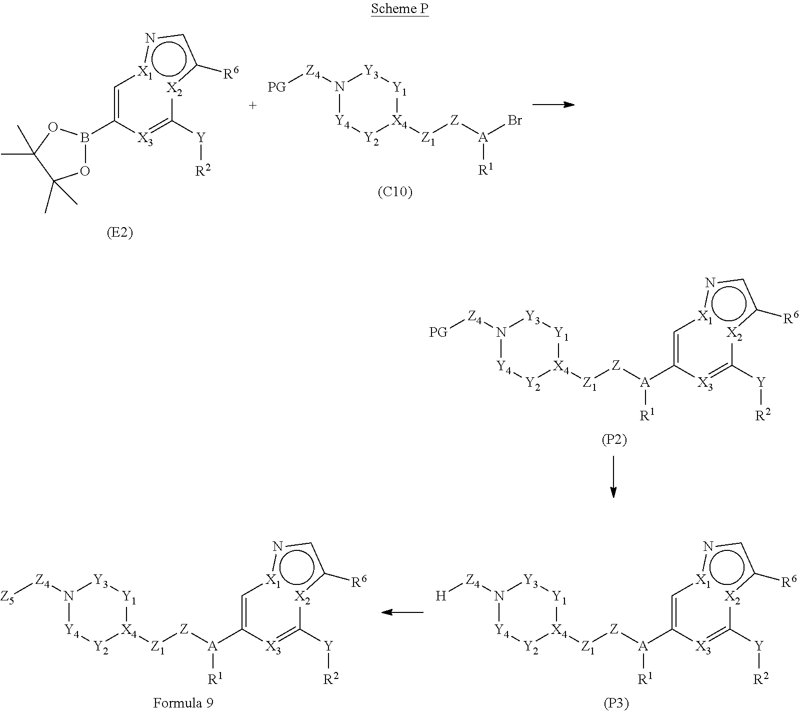

Scheme P depicts the preparation for compounds of Formula 9. Palladium catalyzed coupling of (E2) and (C10) may afford (P2). Deprotection of (B2) under acidic conditions may yield (P3) which is then reacted with an appropriate alkenyl halide to afford compounds of Formula 9.

Certain stereochemical centers have been left unspecified and certain substituents have been eliminated in the following schemes for the sake of clarity and are not intended to limit the teaching of the schemes in any way. Furthermore, individual isomers, enantiomers, and diastereomers may be separated or resolved by one of ordinary skill in the art at any convenient point in the synthesis of compounds of the invention, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994). The designations “isomer 1” and “isomer 2” refer to the compounds that elute from chiral chromatography first and second, respectively, under the conditions described herein and if chiral chromatography is initiated early in the synthesis, the same designation is applied to subsequent intermediates and examples. The designations “P1” and “P2” refer to the compounds that elute from chromatography first and second, respectively, under the conditions described herein and if chromatography is initiated early in the synthesis, the same designation is applied to subsequent intermediates and examples. Additionally, the intermediates described in the following schemes may contain a number of nitrogen or oxygen protecting groups. The variable protecting group may be the same or different in each occurrence depending on the particular reaction conditions and the particular transformations to be performed. The protection and deprotection conditions are well known to the skilled artisan and are described in the literature (See for example “Greene's Protective Groups in Organic Synthesis”, Fourth Edition, by Peter G. M. Wuts and Theodora W. Greene, John Wiley and Sons, Inc. 2007).
“AcOH” refers to acetic acid; “ACN” refers to acetonitrile; “aq” refers to aqueous; “HATU” refers to 1-[bis(dimethylamino) methylene]-1H-1,2,3-triazolo[4,5-b]pyridinium 3-oxide hexafluorophosphate; “Pd(DtBPF)Cl₂” refers to [1,1′-bis(di-tert-butylphosphino) ferrocene]dichloropalladium(II); “TBDMSCl” refers to tert-butyldimethylsilyl chloride; “XPhos Pd G2” refers to chloro(2-dicyclohexylphosphino-2′,4′,6′-triisopropyl-1,1′-biphenyl)[2-(2′-amino-1,1′-biphenyl)]palladium(II); “DCM” refers to dichloromethane; “DMA” refers to dimethylacetamide; “DMAP” refers to N,N-dimethylpyridin-4-amine; “DPPA” refers to diphenylphosphoryl azide; “NDM” refers to 1-dodecanethiol; “EA” refers to ethyl acetate; “DEAD” refers to diethyl azodicarboxylate; “Et₂O” refers to diethylether; “DIAD” refers to diisopropyl azodicarboxylate; “DIPEA” refers to N,N-diisopropylethylamine; “DMF” refers to N,N-dimethylformamide; “FA” refers to formic acid; “hr” refers to hour; “NMI” refers to 1-methyl-1H-imidazole; “IPA” refers to isopropyl alcohol; “iPrMgCl” refers to isopropylmagnesium chloride; “MeMgBr” refers to methylmagnesium bromide; “MeOH” refers to methanol; “NaBH₄” refers to sodium borohydride; “NBS” refers to N-bromosuccinimide; “NCS” refers to N-chlorosuccinimide; “M” refers to molar; “mmol” refers to millimole; “mL” refers to milliliters; “MTBE” refers to methyl tert-butyl ether; “min” refers to minute; “MS” refers to molecular sieves; “NMR” refers to nuclear magnetic resonance; “PdCl₂(dppf)’ refers to [1,1′-bis(diphenylphosphino)ferrocene]dichloropalladium(II); “PE” refers to petroleum ether; “KOAc” refers to potassium acetate; “PPTS” refers to pyridinium p-toluenesulfonate; “RT” refers to room temperature; “rxn” refers to reaction; “sat.” refers to saturated; “soln” refers to solution; “Pd(PPh₃)₄” refers to tetrakis(triphenylphosphine) palladium(0); “PPTS” refers top-toluenesulfonic acid pyridine salt; “NaBH(OAc)₃” refers to sodium triacetoxyborohydride; “TCFH” refers to N,N,N′,N′-tetramethylchloro-formamidinium hexafluorophosphate; “NEt₃” refers to triethylamine; “PPh₃” refers to triphenylphosphine; “T3P” refers to 1-propanephosphonic anhydride; “TFA” refers to 2,2,2-trifluoroacetic acid; “THF” refers to tetrahydrofuran.

For compounds where the amine salt was isolated, the formation of the mono-, di-, or trivalent salt is dependent on the pKa of the amine and the acid used to form the salt. The exact mono-, di-, or trivalent salt form for each compound was not identified.

### Preparation 1

### 2-Benzyloxy-1-(5-fluoro-2-pyridyl)ethanone

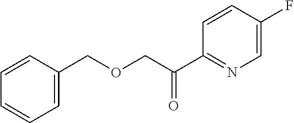 [SMILES: O=C(COCc1ccccc1)c1ccc(F)cn1]

To a soln of 2-bromo-5-fluoropyridine (50 g, 284 mmol) in toluene (100 mL) was added dropwise iPrMgCl (2.0 M soln in THF) (170.47 mL, 341 mmol) at 0° C. under N₂. The mixture was stirred for 2 hr at RT then 2-(benzyloxy)-N-methoxy-N-methylacetamide (65.4 g, 313 mmol) was added dropwise over a 10 min period at 0° C. The reaction was stirred for 1 hr at RT, cooled to 0° C., then quenched with sat. aq NH₄Cl (250 mL). The mixture was extracted with EA (3×500 mL), organic layers were combined, washed with brine (3×500 mL), dried over Na₂SO₄, filtered, and concentrated to afford the title compound (79 g, crude) as a light brown oil which was used directly in the next step. ES/MS m/z 246 [M+H]⁺.

### Preparation 2

### 2-Benzyloxy-1-(5-fluoro-2-pyridyl)ethanol

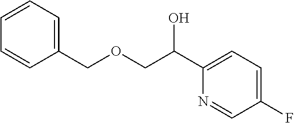 [SMILES: OC(COCc1ccccc1)c1ccc(F)cn1]

To a stirred soln of 2-benzyloxy-1-(5-fluoro-2-pyridyl)ethanone (79 g, 322 mmol) in MeOH (273 mL) was added NaBH₄ (6.09 g, 161 mmol) in portions at 0° C. under N₂. The resulting mixture was stirred at 0° C. for 1 hr. The reaction was quenched by the addition of H₂O (200 mL) at RT then concentrated. The mixture was extracted with EA (3×300 mL). The combined organic layers were dried over Na₂SO₄, filtered, and the filtrate was concentrated. The residue was purified by silica gel chromatography and eluted with PE/EA (1:1) to afford the title compound (64.6 g, 81%) as a light-yellow solid. ES/MS m/z 248 [M+H]⁺.

### Preparation 3

### 2-Benzyloxy-1-(5-fluoro-2-pyridyl)ethanol

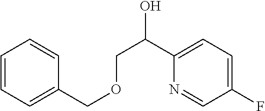 [SMILES: OC(COCc1ccccc1)c1ccc(F)cn1]

To a soln of 2-bromo-5-fluoropyridine (1.2 g, 6.8 mmol) in toluene (10 mL) was added iPrMgCl (2 M) (1.1 g, 10 mmol) dropwise at 0° C. under N₂. The mixture was stirred for 30 min at 0° C. Next, a soln of 2-(benzyloxy)acetaldehyde (1.8 g, 12 mmol) in toluene (2 mL) was added dropwise over 10 min at 0° C. The reaction was stirred for 2 hr at 0° C. then quenched with sat. aq NH₄Cl solution and extracted with EA (3×100 mL). The combined organic layers were dried over Na₂SO₄, filtered, and concentrated to an oily residue. The residue was purified by silica gel chromatography and eluted with 0% to 100% EA in heptane. The isolated product was re-purified by silica gel chromatography and eluted with 10% EA in DCM to afford the title compound (0.9 g, 53%) as a colorless oil that solidified upon standing. ¹H NMR (300 MHz, DMSO-d6) δ 3.56 (dd, J=10.03, 6.85 Hz, 1H), 3.71 (dd, J=10.03 Hz, 3.91 Hz, 1H), 4.50 (s, 2H), 4.80 (dt, J=6.66, 4.49 Hz, 1H), 5.69 (d, 5.01 Hz, 1H), 7.23-7.29 (m, 3H), 7.29-7.35 (m, 2H), 7.57 (dd, J=8.74, 4.71 Hz, 1H), 7.71 (td, J=8.86, 2.93 Hz, 1H), 8.48 (d, J=2.93 Hz, 1H).

### Preparation 4

### 2-Benzyloxy-1-(5-fluoro-2-pyridyl)ethanol, Isomer 1

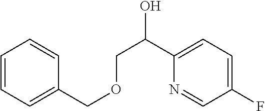 [SMILES: OC(COCc1ccccc1)c1ccc(F)cn1]

### and

### Preparation 5

### 2-Benzyloxy-1-(5-fluoro-2-pyridyl)ethanol, Isomer 2

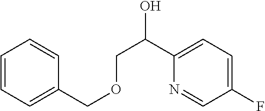 [SMILES: OC(COCc1ccccc1)c1ccc(F)cn1]

Separation of the 2-benzyloxy-1-(5-fluoro-2-pyridyl)ethanol enantiomers (100 mg) was performed using the following conditions: SFC; Stationary phase, Chiralpak AD-H, 21×150 mm, 5 μm; eluted with an 80% isocratic solution of CO₂ in MeOH (0.5% DMEA); UV at 265 nm; to afford 2-benzyloxy-1-(5-fluoro-2-pyridyl)ethanol, Isomer 1, t_{(R)} is 1.5 minutes (36.5 mg, %) with 97.4% ee, ES/MS m/z 248 [M+H]⁺; and 2-benzyloxy-1-(5-fluoro-2-pyridyl)ethanol, Isomer 2, t_{(R)} is 1.84 minutes (36.8 mg, %) with 97% ee, ES/MS m/z 248 [M+H]⁺.

### Preparation 6

### [2-Benzyloxy-1-(5-fluoro-2-pyridyl)ethyl]4-methylbenzenesulfonate, Isomer 1

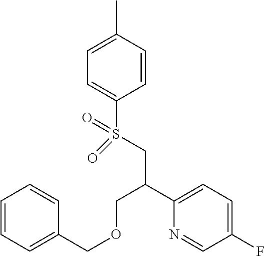 [SMILES: Cc1ccc(S(=O)(=O)CC(COCc2ccccc2)c2ccc(F)cn2)cc1]

To a soln of 2-benzyloxy-1-(5-fluoro-2-pyridyl)ethanol, Isomer 1 (3.0 g, 12.13 mmol) in DCM (30 mL) cooled to 0° C. was added DMAP (0.15 g, 1.21 mmol) and NEt₃ (2.46 g, 24.26 mmol). The soln was stirred at 0° C. for 5 minutes, then 4-methylbenzenesulfonyl chloride (3.01 g, 15.77 mmol) was added and stirred for 3 hr. The reaction was quenched by the addition of H₂O, and the organic layer was separated. The organic layer was dried over Na₂SO₄, filtered, and concentrated. The residue was purified by silica gel chromatography eluting with 0% to 100% EA in heptane to afford the title compound (4.37 g, 90%) as an amber oil.

### Preparation 7

### [(1R)-2-[tert-Butyl(dimethyl)silyl]oxy-1-(2-pyridyl)ethyl]4-methylbenzenesulfonate

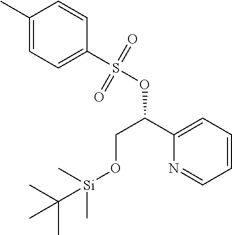 [SMILES: Cc1ccc(S(=O)(=O)O[C@@H](CO[Si](C)(C)C(C)(C)C)c2ccccn2)cc1]

To a soln of (R)-2-((tert-butyldimethylsilyl)oxy)-1-(pyridin-2-yl)ethan-1-ol (25 g, 99 mmol), NEt₃ (20 g, 200 mmol), and DMAP (1.2 g, 9.9 mmol) in DCM (250 mL) cooled to 0° C. was added 4-methylbenzenesulfonylchloride (24 g, 130 mmol). The mixture was stirred at 0° C. for 8 hr then stored at 4° C. overnight. The solids were filtered off and H₂O (5 mL) was added to the filtrate, concentrated to dryness and the crude residue was purified by silica gel chromatography and eluted with 0% to 100% EA in heptane to afford the title compound (34.9 g, 87%) as a light brown oil that solidified upon standing. ES/MS m/z 408 [M+H]⁺.

### Preparation 8

### 2-Bromo-N-(2,2-difluoroethyl)-2-methyl-propan-1-imine

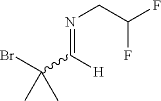

A soln of 2-bromo-2-methylpropanal (2.00 g, 13.3 mmol) and 2,2-difluoroethanamine (1.07 g, 13.3 mmol) in Et₂O (30 mL) was treated with 4 Å molecular sieves (100 mg) and stirred at RT for 2 hr. The resulting mixture was filtered, and the solids washed with Et₂O (3×50 mL). The filtrate was concentrated to afford the title compound (2.1 g, 74%) as a white solid. ES/MS m/z (⁷⁹Br/⁸¹Br) 214/216 [M+H]⁺.

### Preparation 9

### N-(2,2-Difluoroethyl)-1,1-dimethoxy-2-methyl-propan-2-amine

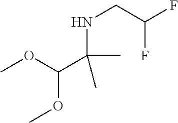

A soln of 2-bromo-N-(2,2-difluoroethyl)-2-methyl-propan-1-imine (2.10 g, 9.8 mmol) in MeOH (20 mL) was stirred at 80° C. for 2 hr. After cooling to RT, the soln was diluted with H₂O (10 mL) and the pH adjusted to 8 with aq NaOH (2M). The reaction was extracted with DCM (3×20 mL). The combined organics were washed with brine (3×50 mL), collected, dried over Na₂SO₄, filtered, and concentrated to afford the title compound (1.1 g, 57%) as a brown oil. ¹H NMR (300 MHz, DMSO-d₆) δ 5.89 (tt, J=56.4, 3.8 Hz, 1H), 3.99 (s, 1H), 3.45 (s, 6H), 2.95-2.80 (m, 2H), 0.93 (s, 6H).

### Preparation 10

### 2-(2,2-Difluoroethylamino)-2-methyl-propanal

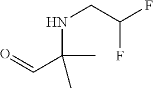

A soln of N-(2,2-difluoroethyl)-1,1-dimethoxy-2-methyl-propan-2-amine (0.50 g, 2.5 mmol) in q HCl (6 M, 15 mL) was stirred at 50° C. for 2 hr. After cooling to RT, the reaction was lyophilized to afford the title compound (0.56 g, crude) as a brown oil. ¹H NMR (300 MHz, DMSO-d₆) δ 9.01 (s, 1H), 6.33 (tt, J=54.2, 3.8 Hz, 1H), 3.42-3.25 (m, 2H), 1.18 (d, 6H).

### Preparation 11

### 2-Methyl-2-[methyl(oxetan-3-yl)amino]propanal

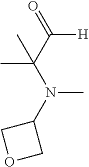

A soln of 2-bromo-2-methylpropanal (250 mg, 1.66 mmol), N-methyloxetan-3-amine (144 mg, 1.66 mmol), and K₂CO₃ (686 mg, 4.97 mmol) in DMF (5 mL) was stirred for 3 hr at 80° C. Upon cooling to RT, H₂O (10 mL) was added, and the mixture was extracted with EA (3×20 mL). The combined organic layers were dried over Na₂SO₄, filtered, and concentrated to afford the title compound (414 mg, crude) as a light-yellow oil which was used directly in the next step.

The following compound was prepared essentially as described in Preparation 11 using the appropriate reagents, adjusting temperature, adjusting the reaction times to determine completion of the reactions, and adjusting the purification system as appropriate.

TABLE 1

| | | | ¹H NMR (300 |
|---|---|---|---|
| Prep | | | MHz, DMSO-d₆) |
| # | Chemical Name | Structure | δ |
| 12 | tert-Butyl 2-(1,1- dimethyl-2-oxo- ethyl)-2,7-diazaspiro [3.5]nonane-7- carboxylate | 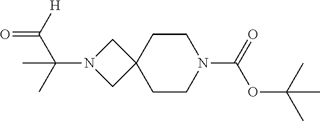 [SMILES: CC(C)(C)OC(=O)N1CCC2(CC1)CN(C(C)(C)C=O)C2] | 9.45 (s, 1H), 3.27- 3.20 (m, 8H), 1.64-1.59 (m, 4H), 1.39 (s, 9H), 0.99 (s, 6 H) |

### Preparation 13

### tert-Butyl (3 S,4R)-4-azido-3-hydroxy-piperidine-1-carboxylate

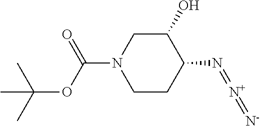 [SMILES: CC(C)(C)OC(=O)N1CC[C@@H](N=[N+]=[N-])[C@@H](O)C1]

To a mixture of tert-butyl (3 S,4R)-4-amino-3-hydroxypiperidine-1-carboxylate (3 g, 13.87 mmol), K₂CO₃ (0.96 g, 6.94 mmol), and CuSO₄·5H₂O (0.36 g, 1.39 mmol) in MeOH (30 mL) was added 1H-imidazole-1-sulfonyl azide hydrochloride (3.48 g, 16.65 mmol) at RT under N₂. The mixture was stirred overnight at RT. The reaction was quenched at RT with H₂O (100 mL). The mixture was extracted with EA (3×50 mL). The combined organic layers were washed with brine (3×50 mL), dried over Na₂SO₄, filtered, and concentrated to afford the title compound (4.5 g, crude) as a yellow oil. ¹H NMR (400 MHz, DMSO-d₆) δ 5.39 (s, 1H), 4.17-4.03 (m, 1H), 3.68-3.62 (m, 1H), 3.38-3.30 (m, 4H), 1.80-1.69 (m, 1H), 1.63-1.53 (m, 1H), 1.39 (s, 9H).

### Preparation 14

### (3-Azidocyclobutoxy)methylbenzene

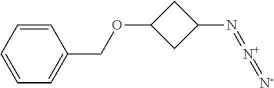 [SMILES: [N-]=[N+]=NC1CC(OCc2ccccc2)C1]

To 3-(benzyloxy)cyclobutan-1-ol (20 g, 112 mmol) and PPh₃ (39.7 g, 151 mmol) in THE (50 mL) was added dropwise DEAD (27.36 g, 157.100 mmol) at 0° C. under N₂. The mixture was stirred at 0° C. for 0.5 hr then DPPA (37.06 g, 134.7 mmol) was added. The mixture was then stirred at RT for 2 hr. The reaction was concentrated. The residue was purified by silica gel chromatography and eluted with PE/EA (20:1) to afford the title compound (21.5 g, 94%) as a pink oil. ¹H NMR (400 MHz, CDCl₃) δ 7.39-7.28 (m, 5H), 4.41 (s, 2H), 4.28-4.21 (m, 1H), 4.16-4.08 (m, 1H), 2.44-2.27 (m, 4H).

### Preparation 15

### Ethyl 1-[(3R)-1-tert-butoxycarbonylpyrrolidin-3-yl]-5-methyl-triazole-4-carboxylate

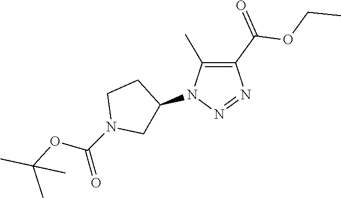 [SMILES: CCOC(=O)c1nnn([C@@H]2CCN(C(=O)OC(C)(C)C)C2)c1C]

A mixture of tert-butyl (3R)-3-azidopyrrolidine-1-carboxylate (45 g, 212 mmol) and K₂CO₃ (87.90 g, 636 mmol) at 80° C. was treated with ethyl acetoacetate dropwise overnight. The resulting mixture was filtered, and solids washed with DCM (3×50 mL). The filtrate was concentrated, and the residue purified by silica gel chromatography and eluted with PE/EA (10:1 to 1:1) to afford the title compound (32 g, crude) as a yellow oil. ES/MS m/z 325 [M+H]⁺.

The following compound was prepared essentially as described in Preparation 15 using the appropriate reagents, adjusting temperature, adjusting the reaction times to determine completion of the reactions, and adjusting the purification system as

TABLE 2

| | | | ES/MS |
|---|---|---|---|
| Prep | | | m/z |
| # | Chemical Name | Structure | [M + H]⁺ |
| 16¹ | Ethyl 1-[(3S)-1-tert- butoxycarbonylpyrrolidin- 3-yl]-5-methyl-triazole-4- carboxylate | 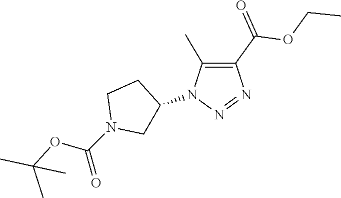 [SMILES: CCOC(=O)c1nnn([C@H]2CCN(C(=O)OC(C)(C)C)C2)c1C] | 325 |
| 17^{2,3} | Ethyl 1-(3- benzyloxycyclobutyl)-5- methyl-triazole-4- carboxylate | 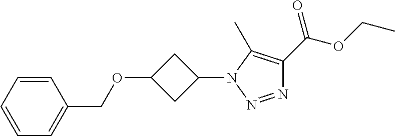 [SMILES: CCOC(=O)c1nnn(C2CC(OCc3ccccc3)C2)c1C] | 316 |

¹Purified by silica gel chromatography and eluted with PE/EA (10:1 to 1:1).
²Work up: Upon cooling to RT, reaction concentrated. Mixture diluted with H₂O and extracted with EA (3×). Combined organic layers were washed with brine, dried over Na₂SO₄, filtered and concentrated.
³Residue purified by silica gel column chromatography and eluted with 50% EA in PE.

### Preparation 18

### tert-Butyl (1R,3r,5S)-3-(4-bromo-1H-pyrazol-1-yl)-8-azabicyclo[3.2.1]octane-8-carboxylate

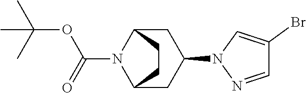 [SMILES: CC(C)(C)OC(=O)N1[C@@H]2CC[C@H]1C[C@@H](n1cc(Br)cn1)C2]

Cs₂CO₃ (19.20 g, 58.941 mmol) was added in portions to a stirred RT mixture of 4-bromopyrazole (2.89 g, 19.65 mmol) and tert-butyl (1R,3s,5S)-3-((methylsulfonyl)oxy)-8-azabicyclo[3.2.1]octane-8-carboxylate (6.00 g, 19.65 mmol) in DMF (50 ml) and the mixture was stirred overnight at 70° C. under N₂. The mixture was concentrated, and the residue was purified by silica gel chromatography and eluted with a gradient of PE/EA (10:1 to 5:1) to give a crude product (5.2 g). The crude product was purified by reverse phase flash chromatography with the following conditions: Column, C18; eluted with a gradient of 40% to 80% ACN in H₂O (0.1% FA), 220 nm to give the title compound as an off-white solid (3.5 g, 47.5%). ES/MS m/z 341/343 [M+H-tBu+ACN]⁺.

The following compound was prepared essentially as described in Preparation 18 using the appropriate reagents, adjusting temperature, adjusting the reaction times to determine completion of the reactions, and adjusting the purification system as

TABLE 3

| | | | ¹H NMR (400 |
|---|---|---|---|
| Prep | | | MHz, DMSO- |
| # | Chemical Name | Structure | d6) δ |
| 19¹ | tert-Butyl (1R,3s,5S)-3-(4- bromo-1H-pyrazol- 1-yl)-8-azabicyclo [3.2.1]octane-8- carboxylate | 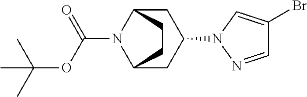 [SMILES: CC(C)(C)OC(=O)N1[C@@H]2CC[C@H]1C[C@H](n1cc(Br)cn1)C2] | 7.98 (s, 1H), 7.54 (s, 1H), 4.82-4.64 (m, 1H), 4.23- 4.09 (m, 2H), 2.04-1.82 (m, 6H), 1.81- |
| | | | 1.72 (m, 2H), |
| | | | 1.42 (s, 9H) |

¹The mixture was concentrated, diluted with H₂O, and extracted with EA (3 × 200 mL). The combined organics were washed with brine, dried over Na₂SO₄, filtered, and concentrated to afford the title compound.

### Preparation 20

### tert-Butyl 2-(4-bromopyrazol-1-yl)-7-azaspiro[3.5]nonane-7-carboxylate

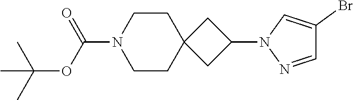 [SMILES: CC(C)(C)OC(=O)N1CCC2(CC1)CC(n1cc(Br)cn1)C2]

Cs₂CO₃ (18.36 g, 56.35 mmol) was added in portions at RT under N₂ to a stirred mixture of tert-butyl 2-(methanesulfonyloxy)-7-azaspiro[3.5]nonane-7-carboxylate (6.00 g, 18.78 mmol) and 4-bromopyrazole (2.76 g, 18.78 mmol) in DMF (50 mL) and the mixture was stirred for 2 hr at 100° C. under N₂. The mixture was cooled to RT, diluted with H₂O (100 mL), and extracted with EA (3×150 mL). The combined organic layers were washed with brine (2×200 mL), dried over anhydrous Na₂SO₄, filtered, and the filtrate was concentrated under reduced pressure. The residue was purified by reverse phase flash chromatography with the following conditions: Column, C18 eluted with a gradient of 40% to 50% ACN in H₂O (0.1% FA), 220 nm, to give the title compound (5 g). The product was dissolved in DCM (100 mL), washed with brine (2×150 mL), dried over anhydrous Na₂SO₄, filtered, and the filtrate was concentrated to give the title compound as an off-white solid (4.5 g, 64.7%). ¹H NMR (300 MHz, CDCl₃) δ 7.50 (s, 1H), 7.45 (s, 1H), 4.80-4.69 (m, 1H), 3.47-3.38 (m, 2H), 3.38-3.29 (m, 2H), 2.51-2.38 (m, 2H), 2.38-2.25 (m, 2H), 1.69-1.61 (m, 4H), 1.47 (s, 9H).

### Preparation 21

### 1-(1-tert-Butoxycarbonylazetidin-3-yl)-5-methyl-triazole-4-carboxylic acid

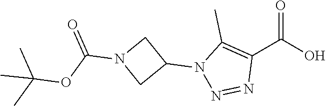 [SMILES: Cc1c(C(=O)O)nnn1C1CN(C(=O)OC(C)(C)C)C1]

A mixture of ethyl 1-[1-(tert-butoxycarbonyl)azetidin-3-yl]-5-methyl-1,2,3-triazole-4-carboxylate (20.00 g, 64.44 mmol) and KOH (7.23 g, 128.89 mmol) in H₂O (100 mL) was stirred for 2 hr at 50° C. under N₂. Upon cooling to RT, the mixture was cooled to 0° C. and acidified to pH 4 with 1M aq HCl. The aqueous layer was extracted with EA (3×400 mL). The combined organic layers were washed with brine (2×300 mL), dried over Na₂SO₄, filtered, and concentrated to afford the title compound (14 g, crude) as a yellow oil, which was used in the next step directly without purification. ES/MS m/z 283 [M+H]⁺.

### Preparation 22

### 1-[(3R)-1-tert-Butoxycarbonylpyrrolidin-3-yl]-5-methyl-triazole-4-carboxylic acid

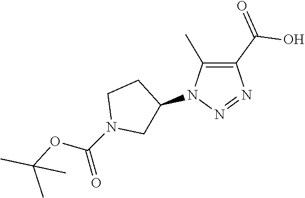 [SMILES: Cc1c(C(=O)O)nnn1[C@@H]1CCN(C(=O)OC(C)(C)C)C1]

A soln of KOH (11.07 g, 197.3 mmol) in H₂O (100 mL) was treated with ethyl 1-[(3R)-1-tert-butoxycarbonylpyrrolidin-3-yl]-5-methyl-triazole-4-carboxylate (32 g, crude) at RT. The reaction was stirred at 50° C. for 2 hr. After cooling, the reaction was extracted with EA (2×100 mL). The aqueous layer was acidified to pH ~3-4 with aq HCl (1 M) at 0° C. which resulted in a white suspension. The solid was collected by filtration and washed with H₂O (3×20 mL) and dried in vacuo to afford the product (23 g, 44%) as a white solid. ES/MS m/z 297 [M+H]⁺.

The following compound was prepared essentially as described in Preparation 22 using the appropriate reagents, adjusting temperature, adjusting the reaction times to determine completion of the reactions, and adjusting the purification system as appropriate.

TABLE 4

| | | | ES/MS |
|---|---|---|---|
| | | | m/z |
| Prep | | | [M + 2H − |
| # | Chemical Name | Structure | tBu]⁺ |
| 23 | 1-[(3S)-1-tert- Butoxycarbonylpyrrolidin- 3-yl]-5-methyl-triazole-4- carboxylic acid | 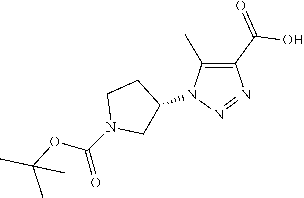 [SMILES: Cc1c(C(=O)O)nnn1[C@H]1CCN(C(=O)OC(C)(C)C)C1] | 241 |
| 24^{1,2} | 1-(3- benzyloxycyclobutyl)-5- methyl-triazole-4- carboxylic acid | 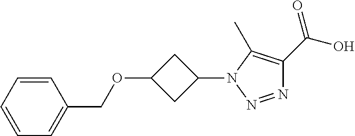 [SMILES: Cc1c(C(=O)O)nnn1C1CC(OCc2ccccc2)C1] | 288 |

¹MeOH used as co-solvent.
²Upon cooling to RT, reaction used in subsequent step as is.

### Preparation 25

### tert-Butyl (3S,4R)-3-hydroxy-4-(5-methyl-4-trimethylsilyl-triazol-1-yl)piperidine-1-carboxylate

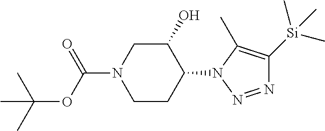 [SMILES: Cc1c([Si](C)(C)C)nnn1[C@@H]1CCN(C(=O)OC(C)(C)C)C[C@@H]1O]

A mixture of tert-butyl (3S,4R)-4-azido-3-hydroxy-piperidine-1-carboxylate (4.5 g) and trimethyl(prop-1-yn-1-yl)silane (2 mL) in toluene (2 mL) was irradiated with microwave radiation for 1 hr at 140° C. Upon cooling to RT, the mixture was concentrated. The residue was purified by silica gel chromatography and eluted with PE/EA (4:1) to afford the title compound (1.54 g, 23%) as a white solid. ES/MS m/z 355 [M+H]⁺.

### Preparation 26

### tert-Butyl (3S,4R)-4-(4-bromo-5-methyl-triazol-1-yl)-3-hydroxy-piperidine-1-carboxylate

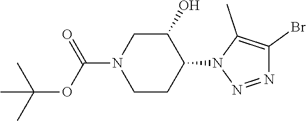 [SMILES: Cc1c(Br)nnn1[C@@H]1CCN(C(=O)OC(C)(C)C)C[C@@H]1O]

To a soln of tert-butyl (3S,4R)-3-hydroxy-4-(5-methyl-4-trimethylsilyl-triazol-1-yl)piperidine-1-carboxylate (1.54 g, 4.34 mmol) in ACN (50 mL) was added SiO₂ (0.52 g, 8.69 mmol) and NBS (1.16 g, 6.52 mmol) at RT under N₂. The resulting mixture was stirred for 2 hr at 80° C. Upon cooling to RT, the mixture was concentrated. The residue was purified by silica gel chromatography and eluted with PE/EA (4:1) to afford the title compound (1.2 g, 76%) as a white solid. ES/MS m/z (⁷⁹Br/⁸¹Br) 361/363 [M+H]⁺.

### Preparation 27

### tert-Butyl 3-(4-bromo-5-methyl-triazol-1-yl)azetidine-1-carboxylate

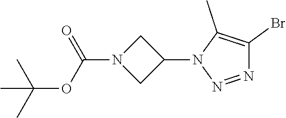 [SMILES: Cc1c(Br)nnn1C1CN(C(=O)OC(C)(C)C)C1]

To a soln of 1-(1-tert-butoxycarbonylazetidin-3-yl)-5-methyl-triazole-4-carboxylic acid (14.00 g, 49.59 mmol) and KOH (6.96 g, 123.98 mmol) in H₂O (150 mL) was added Br₂ (10.30 g, 64.47 mmol) dropwise at 0° C. under N₂. The mixture was stirred for 2 hr at RT. The mixture was extracted with EA (2×400 mL). The combined organic layers were washed with brine (2×300 mL), dried over Na₂SO₄, filtered, and concentrated to afford the title compound (14 g, crude) as a yellow solid which was used in the next step without purification. ES/MS m/z (⁷⁹Br/⁸¹Br) 317/319 [M+H]⁺.

The following compounds were prepared essentially as described in Preparation 27 using the appropriate reagents, adjusting temperature, adjusting the reaction times to determine completion of the reactions, and adjusting the purification system as appropriate.

TABLE 5

| | | | ES/MS m/z |
|---|---|---|---|
| Prep | | | (⁷⁹Br/⁸¹Br) |
| # | Chemical Name | Structure | [M + H]⁺ |
| 28 | tert-Butyl (3R)-3-(4-bromo-5- methyl-triazol-1- yl)pyrrolidine-1-carboxylate | 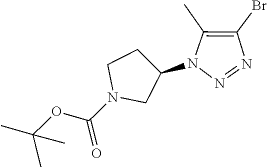 [SMILES: Cc1c(Br)nnn1[C@@H]1CCN(C(=O)OC(C)(C)C)C1] | 331/333 |
| 29 | tert-Butyl (3S)-3-(4-bromo-5- methyl-triazol-1- yl)pyrrolidine-1-carboxylate | 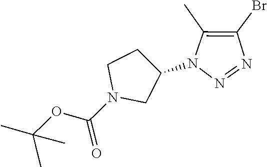 [SMILES: Cc1c(Br)nnn1[C@H]1CCN(C(=O)OC(C)(C)C)C1] | 331/333 |
| 30¹ | 1-(3-Benzyloxycyclobutyl)-4- bromo-5-methyl-triazole | 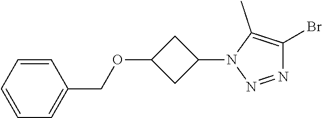 [SMILES: Cc1c(Br)nnn1C1CC(OCc2ccccc2)C1] | ^{a} |

^{a1}H NMR (400 MHz, DMSO-d6) δ 7.40-7.34 (m, 4H), 7.33-7.27 (m, 1H), 5.14-5.06 (m, 1H), 4.45 (s, 2H), 4.42-4.34 (m, 1H), 2.81-2.72 (m, 2H), 2.66-2.56 (m, 2H), 2.22 (s, 3H).
¹Purified by C18 reverse flash chromatography and eluted with 505 to 80% ACN in H₂O (0.1% FA).

### Preparation 31

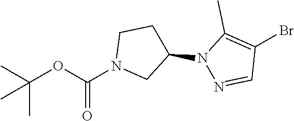

A soln of tert-butyl (3R)-3-(4-bromopyrazol-1-yl)pyrrolidine-1-carboxylate (2.00 g, 6.33 mmol) and CH₃I (2.69 g, 18.98 mmol) in THE (50 mL) at −5° C. under N₂ was treated dropwise with LDA (18.98 mL, 37.95 mmol). The mixture was stirred for 3 hr at −5° C. Next, the reaction was quenched with sat. aq NH₄Cl (100 mL) then extracted with EA (3×100 mL). The combined organic layers were washed with brine (2×50 mL) and dried over Na₂SO₄. After filtration, the filtrate was concentrated to afford the title compound (2.3 g) as a brown oil which was used directly in the next step without further purification. ¹H NMR (300 MHz, DMSO-d₆) δ 7.53 (s, 1H), 5.00 (d, 1H), 3.67 (d, 1H), 3.50-3.42 (m, 2H), 2.27 (s, 3H), 2.24-2.10 (m, 2H), 1.40 (d, 9H).

The following compounds were prepared essentially as described in Preparation 31 using the appropriate reagents, adjusting temperature, adjusting the reaction times to determine completion of the reactions, and adjusting the purification system as appropriate.

TABLE 6

| | | | ¹H NMR (300 |
|---|---|---|---|
| Prep | | | MHz, CDCl₃) |
| # | Chemical Name | Structure | δ |
| 32¹ | tert-Butyl (3S)-3-(4- bromo-5-methyl- pyrazol-1-yl)piperidine- 1-carboxylate | 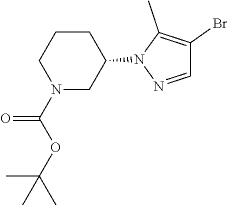 [SMILES: Cc1c(Br)cnn1[C@H]1CCCN(C(=O)OC(C)(C)C)C1] | 7.42 (d, 1H), 4.05-3.95 (m, 1H), 3.20- 3.00 (m, 1H), 2.77-2.68 (m, 1H), 2.29 (s, 3H), 2.20- 2.10 (m, 3H), 2.07-2.04 (m, 1H), 1.89- 1.82 (m, 1H), 1.67-1.57 (m, 1H), 1.45 (s, 9H) |
| 33 | tert-Butyl (1R,5S)-3-(4- bromo-5-methyl- pyrazol-1-yl)-8- azabicyclo[3.2.1]octane- 8-carboxylate | 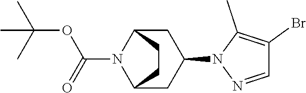 [SMILES: Cc1c(Br)cnn1[C@H]1C[C@H]2CC[C@@H](C1)N2C(=O)OC(C)(C)C] | 7.41 (s, 1H), 4.55-4.53 (m, 1H), 4.46- 4.26 (m, 2H), 2.40-2.30 (m, 2H), 2.29 (s, 3H), 2.09 (m, |
| | | | 2H), 1.83- |
| | | | 1.71(m, 4H), |
| | | | 1.51 (s, 9H) |
| 34 | tert-Butyl (1R,3s,5S)-3- (4-bromo-5-methyl-1H- pyrazol-1-yl)-8- azabicyclo[3.2.1]octane- 8-carboxylate | 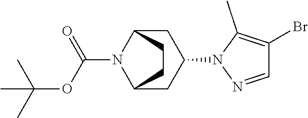 [SMILES: Cc1c(Br)cnn1[C@@H]1C[C@H]2CC[C@@H](C1)N2C(=O)OC(C)(C)C] | ^{a} |
| 35² | tert-Butyl (3S)-3-(4- bromo-5-methyl- pyrazol-1- yl)pyrrolidine-1- carboxylate | 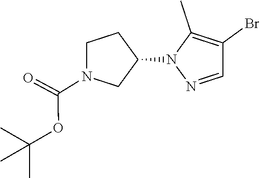 [SMILES: Cc1c(Br)cnn1[C@H]1CCN(C(=O)OC(C)(C)C)C1] | 7.53 (d, 1H), 5.00 (s, 1H), 3.71-3.65 (m, 1H), 3.54-3.42 (m, 3H), 2.27 (s, 3H), 2.19- 2.06 (m, 2H), 1.41-1.39 (m, 9H) |
| 36^{3,4} | tert-Butyl 2-(4-bromo- 5-methyl-pyrazol-1-yl)- 7-azaspiro[3.5]nonane- 7-carboxylate | 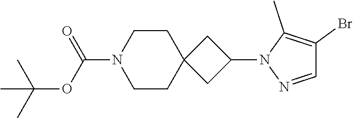 [SMILES: Cc1c(Br)cnn1C1CC2(CCN(C(=O)OC(C)(C)C)CC2)C1] | 7.53 (s, 1H), 4.91-4.86 (m, 1H), 3.33- 3.18 (m, 4H), 2.40-2.14 (m, 7H), 1.64- 1.59 (m, 2H), 1.52-1.47 (m, |
| | | | 2H), 1.40 (s, |
| | | | 9H) |
| 37⁵ | tert-Butyl (3R)-3-(4- bromo-5-methyl- pyrazol-1-yl)piperidine- 1-carboxylate | 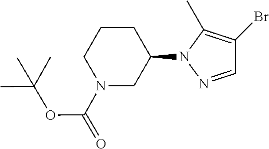 [SMILES: Cc1c(Br)cnn1[C@@H]1CCCN(C(=O)OC(C)(C)C)C1] | ^{b} |
| 38⁶ | tert-Butyl 3-(4-bromo- 5-methyl-pyrazol-1- yl)azetidine-1- carboxylate | 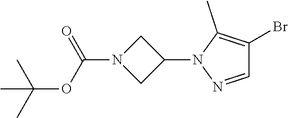 [SMILES: Cc1c(Br)cnn1C1CN(C(=O)OC(C)(C)C)C1] | 7.53 (s, 1H), 5.04-4.93 (m, 1H), 4.48- 4.40 (m, 2H), 4.36-4.28 (m, 2H), 2.27 (s, 3H), 1.48 (s, 9H) |

¹Purified by silica gel column chromatography, eluted with PE then PE/EA (1:10).
²¹H NMR (400 MHz, DMSO-d₆).
³Purified by silica gel chromatography and eluted with PE/EA (5/1 to 4/1).
⁴¹H NMR (300 MHz, DMSO-d₆).
⁵Purified by silica gel chromatography and eluted with PE/EA (15:1 to 12:1).
⁶Purified by silica gel chromatography and eluted with PE/EA (9:1 to 5:1).
^{a}ES/MS m/z (⁷⁹Br/⁸¹Br) 370/372 [M + H]⁺.
^{b}ES/MS m/z (⁷⁹Br/⁸¹Br) 344/346 [M + H]⁺.

### Preparation 39

### 3-(4-Bromo-5-methyl-triazol-1-yl)cyclobutanol

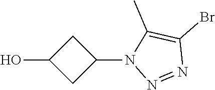

A mixture of 1-(3-benzyloxycyclobutyl)-4-bromo-5-methyl-triazole (8.5 g, 26.38 mmol) and FeCl₃ (8.56 g, 52.76 mmol) in DCM (100 mL) was stirred for 2 hr at 50° C. under N₂ then allowed to cool down to RT. The mixture was diluted with H₂O (50 mL) and extracted with EA (3×100 mL). The combined organic layers were washed with brine (2×100 mL), dried over Na₂SO₄, filtered and concentrated to afford the title compound (8 g, crude) as a brown solid. ¹H NMR (400 MHz, DMSOd₆) δ 5.09-4.99 (m, 1H), 4.55-4.42 (m, 1H), 2.80-2.71 (m, 2H), 2.48-2.37 (m, 2H), 2.21 (s, 3H).

### Preparation 40

### 4-(4-Bromo-5-methyl-pyrazol-1-yl)piperidine hydrochloride

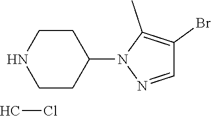

A soln of tert-butyl 4-(4-bromo-5-methyl-pyrazol-1-yl)piperidine-1-carboxylate (1.50 g, 4.4 mmol) in DCM (10 mL) was treated with HCl in 1,4-dioxane (4M, 10 mL) in portions at RT. After stirring at RT for 12 hr, the reaction was concentrated and the resulting solid triturated in Et₂O (15 mL). The solids were collected by filtration and washed with Et₂O (3×20 mL) to afford the title compound (1.2 g, 98%) as an off-white solid. ¹H NMR (300 MHz, DMSO-d6) δ 9.20 (brs, 1H), 8.85 (brs, 1H), 7.54 (s, 1H), 4.61-4.47 (m, 1H), 3.42-3.37 (m, 2H), 3.11-3.00 (m, 2H), 2.28 (s, 3H), 2.19-2.09 (m, 2H), 2.05-1.95 (m, 2H).

### Preparation 41

### 1-(Azetidin-3-yl)-4-bromo-5-methyl-pyrazole, 2,2,2-trifluoroacetic acid

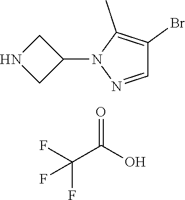

A soln of tert-butyl 3-(4-bromo-5-methyl-pyrazol-1-yl)azetidine-1-carboxylate (3.00 g, 9.5 mmol) in DCM (20 mL) was treated with TFA (10 mL) and stirred at RT for 2 hr. The reaction was concentrated to afford the title compound (4 g, crude) as a brown oil that was used without purification. ¹H NMR (300 MHz, CDCl₃) δ 7.50 (s, 1H), 5.16-5.06 (m, 1H), 4.30 (t, 2H), 3.88 (t, 2H), 2.25 (s, 3H).

### Preparation 42

### tert-Butyl (3S)-3-[3-(4-bromo-5-methyl-pyrazol-1-yl)azetidin-1-yl]pyrrolidine-1-carboxylate

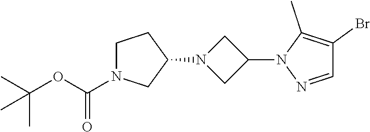 [SMILES: Cc1c(Br)cnn1C1CN([C@H]2CCN(C(=O)OC(C)(C)C)C2)C1]

A soln of tert-butyl (3R)-3-hydroxypyrrolidine-1-carboxylate (2.00 g, 10.7 mmol) and DIPEA (4.14 g, 32 mmol) in DCM (30 mL) at −40° C. was treated with trifluoromethanesulfonic anhydride (3.62 g, 12.8 mmol) and then stirred at −40° C. for 2 hr. The resulting mixture was added dropwise to a −40° C. soln of 1-(azetidin-3-yl)-4-bromo-5-methyl-pyrazole, 2,2,2-trifluoroacetic acid (4 g, crude) in DCM (30 mL) basified to pH ~10 with DIPEA (3 mL). The reaction was stirred at −40° C. for 1 hr and then at RT overnight. The reaction was concentrated and purified by C18 reverse phase chromatography, eluted with 20% to 30% ACN in H₂O (0.1% FA) to afford the title compound (0.50 g, 12% crude) as a light-yellow solid. ES/MS m/z (⁷⁹Br/⁸¹Br) 385/387 [M+H]⁺.

The following compound was prepared essentially as described in Preparation 42 using the appropriate reagents, adjusting temperature, adjusting the reaction times to determine completion of the reactions, and adjusting the purification system as appropriate.

TABLE 7

| | | | ES/MS m/z |
|---|---|---|---|
| Prep # | Chemical Name | Structure | [M + H]⁺ |
| 43¹ | tert-Butyl (3R)-3-[3-(4- bromo-5-methyl- pyrazol-1-yl)azetidin-1- yl]pyrrolidine-1- carboxylate | 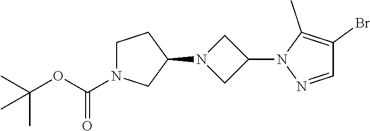 [SMILES: Cc1c(Br)cnn1C1CN([C@@H]2CCN(C(=O)OC(C)(C)C)C2)C1] | (⁷⁹Br/⁸¹Br) 385/387 |

¹Purified by C18 reverse phase flash chromatography eluted with 30% to 50% ACN in H₂O (0.1% FA).

### Preparation 44

### tert-Butyl N-[2-[4-(4-bromo-5-methyl-pyrazol-1-yl)-1-piperidyl]-1,1-dimethyl-ethyl]carbamate

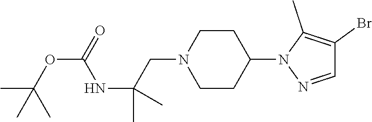 [SMILES: Cc1c(Br)cnn1C1CCN(CC(C)(C)NC(=O)OC(C)(C)C)CC1]

A soln of 4-(4-bromo-5-methyl-pyrazol-1-yl)piperidine hydrochloride (1.20 g, 4.9 mmol) and tert-butyl N-(1,1-dimethyl-2-oxo-ethyl)carbamate (1.38 g, 7.4 mmol) in MeOH (10 mL) was treated with AcOH (14.76 mg, 0.25 mmol) and the reaction stirred at RT for 30 min. The reaction was treated with NaBH₃CN (617.77 mg, 9.8 mmol) in portions at RT and then stirred for 12 hr at 50° C. After cooling to RT, the reaction was quenched with aqueous NH₄Cl (20 mL) and extracted with EA (3×50 mL). The combined organics were washed with brine (2×100 mL), collected, dried over Na₂SO₄, filtered, and concentrated. The residue was purified by silica gel chromatography and eluted with PE/EA (6:1 to 5:1) to afford the title compound (0.40 g, 20%) as an off-white solid. ¹H NMR (300 MHz, CDCl₃) δ 7.45 (s, 1H), 4.84 (brs, 1H), 4.06-3.88 (m, 1H), 3.02-2.98 (m, 2H), 2.54-2.38 (m, 4H), 2.29-2.21 (m, 5H), 1.82-1.77 (m, 2H), 1.45 (s, 9H), 1.27 (s, 6H).

### Preparation 45

### 7-Chloro-5-methoxyimidazo[1,2-a]pyridine

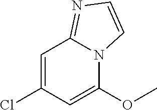

A soln of 4-chloro-6-methoxypyridin-2-amine (7.00 g, 44.14 mmol), chloroacetaldehyde (8.32 g, 52.99 mmol, 50%), and NaHCO₃ (11.12 g, 132.42 mmol) in n-butanol (140 mL) was divided into fourteen batches and stirred overnight at 65° C. in sealed tubes. The soln was cooled to RT, diluted with H₂O (200 mL) and extracted with EA (3×200 mL). The organic extracts were dried over Na₂SO₄ and concentrated. The reside was purified by silica gel chromatography and eluted with 50% EA in PE to afford the title compound as a light-brown solid (6.1 g, 76%). ES/MS m/z 183 [M+H]⁺.

### Preparation 46

### 6-Bromo-3-fluoro-4-methoxy-pyrazolo[1,5-a]pyridine

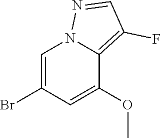

A soln of 6-bromo-4-methoxypyrazolo[1,5-a]pyridine (5 g, 22 mmol) and 1-chloromethyl-4-fluoro-1,4-diazoniabicyclo[2.2.2]octane bis(tetrafluoroborate) (7.04 g, 22 mmol) in ACN (50 mL) was stirred overnight at RT under N₂. The mixture was diluted with H₂O (50 mL) then extracted with EA (3×100 mL). The combined organic layers were washed with brine (2×100 mL), dried over Na₂SO₄, and filtered. The filtrate was concentrated in vacuo. The residue was purified by reverse phase chromatography with the following conditions: Column, C18; mobile phase, 30% to 40% ACN in H₂O (0.1% FA), over a 10 min. period to afford the title compound (810 mg, 15%) as a yellow solid. H NMR (300 MHz, DMSO-d₆) δ 8.56 (t, 1H), 8.04 (d, 1H), 6.77 (d, 1H), 3.97 (s, 3H).

### Preparation 47

### 6-Bromo-3-iodo-4-methoxy-pyrazolo[1,5-a]pyridine

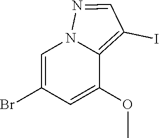

6-Bromo-4-methoxypyrazolo[1,5-a]pyridine (0.41 g, 1.8 mmol) and NIS (609 mg, 2.7 mmol) was dissolved in ACN (8 mL) and stirred at RT for 1 hr. The suspension was filtered, and the filtrate concentrated. The residue was purified by silica gel chromatography and eluted with a linear gradient of 0% to 100% EA in heptane. The fractions containing the title compound were concentrated in vacuo and combined with the filtered solids to afford the title compound (0.60 g, 94%). ES/MS m/z (⁷⁹Br/⁸¹Br) 353/355 [M+H]⁺.

### Preparation 48

### 6-Bromo-4-methoxy-3-(trifluoromethyl)pyrazolo[1,5-a]pyridine

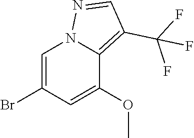 [SMILES: COc1cc(Br)cn2ncc(C(F)(F)F)c12]

A mixture of 6-bromo-3-iodo-4-methoxypyrazolo[1,5-a]pyridine (3.0 g, 8 mmol), methyl 2,2-difluoro-2-(fluorosulfonyl)acetate (2.0 g, 0.1 mol), and CuI (2.0 g, 0.1 mol) in DMF (36 mL) was heated for 2 hr at 80° C. The mixture was diluted with EA, filtered through DE, and placed in a separatory funnel. Additional EA (250 mL) was added, wash with 10% aq LiCl soln (20 mL×3), sat. aq NaCl, dried over Na₂SO₄ and filtered. The filtrate was concentrated, SiO₂ was added and the solid was loaded onto a silica gel column. The column was eluted with 0% to 30% EA in heptane. To afford the title compound (0.76 g, 2.57 mmol) as a white, amorphous solid. ES/MS m/z (⁷⁹Br/⁸¹Br) 295/297 [M+H]⁺.

### Preparation 49

### 6-Bromo-4-isopropoxy-pyrazolo[1,5-a]pyridine-3-carbonitrile

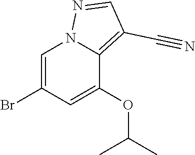 [SMILES: CC(C)Oc1cc(Br)cn2ncc(C#N)c12]

To a soln of 6-bromo-4-hydroxypyrazolo[1,5-a]pyridine-3-carbonitrile (5.00 g, 21.01 mmol) and 2-iodopropane (7.14 g, 42.01 mmol) in DMF (100 mL) was added K₂CO₃ (8.71 g, 63.01 mmol) at RT under N₂. The mixture was stirred for 1 hr at 80° C. The mixture was diluted with H₂O (150 mL) and extracted with EA (3×150 mL). The combined organic layers were washed with brine (3×300 mL), dried over Na₂SO₄, filtered and concentrated. The residue was purified by silica gel chromatography and eluted with PE/EA (6:1 to 5:1) to afford the title compound (5.0 g, 85%) as an off-white solid. ES/MS m/z (⁷⁹Br/⁸¹Br) 280/282 [M+H]⁺.

### Preparation 50

### 4-[2-Benzyloxy-1-(5-fluoro-2-pyridyl)ethoxy]-6-bromo-pyrazolo[1,5-a]pyridine

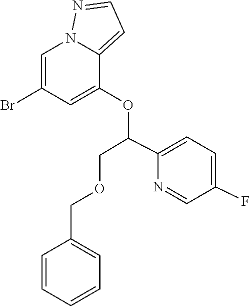 [SMILES: Fc1ccc(C(COCc2ccccc2)Oc2cc(Br)cn3nccc23)nc1]

To a mixture of PPh₃ (22.16 g, 84.5 mmol) in THE (25 mL) was added dropwise DIAD (15.38 g, 76 mmol) at 0° C. under N₂. The mixture was stirred for 0.5 hr then 2-benzyloxy-1-(5-fluoro-2-pyridyl)ethanol (12.54 g, 50.7 mmol) was added along with 6-bromopyrazolo[1,5-a]pyridin-4-ol (9 g, 42.2 mmol) in THE (25 mL). The mixture was stirred for 2 hr at RT then concentrated. The residue was purified by C18 reverse flash chromatography and eluted with 65% to 70% ACN in H₂O to afford the title compound (14 g, 75%) as a white solid. ES/MS m/z (⁷⁹Br/⁸¹Br) 440/442 [M+H]⁺.

The following compounds were prepared essentially as described in Preparation 50 using the appropriate reagents, adjusting temperature, adjusting the reaction times to determine completion of the reactions, and adjusting the purification system as appropriate.

TABLE 8

| | | | ES/MS m/z |
|---|---|---|---|
| Prep # | Chemical Name | Structure | [M + H]⁺ |
| 51¹ | 6-Bromo-4-[(1R)-1- cyclobutylethoxy]pyrazolo[1,5- a]pyridine-3-carbonitrile | 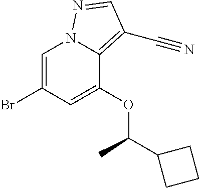 [SMILES: C[C@@H](Oc1cc(Br)cn2ncc(C#N)c12)C1CCC1] | (⁷⁹Br/⁸¹Br) 320/322 |
| 52² | 6-Bromo-4-[(1R)-1-(2- pyridyl)ethoxy]pyrazolo[1,5- a]pyridine-3-carbonitrile | 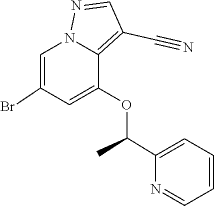 [SMILES: C[C@@H](Oc1cc(Br)cn2ncc(C#N)c12)c1ccccn1] | (⁷⁹Br/⁸¹Br) 343/345 |

¹Purified by silica gel chromatography eluted with PE/EA (4:1 to 1:2).
²DEAD used instead of DIAD.
³Purified by silica gel chromatography eluted with PE/EA (3:1).

### Preparation 53

### 4-Methoxy-6-(4,4,5,5-tetramethyl-1,3,2-dioxaborolan-2-yl)pyrazolo[1,5-a]pyridine

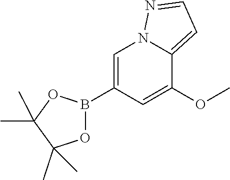

To 6-bromo-4-methoxypyrazolo[1,5-a]pyridine (5.00 g, 22 mmol) and bis(pinacolato)diboron (6.71 g, 26.4 mmol) in dioxane (10 mL) was added KOAc (6.48 g, 66.1 mmol) and Pd(dppf)Cl₂ (0.32 g, 0.44 mmol) at RT under N₂. The resulting mixture was stirred for 2 hr at 80° C. under N₂. The mixture was carried forward without a purification. ES/MS m/z 275 [M+H]⁺.

The following compounds were prepared essentially as described in Preparation 53 using the appropriate reagents, adjusting temperature, adjusting the reaction times to determine completion of the reactions, and adjusting the purification system as appropriate.

TABLE 9

| | | | ES/MS m/z |
|---|---|---|---|
| Prep # | Chemical Name | Structure | [M + H]⁺ |
| 54 | 3-Fluoro-4-methoxy-6- (4,4,5,5-tetramethyl-1,3,2- dioxaborolan-2- yl)pyrazolo[1,5-a]pyridine | 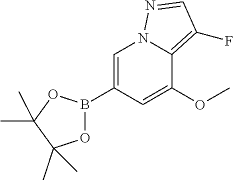 | 293 |
| 55 | 4-Isopropoxy-6-(4,4,5,5- tetramethyl-1,3,2- dioxaborolan-2- yl)pyrazolo[1,5-a]pyridine- 3-carbonitrile | 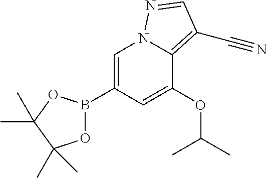 [SMILES: CC(C)Oc1cc(B2OC(C)(C)C(C)(C)O2)cn2ncc(C#N)c12] | 328 |
| 56 | 4-[(1R)-1- Cyclobutylethoxy]-6- (4,4,5,5-tetramethyl-1,3,2- dioxaborolan-2- yl)pyrazolo[1,5-a]pyridine- 3-carbonitrile | 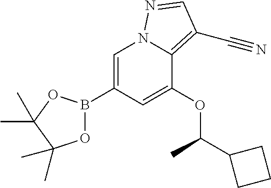 [SMILES: C[C@@H](Oc1cc(B2OC(C)(C)C(C)(C)O2)cn2ncc(C#N)c12)C1CCC1] | 368 |
| 57^{2,3} | 4-[(1R)-1-(2- Pyridyl)ethoxy]-6-(4,4,5,5- tetramethyl-1,3,2- dioxaborolan-2- yl)pyrazolo[1,5-a]pyridine- 3-carbonitrile | 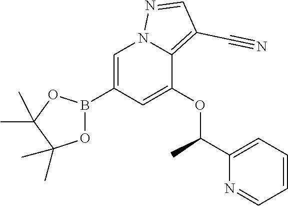 [SMILES: C[C@@H](Oc1cc(B2OC(C)(C)C(C)(C)O2)cn2ncc(C#N)c12)c1ccccn1] | 391 |

¹Workup: Reaction filtered, and the filter cake was washed with 1,4-dioxane.
²Pd(dppf)Cl₂·CH₂Cl₂ used as the catalyst.
³Workup: Reaction filtered, and the filter cake was washed with EA. The filtrate was concentrated to afford title the compound.

### Preparation 58

### [4-Methoxy-3-(trifluoromethyl)pyrazolo[1,5-a]pyridin-6-yl]boronic acid

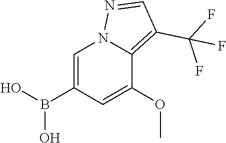 [SMILES: COc1cc(B(O)O)cn2ncc(C(F)(F)F)c12]

A mixture of 6-bromo-4-methoxy-3-(trifluoromethyl)pyrazolo[1,5-a]pyridine (1.69 g, 5.7 mmol), 4,4,4′,4′,5,5,5′,5′-octamethyl-2,2′-bi(1,3,2-dioxaborolane) (1.74 g, 6.9 mmol), KOAc (1.69 g, 17.2 mmol) and PdCl₂(dppf) (0.42 g, 0.6 mmol) in 1,4-dioxane (15 mL) was sparged with argon (15 min). The mixture was heated at 80° C. for 16 hr. The mixture was cooled to RT, diluted with EA, washed with H₂O (100 mL), brine (100 mL), dried over Na₂SO₄, filtered, and concentrated. The residue was purified by silica gel chromatography and eluted with 0% to 50% EA in heptane to afford the title compound (1.55 g, 104%). The title compound was used directly in the next step without further purification. ES/MS m/z 261 [M+H]⁺.

### Preparation 59

### 5-Methoxyimidazo[1,2-a]pyridin-7-ylboronic acid

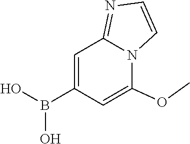

A stirred mixture of 7-chloro-5-methoxyimidazo[1,2-a]pyridine (1.00 g, 5.5 mmol) and bis(pinacolato)diboron (1.67 g, 6.6 mmol) in 1,4-dioxane was treated with KOAc (1.61 g, 16.4 mmol) and Xphos Pd G4 (0.05 g, 0.06 mmol) at RT under N₂ and stirred for 8 hr at 80° C. The mixture was diluted with H₂O (100 mL), acidified to pH 4 with 1 N aq HCl, and extracted with i-PrOH/CHCl₃ (3:1)(3×200 mL). The combined organic extracts were dried over Na₂SO₄ and concentrated in vacuo to give the title compound as a light-pink solid (1.4 g, crude). ES/MS m/z 193 [M+H]⁺.

### Preparation 60

### (1r, 3r)-3-[4-[4-[2-Benzyloxy-1-(5-fluoro-2-pyridyl)ethoxy]pyrazolo[1,5-a]pyridin-6-yl]-5-methyl-triazol-1-yl]cyclobutanol

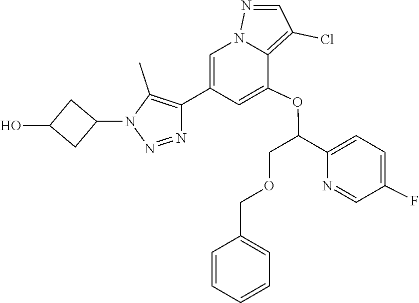 [SMILES: Cc1c(-c2cc(OC(COCc3ccccc3)c3ccc(F)cn3)c3c(Cl)cnn3c2)nnn1C1CC(O)C1]

To a stirred mixture of 4-[2-benzyloxy-1-(5-fluoro-2-pyridyl)ethoxy]-6-bromo-pyrazolo[1,5-a]pyridine (14 g, 27.1 mmol), bis(pinacolato)diboron (8.27 g, 32.6 mmol) and KOAc (7.99 g, 81.4 mmol) in 1,4-dioxane (120 mL) was added Pd(dppf)Cl₂·CH₂Cl₂ (0.99 g, 1.4 mmol) at RT under N₂. The mixture was stirred for 2 hr at 100° C. The reaction was not processed, but directly used in next step. To the above mixture were added K₂CO₃ (11.25 g, 81.4 mmol), (1r, 3r)-3-(4-bromo-5-methyl-triazol-1-yl)cyclobutanol (8.14 g, 35.3 mmol) and Pd(PPh₃)₄ (1.56 g, 1.4 mmol) and H₂O (30 mL). The mixture was stirred for 2 hr at 100° C. under N₂. Upon cooling to RT, the reaction was quenched with H₂O. The mixture was extracted with EA (3×800 mL). The combined organic layers were washed with brine (3×800 mL), dried over Na₂SO₄, filtered and concentrated. The residue was purified by silica gel chromatography and eluted with CH₂Cl₂/MeOH (50:1 to 20:1) to afford the title compound (12 g, 82%) as a white solid. ES/MS m/z 515 [M+H]⁺.

### Preparation 61

### tert-Butyl 4-[4-(4-methoxypyrazolo[1,5-a]pyridin-6-yl)-5-methyl-pyrazol-1-yl]piperidine-1-carboxylate

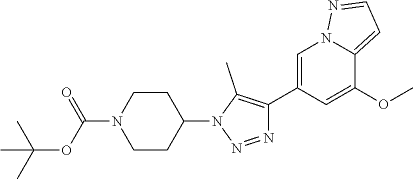 [SMILES: COc1cc(-c2nnn(C3CCN(C(=O)OC(C)(C)C)CC3)c2C)cn2nccc12]

A mixture of tert-butyl 4-[5-methyl-4-(4,4,5,5-tetramethyl-1,3,2-dioxaborolan-2-yl)pyrazol-1-yl]piperidine-1-carboxylate (200 mg, 0.51 mmol), 6-bromo-4-methoxy-pyrazolo[1,5-a]pyridine (239.4 mg, 1 mmol), and Pd(PPh₃)₄ (124 mg, 0.1 mmol) in aq K₂CO₃ (2 M, 0.5 mL) and 1,4-dioxane (3 mL) was heated to 125° C. in a microwave and maintained at that temperature for 3 hr. The reaction was loaded onto a reverse phase cartridge and eluted with 5% to 100% ACN (0.1% TFA) in H₂O (0.1% TFA) to afford the title compound (182 mg, 87%). ES/MS m/z 412 [M+H]⁺.

### Preparation 62

### tert-Butyl 4-[4-(4-methoxypyrazolo[1,5-a]pyridin-6-yl)-5-methyl-triazol-1-yl]piperidine-1-carboxylate

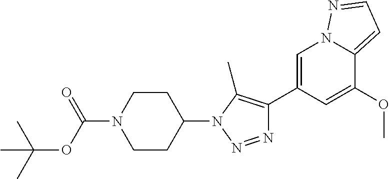 [SMILES: COc1cc(-c2nnn(C3CCN(C(=O)OC(C)(C)C)CC3)c2C)cn2nccc12]

To 4-methoxy-6-(4,4,5,5-tetramethyl-1,3,2-dioxaborolan-2-yl)pyrazolo[1,5-a]pyridine (5 g, 18.2 mmol) and tert-butyl 4-(4-bromo-5-methyl-1,2,3-triazol-1-yl)piperidine-1-carboxylate (6.93 g, 20.1 mmol) in 1,4-dioxane (80 mL) and H₂O (20 mL) was added K₂CO₃ (7.56 g, 54.7 mmol) and Pd(DtBPF)Cl₂ (0.24 g, 0.36 mmol) at RT under N₂. The reaction was stirred overnight at 80° C. Upon cooling to RT, the resultant mixture was diluted with H₂O (50 mL) and extracted with EA (3×100 mL). The combined organic layers are washed with brine (2×80 mL), dried over Na₂SO₄, filtered, and concentrated in vacuo. The residue was purified by silica gel chromatography eluted with a gradient of 30% to 60% EA in PE to afford the title compound as a light-brown solid (4.5 g, 60%). ES/MS m/z 413 [M+H]⁺.

The following compounds were prepared essentially as described in Preparation 62 using the appropriate reagents, adjusting temperature, adjusting the reaction times to determine completion of the reactions, and adjusting the purification system as appropriate.

TABLE 10

| | | | ES/MS m/z |
|---|---|---|---|
| Prep # | Chemical Name | Structure | [M + H]⁺ |
| 63^{1,2} | tert-Butyl 4-[4-(3- fluoro-4-methoxy- pyrazolo[1,5- a]pyridin-6-yl)-5- methyl-triazol-1- yl]piperidine-1- carboxylate | 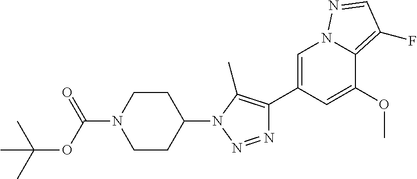 [SMILES: COc1cc(-c2nnn(C3CCN(C(=O)OC(C)(C)C)CC3)c2C)cn2ncc(F)c12] | 431 |

¹KOAc used as base.
²Purified by silica gel chromatography eluted with PE/EA (2:1-1:1)

### Preparation 64

### tert-Butyl 4-[4-(3-cyano-4-methoxy-pyrazolo[1,5-a]pyridin-6-yl)-5-methyl-pyrazol-1-yl]piperidine-1-carboxylate

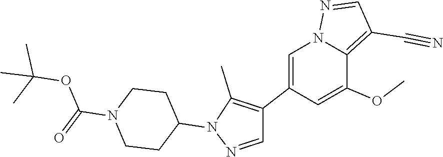 [SMILES: COc1cc(-c2cnn(C3CCN(C(=O)OC(C)(C)C)CC3)c2C)cn2ncc(C#N)c12]

A mixture of tert-butyl 4-(4-bromo-5-methyl-pyrazol-1-yl)piperidine-1-carboxylate (2 g, 5.8 mmol), 4-methoxy-6-(4,4,5,5-tetramethyl-1,3,2-dioxaborolan-2-yl)pyrazolo[1,5-a]pyridine-3-carbonitrile (2.61 g, 8.7 mmol), 2 M aq Na₂CO₃ (6.1 mL, 12.2 mmol), and Pd(PPh₃)₄ (0.67 g, 0.58 mmol) in 1,4-dioxane (19.4 mL) was heated to 90° C. overnight. Upon cooling to RT, the reaction was treated with DCM and H₂O and the layers were separated. The organic layer was washed with brine, dried over Na₂SO₄, filtered, and concentrated. The residue was purified by silica gel chromatography and eluted with 10% to 90% EA in hex to afford the title compound (738 mg, 29% yield). ES/MS m/z 437 [M+H]⁺.

### Preparation 65

### tert-Butyl 3-[4-(3-cyano-4-methoxy-pyrazolo[1,5-a]pyridin-6-yl)-5-methyl-pyrazol-1-yl]azetidine-1-carboxylate

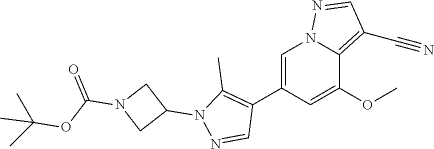 [SMILES: COc1cc(-c2cnn(C3CN(C(=O)OC(C)(C)C)C3)c2C)cn2ncc(C#N)c12]

A soln of tert-butyl 3-(4-bromo-5-methylpyrazol-1-yl)azetidine-1-carboxylate (2.50 g, 7.9 mmol) and 4-methoxy-6-(4,4,5,5-tetramethyl-1,3,2-dioxaborolan-2-yl)pyrazolo[1,5-a]pyridine-3-carbonitrile (2.37 g, 7.9 mmol) in 1,4-dioxane (80 mL) and H₂O (20 mL) were added K₂CO₃ (3.28 g, 23.7 mmol) and Pd(PPh₃)₄ (0.09 g, 0.08 mmol) at RT under N₂. The mixture was stirred overnight at 100° C. Upon cooling to RT, the reaction was quenched with H₂O (100 mL) then extracted with EA (3×150 mL). The combined organic layers were washed with brine (150 mL), dried over Na₂SO₄, filtered, and concentrated. The residue was purified by silica gel chromatography and eluted with PE/EA (10:1 to 1:1) to afford the title compound (1.88 g, 58%) as a white solid. ¹H NMR (300 MHz, DMSO-d₆) δ 8.61-8.51 (m, 2H), 7.95 (s, 1H), 7.09 (d, 1H), 5.41-5.26 (m, 1H), 4.37-4.26 (m, 2H), 4.18 (d, 2H), 4.06 (s, 3H), 2.41 (s, 3H), 1.43 (s, 9H).

The following compounds were prepared essentially as described in Preparation 65 using the appropriate reagents, adjusting temperature, adjusting the reaction times to determine completion of the reactions, and adjusting the purification system as appropriate.

TABLE 11

| Prep | | | ES/MS m/z |
|---|---|---|---|
| # | Chemical Name | Structure | [M + H]⁺ |
| 66¹ | tert-Butyl (3R)-3-[4-(3- cyano-4-methoxy- pyrazolo[1,5-a]pyridin-6- yl)-5-methyl-pyrazol-1- yl]pyrrolidine-1- carboxylate | 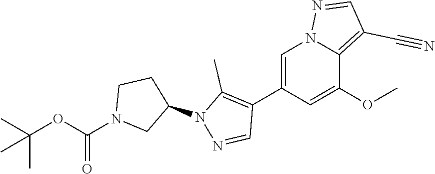 [SMILES: COc1cc(-c2cnn([C@@H]3CCN(C(=O)OC(C)(C)C)C3)c2C)cn2ncc(C#N)c12] | 423 |
| 67² | tert-Butyl 3-[4-(3-cyano- 4-isopropoxy- pyrazolo[1,5-a]pyridin-6- yl)-5-methyl-triazol-1- yl]azetidine-1-carboxylate | 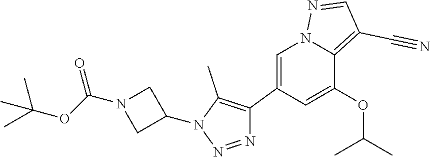 [SMILES: Cc1c(-c2cc(OC(C)C)c3c(C#N)cnn3c2)nnn1C1CN(C(=O)OC(C)(C)C)C1] | 479 [M + ACN + H]⁺ |
| 68³ | tert-Butyl 3-[4-(3-cyano- 4-methoxy-pyrazolo[1,5- a]pyridin-6-yl)-5-methyl- triazol-1-yl]azetidine-1- carboxylate | 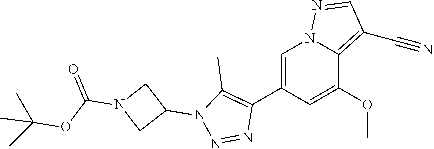 [SMILES: COc1cc(-c2nnn(C3CN(C(=O)OC(C)(C)C)C3)c2C)cn2ncc(C#N)c12] | 410 |
| 69 | tert-Butyl 4-(4-[5- methoxyimidazo[1,2- a]pyridin-7-yl]-5-methyl- 1,2,3-triazol-1- yl)piperidine-1- carboxylate | 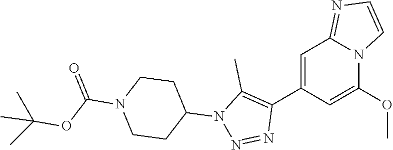 [SMILES: COc1cc(-c2nnn(C3CCN(C(=O)OC(C)(C)C)CC3)c2C)cc2nccn12] | 413 |
| 70^{4,5} | tert-Butyl 3-[(1R)-4-[3- cyano-4-[(1R)-1- cyclobutylethoxy]pyrazolo [1,5-a]pyridin-6-yl]-5- methyl-pyrazol-1- yl]piperidine-1- carboxylate | 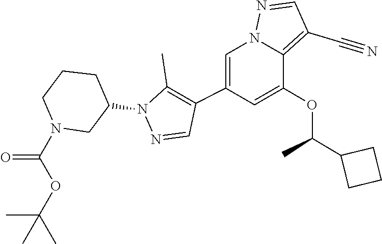 | 505 |
| 71⁶ | tert-Butyl (3S)-3-[4-[3- cyano-4-[(1R)-1-(2- pyridyl)ethoxy]pyrazolo [1,5-a]pyridin-6-yl]-5- methyl-triazol-1- yl]piperidine-1- carboxylate | 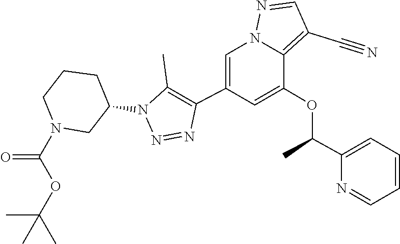 [SMILES: Cc1c(-c2cc(O[C@H](C)c3ccccn3)c3c(C#N)cnn3c2)nnn1[C@H]1CCCN(C(=O)OC(C)(C)C)C1] | 529 |
| 72 | tert-Butyl (1R,3r,5S)-3-(4- (3-cyano-4- methoxypyrazolo[1,5- a]pyridin-6-yl)-5-methyl- 1H-pyrazol-1-yl)-8- azabicyclo[3.2.1]octane-8- carboxylate | 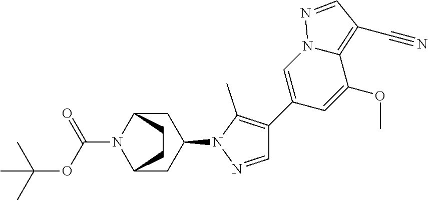 [SMILES: COc1cc(-c2cnn([C@H]3C[C@H]4CC[C@@H](C3)N4C(=O)OC(C)(C)C)c2C)cn2ncc(C#N)c12] | 463 |
| 73 | tert-Butyl (1R,3s,5S)-3- (4-(3-cyano-4- methoxypyrazolo[1,5- a]pyridin-6-yl)-5-methyl- 1H-pyrazol-1-yl)-8- azabicyclo[3.2.1]octane-8- carboxylate | 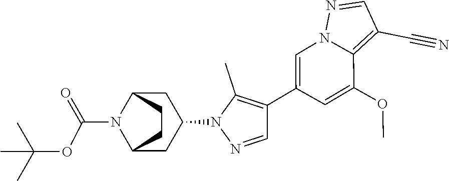 [SMILES: COc1cc(-c2cnn([C@@H]3C[C@H]4CC[C@@H](C3)N4C(=O)OC(C)(C)C)c2C)cn2ncc(C#N)c12] | 463 |
| 74⁷ | tert-Butyl (3S)-3-[4-(3- cyano-4-methoxy- pyrazolo[1,5-a]pyridin-6- yl)-5-methyl-pyrazol-1- yl]pyrrolidine-1- carboxylate | 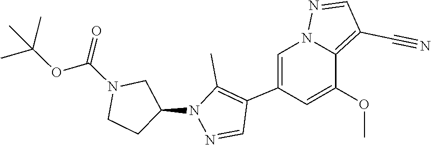 [SMILES: COc1cc(-c2cnn([C@H]3CCN(C(=O)OC(C)(C)C)C3)c2C)cn2ncc(C#N)c12] | 423 |
| 75⁸ | tert-Butyl 4-[4-(3-cyano- 4-isopropoxy- pyrazolo[1,5-a]pyridin-6- yl)-5-methyl-pyrazol-1- yl]piperidine-1- carboxylate | 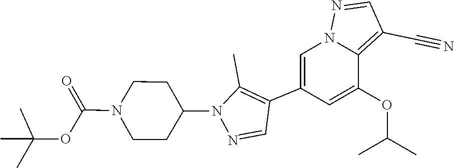 [SMILES: Cc1c(-c2cc(OC(C)C)c3c(C#N)cnn3c2)cnn1C1CCN(C(=O)OC(C)(C)C)CC1] | 465 |
| 76⁹ | tert-Butyl 2-[4-(3-cyano- 4-methoxy-pyrazolo[1,5- a]pyridin-6-yl)-5-methyl- pyrazol-1-yl]-7- azaspiro[3.5]nonane-7- carboxylate | 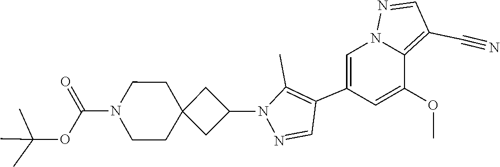 [SMILES: COc1cc(-c2cnn(C3CC4(CCN(C(=O)OC(C)(C)C)CC4)C3)c2C)cn2ncc(C#N)c12] | 462 [M − CH₃ + H]⁺ |
| 77^{4,9} | tert-Butyl N-[2-[4-[4-(3- cyano-4-methoxy- pyrazolo[1,5-a]pyridin-6- yl)-5-methyl-pyrazol-1- yl]-1-piperidyl]-1,1- dimethyl-ethyl]carbamate | 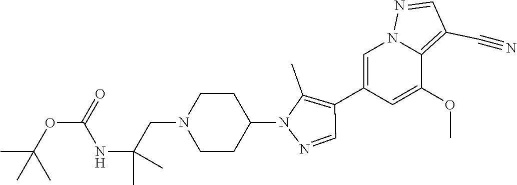 [SMILES: COc1cc(-c2cnn(C3CCN(CC(C)(C)NC(=O)OC(C)(C)C)CC3)c2C)cn2ncc(C#N)c12] | 508 |
| 78¹⁰ | tert-Butyl 4-[4-(3-cyano- 4-methoxy-pyrazolo[1,5- a]pyridin-6-yl)-5-methyl- triazol-1-yl]piperidine-1- carboxylate | 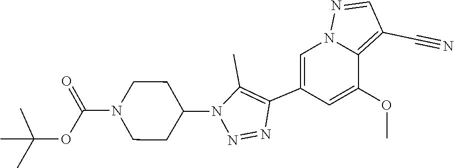 [SMILES: COc1cc(-c2nnn(C3CCN(C(=O)OC(C)(C)C)CC3)c2C)cn2ncc(C#N)c12] | 438 |
| 79¹¹ | tert-Butyl (3S)-3-[4-(3- cyano-4-methoxy- pyrazolo[1,5-a]pyridin-6- yl)-5-methyl-pyrazol-1- yl]piperidine-1- carboxylate | 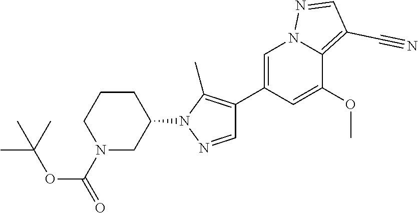 [SMILES: COc1cc(-c2cnn([C@H]3CCCN(C(=O)OC(C)(C)C)C3)c2C)cn2ncc(C#N)c12] | 437 |
| 80¹² | tert-Butyl (3R)-3-[4-(3- cyano-4-methoxy- pyrazolo[1,5-a]pyridin-6- yl)-5-methyl-pyrazol-1- yl]piperidine-1- carboxylate | 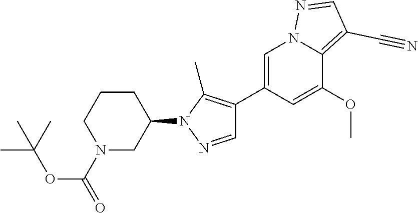 [SMILES: COc1cc(-c2cnn([C@@H]3CCCN(C(=O)OC(C)(C)C)C3)c2C)cn2ncc(C#N)c12] | 437 |
| 81¹³ | tert-Butyl (3S)-3-[3-[4-(3- cyano-4-methoxy- pyrazolo[1,5-a]pyridin-6- yl)-5-methyl-pyrazol-1- yl]azetidin-1- yl]pyrrolidine-1- carboxylate | 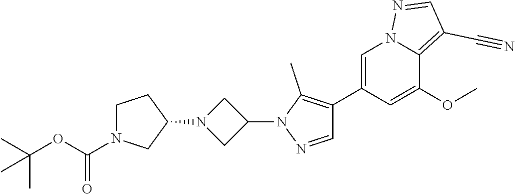 [SMILES: COc1cc(-c2cnn(C3CN([C@H]4CCN(C(=O)OC(C)(C)C)C4)C3)c2C)cn2ncc(C#N)c12] | 478 |
| 82¹⁴ | tert-Butyl (3R)-3-[3-[4-(3- cyano-4-methoxy- pyrazolo[1,5-a]pyridin-6- yl)-5-methyl-pyrazol-1- yl]azetidin-1- yl]pyrrolidine-1- carboxylate | 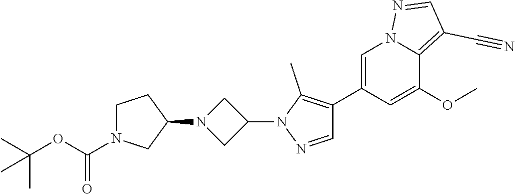 [SMILES: COc1cc(-c2cnn(C3CN([C@@H]4CCN(C(=O)OC(C)(C)C)C4)C3)c2C)cn2ncc(C#N)c12] | 478 |
| 83¹⁵ | tert-Butyl (3R)-3-[4-(3- cyano-4-methoxy- pyrazolo[1,5-a]pyridin-6- yl)-5-methyl-triazol-1- yl]pyrrolidine-1- carboxylate | 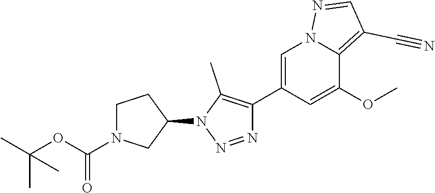 [SMILES: COc1cc(-c2nnn([C@@H]3CCN(C(=O)OC(C)(C)C)C3)c2C)cn2ncc(C#N)c12] | 424 |
| 84¹⁶ | tert-Butyl (3S)-3-[4-(3- cyano-4-methoxy- pyrazolo[1,5-a]pyridin-6- yl)-5-methyl-triazol-1- yl]pyrrolidine-1- carboxylate | 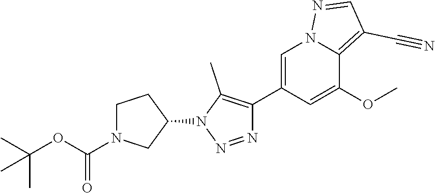 [SMILES: COc1cc(-c2nnn([C@H]3CCN(C(=O)OC(C)(C)C)C3)c2C)cn2ncc(C#N)c12] | 424 |

¹Purified by silica gel chromatography eluted with PE/EA (5:1 to 1:1).
²Purified by C18 reverse flash chromatography and eluted with 60% to 63% ACN in H₂O (0.1% NH₄HCO₃).
³Purified by silica gel chromatography and eluted with PE/EA (5:1 to 1:1).
⁴Pd(dppf)Cl₂ used as catalyst.
⁵Purified by C18 reverse flash chromatography and eluted with 60% to 70% ACN in H₂O.
⁶Purified by silica gel chromatography and eluted with PE/EA (1:2).
⁷Purified by silica gel chromatography and eluted with PE/EA (3:1 to 1:1).
⁸Purified by silica gel chromatography and eluted with PE/EA (4:1 to 3:1).
⁹Purified by silica gel chromatography and eluted with PE/EA (3:1 to 2:1).
¹⁰Purified by preparative TLC eluted with PE/EA (1:1).
¹¹Pd(OAc)₂ and PCy₃ used as catalyst with K₃PO₄ used as base and toluene/H₂O used as solvent. Purified by silica gel chromatography and eluted with PE/EA (5:1).
¹²Pd(OAc)₂ and PCy₃ used as catalyst with K₃PO₄ used as base and toluene/H₂O used as solvent. Purified by silica gel chromatography and eluted with PE/EA (10:1 to 8:1).
¹³KF used as the base. Purified by C18 reverse phase chromatography and eluted with 35% to 45% ACN in H₂O (0.1% NH₃H₂O).
¹⁴KF used as the base. Purified by silica gel chromatography and eluted with PE/EA (3:1 to 1:1).
¹⁵Purified by C18 reverse phase flash chromatography and eluted with 30% to 50% ACN in H₂O (0.1% FA).
¹⁶Purified by C18 reverse phase flash chromatography and eluted with 44% to 49% ACN in H₂O (0.1% NH₄HCO₃).

### Preparation 85

### tert-Butyl (3S,4R)-3-hydroxy-4-[4-[4-methoxy-3-(trifluoromethyl)pyrazolo[1,5-a]pyridin-6-yl]-5-methyl-triazol-1-yl]piperidine-1-carboxylate

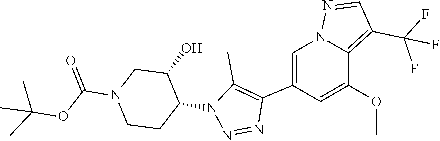 [SMILES: COc1cc(-c2nnn([C@@H]3CCN(C(=O)OC(C)(C)C)C[C@@H]3O)c2C)cn2ncc(C(F)(F)F)c12]

A soln of [4-methoxy-3-(trifluoromethyl)pyrazolo[1,5-a]pyridin-6-yl]boronic acid (178 mg, 0.68 mmol) and tert-butyl (3S,4R)-4-(4-bromo-5-methyl-triazol-1-yl)-3-hydroxy-piperidine-1-carboxylate (225 mg, 0.62 mmol) in DMF (2.5 mL) was added K₃PO₄ in H₂O (4M, 528 mg, 2.5 mmol). The mixture was sparged with N₂ then Xphos Pd G2 was added (24 mg, 0.03 mmol). Mixture was re-sparged with N₂ then heated at 65° C. for 3 hr. The reaction was diluted with H₂O (100 mL), extracted with EA, dried over Na₂SO₄, filtered, and concentrated. The residue was purified by reverse phase chromatography eluted with ACN in H₂O to afford the title compound (214 mg, 69%) as light green solid. ES/MS m/z 497 [M+H].

### Preparation 86

### tert-Butyl 4-[4-(3-chloro-4-methoxy-pyrazolo[1,5-a]pyridin-6-yl)-5-methyl-triazol-1-yl]piperidine-1-carboxylate

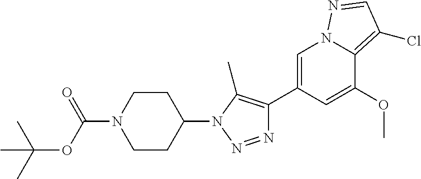 [SMILES: COc1cc(-c2nnn(C3CCN(C(=O)OC(C)(C)C)CC3)c2C)cn2ncc(Cl)c12]

A soln of tert-butyl 4-[4-(4-methoxypyrazolo[1,5-a]pyridin-6-yl)-5-methyl-triazol-1-yl]piperidine-1-carboxylate (16.1 g, 39 mmol) in CHCl₃ (50 mL) was treated with PPTS (981 mg, 3.9 mmol) followed by NCS (5.21 g, 39 mmol). The reaction was stirred at 40° C. for 95 min. Upon cooling to RT, the reaction was concentrated in vacuo. The residue was purified by silica gel chromatography and eluted with 0% to 100% EA in heptane to afford the title compound (17.7 g, 101%) as a colorless solid. ES/MS m/z 391 [M+2H-tBu]⁺.

### Preparation 87

### 3-[4-[4-[2-Benzyloxy-1-(5-fluoro-2-pyridyl)ethoxy]-3-chloro-pyrazolo[1,5-a]pyridin-6-yl]-5-methyl-triazol-1-yl]cyclobutanol

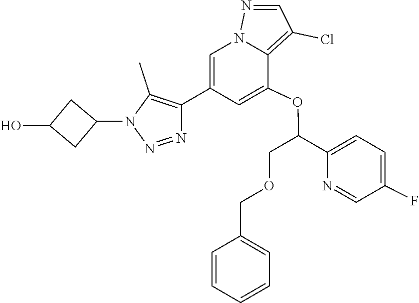 [SMILES: Cc1c(-c2cc(OC(COCc3ccccc3)c3ccc(F)cn3)c3c(Cl)cnn3c2)nnn1C1CC(O)C1]

A soln of 3-[4-[4-[2-benzyloxy-1-(5-fluoro-2-pyridyl)ethoxy]pyrazolo[1,5-a]pyridin-6-yl]-5-methyl-triazol-1-yl]cyclobutanol (12 g, 23.3 mmol) in DCM (120 mL) was treated with NCS (2.96 g, 22.2 mmol) at RT. The mixture was stirred at RT for 2 hr under N₂. The mixture was then diluted with H₂O (500 mL) and extracted with DCM (3×500 mL). The combined organic layers were washed with brine (2×500 mL), dried over Na₂SO₄, filtered, and concentrated. The residue was purified by C18 reverse flash chromatography and eluted with 50% to 60% ACN in H₂O to afford the title compound (12 g, 94%) as a white solid. ES/MS m/z 549 [M+H]⁺.

### Preparation 88

### 4-(2-(Benzyloxy)-1-(5-fluoropyridin-2-yl)ethoxy)-6-(1-(3-((tert-butyldimethylsilyl)oxy)cyclobutyl)-5-methyl-1H-1,2,3-triazol-4-yl)-3-chloropyrazolo[1,5-a]pyridine, Isomer 1

### and

### Preparation 89

### 4-(2-(Benzyloxy)-1-(5-fluoropyridin-2-yl)ethoxy)-6-(1-(3-((tert-butyldimethylsilyl)oxy)cyclobutyl)-5-methyl-1H-1,2,3-triazol-4-yl)-3-chloropyrazolo[1,5-a]pyridine, Isomer 2

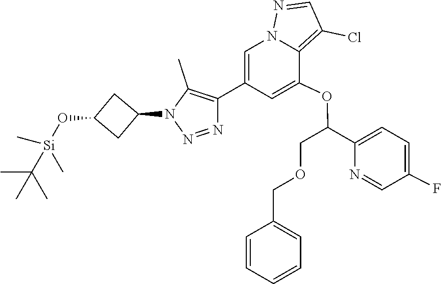 [SMILES: Cc1c(-c2cc(OC(COCc3ccccc3)c3ccc(F)cn3)c3c(Cl)cnn3c2)nnn1[C@H]1C[C@H](O[Si](C)(C)C(C)(C)C)C1]

To a mixture of (1r, 3r)-3-[4-[4-[2-benzyloxy-1-(5-fluoro-2-pyridyl)ethoxy]-3-chloro-pyrazolo[1,5-a]pyridin-6-yl]-5-methyl-triazol-1-yl]cyclobutanol (12 g, 21.86 mmol) and imidazole (2.98 g, 43.72 mmol) in DCM (120 mL) was added TBDMSCl (3.95 g, 26.23 mmol) at RT under N₂. The mixture was stirred for 1 hr. The reaction was quenched with H₂O and extracted with EA (3×500 mL). The combined organic layers were washed with brine (3×500 mL), dried over Na₂SO₄, filtered and concentrated. The residue was purified by silica gel chromatography and eluted with PE/EA (4:1) to afford the title compound as a racemate (9.5 g, 66%) as a white solid. ES/MS m/z 663 [M+H]⁺.

The enantiomers were isolated by prep-chiral HPLC that used the following conditions: Column: NB_ASA CHIRALPAK IG_2, 5×30 cm, 10 m; eluted with 35% MeOH/DCM (1:1)(0.1% DEA) in CO₂ to afford 4-(2-(benzyloxy)-1-(5-fluoropyridin-2-yl)ethoxy)-6-(1-(3-((tert-butyldimethylsilyl)oxy)cyclobutyl)-5-methyl-1H-1,2,3-triazol-4-yl)-3-chloropyrazolo[1,5-a]pyridine, Isomer 1, ES/MS m/z 663 [M+H]*, t_{(R)} is 6.31 min (4.0 g, 42%), ee 98% and 4-(2-(Benzyloxy)-1-(5-fluoropyridin-2-yl)ethoxy)-6-(1-(3-((tert-butyldimethylsilyl)oxy)cyclobutyl)-5-methyl-1H-1,2,3-triazol-4-yl)-3-chloropyrazolo[1,5-a]pyridine, Isomer 2, ES/MS m/z 663, [M+H]*, t_{(R)} is 8.02 min (4.3 g, 45%), ee 98%.

### Preparation 90

### (1r, 3r)-3-[4-[4-[2-Benzyloxy-1-(5-fluoro-2-pyridyl)ethoxy]-3-chloro-pyrazolo[1,5-a]pyridin-6-yl]-5-methyl-triazol-1-yl]cyclobutanol, Isomer 1

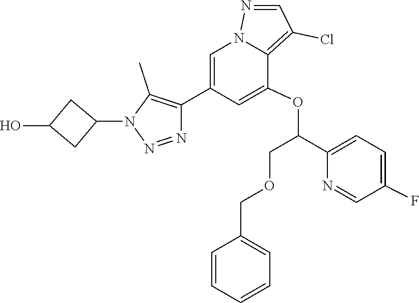 [SMILES: Cc1c(-c2cc(OC(COCc3ccccc3)c3ccc(F)cn3)c3c(Cl)cnn3c2)nnn1C1CC(O)C1]

To a soln of 4-(2-(benzyloxy)-1-(5-fluoropyridin-2-yl)ethoxy)-6-(1-(3-((tert-butyldimethylsilyl)oxy)cyclobutyl)-5-methyl-1H-1,2,3-triazol-4-yl)-3-chloropyrazolo[1,5-a]pyridine, Isomer 1 (4.0 g, 6.03 mmol) in MeOH (40 mL) was added HCl (2.97 g, 30.16 mmol, 37%) at RT under N₂. The mixture was stirred for 2 hr. The reaction was quenched with sat. aq NaHCO₃ (30 mL). The crude product was re-crystallized from H₂O/PE (1:1, 400 mL) to afford the title compound (2.07 g, 63%) as a white solid. ES/MS m/z 549 [M+H]⁺.

### Preparation 91

### tert-Butyl 4-(4-[3-iodo-5-methoxyimidazo[1,2-a]pyridin-7-yl]-5-methyl-1,2,3-triazol-1-yl)piperidine-1-carboxylate

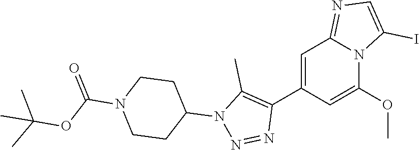 [SMILES: COc1cc(-c2nnn(C3CCN(C(=O)OC(C)(C)C)CC3)c2C)cc2ncc(I)n12]

A stirred RT soln of tert-butyl 4-(4-[5-methoxyimidazo[1,2-a]pyridin-7-yl]-5-methyl-1,2,3-triazol-1-yl)piperidine-1-carboxylate (500 mg, 1.2 mmol) in DCM (6 mL) was treated with NIS (300 mg, 1.3 mmol) and stirred for 8 hr at RT. The mixture was diluted with EA (100 mL), washed with H₂O (20 mL), and brine (20 mL). The combined organic extracts were dried over Na₂SO₄, filtered, and the filtrate concentrated in vacuo to afford the title compound (820 mg, crude) which was used directly without further purification. ES/MS m/z 539 [M+H]⁺.

### Preparation 92

### tert-Butyl 4-(4-[3-cyano-5-methoxyimidazo[1,2-a]pyridin-7-yl]-5-methyl-1,2,3-triazol-1-yl)piperidine-1-carboxylate

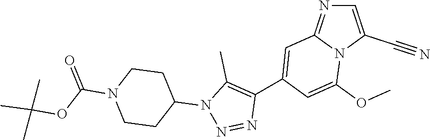 [SMILES: COc1cc(-c2nnn(C3CCN(C(=O)OC(C)(C)C)CC3)c2C)cc2ncc(C#N)n12]

A soln of tert-butyl 4-(4-[3-iodo-5-methoxyimidazo[1,2-a]pyridin-7-yl]-5-methyl-1,2,3-triazol-1-yl)piperidine-1-carboxylate (800 mg, crude), DMF (8 mL), and CuCN (173.01 mg, 1.9 mmol) was stirred for 2 hr at 100° C. under N₂. The mixture was diluted with DCM (100 mL), washed with H₂O (20 mL), and then washed with brine (20 mL). The combined organic layers were dried over Na₂SO₄, filtered, and the filtrate was concentrated under in vacuo. The residue was purified by reverse flash chromatography with the following conditions: Column, C18; eluting with a gradient of 50% to 70% ACN in H₂O (0.1% FA) to give the title compound (450 mg, 46%). ES/MS m/z 438 [M+H]⁺.

### Preparation 93

### tert-Butyl 4-[4-(3-chloro-4-hydroxy-pyrazolo[1,5-a]pyridin-6-yl)-5-methyl-triazol-1-yl]piperidine-1-carboxylate

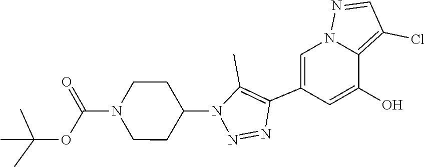 [SMILES: Cc1c(-c2cc(O)c3c(Cl)cnn3c2)nnn1C1CCN(C(=O)OC(C)(C)C)CC1]

A soln of tert-butyl 4-[4-(3-chloro-4-methoxy-pyrazolo[1,5-a]pyridin-6-yl)-5-methyl-triazol-1-yl]piperidine-1-carboxylate (10.24 g, 22.9 mmol) and NDM (23.19 g, 114.6 mmol) in DMA (40 mL) was treated with NaOH (4.58 g, 114.6 mmol). The reaction was stirred overnight at 80° C. Upon cooling to RT, H₂O (300 mL) was added, and the pH was adjusted to 5-6 by addition of FA. The mixture was stirred for 30 min then filtered through a cotton plug to collect the solids. The solids were dissolved in DCM and transferred to separatory funnel and the phases were separated. The organic layer was filtered through Na₂SO₄, diluted with heptane, and concentrated to remove DCM. The residue was triturated in heptane and filtered to collect a pale-yellow solid that was taken up in CHCl₃. The soln was purified by silica gel chromatography and eluted with 0% to 10% MeOH in DCM to afford the title compound (7.53 g, 76%) as a pale-yellow solid. ES/MS m/z 377 [M+2H-tBu]⁺.

The following compounds were prepared essentially as described in Preparation 93 using the appropriate reagents, adjusting temperature, adjusting the reaction times to determine completion of the reactions, and adjusting the purification system as

TABLE 12

| | | | ES/MS |
|---|---|---|---|
| Prep | | | m/z |
| # | Chemical Name | Structure | [M + H]⁺ |
| 94¹ | tert-Butyl 4-[4-(4- hydroxypyrazolo[1,5- a]pyridin-6-yl)-5- methyl-triazol-1- yl]piperidine-1- carboxylate | 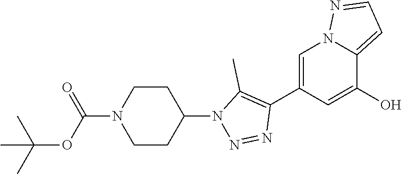 [SMILES: Cc1c(-c2cc(O)c3ccnn3c2)nnn1C1CCN(C(=O)OC(C)(C)C)CC1] | 399 |
| 95² | tert-Butyl 4-[4-(3- cyano-5-hydroxy- imidazo[1,2- a]pyridin-7-yl)-5- methyl-triazol-1- yl]piperidine-1- carboxylate | 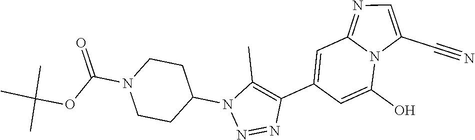 [SMILES: Cc1c(-c2cc(O)n3c(C#N)cnc3c2)nnn1C1CCN(C(=O)OC(C)(C)C)CC1] | 424 |

¹pH of the mixture was acidified to 4 with FA. The mixture was stirred for 3 hr at RT under N₂. The resultant precipitate was collected by filtration then washed with H₂O (3 x 10 mL) and PE (3 x 10 mL).
²Work up: Rxn diluted with H₂O and PE. Mixture acidified to pH 4 with FA. Precipitated solids were collected by filtration.

### Preparation 96

### tert-Butyl (3S,4R)-3-Hydroxy-4-[4-[4-hydroxy-3-(trifluoromethyl)pyrazolo[1,5-a]pyridin-6-yl]-5-methyl-triazol-1-yl]piperidine-1-carboxylate

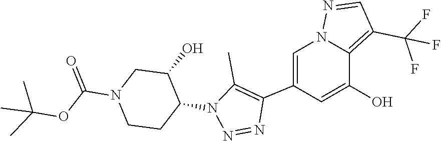 [SMILES: Cc1c(-c2cc(O)c3c(C(F)(F)F)cnn3c2)nnn1[C@@H]1CCN(C(=O)OC(C)(C)C)C[C@@H]1O]

To a soln of tert-butyl (3S,4R)-3-hydroxy-4-[4-[4-methoxy-3-(trifluoromethyl) pyrazolo[1,5-a]pyridin-6-yl]-5-methyl-triazol-1-yl]piperidine-1-carboxylate (214 mg, 0.43 mmol) in DMA (2 mL) was added a freshly prepared soln of NaOH (178 mg, 4.3 mmol) (50% in H₂O) and NDM (437 mg, 2.2 mmol) at RT under N₂. The mixture was stirred for 5 hr at 85° C. Upon cooling to RT, the mixture was diluted with H₂O (5 mL) and acidified to pH 4 with FA. The resultant precipitated solids were collected by filtration and washed with H₂O (3×10 mL) then washed with heptane (3×10 mL). The precipitate was dried under vacuum to afford the title compound (200 mg, 96%) as a yellow solid. ES/MS m/z 483 [M+H]⁺.

The following compounds were prepared essentially as described in Preparation 96 using the appropriate reagents, adjusting temperature, adjusting the reaction times to determine completion of the reactions, and adjusting the purification system as appropriate.

TABLE 13

| | | | ES/MS |
|---|---|---|---|
| Prep | | | m/z |
| # | Chemical Name | Structure | [M + H]⁺ |
| 97 | tert-Butyl 4-[4-(3- fluoro-4-hydroxy- pyrazolo[1,5-a]pyridin- 6-yl)-5-methyl-triazol-1- yl]piperidine-1- carboxylate | 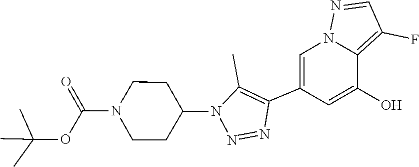 [SMILES: Cc1c(-c2cc(O)c3c(F)cnn3c2)nnn1C1CCN(C(=O)OC(C)(C)C)CC1] | 417 |
| 98¹ | tert-Butyl 3-[4-(3- cyano-4-hydroxy- pyrazolo[1,5-a]pyridin- 6-yl)-5-methyl-triazol-1- yl]azetidine-1- carboxylate | 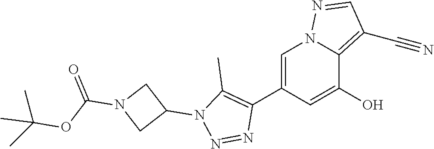 [SMILES: Cc1c(-c2cc(O)c3c(C#N)cnn3c2)nnn1C1CN(C(=O)OC(C)(C)C)C1] | 396 |

¹Acidified to pH 4 with FA and the solids were collected bv filtration.

### Preparation 99

### tert-Butyl (3S,4R)-4-[4-[4-[2-(5-Fluoro-2-pyridyl)-2-oxo-ethoxy]-3-(trifluoromethyl)pyrazolo[1,5-a]pyridin-6-yl]-5-methyl-triazol-1-yl]-3-hydroxy-piperidine-1-carboxylate

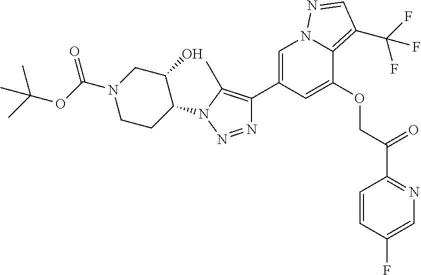 [SMILES: Cc1c(-c2cc(OCC(=O)c3ccc(F)cn3)c3c(C(F)(F)F)cnn3c2)nnn1[C@@H]1CCN(C(=O)OC(C)(C)C)C[C@@H]1O]

To a soln of tert-butyl (3S,4R)-3-hydroxy-4-[4-[4-hydroxy-3-(trifluoromethyl) pyrazolo[1,5-a]pyridin-6-yl]-5-methyl-triazol-1-yl]piperidine-1-carboxylate (130 mg, 0.27 mmol) and Cs₂CO₃ (185 mg, 0.57 mmol) in ACN (4 mL) at RT was slowly added a soln of 2-bromo-1-(5-fluoropyridin-2-yl)ethan-1-one (76 mg, 0.30 mmol) in ACN (2 mL). After stirring 2 hr, the reaction was diluted with EA, filtered, and the filtrate was concentrated. The residue was purified by C18 reverse phase chromatography and eluted with 10% to 100% ACN in H₂O to afford the title compound (62 mg, 37%) as a yellow solid ES/MS m/z 620 [M+H]⁺.

The following compound was prepared essentially as described in Preparation 99 using the appropriate reagents, adjusting temperature, adjusting the reaction times to determine completion of the reactions, and adjusting the purification system as appropriate.

TABLE 14

| | | | ES/MS |
|---|---|---|---|
| Prep | | | m/z |
| # | Chemical Name | Structure | [M + H]⁺ |
| 100 | tert-Butyl 4-[4-[3-fluoro-4- [2-(4-fluorophenyl)-2-oxo- ethoxy]pyrazolo[1,5- a]pyridin-6-yl]-5-methyl- triazol-1-yl]piperidine-1- carboxylate | 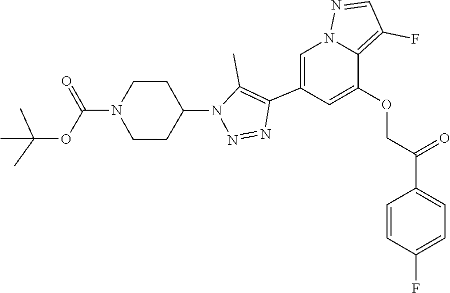 [SMILES: Cc1c(-c2cc(OCC(=O)c3ccc(F)cc3)c3c(F)cnn3c2)nnn1C1CCN(C(=O)OC(C)(C)C)CC1] | 554 |

### Preparation 101

### tert-Butyl 4-[4-[4-[(1S)-2-[tert-butyl(dimethyl)silyl]oxy-1-(2-pyridyl)ethoxy]-3-chloro-pyrazolo[1,5-a]pyridin-6-yl]-5-methyl-triazol-1-yl]piperidine-1-carboxylate

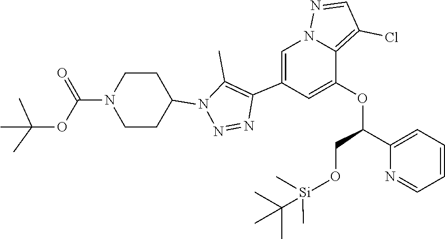 [SMILES: Cc1c(-c2cc(O[C@H](CO[Si](C)(C)C(C)(C)C)c3ccccn3)c3c(Cl)cnn3c2)nnn1C1CCN(C(=O)OC(C)(C)C)CC1]

To [(1R)-2-[tert-butyl(dimethyl)silyl]oxy-1-(2-pyridyl)ethyl]4-methylbenzenesulfonate (1.44 g, 3.5 mmol) in DMF (8.0 mL) at RT was added tert-butyl 4-[4-(3-chloro-4-hydroxy-pyrazolo[1,5-a]pyridin-6-yl)-5-methyl-triazol-1-yl]piperidine-1-carboxylate (1.68 g, 3.9 mmol) and Cs₂CO₃ (1.15 g, 3.5 mmol). The mixture was heated at 55° C. for 4 hr. Upon cooling to RT, the reaction was diluted with H₂O (25 mL) and extracted with DCM (3×25 mL). The organic layers were combined, concentrated, then purified by silica gel chromatography eluted with 0% to 100% EA in heptane to afford the title compound (1.40 g, 59%) as a white foam. ES/MS m/z 668 [M+H]⁺.

### Preparation 102

### tert-Butyl 4-[4-[3-chloro-4-[(1R)-1-(1-methylpyrazol-3-yl)ethoxy]pyrazolo[1,5-a]pyridin-6-yl]-5-methyl-triazol-1-yl]piperidine-1-carboxylate

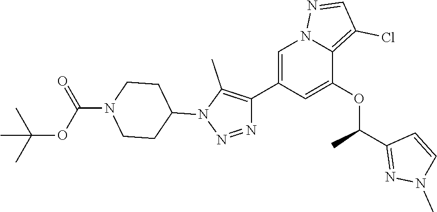 [SMILES: Cc1c(-c2cc(O[C@H](C)c3ccn(C)n3)c3c(Cl)cnn3c2)nnn1C1CCN(C(=O)OC(C)(C)C)CC1]

A mixture of PPh₃ (364 mg, 1.4 mmol) and DIAD (234 mg, 1.2 mmol) in THE (4.0 mL) at 0° C. was stirred for 30 min. Next, a mixture of tert-butyl 4-[4-(3-chloro-4-hydroxy-pyrazolo[1,5-a]pyridin-6-yl)-5-methyl-triazol-1-yl]piperidine-1-carboxylate (200 mg, 0.46 mmol) and (S)-1-(1-methyl-1H-pyrazol-3-yl)ethan-1-ol (69.9 mg, 0.55 mmol) in THE (1 mL) was added and the mixture was allowed to warm to RT and stirred overnight. The mixture was concentrated in vacuo. The residue was purified by reverse phase chromatography and eluted with 0% to 100% ACN in H₂O to afford the title compound (205 mg, 82%). ES/MS m/z 541 [M+H]⁺.

The following compounds were prepared essentially as described in Preparation 102 using the appropriate reagents, adjusting temperature, adjusting the reaction times to determine completion of the reactions, and adjusting the purification system as appropriate.

TABLE 15

| | | | ES/MS |
|---|---|---|---|
| Prep | | | m/z |
| # | Chemical Name | Structure | [M + H]⁺ |
| 103 | tert-Butyl 4-[4-[3-chloro-4-(1- cyclobutylethoxy)pyrazolo[1,5- a]pyridin-6-yl]-5-methyl-triazol- 1-yl]piperidine-1-carboxylate | 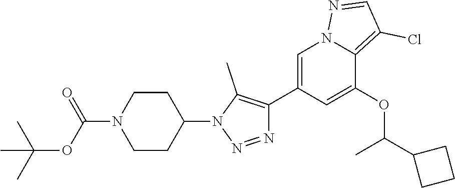 [SMILES: Cc1c(-c2cc(OC(C)C3CCC3)c3c(Cl)cnn3c2)nnn1C1CCN(C(=O)OC(C)(C)C)CC1] | 515 |
| 104¹ | tert-Butyl 4-[4-[3-chloro-4-[1- (4-isoquinolyl)ethoxy] pyrazolo[1,5-a]pyridin-6-yl]-5- methyl-triazol-1-yl]piperidine-1- carboxylate | 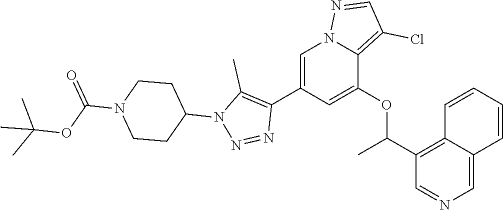 [SMILES: Cc1c(-c2cc(OC(C)c3cncc4ccccc34)c3c(Cl)cnn3c2)nnn1C1CCN(C(=O)OC(C)(C)C)CC1] | 588 |
| 105 | tert-Butyl 4-[4-[3-chloro-4-[1- (5-methyl-1,3,4-thiadiazol-2- yl)ethoxy]pyrazolo[1,5- a]pyridin-6-yl]-5-methyl-triazol- 1-yl]piperidine-1-carboxylate | 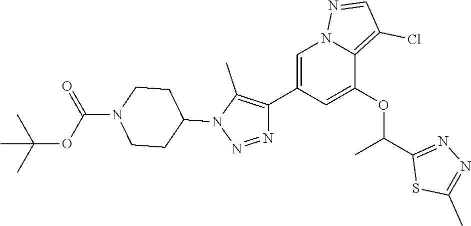 [SMILES: Cc1nnc(C(C)Oc2cc(-c3nnn(C4CCN(C(=O)OC(C)(C)C)CC4)c3C)cn3ncc(Cl)c23)s1] | 559 |
| 106² | tert-Butyl 4-[4-[3-cyano-5- [(1R)-1-(5-fluoro-2- pyridyl)ethoxy]imidazo[1,2- a]pyridin-7-yl]-5-methyl-triazol- 1-yl]piperidine-1-carboxylate | 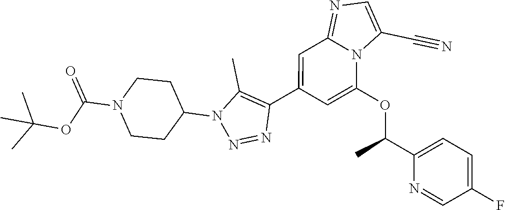 [SMILES: Cc1c(-c2cc(O[C@H](C)c3ccc(F)cn3)n3c(C#N)cnc3c2)nnn1C1CCN(C(=O)OC(C)(C)C)CC1] | 547 |
| 107³ | tert-Butyl 3-[4-[3-cyano-4- [(1R)-1-(2- pyridyl)ethoxy]pyrazolo[1,5- a]pyridin-6-yl]-5-methyl-triazol- 1-yl]azetidine-1-carboxylate | 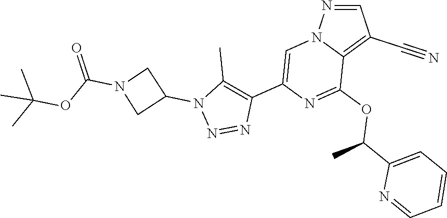 [SMILES: Cc1c(-c2cn3ncc(C#N)c3c(O[C@H](C)c3ccccn3)n2)nnn1C1CN(C(=O)OC(C)(C)C)C1] | 501 |
| 108⁴ | tert-Butyl 4-[4-[3-cyano-5- [(1R)-1-(2- pyridyl)ethoxy]imidazo[1,2- a]pyridin-7-yl]-5-methyl-triazol- 1-yl]piperidine-1-carboxylate | 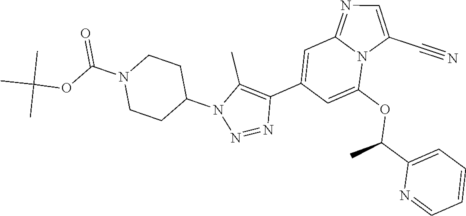 [SMILES: Cc1c(-c2cc(O[C@H](C)c3ccccn3)n3c(C#N)cnc3c2)nnn1C1CCN(C(=O)OC(C)(C)C)CC1] | 529 |

¹Fractions containing title compound from reverse phase chromatography were combined and concentrated to remove ACN. Aqueous soln extracted with DCM (3x) and combined organic layers concentrated. Residue purified by silica gel chromatography and eluted with 0% to 10% MeOH in DCM.
²Purified by C18 reverse flash chromatography and eluted with 505 to 70% ACN in H₂O (0.1% NH₄HCO₃).
³Purified by reverse phase chromatography with the following conditions: Column, C18; eluted with 50% to 60% ACN in H₂O (0.1% FA).
⁴Purified by silica gel chromatography and eluted with PE/EA (2:1 to 0:1).

### Preparation 109

### tert-Butyl (3S,4R)-4-[4-[4-[2-(5-fluoro-2-pyridyl)-2-hydroxy-ethoxy]-3-(trifluoromethyl)pyrazolo[1,5-a]pyridin-6-yl]-5-methyl-triazol-1-yl]-3-hydroxy-piperidine-1-carboxylate

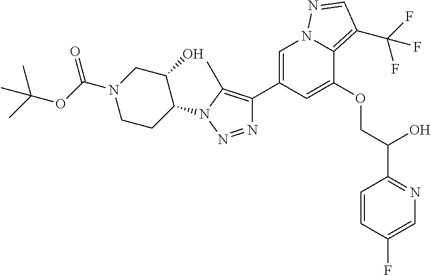 [SMILES: Cc1c(-c2cc(OCC(O)c3ccc(F)cn3)c3c(C(F)(F)F)cnn3c2)nnn1[C@@H]1CCN(C(=O)OC(C)(C)C)C[C@@H]1O]

To a soln of tert-butyl (3S,4R)-4-[4-[4-[2-(5-fluoro-2-pyridyl)-2-oxo-ethoxy]-3-(trifluoromethyl)pyrazolo[1,5-a]pyridin-6-yl]-5-methyl-triazol-1-yl]-3-hydroxy-piperidine-1-carboxylate (62 mg, 0.10 mmol) in MeOH (2 mL) at RT was added NaBH₄ (4.5 mg, 0.12 mmol). After ten minutes, the reaction was concentrated. The residue was treated with EA and H₂O, the phases were separated, and the aqueous layer was extracted with EA (2×20 mL). The combined organic layers were washed with brine, dried over Na₂SO₄, filtered, and concentrated to afford the title compound (58 mg, 94%) as a light-yellow solid. ES/MS m/z 622 [M+H]⁺.

### Preparation 110

### tert-Butyl 4-[4-[3-fluoro-4-[2-(5-fluoro-2-pyridyl)-2-hydroxy-propoxy]pyrazolo[1,5-a]pyridin-6-yl]-5-methyl-triazol-1-yl]piperidine-1-carboxylate

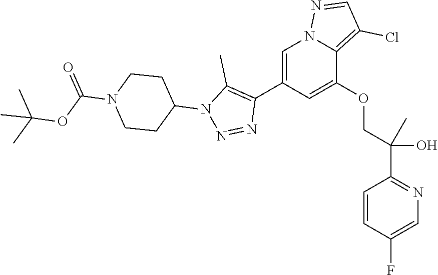 [SMILES: Cc1c(-c2cc(OCC(C)(O)c3ccc(F)cn3)c3c(Cl)cnn3c2)nnn1C1CCN(C(=O)OC(C)(C)C)CC1]

To a soln of tert-butyl 4-[4-[3-fluoro-4-[2-(5-fluoro-2-pyridyl)-2-oxo-ethoxy]pyrazolo[1,5-a]pyridin-6-yl]-5-methyl-triazol-1-yl]piperidine-1-carboxylate (180 mg, 0.33 mmol) in THE (8 mL) at RT is added MeMgBr (3M soln) (58.2 mg, 0.49 mmol) and the reaction was stirred at RT for 30 min. The reaction was quenched with sat. NH₄Cl, extracted into EA (2×), dried over Na₂SO₄, and concentrated. The residue was purified by silica gel chromatography and eluted with 0% to 100% EA in heptane to afford the title compound (90 mg, 49%) as a white solid. ES/MS, m/z 570 [M+H]⁺.

### Preparation 111

### tert-Butyl 4-[4-[4-[2-benzyloxy-1-(5-fluoro-2-pyridyl)ethoxy]-3-chloro-pyrazolo[1,5-a]pyridin-6-yl]-5-methyl-triazol-1-yl]piperidine-1-carboxylate, Isomer 1

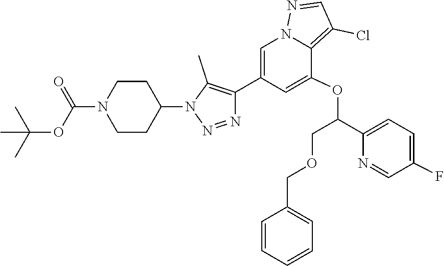 [SMILES: Cc1c(-c2cc(OC(COCc3ccccc3)c3ccc(F)cn3)c3c(Cl)cnn3c2)nnn1C1CCN(C(=O)OC(C)(C)C)CC1]

To tert-butyl 4-[4-(3-chloro-4-hydroxy-pyrazolo[1,5-a]pyridin-6-yl)-5-methyl-triazol-1-yl]piperidine-1-carboxylate (300 mg, 0.69 mmol) were added [2-benzyloxy-1-(5-fluoro-2-pyridyl)ethyl]4-methylbenzenesulfonate, Isomer 1 (320 mg, 0.80 mmol) and Cs₂CO₃ (339 mg, 1.04 mmol) in DMF (3 mL). The mixture was flushed with N₂ and stirred at 60° C. for 2.5 hr. The reaction was cooled down to RT, diluted with H₂O, extracted with EA, and washed with 10% aq soln of LiCl. The organic layer was dried over Na₂SO₄, filtered and concentrated. The residue was purified by silica gel chromatography and eluted with 0% to 100% EA in heptane to afford the title compound (0.43 g, 93%). ES/MS, m/z 662 [M+H]⁺.

### Preparation 112

### 4-Methoxy-6-[5-methyl-1-(4-piperidyl)pyrazol-4-yl]pyrazolo[1,5-a]pyridine-3-carbonitrile

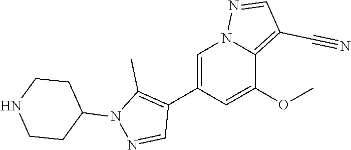 [SMILES: COc1cc(-c2cnn(C3CCNCC3)c2C)cn2ncc(C#N)c12]

tert-Butyl 4-[4-(3-cyano-4-methoxy-pyrazolo[1,5-a]pyridin-6-yl)-5-methyl-pyrazol-1-yl]piperidine-1-carboxylate (738 mg, 1.7 mmol) was stirred at RT in TFA (5 mL) and DCM (5.6 mL) for 30 m then concentrated. To the reaction was added DCM and sat. aq NaHCO₃. The layers were separated. The organic layer was washed with brine, dried over Na₂SO₄, filtered, and concentrated to afford the title compound (0.56 g, 98% o). ES/MS m/z 337 [M+H]⁺.

The following compounds were prepared essentially as described in Preparation 112 using the appropriate reagents, adjusting temperature, adjusting the reaction times to determine completion of the reactions, and adjusting the purification system as appropriate.

TABLE 16

| | | | ES/MS |
|---|---|---|---|
| Prep | | | m/z |
| # | Chemical Name | Structure | [M + H]⁺ |
| 113 | 6-[1-(Azetidin-3-yl)-5- methyl-triazol-4-yl]-4- [(1R)-1-(2- pyridyl)ethoxy] pyrazolo[1,5-a]pyridine- 3-carbonitrile; 2,2,2- trifluoroacetic acid | 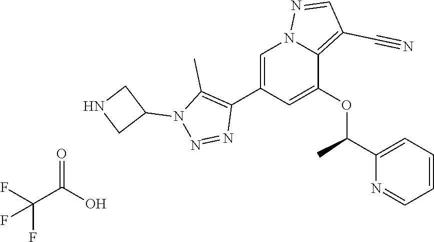 [SMILES: O=C(O)C(F)(F)F ; Cc1c(-c2cc(O[C@H](C)c3ccccn3)c3c(C#N)cnn3c2)nnn1C1CNC1] | 401 |
| 114¹ | 4-Isopropoxy-6-[5- methyl-1-(4- piperidyl)pyrazol-4- yl]pyrazolo[1,5-a] pyridine-3-carbonitrile | 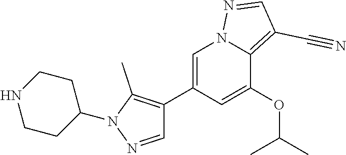 [SMILES: Cc1c(-c2cc(OC(C)C)c3c(C#N)cnn3c2)cnn1C1CCNCC1] | 365 |
| 115² | 4-Methoxy-6-[5-methyl- 1-(4-piperidyl)triazol-4- yl]pyrazolo[1,5-a] pyridine-3-carbonitrile; 2,2,2-trifluoroacetic acid | 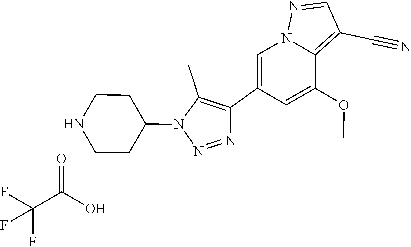 | 338 |
| 116³ | 4-Methoxy-6-[5-methyl- 1-(4-piperidyl)pyrazol- 4-yl]pyrazolo[1,5-a] pyridine, 2,2,2- trifluoroacetic acid | 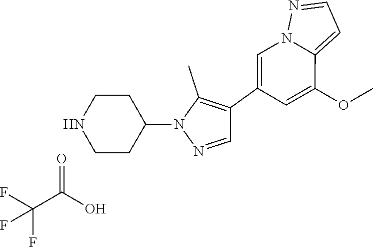 | 312 |

¹After concentration, reaction basified with Na₂CO₃ and extracted with EA to afford product.
²After concentration, solid triturated with Et₂O, and collected by filtration to afford product.
³Reaction was concentrated, and residue taken on without purification.

### Preparation 117

### 6-[1-(Azetidin-3-yl)-5-methyl-pyrazol-4-yl]-4-methoxy-pyrazolo[1,5-a]pyridine-3-carbonitrile

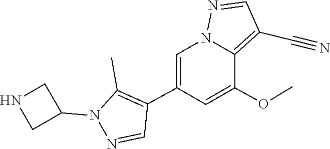 [SMILES: COc1cc(-c2cnn(C3CNC3)c2C)cn2ncc(C#N)c12]

A soln of tert-butyl 3-[4-(3-cyano-4-methoxy-pyrazolo[1,5-a]pyridin-6-yl)-5-methyl-pyrazol-1-yl]azetidine-1-carboxylate (480 mg, 1.18 mmol) and TFA (20 mL) in DCM (20 mL) was stirred for 30 min at RT under N₂. The mixture was concentrated, and the residue was basified to pH 8 with sat. aq NaHCO₃. The resulting mixture was extracted with DCM/IPA (3:1) (3×100 mL). The combined organic layers were washed with brine (10 mL), dried over Na₂SO₄, filtered, and concentrated. The residue was purified by C18 reverse flash chromatography and eluted with 0% to 30% ACN in H₂O (0.1% FA) to afford the title compound (210 mg, 57%) as a white solid. ES/MS m/z 309 [M+H]⁺.

### Preparation 118

### tert-Butyl (3R)-3-[4-[4-(3-cyano-4-methoxy-pyrazolo[1,5-a]pyridin-6-yl)-5-methyl-pyrazol-1-yl]-1-piperidyl]pyrrolidine-1-carboxylate

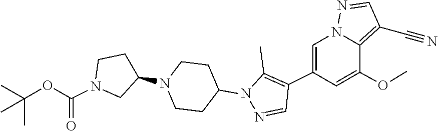 [SMILES: COc1cc(-c2cnn(C3CCN([C@@H]4CCN(C(=O)OC(C)(C)C)C4)CC3)c2C)cn2ncc(C#N)c12]

A soln of tert-butyl (3S)-3-(methanesulfonyloxy)pyrrolidine-1-carboxylate (400 mg, 1.5 mmol), 4-methoxy-6-[5-methyl-1-(4-piperidyl)pyrazol-4-yl]pyrazolo[1,5-a]pyridine-3-carbonitrile (101 mg, 0.30 mmol), and K₂CO₃ (125 mg, 0.91 mmol) in toluene (8 mL) was stirred for 12 hr at 150° C. Upon cooling to RT, the mixture was concentrated. The residue was purified by silica gel chromatography eluted with PE/EA (1:1 to 3:7) to afford the title compound (95 mg, 62%) as a yellow solid. ES/MS m/z 506 [M+H]⁺.

The following compound was prepared essentially as described in Preparation 118 using the appropriate reagents, adjusting temperature, adjusting the reaction times to determine completion of the reactions, and adjusting the purification system as

TABLE 17

| | | | ES/MS |
|---|---|---|---|
| | | | m/z |
| Prep | | | Structure |
| # | Chemical Name | Structure | [M + H]⁺ |
| 119¹ | tert-Butyl (3S)-3-[4- [4-(3-cyano-4- methoxy- pyrazolo[1,5- a]pyridin-6-yl)-5- methyl-pyrazol-1- yl]-1- piperidyl]pyrrolidine- 1-carboxylate | 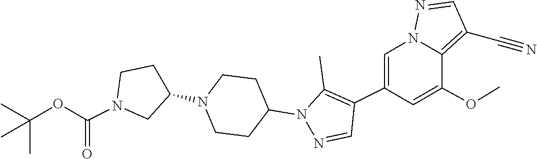 [SMILES: COc1cc(-c2cnn(C3CCN([C@H]4CCN(C(=O)OC(C)(C)C)C4)CC3)c2C)cn2ncc(C#N)c12] | 506 |

¹Purified by C18 reverse flash chromatography, eluted with 50% to 60% ACN in H₂O (0.1% NH₃H₂O).

### Preparation 120

### tert-Butyl N-[1-[[4-[4-(3-cyano-4-methoxy-pyrazolo[1,5-a]pyridin-6-yl)-5-methyl-pyrazol-1-yl]-1-piperidyl]methyl]cyclopropyl]carbamate

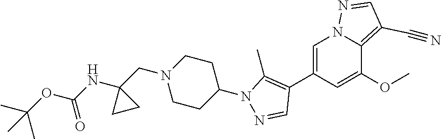 [SMILES: COc1cc(-c2cnn(C3CCN(CC4(NC(=O)OC(C)(C)C)CC4)CC3)c2C)cn2ncc(C#N)c12]

A mixture of 4-methoxy-6-[5-methyl-1-(4-piperidyl)pyrazol-4-yl]pyrazolo[1,5-a]pyridine-3-carbonitrile (100 mg, 0.30 mmol), ACN (4 mL), tert-butyl N-[1-(bromomethyl) cyclopropyl]carbamate (148 mg, 0.60 mmol), K₂CO₃ (102 mg, 0.74 mmol), and KI (74 mg, 0.45 mmol) was stirred at 80° C. for 5 hr. Upon cooling to RT, EA (100 mL) was added, and the mixture was washed with H₂O (3×30 mL). The layers were separated, and the organic layer was concentrated. The residue was purified by silica gel chromatography and eluted with PE/EA (4:1 to 2:1) to afford the title compound (65 mg, 43%) as a white solid. ES/MS m/z 506 [M+H]⁺.

The following compound was prepared essentially as described in Preparation 120 using the appropriate reagents, adjusting temperature, adjusting the reaction times to determine completion of the reactions, and adjusting the purification system as appropriate.

TABLE 18

| | | | ES/MS |
|---|---|---|---|
| Prep | | | m/z |
| # | Chemical Name | Structure | [M + H]⁺ |
| 121¹ | tert-Butyl N-[1-[[4-[4-(3-cyano-4- isopropoxy-pyrazolo[1,5-a]pyridin-6- yl)-5-methyl-pyrazol-1-yl]-1- piperidyl]methyl]cyclopropyl] carbamate | 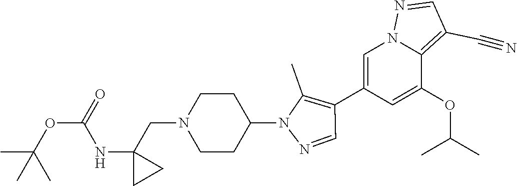 [SMILES: Cc1c(-c2cc(OC(C)C)c3c(C#N)cnn3c2)cnn1C1CCN(CC2(NC(=O)OC(C)(C)C)CC2)CC1] | 534 |

¹Product purified by silica gel chromatography and eluted with PE/EA (1:1 to 1:3).

### Preparation 122

### tert-Butyl 4-((1s,3s)-3-(4-(4-(2-(benzyloxy)-1-(5-fluoropyridin-2-yl)ethoxy)-3-chloropyrazolo[1,5-a]pyridin-6-yl)-5-methyl-1H-1,2,3-triazol-1-yl)cyclobutyl)piperazine-1-carboxylate, Isomer 1

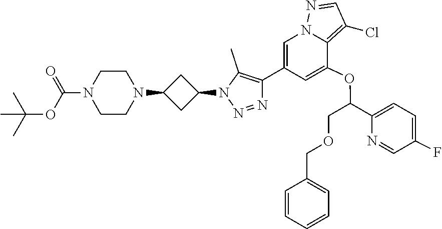 [SMILES: Cc1c(-c2cc(OC(COCc3ccccc3)c3ccc(F)cn3)c3c(Cl)cnn3c2)nnn1[C@H]1C[C@@H](N2CCN(C(=O)OC(C)(C)C)CC2)C1]

A soln of 3-[4-[4-[2-benzyloxy-1-(5-fluoro-2-pyridyl)ethoxy]-3-chloro-pyrazolo[1,5-a]pyridin-6-yl]-5-methyl-triazol-1-yl]cyclobutanol, Isomer 1 (150 mg, 0.27 mmol) and DIPEA (177 mg, 0.23 mmol) in DCM (2 mL) was cooled to −78° C., then treated with trifluoromethanesulfonic anhydride (1.16 g, 0.41 mmol). The reaction was stirred at −78° C. for 15 min. Next, the reaction was treated with a mixture of DIPEA (177 mg, 1.4 mmol) and tert-butyl-1-piperazinecarboxylate (56.0 mg, 0.30 mmol) in DCM (2 mL). The mixture was heated at 40° C. overnight. The mixture was purified by silica gel chromatography eluted with 0% to 100% EA in DCM 12 cv) followed by 0% to 30% MeOH in DCM (8 cv) to afford the title compound (196 mg, 100%) as a brown residue. ES/MS m/z 717 [M+H]⁺.

### Preparation 123

### tert-Butyl N-[3-[4-[4-(3-cyano-4-methoxy-pyrazolo[1,5-a]pyridin-6-yl)-5-methyl-pyrazol-1-yl]-1-piperidyl]cyclobutyl]carbamate, P1

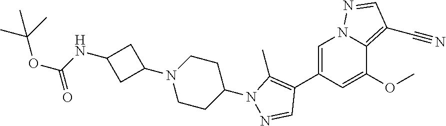 [SMILES: COc1cc(-c2cnn(C3CCN(C4CC(NC(=O)OC(C)(C)C)C4)CC3)c2C)cn2ncc(C#N)c12]

### and

### Preparation 124

### tert-Butyl N-[3-[4-[4-(3-cyano-4-methoxy-pyrazolo[1,5-a]pyridin-6-yl)-5-methyl-pyrazol-1-yl]-1-piperidyl]cyclobutyl]carbamate, P2

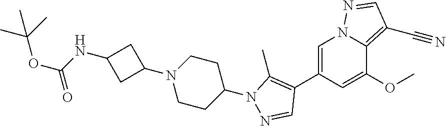 [SMILES: COc1cc(-c2cnn(C3CCN(C4CC(NC(=O)OC(C)(C)C)C4)CC3)c2C)cn2ncc(C#N)c12]

NaBH(OAc)₃ (76 mg, 0.36 mmol) was added to tert-butyl N-(3-oxocyclobutyl)carbamate (22 mg, 0.12 mmol) and 4-methoxy-6-[5-methyl-1-(4-piperidyl)pyrazol-4-yl]pyrazolo[1,5-a]pyridine-3-carbonitrile (40 mg, 0.12 mmol) in DCM (0.7 mL) at RT and was stirred overnight. The mixture was loaded onto a silica gel column and eluted with 1% to 10% MeOH (with 1% NH₄₀H) in DCM to afford trans-tert-butyl N-[3-[4-[4-(3-cyano-4-methoxy-pyrazolo[1,5-a]pyridin-6-yl)-5-methyl-pyrazol-1-yl]-1-piperidyl]cyclobutyl]carbamate, P1 (25 mg, 42%), %), ¹H NMR (400 MHz, CDCl3) δ ppm 1.44 (s, 9H) 1.65-1.75 (m, 2H) 1.87-1.99 (m, 4H) 2.19-2.38 (m, 2H) 2.38-2.47 (m, 4H), 2.56 (m, 1H), 3.03 (m, 2H), 3.82-3.95 (m, 1H), 4.05 (m, 4H), 4.59-4.71 (m, 1H), 6.67 (s, 1H), 7.61 (s, 1H), 8.12 (s, 1H), 8.16 (s, 1H), and tert-butyl N-[3-[4-[4-(3-cyano-4-methoxy-pyrazolo[1,5-a]pyridin-6-yl)-5-methyl-pyrazol-1-yl]-1-piperidyl]cyclobutyl]carbamate, P2 (23 mg, 38%), ¹H NMR (400 MHz, CDCl3) δ ppm 1.38-1.51 (s, 9H), 1.87-1.99 (m, 4H), 2.02 (m, 2H), 2.24-2.38 (m, 4H), 2.41 (s, 3H), 2.93-3.02 (m, 1H), 3.06 (m, 2H), 4.05 (m, 4H), 4.63-4.84 (m, 1H), 6.68 (s, 1H), 7.61 (s, 1H), 8.12 (s, 1H), 8.16 (s, 1H).

The following compounds were prepared essentially as described in preparation 123 and 124 using the appropriate reagents, adjusting the reaction times to determine completion of the reactions, and adjusting the purification system as appropriate.

TABLE 19

| | | | ES/MS |
|---|---|---|---|
| Prep | | | m/z |
| # | Chemical Name | Structure | [M + H]⁺ |
| 125 | tert-butyl 4-[4-[4-(3- cyano-4-methoxy- pyrazolo[1,5- a]pyridin-6-yl)-5- methyl-pyrazol-1-yl]- 1-piperidyl]piperidine- 1-carboxylate | 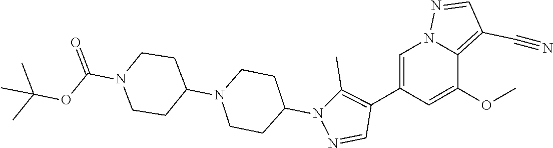 [SMILES: COc1cc(-c2cnn(C3CCN(C4CCN(C(=O)OC(C)(C)C)CC4)CC3)c2C)cn2ncc(C#N)c12] | 520 |
| 126¹ | tert-butyl N-[(1S)-3- [4-[4-(3-cyano-4- methoxy- pyrazolo[1,5- a]pyridin-6-yl)-5- methyl-pyrazol-1-yl]- 1- piperidyl]cyclopentyl] carbamate, P1 | 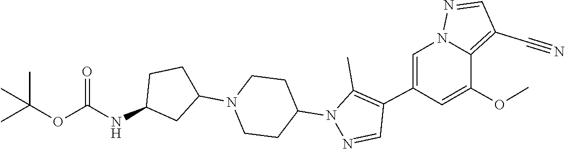 [SMILES: COc1cc(-c2cnn(C3CCN(C4CC[C@H](NC(=O)OC(C)(C)C)C4)CC3)c2C)cn2ncc(C#N)c12] | 520 |
| 127² | tert-butyl N-[(1S)-3- [4-[4-(3-cyano-4- methoxy- pyrazolo[1,5- a]pyridin-6-yl)-5- methyl-pyrazol-1-yl]- 1- piperidyl]cyclopentyl] carbamate, P2 | 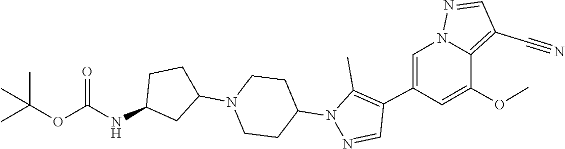 [SMILES: COc1cc(-c2cnn(C3CCN(C4CC[C@H](NC(=O)OC(C)(C)C)C4)CC3)c2C)cn2ncc(C#N)c12] | 520 |
| 128³ | tert-Butyl N-[(1R)-3- [4-[4-(3-cyano-4- methoxy- pyrazolo[1,5- a]pyridin-6-yl)-5- methyl-pyrazol-1-yl]- 1- piperidyl]cyclopentyl] carbamate, P1 | 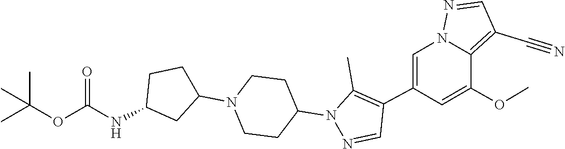 [SMILES: COc1cc(-c2cnn(C3CCN(C4CC[C@@H](NC(=O)OC(C)(C)C)C4)CC3)c2C)cn2ncc(C#N)c12] | 520 |
| 129⁴ | -tert-Butyl N-[(1R)-3- [4-[4-(3-cyano-4- methoxy- pyrazolo[1,5- a]pyridin-6-yl)-5- methyl-pyrazol-1-yl]- 1- piperidyl]cyclopentyl] carbamate, P2 | 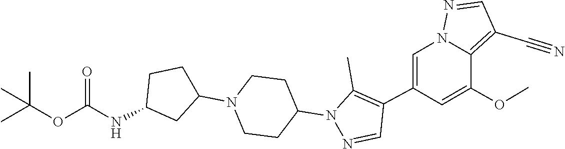 [SMILES: COc1cc(-c2cnn(C3CCN(C4CC[C@@H](NC(=O)OC(C)(C)C)C4)CC3)c2C)cn2ncc(C#N)c12] | 520 |

¹¹H NMR (400 MHz, CDCl3) δ ppm 1.45 (s, 9 H), 1.62 (m, 2 H), 1.91 (m, 2 H), 2.0-2.1 (m, 2H) 2.11-2.19 (m, 1 H), 2.34 (m, 3 H), 2.41 (s, 3 H), 2.44-2.55 (m, 1 H), 2.83 (m, 1 H), 3.32 (m, 2 H), 4.05 (m, 4 H), 4.20 (m, 1 H), 5.15 (m, 1 H), 6.66 (s, 1H), 7.60 (s, 1 H), 8.10-8.18 (m, 2 H).
²¹H NMR(400 MHz, CDCl3) δ ppm 1.45 (s, 9 H), 1.54-1.72 (m, 2 H), 1.78 (m, 1 H), 1.98-2.05 (m, 4 H), 2.10-2.19 (m, 1 H), 2.27-2.38 (m, 4 H), 2.40-2.44 (m, 3 H), 3.02 (m, 1 H), 3.25 (m, 2 H), 4.05 (m, 4 H,) 4.14 (m, 1 H), 6.67 (s, 1 H), 7.61 (s, 1 H), 8.10-8.17 (m, 2 H).
³¹H NMR (400 MHz, CDCl3) δ ppm 1.45 (s, 9 H), 1.59-1.73 (m, 1 H), 1.73-1.84 (m, 1 H), 1.95-2.06 (m, 4 H), 2.13-2.23 (m, 1 H), 2.24-2.45 (m, 7 H), 3.04 (m, 1 H), 3.26 (m, 2 H), 4.05 (m, 4 H), 4.11-4.21 (m, 1 H), 4.49 - 4.67 (m, 1 H), 6.66 (s, 1 H), 7.61 (s, 1 H), 8.08-8.17 (m, 2 H).
⁴¹H NMR (400 MHz, CDCl3) δ ppm 1.45 (s, 9 H), 1.59-1.73 (m, 2 H), 1.85-1.98 (m, 3 H), 2.0-2.1 (m, 1H), 2.15-2.22 (m, 1 H), 2.35 (s, 3H), 2.39-2.45 (m, 3 H), 2.60 (m, 1 H), 2.91 (m, 1 H), 3.36 (m, 2 H), 4.05 (m, 4 H), 4.24 (m, 1 H), 5.28 (m, 1 H), 6.66 (s, 1 H), 7.60 (s, 1 H), 8.09-8.19 (m, 2 H).

### Preparation 130

### tert-Butyl 4-[3-[4-[3-cyano-4-[(1R)-1-(2-pyridyl)ethoxy]pyrazolo[1,5-a]pyridin-6-yl]-5-methyl-triazol-1-yl]azetidin-1-yl]piperidine-1-carboxylate

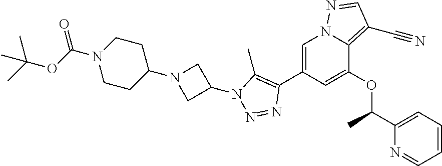 [SMILES: Cc1c(-c2cc(O[C@H](C)c3ccccn3)c3c(C#N)cnn3c2)nnn1C1CN(C2CCN(C(=O)OC(C)(C)C)CC2)C1]

To a soln of 6-[1-(azetidin-3-yl)-5-methyl-triazol-4-yl]-4-[(1R)-1-(2-pyridyl)ethoxy]pyrazolo[1,5-a]pyridine-3-carbonitrile; 2,2,2-trifluoroacetic acid (363 mg) and tert-butyl 4-oxopiperidine-1-carboxylate (722 mg, 3.63 mmol) in MeOH (5 mL) was added AcOH (54 mg, 0.91 mmol) at RT under N₂. The mixture was stirred for 30 min at 50° C. Upon cooling to RT, NaBH₃CN (85 mg, 1.4 mmol) was added. The reaction was stirred for 3 hr at 50° C. Upon cooling to RT, the reaction was quenched with sat aq NaHCO₃ (30 mL). The mixture was extracted with CHCl₃/i-PrOH (3:1) (3×40 mL). The combined organic layers were washed with brine (2×20 mL), dried over Na₂SO₄, filtered, and concentrated. The residue was purified by C18 reverse phase flash chromatography and eluted with 5800 to 65% o ACN in H₂O (0.100 FA) to afford the title compound (297 mg, 560%) as a light-yellow solid. ES/MS m/z 584 [M+H]⁺.

The following compounds were prepared essentially as described in Preparation 130 using the appropriate reagents, adjusting the reaction times to determine completion of the reactions, and adjusting the purification system as appropriate.

TABLE 20

| | | | ES/MS |
|---|---|---|---|
| Prep | | | m/z |
| # | Chemical Name | Structure | [M + H]⁺ |
| 131¹ | tert-Butyl 4-[3-[4-(3- cyano-4-methoxy- pyrazolo[1,5-a]pyridin-6- yl)-5-methyl-pyrazol-1- yl]azetidin-1-yl]piperidine- 1-carboxylate | 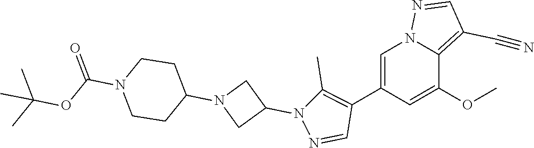 [SMILES: COc1cc(-c2cnn(C3CN(C4CCN(C(=O)OC(C)(C)C)CC4)C3)c2C)cn2ncc(C#N)c12] | 492 |
| 132² | tert-Butyl N-[1-[[4-[4-(3- cyano-4-methoxy- pyrazolo[1,5-a]pyridin-6- yl)-5-methyl-triazol-1-yl]- 1-piperidyl]methyl] cyclopropyl]carbamate | 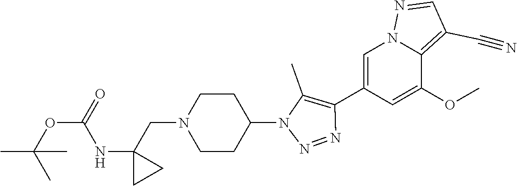 [SMILES: COc1cc(-c2nnn(C3CCN(CC4(NC(=O)OC(C)(C)C)CC4)CC3)c2C)cn2ncc(C#N)c12] | 507 |
| 133³ | tert-Butyl N-[1-[[4-[4-(3- cyano-4-methoxy- pyrazolo[1,5-a]pyridin-6- yl)-5-methyl-pyrazol-1-yl]- 1-piperidyl]methyl] cyclobutyl]carbamate | 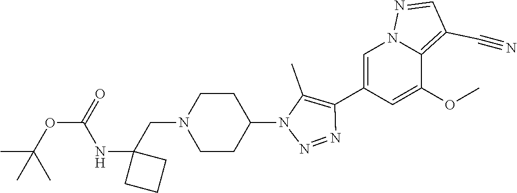 [SMILES: COc1cc(-c2nnn(C3CCN(CC4(NC(=O)OC(C)(C)C)CCC4)CC3)c2C)cn2ncc(C#N)c12] | 520 |

¹Work up: Rxn quenched with H₂O, extracted with EA (3×), combined organic layers, dried over Na₂SO₄, filtered, and concentrated.
²Work up: Rxn quenched with H₂O, extracted with EA (3×), combined organic layers, dried over Na₂SO₄, filtered, and concentrated. Residue purified by C18 reverse phase flash chromatography and eluted with 45% ACN in H₂O (0.1% NH₄HCO₃).
³Completed reaction was basified with Na₂CO₃ and concentrated before purification by prep-TLC eluted with PE/EA (1:1).

### Preparation 134

### 6-[1-[1-(3-Aminocyclobutyl)-4-piperidyl]-5-methyl-pyrazol-4-yl]-4-methoxy-pyrazolo[1,5-a]pyridine-3-carbonitrile; 2,2,2-trifluoroacetic acid, P1

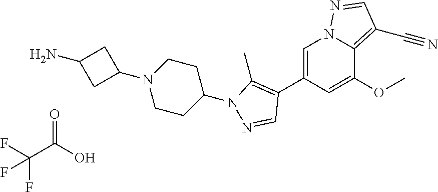 [SMILES: O=C(O)C(F)(F)F ; COc1cc(-c2cnn(C3CCN(C4CC(N)C4)CC3)c2C)cn2ncc(C#N)c12]

trans-tert-Butyl N-[3-[4-[4-(3-cyano-4-methoxy-pyrazolo[1,5-a]pyridin-6-yl)-5-methyl-pyrazol-1-yl]-1-piperidyl]cyclobutyl]carbamate (23 mg, 0.05 mmol) was stirred at RT in TFA (0.13 mL, 1.8 mmol) and DCM (5.6 mL) for 30 min then concentrated to afford the title compound (18 mg, 76%). ES/MS m/z 406 [M+H]⁺.

The following compounds were prepared essentially as described in Preparation 134 using the appropriate reagents, adjusting the reaction times to determine completion of the reactions, and adjusting the purification system as appropriate.

TABLE 21

| | | | ES/MS |
|---|---|---|---|
| Prep | | | m/z |
| # | Chemical name | Structure | [M + H]⁺ |
| 135¹ | 6-[1-[1-(3- Aminocyclobutyl)-4- piperidyl]-5-methyl- pyrazol-4-yl]-4-methoxy- pyrazolo[1,5-a]pyridine-3- carbonitrile, P2 | 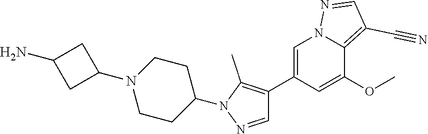 [SMILES: COc1cc(-c2cnn(C3CCN(C4CC(N)C4)CC3)c2C)cn2ncc(C#N)c12] | 406 |
| 136 | 4-Methoxy-6-[5-methyl-1- [1-(4-piperidyl)-4- piperidyl]pyrazol-4- yl]pyrazolo[1,5-a]pyridine- 3-carbonitrile; 2,2,2- trifluoroacetic acid | 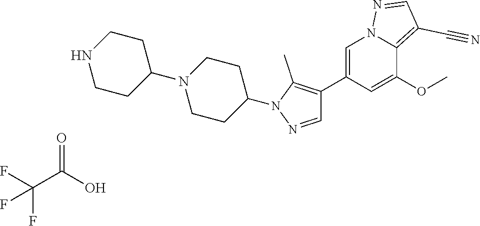 [SMILES: COc1cc(-c2cnn(C3CCN(C4CCNCC4)CC3)c2C)cn2ncc(C#N)c12 ; O=C(O)C(F)(F)F] | 420 |
| 137 | 6-[1-[1-[(3S)-3- Aminocyclopentyl]-4- piperidyl]-5-methyl- pyrazol-4-yl]-4-methoxy- pyrazolo[1,5-a]pyridine-3- carbonitrile; 2,2,2- trifluoroacetic acid, P1 | 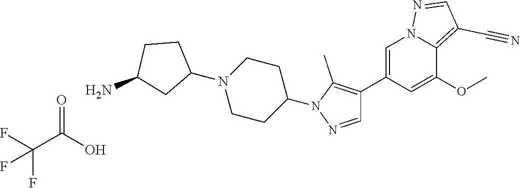 [SMILES: O=C(O)C(F)(F)F ; COc1cc(-c2cnn(C3CCN(C4CC[C@H](N)C4)CC3)c2C)cn2ncc(C#N)c12] | 420 |
| 138 | 6-[1-[1-[(3S)-3- Aminocyclopentyl]-4- piperidyl]-5-methyl- pyrazol-4-yl]-4-methoxy- pyrazolo[1,5-a]pyridine-3- carbonitrile; 2,2,2- trifluoroacetic acid, P2 | 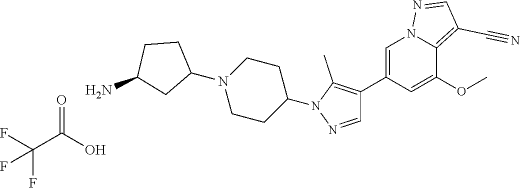 [SMILES: O=C(O)C(F)(F)F ; COc1cc(-c2cnn(C3CCN(C4CC[C@H](N)C4)CC3)c2C)cn2ncc(C#N)c12] | 420 |
| 139 | 6-[1-[1-[(3R)-3- Aminocyclopentyl]-4- piperidyl]-5-methyl- pyrazol-4-yl]-4-methoxy- pyrazolo[1,5-a]pyridine-3- carbonitrile; 2,2,2- trifluoroacetic acid, P2 | 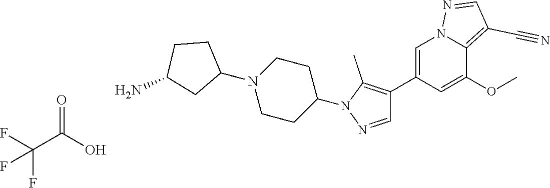 [SMILES: O=C(O)C(F)(F)F ; COc1cc(-c2cnn(C3CCN(C4CC[C@@H](N)C4)CC3)c2C)cn2ncc(C#N)c12] | 420 |
| 140² | 6-[1-[1-[(3R)-3- Aminocyclopentyl]-4- piperidyl]-5-methyl- pyrazol-4-yl]-4-methoxy- pyrazolo[1,5-a]pyridine-3- carbonitrile, P1 | 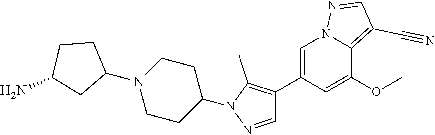 [SMILES: COc1cc(-c2cnn(C3CCN(C4CC[C@@H](N)C4)CC3)c2C)cn2ncc(C#N)c12] | 420 |
| 141 | 6-[1-[1-[(1- Aminocyclopropyl)methyl]- 4-piperidyl]-5-methyl- pyrazol-4-yl]-4-methoxy- pyrazolo[1,5-a]pyridine-3- carbonitrile; 2,2,2- trifluoroacetic acid | 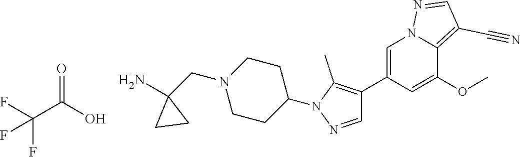 [SMILES: O=C(O)C(F)(F)F ; COc1cc(-c2cnn(C3CCN(CC4(N)CC4)CC3)c2C)cn2ncc(C#N)c12] | 406 |
| 142 | 4-Methoxy-6-[5-methyl-1- [1-[(3R)-pyrrolidin-3-yl]-4- piperidyl]pyrazol-4- yl]pyrazolo[1,5-a]pyridine- 3-carbonitrile; 2,2,2- trifluoroacetic acid | 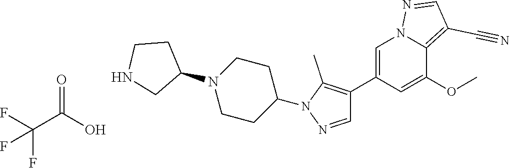 [SMILES: O=C(O)C(F)(F)F ; COc1cc(-c2cnn(C3CCN([C@@H]4CCNC4)CC3)c2C)cn2ncc(C#N)c12] | 406 |
| 143^{3,4} | 4-Methoxy-6-[5-methyl-1- [(3R)-pyrrolidin-3- yl]pyrazol-4- yl]pyrazolo[1,5-a]pyridine- 3-carbonitrile | 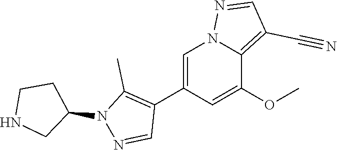 [SMILES: COc1cc(-c2cnn([C@@H]3CCNC3)c2C)cn2ncc(C#N)c12] | 323 |
| 144 | 6-[1-(Azetidin-3-yl)-5- methyl-triazol-4-yl]-4- isopropoxy-pyrazolo[1,5- a]pyridine-3-carbonitrile; 2,2,2-trifluoroacetic acid | 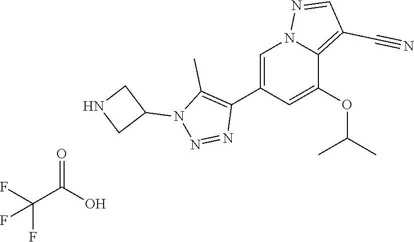 [SMILES: O=C(O)C(F)(F)F ; Cc1c(-c2cc(OC(C)C)c3c(C#N)cnn3c2)nnn1C1CNC1] | 338 |
| 145⁵ | 6-[1-(Azetidin-3-yl)-5- methyl-triazol-4-yl]-4- methoxy-pyrazolo[1,5- a]pyridine-3-carbonitrile; 2,2,2-trifluoroacetic acid | 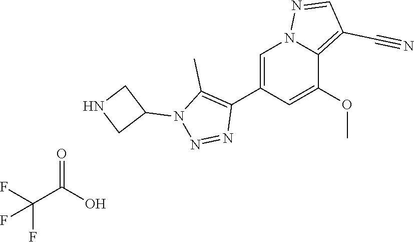 [SMILES: COc1cc(-c2nnn(C3CNC3)c2C)cn2ncc(C#N)c12 ; O=C(O)C(F)(F)F] | 310 |
| 146⁶ | 4-Methoxy-6-[5-methyl-1- [(3S)-pyrrolidin-3- yl]pyrazol-4- yl]pyrazolo[1,5-a]pyridine- 3-carbonitrile; 2,2,2- trifluoroacetic acid | 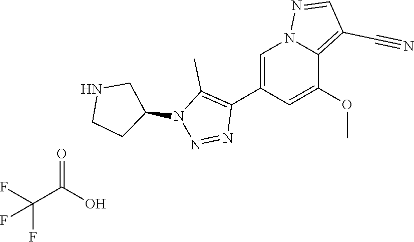 | 323 |
| 147⁷ | 4-Methoxy-6-[5-methyl-1- [1-(4-piperidyl)azetidine-3- yl]pyrazol-4- yl]pyrazolo[1,5-a]pyridine- 3-carbonitrile; 2,2,2- trifluoroacetic acid | 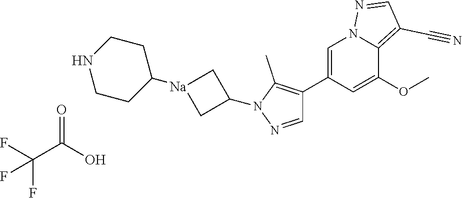 | 392 |
| 148⁸ | 6-[1-[1-[(1- Aminocyclopropyl)methyl]- 4-piperidyl]-5-methyl- pyrazol-4-yl]-4-isopropoxy- pyrazolo[1,5-a]pyridine-3- carbonitrile | 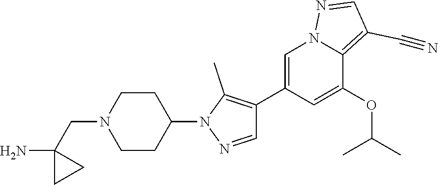 [SMILES: Cc1c(-c2cc(OC(C)C)c3c(C#N)cnn3c2)cnn1C1CCN(CC2(N)CC2)CC1] | 434 |
| 149⁹ | 4-Methoxy-6-[5-methyl-1- [(3S)-3-piperidyl]pyrazol-4- yl]pyrazolo[1,5-a]pyridine- 3-carbonitrile; formic acid | 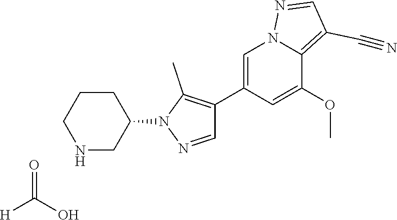 [SMILES: COc1cc(-c2cnn([C@H]3CCCNC3)c2C)cn2ncc(C#N)c12 ; O=CO] | 337 |
| 150 | 6-[1-[1-[(1- Aminocyclobutyl)methyl]- 4-piperidyl]-5-methyl- pyrazol-4-yl]-4-methoxy- pyrazolo[1,5-a]pyridine-3- carbonitrile | 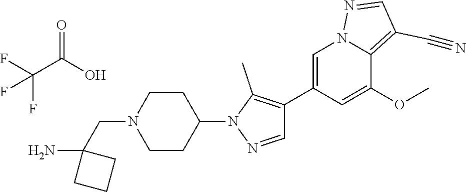 | 420 |
| 151¹⁰ | 4-Methoxy-6-[5-methyl-1- [1-[(3S)-pyrrolidin-3- yl]azetidine-3-yl]pyrazol-4- yl]pyrazolo[1,5-a]pyridine- 3-carbonitrile | 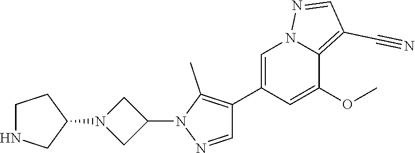 [SMILES: COc1cc(-c2cnn(C3CN([C@H]4CCNC4)C3)c2C)cn2ncc(C#N)c12] | 378 |
| 152¹¹ | 4-Methoxy-6-[5-methyl-1- [1-[(3R)-pyrrolidin-3- yl]azetidine-3-yl]pyrazol-4- yl]pyrazolo[1,5-a]pyridine- 3-carbonitrile | 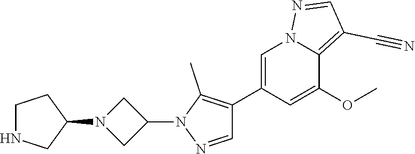 [SMILES: COc1cc(-c2cnn(C3CN([C@@H]4CCNC4)C3)c2C)cn2ncc(C#N)c12] | 378 |
| 153² | 4-methoxy-6-[5-methyl-1- [1-[(3S)-pyrrolidin-3-yl]-4- piperidyl]pyrazol-4- yl]pyrazolo[1,5-a]pyridine- 3-carbonitrile | 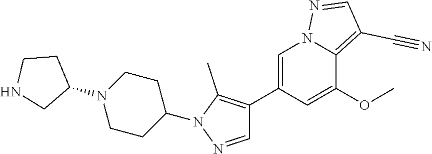 [SMILES: COc1cc(-c2cnn(C3CCN([C@H]4CCNC4)CC3)c2C)cn2ncc(C#N)c12] | 406 |

¹Reaction was concentrated and diluted with DCM and sat. aq NaHCO₃. Layers were separated and the organic layer washed with brine, dried with Na₂SO₄, filtered, and concentrated.
²Reaction was concentrated and diluted with CHCl₃/IPA (3:1) and sat. aq NaHCO₃. Layers were separated and the organic layer washed with brine, dried with Na₂SO₄, filtered, and concentrated.
³Purified by C18 reverse flash chromatography and eluted with 25% to 30% ACN in H₂O (0.1% TFA). Residue neutralized to pH 10 with sat. aq Na₂CO₃ and extracted with EA. Combined organic layers were washed with brine, dried over Na₂SO₄, filtered, and concentrated.
⁴Purified by C18 reverse phase flash chromatography and eluted with 60% to 70% ACN in H₂O (0.1% NH₄HCO₃).
⁵Product was re-crystallized from EA/PE.
⁶Purified by C18 reverse phase flash chromatography and eluted with 20% to 30% ACN in H₂O (0.1% NH₄HCO₃).
⁷Purified by C18 reverse phase flash chromatography and eluted with 10% to 30% ACN in H₂O (0.1% TFA).
⁸Reaction was concentrated, pH adjusted to 9 with sat aq Na₂CO₃, extracted with EA (3 × 200 mL), and concentrated. Residue purified by C18 reverse phase flash chromatography and eluted with 40% to 60% ACN in H₂O (0.1% NH₃H₂O).
⁹Reaction was concentrated, pH adjusted to 9 with saturated aqueous Na₂CO₃, extracted with EA (3 × 30 mL), and concentrated. Residue was purified by C18 reverse phase flash chromatography and eluted with 50% to 70% ACN in H₂O (0.1% FA).
¹⁰Purified by prep-HPLC. Column: Xbridge Shield RP18 OPD, 19 × 150 mm, 5 μm; eluted with 20% to 35% ACN in H₂O (10 mmol/L NH₄HCO₃).
¹¹Purified by prep-HPLC. Column: Xbridge Shield RP18 OPD, 19 × 150 mm, 5 μm; eluted with 18% to 33% ACN in H₂O (10 mmol/L NH₄HCO₃).

### Preparation 154

### (3S,4R)-4-[4-[4-[2-(5-Fluoro-2-pyridyl)-2-hydroxy-ethoxy]-3-(trifluoromethyl)pyrazolo[1,5-a]pyridine-6-yl]-5-methyl-triazol-1-yl]piperidin-3-ol; dihydrochloride

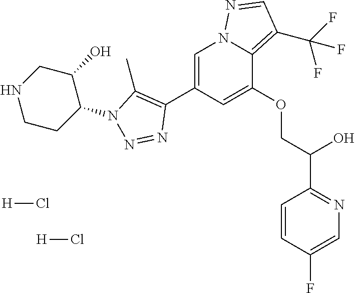

To tert-butyl (3 S,4R)-4-[4-[4-[2-(5-fluoro-2-pyridyl)-2-hydroxy-ethoxy]-3-(trifluoro-methyl)pyrazolo[1,5-a]pyridine-6-yl]-5-methyl-triazol-1-yl]-3-hydroxy-piperidine-1-carboxylate (58 mg, 0.09 mmol) was added DCM (1 mL) followed by a soln of HCl in 1,4-dioxane (13.7 mg, 0.38 mmol, 4M) at RT. After stirring for 30 minutes the reaction was concentrated to afford the title compound (52 mg, 100%) ES/MS m/z 522 [M+H]⁺.

The following compounds were prepared essentially as described in Preparation 154 using the appropriate reagents, adjusting the reaction times to determine completion of the reactions, and adjusting the purification system as appropriate.

TABLE 22

| | | | ES/MS |
|---|---|---|---|
| Prep | | | m/z |
| # | Chemical Name | Structure | [M + H]⁺ |
| 155¹ | (2S)-2-[3-Chloro-6-[5- methyl-1-(4- piperidyl)triazol-4- yl]pyrazolo[1,5-a]pyridine- 4-yl]oxy-2-(2- pyridyl)ethanol; dihydrochloride | 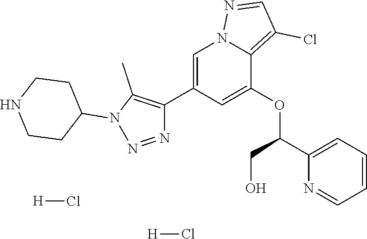 [SMILES: Cc1c(-c2cc(O[C@H](CO)c3ccccn3)c3c(Cl)cnn3c2)nnn1C1CCNCC1 ; Cl ; Cl] | 454 |
| 156² | 3-Chloro-6-[5-methyl-1-(4- piperidyl)triazol-4-yl]-4- [(1R)-1-(1-methylpyrazol-3- yl)ethoxy]pyrazolo[1,5- a]pyridine; hydrochloride | 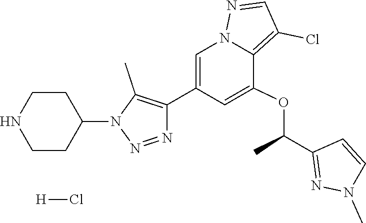 [SMILES: Cc1c(-c2cc(O[C@H](C)c3ccn(C)n3)c3c(Cl)cnn3c2)nnn1C1CCNCC1 ; Cl] | 441 |
| 157 | 3-Chloro-4-(1- cyclobutylethoxy)-6-[5- methyl-1-(4- piperidyl)triazol-4- yl]pyrazolo[1,5- a]pyridine; hydrochloride | 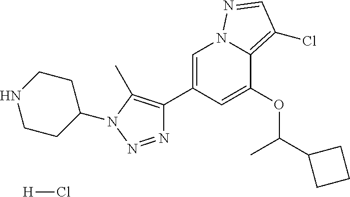 [SMILES: Cc1c(-c2cc(OC(C)C3CCC3)c3c(Cl)cnn3c2)nnn1C1CCNCC1 ; Cl] | 415 |
| 158 | 4-[1-[3-Chloro-6-[5-methyl- 1-(4-piperidyl)triazol-4- yl]pyrazolo[1,5-a]pyridine- 4-yl]oxyethyl]isoquinoline; dihydrochloride | 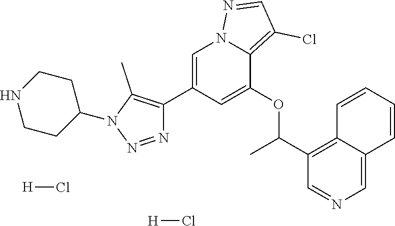 [SMILES: Cc1c(-c2cc(OC(C)c3cncc4ccccc34)c3c(Cl)cnn3c2)nnn1C1CCNCC1 ; Cl ; Cl] | 488 |
| 159 | 1-[3-Fluoro-6-[5-methyl-1- (4-piperidyl)triazol-4- yl]pyrazolo[1,5- a]137yridine-4-yl]oxy-2-(5- fluoro-2-pyridyl)propan-2- ol; hydrochloride | 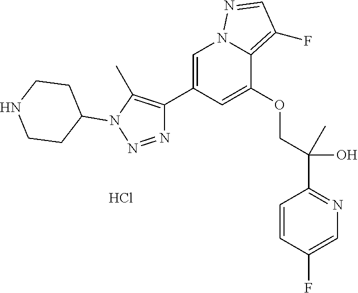 [SMILES: Cc1c(-c2cc(OCC(C)(O)c3ccc(F)cn3)c3c(F)cnn3c2)nnn1C1CCNCC1 ; Cl] | 470 |
| 160 | 2-[1-[3-Chloro-6-[5-methyl- 1-(4-piperidyl)triazol-4- yl]pyrazolo[1,5-a]pyridine- 4-yl]oxyethyl]-5-methyl- 1,3,4-thiadiazole; hydrochloride | 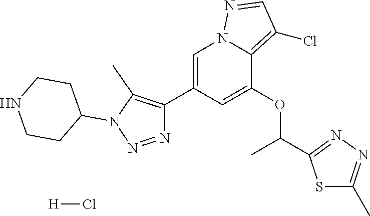 [SMILES: Cc1nnc(C(C)Oc2cc(-c3nnn(C4CCNCC4)c3C)cn3ncc(Cl)c23)s1 ; Cl] | 459 |
| 161³ | 5-[(1R)-1-(5-Fluoro-2- pyridyl)ethoxy]-7-[5- methyl-1-(4- piperidyl)triazol-4- yl]imidazo[1,2-a]pyridine- 3-carbonitrile; hydrochloride | 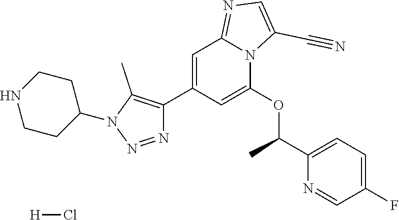 [SMILES: Cc1c(-c2cc(O[C@H](C)c3ccc(F)cn3)n3c(C#N)cnc3c2)nnn1C1CCNCC1 ; Cl] | 447 |
| 162⁴ | 6-[5-Methyl-1-[1-(4- piperidyl)azetidin-3- yl]triazol-4-yl]-4-[(1R)-1- (2- pyridyl)ethoxy]pyrazolo[1,5- a]pyridine-3-carbonitrile | 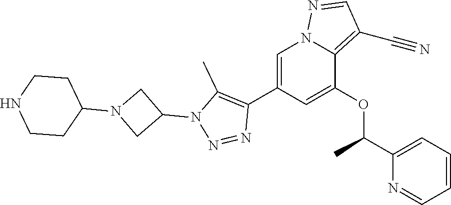 [SMILES: Cc1c(-c2cc(O[C@H](C)c3ccccn3)c3c(C#N)cnn3c2)nnn1C1CN(C2CCNCC2)C1] | 484 |
| 163⁵ | 7-[5-Methyl-1-(4- piperidyl)triazol-4-yl]-5- [(1R)-1-(2- pyridyl)ethoxy]imidazo[1,2- a]pyridine-3-carbonitrile | 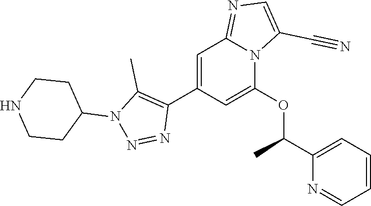 [SMILES: Cc1c(-c2cc(O[C@H](C)c3ccccn3)n3c(C#N)cnc3c2)nnn1C1CCNCC1] | 429 |
| 164 | 4-[(1R)-1- Cyclobutylethoxy]-6-[(1R)- 5-methyl-1-(3- piperidyl)pyrazol-4- yl]pyrazolo[1,5-a]pyridine- 3-carbonitrile; hydrochloride | 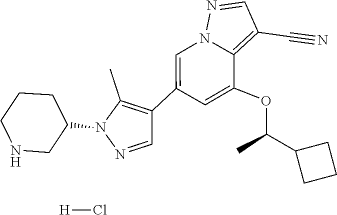 | 405 |
| 165^{6,7} | 6-[5-Methyl-1-[(3S)-3- piperidyl]triazol-4-yl]-4- [(1R)-1-(2- pyridyl)ethoxy]pyrazolo[1,5- a]pyridine-3-carbonitrile; hydrochloride | 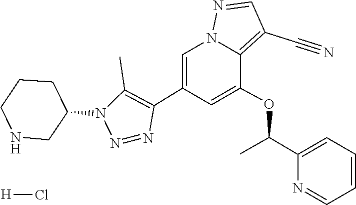 [SMILES: Cc1c(-c2cc(O[C@H](C)c3ccccn3)c3c(C#N)cnn3c2)nnn1[C@H]1CCCNC1 ; Cl] | 429 |
| 166^{6,8} | 6-(1-((1R,3r,5S)-8- Azabicyclo[3.2.1]octan-3- yl)-5-methyl-1H-pyrazol-4- yl)-4-methoxypyrazolo[1,5- a]pyridine-3-carbonitrile; hydrochloride | 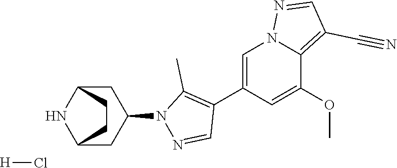 | 363 |
| 167⁶ | 6-(1-((1R,3s,5S)-8- Azabicyclo[3.2.1]octan-3- yl)-5-methyl-1H-pyrazol-4- yl)-4-methoxypyrazolo[1,5- a]pyridine-3-carbonitrile; hydrochloride | 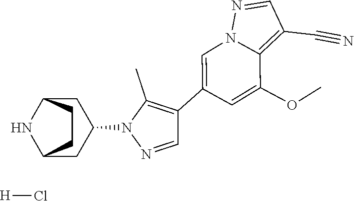 | 363 |
| 168⁹ | 6-[1-(7- Azaspiro[3.5]nonan-2-yl)-5- methyl-pyrazol-4-yl]-4- methoxy-pyrazolo[1,5- a]pyridine-3-carbonitrile; hydrochloride | 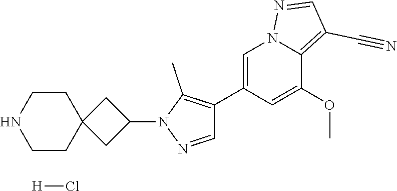 | 377 |
| 169¹⁰ | 6-[1-[1-(2-Amino-2-methyl- propyl)-4-piperidyl]-5- methyl-pyrazol-4-yl]-4- methoxy-pyrazolo[1,5- a]pyridine-3-carbonitrile; hydrochloride | 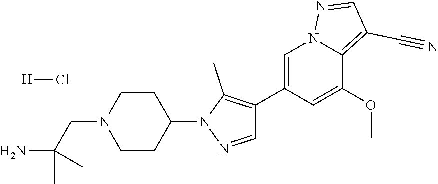 | 408 |
| 170¹¹ | 6-[1-[1-[(1- Aminocyclopropyl)methyl]- 4-piperidyl]-5-methyl- triazol-4-yl]-4-methoxy- pyrazolo[1,5-a]pyridine-3- carbonitrile; hydrochloride | 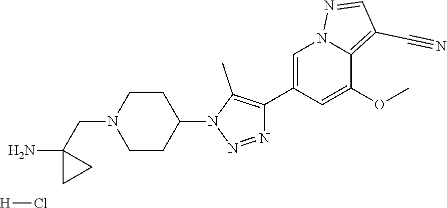 | 407 |
| 171¹² | 4-Methoxy-6-[5-methyl-1- [(3R)-3-piperidyl]pyrazol-4- yl]pyrazolo[1,5-a]pyridine- 3-carbonitrile; formic acid | 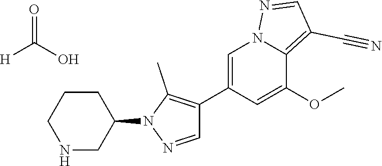 | 337 |
| 172 | 4-Methoxy-6-[5-methyl-1- [(3R)-pyrrolidin-3- yl]triazol-4-y1]pyrazolo[1,5- a]pyridine-3-carbonitrile; hydrochloride | 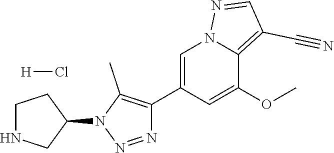 | 324 |
| 173¹³ | 4-Methoxy-6-[5-methyl-1- [(3S)-pyrrolidin-3- yl]triazol-4-yl]pyrazolo[1,5- a]pyridine-3-carbonitrile; hydrochloride | 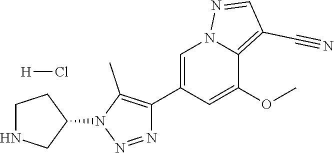 | 324 |

¹After addition of HCl, rxn was stirred 1 hr, cooled to 0° C., and MTBE was added. The precipitate was collected by filtration, dried under a blanket of N₂ until the solid reached ambient temperature.
²Rxn concentrated, residue was purified by reverse phase chromatography and eluted with 0% to 100% ACN in H₂O.
³Residue was washed with EA (3×).
⁴Rxn concentrated and the residue purified by reverse phase chromatography. Column: Xbridge Prep C18 OBD, 19 × 150 mm, 5 μm; eluted with 20% to 58% ACN in H₂O (0.05% NH₃H₂O).
⁵Purified by Prep-HPLC. Column: Gemini-NXC18 AXAI, 21.2 × 150 mm, 5 μm; eluted with 18% to 37% ACN in H₂O (10 mmol/L NH₄HCO₃).
⁶1,4-Dioxane used as solvent.
⁷Work up: Suspension was filtered, filter cake washed with EA to afford title compound.
⁸Crude product recrystallized from EA.
⁹Purified by reverse phase chromatography on C18 column and eluted with 20% to 30% ACN in H₂O (0.1% HCl)
¹⁰Purified by Prep-HPLC. Column: Xbridge Shield RP18 OBD, 19 × 150 mm, 5 μm; eluted with 5% to 25% ACN in H₂O (0.1% FA).
¹¹Reaction was concentrated to afford the title compound and it was used without purification.
¹²Rxn concentrated and the residue purified by reverse phase chromatography. Column: Xbridge Shield RP18 OBD, 19 × 150 mm, 5 μm; eluted with 5% to 25% ACN in H₂O (0.1% formic acid).
¹³Crude product recrystallized from MeOH/EA (4 mL:80 mL).

### Preparation 174

### 2-[3-Chloro-6-[5-methyl-1-(4-piperidyl)triazol-4-yl]pyrazolo[1,5-a]pyridin-4-yl]oxy-2-(5-fluoro-2-pyridyl)ethanol, hydrochloride, Isomer 1

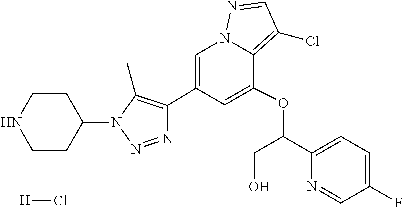 [SMILES: Cc1c(-c2cc(OC(CO)c3ccc(F)cn3)c3c(Cl)cnn3c2)nnn1C1CCNCC1 ; Cl]

tert-Butyl 4-[4-[4-[2-benzyloxy-1-(5-fluoro-2-pyridyl)ethoxy]-3-chloro-pyrazolo[1,5-a]pyridin-6-yl]-5-methyl-triazol-1-yl]piperidine-1-carboxylate, Isomer 1 (1.7 g, 2.6 mmol) was dissolved in DCM (20 mL) and cooled to −78° C. After 20 minutes, a soln of BCl₃ (1 M in DCM) (0.90 g, 7.7 mmol) was added over a 5-minute period and the temperature was maintained at −78° C. for 5 hr. At this point additional BCl₃ (1 M in DCM) (0.90 g, 7.7 mmol) was added. After stirring an additional 1 hr, the reaction was quenched slowly with DCM/MeOH (1:1, 30 mL). The reaction was allowed to warm to RT then the reaction was concentrated in vacuo. The residue was dissolved into a small amount of MeOH and purified by C18 reverse phase chromatography to afford the title compound (1.2 g, 92%) as white solid

### Preparation 175

### 4-(2-(Benzyloxy)-1-(5-fluoropyridin-2-yl)ethoxy)-3-chloro-6-(5-methyl-1-(3-(piperazin-1-yl)cyclobutyl)-1H-1,2,3-triazol-4-yl)pyrazolo[1,5-a]pyridine, Isomer 1

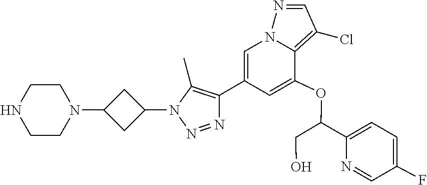 [SMILES: Cc1c(-c2cc(OC(CO)c3ccc(F)cn3)c3c(Cl)cnn3c2)nnn1C1CC(N2CCNCC2)C1]

A soln of tert-butyl 4-(3-(4-(4-(2-(benzyloxy)-1-(5-fluoropyridin-2-yl)ethoxy)-3-chloropyrazolo[1,5-a]pyridin-6-yl)-5-methyl-1H-1,2,3-triazol-1-yl)cyclobutyl)piperazine-1-carboxylate, P1 (196 mg, 0.27 mmol) in DCM (2 mL) was cooled to −78° C., then treated with BCl₃ (96 mg, 0.82 mmol) and allowed to warm to RT while stirring for 3 hr. The reaction was quenched with MeOH and concentrated to afford the title compound (144 mg, 100%) as an off-white solid. ES/MS m/z 527 [M+H]⁺.

### Preparation 176

### 6-[1-[1-(2-Cyanoacetyl)-4-piperidyl]-5-methyl-pyrazol-4-yl]-4-methoxy-pyrazolo[1,5-a]pyridine-3-carbonitrile

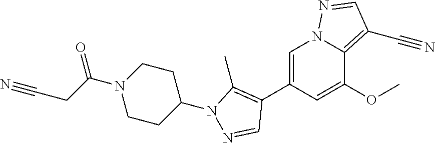 [SMILES: COc1cc(-c2cnn(C3CCN(C(=O)CC#N)CC3)c2C)cn2ncc(C#N)c12]

DIPEA (0.68 mL, 3.89 mmol) was added to 4-methoxy-6-[5-methyl-1-(4-piperidyl)pyrazol-4-yl]pyrazolo[1,5-a]pyridine-3-carbonitrile (654 mg, 1.9 mmol), 2-cyanoacetic acid (182 mg, 2.1 mmol) and HATU (813 mg, 2.1 mmol) in DCM (13 mL) and was stirred at RT overnight. The mixture was concentrated. The residue was purified by silica gel chromatography and eluted with 1% to 10% MeOH in DCM to afford the title compound (775 mg, 98%). ES/MS m/z 404 [M+H]⁺.

### Preparation 177

### tert-Butyl 2-[3-cyano-4-[4-[4-(3-cyano-4-methoxy-pyrazolo[1,5-a]pyridin-6-yl)-5-methyl-pyrazol-1-yl]-1-piperidyl]-1,1-dimethyl-4-oxo-but-2-enyl]-2,7-diazaspiro[3.5]nonane-7-carboxylate

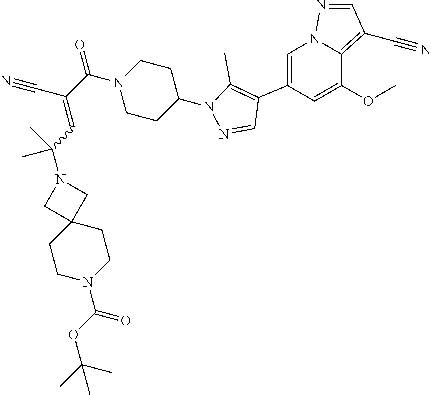 [SMILES: COc1cc(-c2cnn(C3CCN(C(=O)C(C#N)=CC(C)(C)N4CC5(CCN(C(=O)OC(C)(C)C)CC5)C4)CC3)c2C)cn2ncc(C#N)c12]

A soln of 6-[1-[1-(2-cyanoacetyl)-4-piperidyl]-5-methyl-pyrazol-4-yl]-4-methoxy-pyrazolo[1,5-a]pyridine-3-carbonitrile (100 mg, 0.25 mmol), tert-butyl 2-(1,1-dimethyl-2-oxo-ethyl)-2,7-diazaspiro[3.5]nonane-7-carboxylate (220 mg, 0.74 mmol) in DCM (3 mL) was added pyrrolidine (88 mg, 1.2 mmol), and TMSCl (134 mg, 1.2 mmol). The soln was stirred for 1 hr at RT then concentrated. H₂O (10 mL) was added, and the mixture extracted with EA (3×20 mL). The organic layers were combined and washed with brine, dried over Na₂SO₄, filtered, and concentrated. The residue was purified by silica gel chromatography and eluted with DCM/IPA (13:1 to 10:1) to afford the title compound (60 mg, 35%) as a yellow solid. ES/MS m/z 682 [M+H]⁺.

### Preparation 178

### 6-[1-[1-[4-[tert-Butyl(dimethyl)silyl]oxy-2-cyano-4-methyl-pent-2-enoyl]-4-piperidyl]-5-methyl-pyrazol-4-yl]-4-methoxy-pyrazolo[1,5-a]pyridine-3-carbonitrile

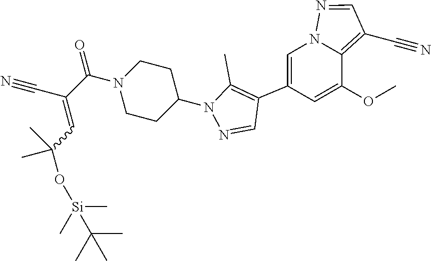 [SMILES: COc1cc(-c2cnn(C3CCN(C(=O)C(C#N)=CC(C)(C)O[Si](C)(C)C(C)(C)C)CC3)c2C)cn2ncc(C#N)c12]

To a soln of 6-[1-[1-(2-cyanoacetyl)-4-piperidyl]-5-methyl-pyrazol-4-yl]-4-methoxy-pyrazolo[1,5-a]pyridine-3-carbonitrile (80 mg, 0.2 mmol) and 2-[(tert-butyldimethylsilyl) oxy]-2-methylpropanal (60 mg, 0.3 mmol) in DCM (5 mL) was added TMSCl (107 mg, 0.99 mmol), pyrrolidine (71 mg, 0.99 mmol) and 4 Å MS (100 mg) at RT under N₂. The mixture was stirred for 2 hr at RT then concentrated. The residue was purified by prep-TLC and eluted with 50% EA in PE to afford the title compound (90 mg, 77%) as a white solid. ES/MS m/z 588 [M+H]⁺.

### Example 1

### N-[3-[4-[4-(3-Cyano-4-methoxy-pyrazolo[1,5-a]pyridin-6-yl)-5-methyl-pyrazol-1-yl]-1-piperidyl]cyclobutyl]prop-2-enamide, P1

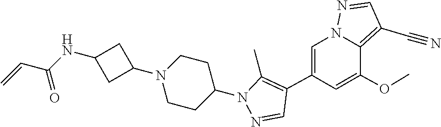 [SMILES: C=CC(=O)NC1CC(N2CCC(n3ncc(-c4cc(OC)c5c(C#N)cnn5c4)c3C)CC2)C1]

DIPEA (0.03 mL, 0.17 mmol) was added to trans-6-[1-[1-(3-aminocyclobutyl)-4-piperidyl]-5-methyl-pyrazol-4-yl]-4-methoxy-pyrazolo[1,5-a]pyridine-3-carbonitrile; 2,2,2-trifluoroacetic acid (18 mg, 0.03 mmol) and acryloyl chloride (0.003 mL, 0.04 mmol) in DCM (0.35 mL) at RT. After being stirred at RT for 2 hr, the reaction was concentrated. The residue was purified on a C18 column and was eluted with 5% to 95% ACN in H₂O (1% TFA). Fractions containing the title compound were combined, concentrated, then treated with DCM and sat. aq Na₂CO₃ and separated. The organic layer was washed with brine, dried with Na₂SO₄, filtered, and concentrated to afford the title compound (3.1 mg, 18%). ES/MS m/z 460 [M+H]⁺.

### Example 2

### 4-Methoxy-6-[5-methyl-1-(1-prop-2-enoyl-4-piperidyl)pyrazol-4-yl]pyrazolo[1,5-a]pyridine-3-carbonitrile

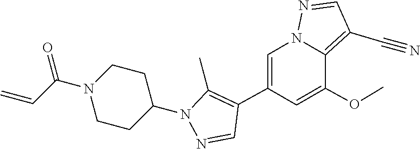 [SMILES: C=CC(=O)N1CCC(n2ncc(-c3cc(OC)c4c(C#N)cnn4c3)c2C)CC1]

Acryloyl chloride (0.01 mL, 0.1 mmol) was added to 4-methoxy-6-[5-methyl-1-(4-piperidyl)pyrazol-4-yl]pyrazolo[1,5-a]pyridine-3-carbonitrile (27 mg, 0.08 mmol) and DIPEA (0.02 mL, 0.1 mmol) in DCM (0.8 mL) at RT. After 30 min, the mixture was concentrated. The residue was purified by silica gel chromatography and eluted with 1% to 10% MeOH in DCM to afford the title compound (8.9 mg, 28%). ES/MS m/z 391[M+H]⁺.

The following compounds were prepared essentially as described in Example 2 using the appropriate reagents, adjusting the temperature, and the reaction times to determine completion of the reactions, and adjusting the purification system as appropriate.

TABLE 23

| | | | ES/MS |
|---|---|---|---|
| Example | | | m/z |
| # | Chemical Name | Structure | [M + H]⁺ |
| 3¹ | 4-Methoxy-6-[5-methyl- 1-(1-prop-2- enoylazetidin-3- yl)pyrazol-4- yl]pyrazolo[1,5- a]pyridine-3-carbonitrile | 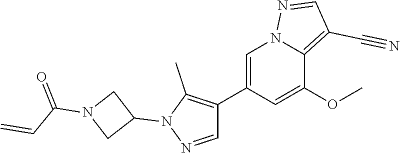 [SMILES: C=CC(=O)N1CC(n2ncc(-c3cc(OC)c4c(C#N)cnn4c3)c2C)C1] | 363 |
| 4^{2,3} | 4-Isopropoxy-6-[5- methyl-1-(1-prop-2- enoylazetidin-3- yl)triazol-4- yl]pyrazolo[1,5- a]pyridine-3-carbonitrile | 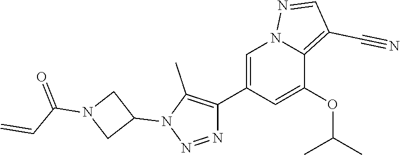 [SMILES: C=CC(=O)N1CC(n2nnc(-c3cc(OC(C)C)c4c(C#N)cnn4c3)c2C)C1] | 392 |
| 5^{2,4} | 4-Methoxy-6-[5-methyl- 1-(1-prop-2- enoylazetidin-3- yl)triazol-4- yl]pyrazolo[1,5- a]pyridine-3-carbonitrile | 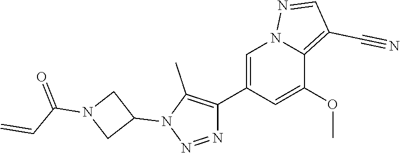 [SMILES: C=CC(=O)N1CC(n2nnc(-c3cc(OC)c4c(C#N)cnn4c3)c2C)C1] | 364 |
| 6^{2,5} | 1-[4-[4-[3-Chloro-4-[1- (5-fluoro-2-pyridyl)-2- hydroxy- ethoxy]pyrazolo[1,5- a]pyridin-6-yl]-5-methyl- triazol-1-yl]-1- piperidyl]prop-2-en-1- one, Isomer 1 | 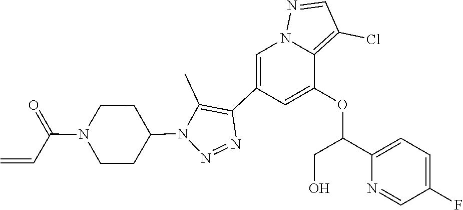 [SMILES: C=CC(=O)N1CCC(n2nnc(-c3cc(OC(CO)c4ccc(F)cn4)c4c(Cl)cnn4c3)c2C)CC1] | 526 |
| 7⁶ | 6-[5-Methyl-1-[1-(1- prop-2-enoyl-4- piperidyl)pyridine-3- yl]triazol-4-yl]-4-[(1R)- 1-(2- pyridyl)ethoxy]pyrazolo[1,5- a]pyridine-3- carbonitrile | 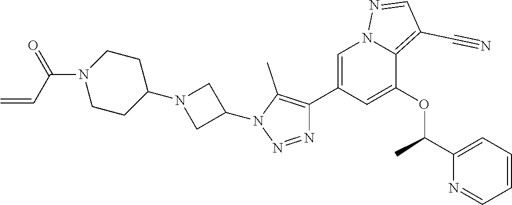 [SMILES: C=CC(=O)N1CCC(N2CC(n3nnc(-c4cc(O[C@H](C)c5ccccn5)c5c(C#N)cnn5c4)c3C)C2)CC1] | 538 |
| 8⁷ | 7-[5-Methyl-1-(1-prop-2- enoyl-4-piperidyl)triazol- 4-y1]-5-[(1R)-1-(2- pyridyl)ethoxy]imidazo[1,2- a]pyridine-3- carbonitrile | 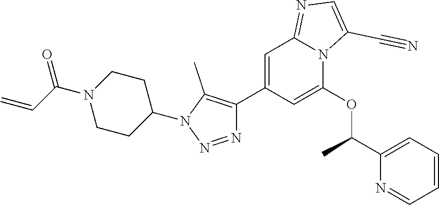 [SMILES: C=CC(=O)N1CCC(n2nnc(-c3cc(O[C@H](C)c4ccccn4)n4c(C#N)cnc4c3)c2C)CC1] | 483 |
| 9⁸ | 4-[(1R)-1- Cyclobutylethoxy]-6- [(1R)-5-methyl-1-(1- prop-2-enoy1-3- piperidyl)pyrazol-4- yl]pyrazolo[1,5- a]pyridine-3-carbonitrile | 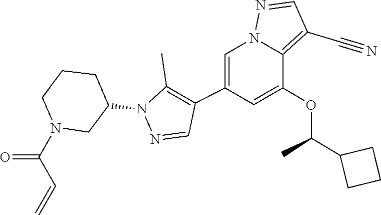 | 459 |
| 10⁹ | 6-[5-Methyl-1-[(3S)-1- prop-2-enoyl-3- piperidyl]triazol-4-yl]-4- [(1R)-1-(2- pyridyl)ethoxy]pyrazolo[1,5- a]pyridine-3- carbonitrile | 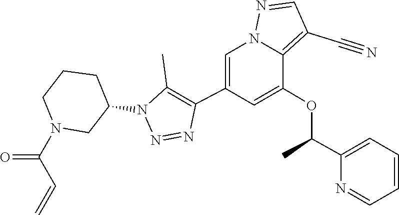 [SMILES: C=CC(=O)N1CCC[C@H](n2nnc(-c3cc(O[C@H](C)c4ccccn4)c4c(C#N)cnn4c3)c2C)C1] | 483 |
| 11¹⁰ | 6-(1-((1R,3r,5S)-8- Acryloyl-8- azabicyclo[3.2.1]octan-3- yl)-5-methyl-1H-pyrazol- 4-yl)-4- methoxypyrazolo[1,5- a]pyridine-3-carbonitrile | 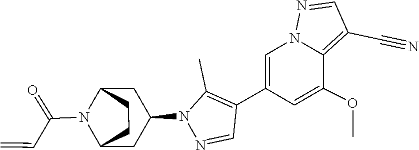 [SMILES: C=CC(=O)N1[C@@H]2CC[C@H]1C[C@@H](n1ncc(-c3cc(OC)c4c(C#N)cnn4c3)c1C)C2] | 417 |
| 12¹¹ | 4-Methoxy-6-[5-methyl- 1-[(3S)-1-prop-2- enoylpyrrolidin-3- yl]pyrazol-4- yl]pyrazolo[1,5- a]pyridine-3-carbonitrile | 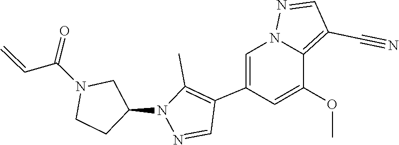 [SMILES: C=CC(=O)N1CC[C@H](n2ncc(-c3cc(OC)c4c(C#N)cnn4c3)c2C)C1] | 377 |
| 13^{2,12} | N-[1-[[4-[4-(3-Cyano-4- isopropoxy-pyrazolo[1,5- a]pyridine-6-yl)-5- methyl-pyrazol-1-yl]-1- piperidyl]methyl]cyclopro- pyl]prop-2-enamide | 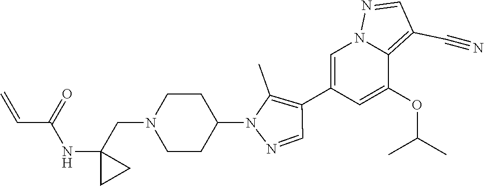 [SMILES: C=CC(=O)NC1(CN2CCC(n3ncc(-c4cc(OC(C)C)c5c(C#N)cnn5c4)c3C)CC2)CC1] | 488 |
| 14^{2,13} | 4-Methoxy-6-[5-methyl- 1-(7-prop-2-enoyl-7- azaspiro[3.5]nonan-2- yl)pyrazol-4- yl]pyrazolo[1,5- a]pyridine-3-carbonitrile | 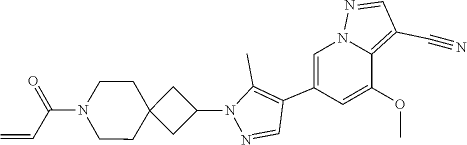 [SMILES: C=CC(=O)N1CCC2(CC1)CC(n1ncc(-c3cc(OC)c4c(C#N)cnn4c3)c1C)C2] | 431 |
| 15^{2,13} | N-[2-[4-[4-(3-Cyano-4- methoxy-pyrazolo[1,5- a]pyridin-6-yl)-5-methyl- pyrazol-1-yl]-1- piperidyl]-1,1-dimethyl- ethyl]prop-2-enamide | 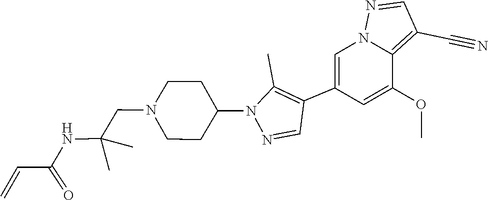 [SMILES: C=CC(=O)NC(C)(C)CN1CCC(n2ncc(-c3cc(OC)c4c(C#N)cnn4c3)c2C)CC1] | 462 |
| 16^{2,14} | N-[1-[[4-[4-(3-Cyano-4- methoxy-pyrazolo[1,5- a]pyridine-6-yl)-5- methyl-triazol-1-yl]-1- piperidyl]methyl]cyclopro- pyl]prop-2-enamide | 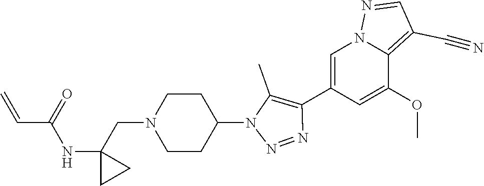 [SMILES: C=CC(=O)NC1(CN2CCC(n3nnc(-c4cc(OC)c5c(C#N)cnn5c4)c3C)CC2)CC1] | 461 |
| 17^{2,15} | 4-Methoxy-6-[5-methyl- 1-[(3S)-1-prop-2-enoyl- 3-piperidyl]pyrazol-4- yl]pyrazolo[1,5- a]pyridine-3-carbonitrile | 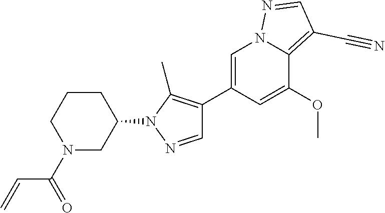 [SMILES: C=CC(=O)N1CCC[C@H](n2ncc(-c3cc(OC)c4c(C#N)cnn4c3)c2C)C1] | 391 |
| 18^{2,16} | 4-Methoxy-6-[5-methyl- 1-[(3R)-1-prop-2-enoyl- 3-piperidyl]pyrazol-4- yl]pyrazolo[1,5- a]pyridine-3-carbonitrile | 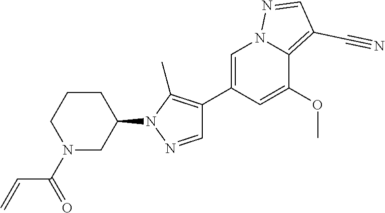 [SMILES: C=CC(=O)N1CCC[C@@H](n2ncc(-c3cc(OC)c4c(C#N)cnn4c3)c2C)C1] | 391 |
| 19^{2,17} | N-[1-[[4-[4-(3-Cyano-4- methoxy-pyrazolo[1,5- a]pyridin-6-yl)-5-methyl- pyrazol-1-yl]-1- piperidyl]methyl]cyclobu- tyl]prop-2-enamide | 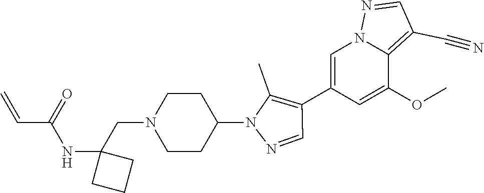 [SMILES: C=CC(=O)NC1(CN2CCC(n3ncc(-c4cc(OC)c5c(C#N)cnn5c4)c3C)CC2)CCC1] | 474 |
| 20¹⁸ | 4-Methoxy-6-[5-methyl- 1-[1-[(3S)-1-prop-2- enoylpyrrolidin-3- yl]azetidine-3-yl]pyrazol- 4-yl]pyrazolo[1,5- a]pyridine-3-carbonitrile | 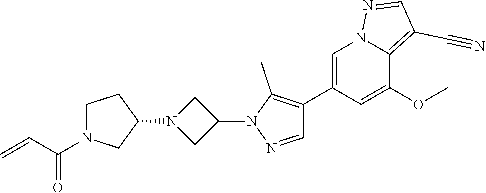 [SMILES: C=CC(=O)N1CC[C@H](N2CC(n3ncc(-c4cc(OC)c5c(C#N)cnn5c4)c3C)C2)C1] | 432 |
| 21¹⁸ | 4-Methoxy-6-[5-methyl- 1-[1-[(3R)-1-prop-2- enoylpyrrolidin-3- yl]azetidine-3-yl]pyrazol- 4-yl]pyrazolo[1,5- a]pyridine-3-carbonitrile | 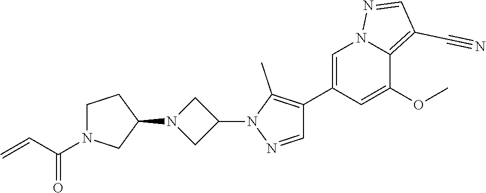 [SMILES: C=CC(=O)N1CC[C@@H](N2CC(n3ncc(-c4cc(OC)c5c(C#N)cnn5c4)c3C)C2)C1] | 432 |
| 22¹⁹ | 4-Methoxy-6-[5-methyl- 1-[(3R)-1-prop-2- enoylpyrrolidin-3- yl]triazol-4- yl]pyrazolo[1,5- a]pyridine-3-carbonitrile | 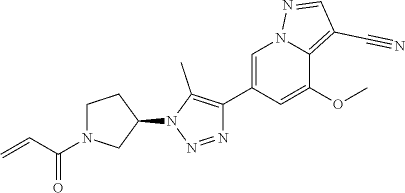 [SMILES: C=CC(=O)N1CC[C@@H](n2nnc(-c3cc(OC)c4c(C#N)cnn4c3)c2C)C1] | 378 |
| 23²⁰ | 4-Methoxy-6-[5-methyl- 1-[(3S)-1-prop-2- enoylpyrrolidin-3- yl]triazol-4- yl]pyrazolo[1,5- a]pyridine-3-carbonitrile | 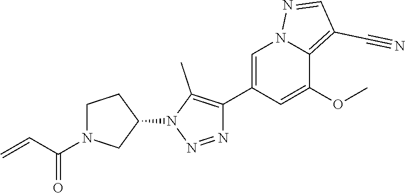 [SMILES: C=CC(=O)N1CC[C@H](n2nnc(-c3cc(OC)c4c(C#N)cnn4c3)c2C)C1] | 378 |
| 24²¹ | 6-(1-((1R,3s,5S)-8- Acryloyl-8- azabicyclo[3.2.1]octan-3- yl)-5-methyl-1H-pyrazol- 4-yl)-4- methoxypyrazolo[1,5- a]pyridine-3-carbonitrile | 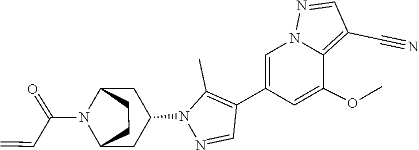 [SMILES: C=CC(=O)N1[C@@H]2CC[C@H]1C[C@H](n1ncc(-c3cc(OC)c4c(C#N)cnn4c3)c1C)C2] | 417 |
| 25²² | 4-Methoxy-6-[5-methyl- 1-[1-(1-prop-2-enoyl-4- piperidyl)-4- piperidyl]pyrazol-4- yl]pyrazolo[1,5- a]pyridine-3-carbonitrile | 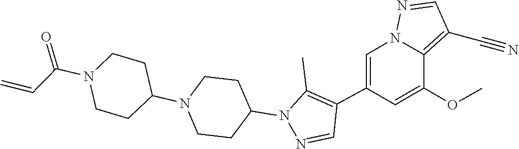 [SMILES: C=CC(=O)N1CCC(N2CCC(n3ncc(-c4cc(OC)c5c(C#N)cnn5c4)c3C)CC2)CC1] | 474 |
| 26²² | N-[(1S)-3-[4-[4-(3- Cyano-4-methoxy- pyrazolo[1,5-a]pyridin-6- yl)-5-methyl-pyrazol-1- yl]-1-piperidyl] cyclopentyl]prop-2- enamide, P1 | 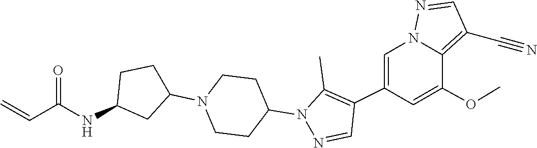 [SMILES: C=CC(=O)N[C@H]1CCC(N2CCC(n3ncc(-c4cc(OC)c5c(C#N)cnn5c4)c3C)CC2)C1] | 474 |
| 27²² | N-[(1S)-3-[4-[4-(3- Cyano-4-methoxy- pyrazolo[1,5-a]pyridin-6- yl)-5-methyl-pyrazol-1- yl]-1-piperidyl] cyclopentyl]prop-2- enamide, P2 | 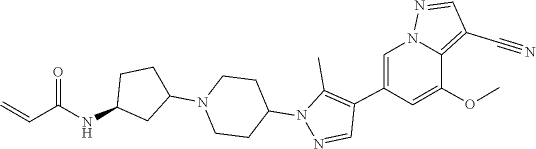 [SMILES: C=CC(=O)N[C@H]1CCC(N2CCC(n3ncc(-c4cc(OC)c5c(C#N)cnn5c4)c3C)CC2)C1] | 474 |
| 28²² | N-[(1R)-3-[4-[4-(3- Cyano-4-methoxy- pyrazolo[1,5-a]pyridin-6- yl)-5-methyl-pyrazol-1- yl]-1- piperidyl]cyclopentyl]prop- 2-enamide, P2 | 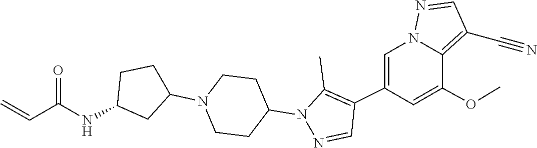 [SMILES: C=CC(=O)N[C@@H]1CCC(N2CCC(n3ncc(-c4cc(OC)c5c(C#N)cnn5c4)c3C)CC2)C1] | 474 |
| 29^{2,23} | N-[(1R)-3-[4-[4-(3- Cyano-4-methoxy- pyrazolo[1,5-a]pyridin-6- yl)-5-methyl-pyrazol-1- yl]-1- piperidyl]cyclopentyl]prop- 2-enamide, P1 | 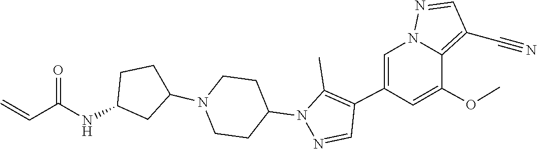 [SMILES: C=CC(=O)N[C@@H]1CCC(N2CCC(n3ncc(-c4cc(OC)c5c(C#N)cnn5c4)c3C)CC2)C1] | 474 |
| 30^{2,24} | N-[1-[[4-[4-(3-Cyano-4- methoxy-pyrazolo[1,5- a]pyridin-6-yl)-5-methyl- pyrazol-1-yl]-1- piperidyl]methyl]cyclopro- pyl]prop-2-enamide | 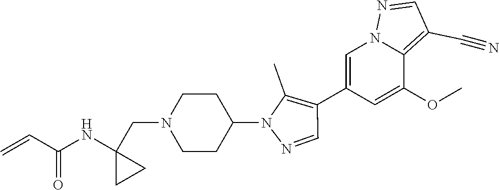 [SMILES: C=CC(=O)NC1(CN2CCC(n3ncc(-c4cc(OC)c5c(C#N)cnn5c4)c3C)CC2)CC1] | 460 |
| 31²⁵ | 4-Methoxy-6-[5-methyl- 1-[1-[(3R)-1-prop-2- enoylpyrrolidin-3-yl]-4- piperidyl]pyrazol-4- yl]pyrazolo[1,5- a]pyridine-3-carbonitrile | 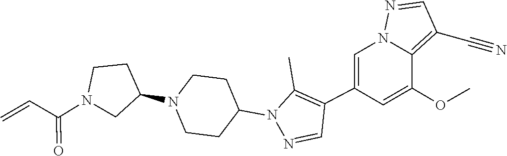 [SMILES: C=CC(=O)N1CC[C@@H](N2CCC(n3ncc(-c4cc(OC)c5c(C#N)cnn5c4)c3C)CC2)C1] | 460 |
| 32^{2,26} | 4-Methoxy-6-[5-methyl- 1-[1-(1-prop-2-enoyl-4- piperidyl)azetidin-3- yl]pyrazol-4- yl]pyrazolo[1,5- a]pyridine-3-carbonitrile | 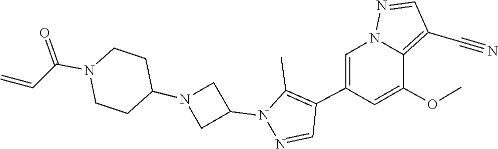 [SMILES: C=CC(=O)N1CCC(N2CC(n3ncc(-c4cc(OC)c5c(C#N)cnn5c4)c3C)C2)CC1] | 446 |
| 33^{1,27} | 4-Methoxy-6-[5-methyl- 1-[1-[(3S)-1-prop-2- enoylpyrrolidin-3-yl]-4- piperidyl]pyrazol-4- yl]pyrazolo[1,5- a]pyridine-3-carbonitrile | 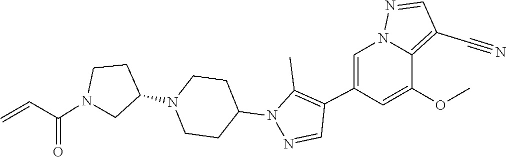 [SMILES: C=CC(=O)N1CC[C@H](N2CCC(n3ncc(-c4cc(OC)c5c(C#N)cnn5c4)c3C)CC2)C1] | 460 |
| 34²⁸ | 4-Methoxy-6-[5-methyl- 1-[(3R)-1-prop-2- enoylpyrrolidin-3- yl]pyrazol-4- yl]pyrazolo[1,5- a]pyridine-3-carbonitrile | 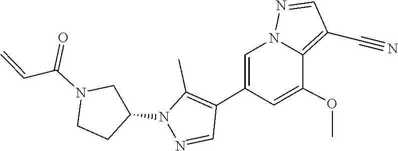 [SMILES: C=CC(=O)N1CC[C@@H](n2ncc(-c3cc(OC)c4c(C#N)cnn4c3)c2C)C1] | 377 |
| 35²² | N-[3-[4-[4-(3-Cyano-4- methoxy-pyrazolo[1,5- a]pyridin-6-yl)-5-methyl- pyrazol-1-yl]-1- piperidyl]cyclobutyl]prop- 2-enamide, P2 | 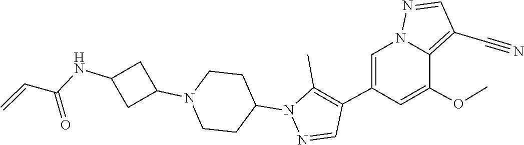 [SMILES: C=CC(=O)NC1CC(N2CCC(n3ncc(-c4cc(OC)c5c(C#N)cnn5c4)c3C)CC2)C1] | 460 |

¹Purified by C18 reverse phase flash chromatography and eluted with 10% to 50% ACN in H₂O (0.1% NH₄HCO₃).
²NEt₃ used as base.
³Purified by Prep-HPLC that used the following conditions: Column: YMC-Actus Triart C18, 20 × 250 mm, 5 μm; eluted with 34% to 53% ACN in H₂O.
⁴Purified by Prep-HPLC that used the following conditions: Column: SunFire Prep C18 OBD, 19 × 150 mm, 5 μm; eluted with 15% to 42% ACN in H₂O (0.1% FA).
⁵Purified by prep HPLC that used the following conditions: Column: Phenomenx Luna, 5 μm C18 100A LC; 250 × 21.2 mm; eluted with 10% to 100% ACN in H₂O.
⁶Purified by C18 reverse flash chromatography and eluted with 20% to 30% ACN in H₂O (0.1% FA).
⁷Purified by Prep-HPLC that used the following conditions: Column: Gemini-NX C18 AXAI, 21.2 × 150 mm, 5 μm; eluted with 24% to 38% ACN in H₂O (0.05% FA).
⁸Purified by C18 reverse phase flash chromatography and eluted with 45% to 50% ACN in H₂O.
⁹Purified by C18 reverse phase flash chromatography and eluted with 30% to 60% ACN in H₂O (0.1% FA).
¹⁰Purified by Prep-HPLC that used the following conditions: Column: SunFire Prep C18 OBD, 19 × 150 mm, 5 μm; eluted with 32% to 46% ACN in H₂O.
¹¹Purified by C18 reverse phase flash chromatography and eluted with 30% to 50% ACN in H₂O.
¹²Purified by C18 reverse phase flash chromatography and eluted with 40% to 60% ACN in H₂O (0.1% NH₃—H₂O).
¹³Purified by C18 reverse phase flash chromatography and eluted with 40% to 50% ACN in H₂O (0.1% NH₄HCO₃).
¹⁴Purified by Prep-HPLC that used the following conditions: Column: XBridge Prep C18 OBD, 19 × 150 mm, 5 μm, eluted with 28% to 31% ACN in H₂O (10 mmol/L NH₄HCO₃).
¹⁵Purified by C18 reverse phase chromatography and eluted with 0% to 100% ACN in H₂O (0.1% FA).
¹⁶Purified by Prep-HPLC that used the following conditions: Column: XBridge Prep C18 OBD, 19 × 150 mm, 5 μm, eluted with 26% to 40% ACN in H₂O (10 mmol/L NH₄HCO₃).
¹⁷Purified by Prep-HPLC that used the following conditions: Column: XBridge Prep C18 OBD, 19 × 150 mm, 5 μm, eluted with 31% to 46% ACN in H₂O (10 mmol/L NH₄HCO₃).
¹⁸Purified by silica gel chromatography eluted with MeOH/DCM (25:1 to 20:1) and then recrystallized in hexanes/DCM (200:1).
¹⁹Purified by C18 reverse phase flash chromatography and eluted with 30% to 40% ACN in H₂O (0.1% NH₃—H₂O).
²⁰Purified by Prep-HPLC that used the following conditions: Column: XBridge Prep C18 OBD, 19 × 150 mm, 5 μm, eluted with 23% to 34% ACN in H₂O (10 mmol/L NH₄HCO₃).
²¹Purified by reverse phase C18 flash chromatography and eluted with 30% to 50% ACN in H₂O (0.1% NH₄HCO₃).
²²Purified by C18 reverse phase chromatography and eluted with 5% to 95% ACN in H₂O (1% TFA). Fractions concentrated, residue treated with DCM and washed with sat. aq Na₂CO₃. Organic layer washed with brine, dried over Na₂SO₄, filtered and concentrated.
²³Reaction diluted with DCM/IPA (3:1) and washed with H₂O and brine. Organic layer was dried over Na₂SO₄, filtered, and concentrated. Residue was dissolved into ACN/H₂O (2% TFA)(1:1) and purified on C18 column. Product was eluted with 5% to 95% ACN in H₂O. Fractions containing title compound were combined and pH was adjusted to ~8.0 with sat. aq NaHCO₃. Soln extracted with CHCl₃/IPA (3:1). Organic layer was washed with H₂O, brine, dried over Na₂SO₄, filtered, and concentrated to affotd the title compound.
²⁴Purified by reverse phase flash C18 chromatography and eluted with 30% to 40% ACN in H₂O (0.1% NH₄HCO₃).
²⁵Purified by prep-HPLC: Column: SunFire Prep C18 OBD Column, 19 × 150 mm 5 μm; eluted with 12% to 22% ACN in H2O (0.1% FA).
²⁶Purified by reverse phase flash C18 chromatography and eluted with 15% to 30% ACN in H₂O (0.1% FA).
²⁷Purified by silica gel chromatography and eluted with 0% to 10% MeOH in DCM.
²⁸Purified by C18 reverse phase flash chromatography eluted with 25% to 40% ACN in H₂O (0.1% FA).

### Example 36

### 1-[4-[4-[3-Chloro-4-[(1R)-1-(1-methylpyrazol-3-yl)ethoxy]pyrazolo[1,5-a]pyridin-6-yl]-5-methyl-triazol-1-yl]-1-piperidyl]prop-2-en-1-one

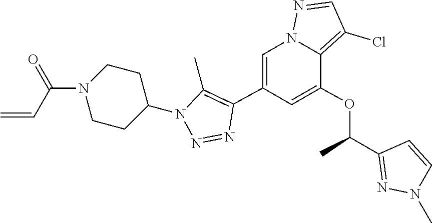 [SMILES: C=CC(=O)N1CCC(n2nnc(-c3cc(O[C@H](C)c4ccn(C)n4)c4c(Cl)cnn4c3)c2C)CC1]

To a mixture of 3-chloro-6-[5-methyl-1-(4-piperidyl)triazol-4-yl]-4-[(1R)-1-(1-methylpyrazol-3-yl)ethoxy]pyrazolo[1,5-a]pyridine hydrochloride (77 mg, 0.17 mmol) and DIPEA (0.11 g, 0.87 mmol) in DCM (3 mL) was added acrylic anhydride (24 mg, 0.19 mmol) and the mixture was stirred for 20 min. The volatiles were removed under reduced pressure. The residue was purified by reverse phase chromatography and eluted with 0% to 100% ACN in H₂O to afford the title compound (29 mg, 34%). ES/MS m/z 495 [M+H]⁺.

The following compounds were prepared essentially as described in Example 36 using the appropriate reagents, adjusting the reaction times to determine completion of the reactions, and adjusting the purification system as appropriate.

TABLE 24

| | | | ES/MS |
|---|---|---|---|
| Example | | | m/z |
| # | Chemical Name | Structure | [M + H]⁺ |
| 37¹ | 1-[4-[4-[3-Chloro-4-(1- cyclobutylethoxy)pyrazolo [1,5-a]pyridin-6-yl]-5- methyl-triazol-1-yl]-1- piperidyl]prop-2-en-1- one | 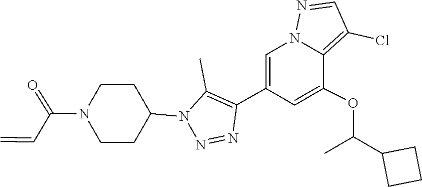 [SMILES: C=CC(=O)N1CCC(n2nnc(-c3cc(OC(C)C4CCC4)c4c(Cl)cnn4c3)c2C)CC1] | 469 |
| 38¹ | 1-[4-[4-[3-Chloro-4-[1- (4- isoquinolyl)ethoxy]pyrazo- lo[1,5-a]pyridin-6-yl]-5- methyl-triazol-1-yl]-1- piperidyl]prop-2-en-1- one | 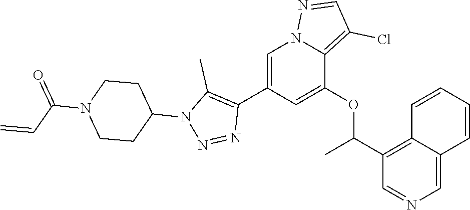 [SMILES: C=CC(=O)N1CCC(n2nnc(-c3cc(OC(C)c4cncc5ccccc45)c4c(Cl)cnn4c3)c2C)CC1] | 542 |
| 39¹ | 1-(4-((1s,3s)-3-(4-(4-(2- (Benzyloxy)-1-(5- fluoropyridin-2- yl)ethoxy)-3- chloropyrazolo[1,5- a]pyridin-6-yl)-5-methyl- 1H-1,2,3-triazol-1- yl)cyclobutyl)piperazin- 1-yl)prop-2-en-1-one, Isomer 1 | 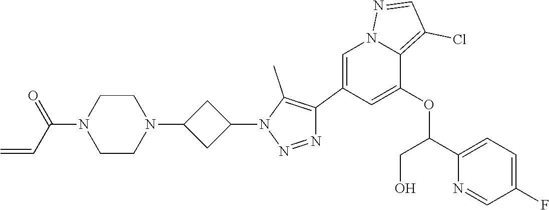 [SMILES: C=CC(=O)N1CCN(C2CC(n3nnc(-c4cc(OC(CO)c5ccc(F)cn5)c5c(Cl)cnn5c4)c3C)C2)CC1] | 581 |
| 40² | 1-[4-[4-[3-Fluoro-4-[2- (5-fluoro-2-pyridyl)-2- hydroxy- propoxy]pyrazolo[1,5- a]pyridin-6-yl]-5-methyl- triazol-1-yl]-1- piperidyl]prop-2-en-1- one | 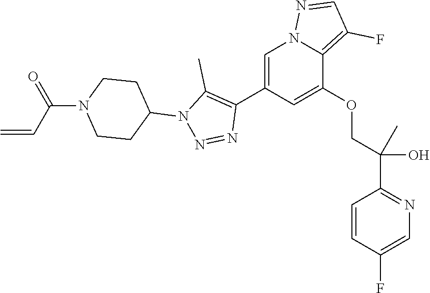 [SMILES: C=CC(=O)N1CCC(n2nnc(-c3cc(OCC(C)(O)c4ccc(F)cn4)c4c(F)cnn4c3)c2C)CC1] | 524 |
| 41 | 1-[4-[4-[3-Chloro-4-[1- (5-methyl-1,3,4- thiadiazol-2- yl)ethoxy]pyrazolo[1,5- a]pyridin-6-yl]-5-methyl- triazol-1-yl]-1- piperidyl]prop-2-en-1- one | 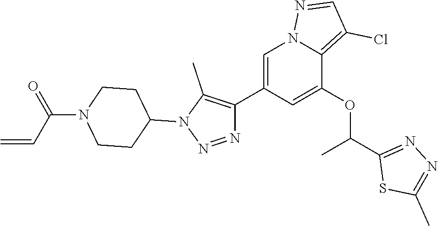 [SMILES: C=CC(=O)N1CCC(n2nnc(-c3cc(OC(C)c4nnc(C)s4)c4c(Cl)cnn4c3)c2C)CC1] | 513 |

¹Purified by reverse phase chromatography and eluted with 10% to 100% ACN in H₂O (0.1% FA).
²Purified by reverse phase chromatography and eluted with 10% to 100% ACN in H₂O.

### Example 42

### 1-[4-[4-(4-Methoxypyrazolo[1,5-a]pyridin-6-yl)-5-methyl-pyrazol-1-yl]-1-piperidyl]prop-2-en-1-one

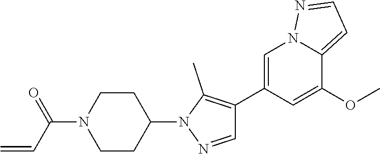 [SMILES: C=CC(=O)N1CCC(n2ncc(-c3cc(OC)c4ccnn4c3)c2C)CC1]

A soln of 4-methoxy-6-[5-methyl-1-(4-piperidyl)pyrazol-4-yl]pyrazolo[1,5-a]pyridine; 2,2,2-trifluoroacetic acid (44.27 mg, 0.14 mmol) and acrylic acid (38.2 mg, 0.53 mmol) in DMF (2 mL) was treated with HATU (83.5 mg, 0.22 mmol) and DIPEA (0.1 mL, 0.57 mmol) and stirred at RT for 1 hr. The reaction was filtered and loaded onto a reverse phase cartridge eluted with 10% to 100% ACN in H₂O to give an off-white solid. This solid was dissolved in 50% MeOH in DCM (3 mL) and loaded onto a bicarbonate cartridge (Agilent StratoSphere SPE). The cartridge was washed with 50% MeOH in DCM (3 mL) and then 100% MeOH to afford the title compound (18.3 mg, 33%). ES/MS m/z 366 [M+H]⁺.

### Example 43

### 1-[4-[4-[3-Chloro-4-[(1S)-2-hydroxy-1-(2-pyridyl)ethoxy]pyrazolo[1,5-a]pyridin-6-yl]-5-methyl-triazol-1-yl]-1-piperidyl]-2-fluoro-prop-2-en-1-one

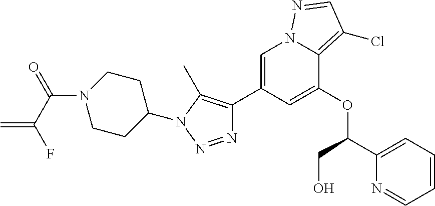 [SMILES: C=C(F)C(=O)N1CCC(n2nnc(-c3cc(O[C@H](CO)c4ccccn4)c4c(Cl)cnn4c3)c2C)CC1]

(2S)-2-[3-Chloro-6-[5-methyl-1-(4-piperidyl)triazol-4-yl]pyrazolo[1,5-a]pyridin-4-yl]oxy-2-(2-pyridyl)ethanol; dihydrochloride (55 mg, 0.10 mmol) and 2-fluoroacrylic acid (9.2 mg, 0.10 mmol) were added to a vial then diluted with DCM (1 mL). DIPEA (53 mg, 0.41 mmol) was added, and the mixture immediately became homogenous. T₃P (0.13 g, 0.2 mmol) was added to the mixture and the reaction was stirred at RT for 30 min. The reaction was concentrated in vacuo. The residue was purified by C18 reverse phase chromatography and eluted with 10% to 100% ACN in H₂O to afford the title compound (38 mg, 71%). ES/MS m/z 526 [M+H]⁺.

### Example 44

### 1-[(3S,4R)-4-[4-[4-[2-(5-Fluoro-2-pyridyl)-2-hydroxy-ethoxy]-3-(trifluoromethyl)pyrazolo[1,5-a]pyridin-6-yl]-5-methyl-triazol-1-yl]-3-hydroxy-1-piperidyl]prop-2-en-1-one

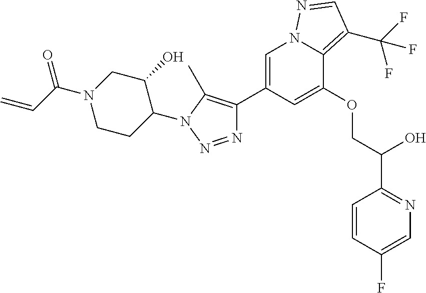 [SMILES: C=CC(=O)N1CCC(n2nnc(-c3cc(OCC(O)c4ccc(F)cn4)c4c(C(F)(F)F)cnn4c3)c2C)[C@@H](O)C1]

(3S,4R)-4-[4-[4-[2-(5-Fluoro-2-pyridyl)-2-hydroxy-ethoxy]-3-(trifluoromethyl) pyrazolo[1,5-a]pyridin-6-yl]-5-methyl-triazol-1-yl]piperidin-3-ol; dihydrochloride (52 mg, 0.09 mmol) was suspended in DCM (2 mL) then DIPEA (114 mg, 0.88 mmol) was added. After being stirred for 5 min, perfluorophenyl acrylate (20 mg, 0.08 mmol) was added. The reaction was stirred for an additional five min and concentrated. The residue was purified by C18 reverse phase chromatography and eluted with 10% to 100% ACN in H₂O to afford (14 mg, 28%) as a white powder. ES/MS m/z 576 [M+H]⁺.

### Example 45

### 6-[1-(1-But-2-ynoyl-4-piperidyl)-5-methyl-pyrazol-4-yl]-4-methoxy-pyrazolo[1,5-a]pyridine-3-carbonitrile

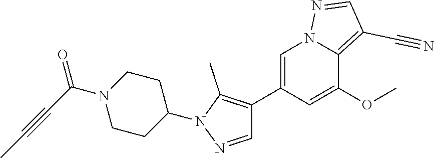 [SMILES: CC#CC(=O)N1CCC(n2ncc(-c3cc(OC)c4c(C#N)cnn4c3)c2C)CC1]

A soln of 4-methoxy-6-[5-methyl-1-(4-piperidyl)pyrazol-4-yl]pyrazolo[1,5-a]pyridine-3-carbonitrile (100 mg, 0.3 mmol), 2-butynoic acid (38 mg, 0.45 mmol) and NMI (74 mg, 0.9 mmol) in ACN (5 mL), was added TCFH (127 mg, 0.45 mmol) and the mixture was stirred for 2 hr at 50° C. under N₂. The soln was purified by C18 reverse phase flash chromatography and eluted with 30% to 50% ACN in H₂O (0.1% NH₄HCO₃) to afford the title compound (50 mg, 42%) as white solid. ES/MS m/z 403 [M+H]⁺.

### Example 46

### 7-[1-(1-But-2-ynoyl-4-piperidyl)-5-methyl-triazol-4-yl]-5-[(1R)-1-(5-fluoro-2-pyridyl)ethoxy]imidazo[1,2-a]pyridine-3-carbonitrile

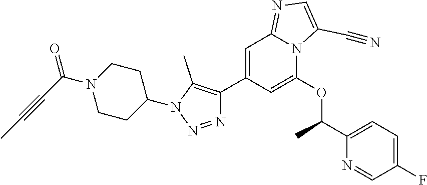 [SMILES: CC#CC(=O)N1CCC(n2nnc(-c3cc(O[C@H](C)c4ccc(F)cn4)n4c(C#N)cnc4c3)c2C)CC1]

To a soln of 5-[(1R)-1-(5-fluoro-2-pyridyl)ethoxy]-7-[5-methyl-1-(4-piperidyl)triazol-4-yl]imidazo[1,2-a]pyridine-3-carbonitrile; hydrochloride (310 mg, 0.7 mmol) and 2-butynoic acid (88 mg, 1 mmol) in DCM (5 mL) was added HATU (1.32 g, 3.5 mmol) and DIPEA (269 mg, 2.1 mmol) in portions at RT under N₂. The resulting mixture was stirred for 2 hr. The mixture was diluted with H₂O (20 mL) and extracted with DCM (3×20 mL). The combined organic layers were washed with brine (3×10 mL), dried over Na₂SO₄, filtered, and concentrated. The residue was purified by C18 reverse phase flash chromatography and eluted with 60% to 70% ACN in H₂O (0.1% NH₄HCO₃) to afford the title compound (103 mg, 28%) as a white solid. ES/MS m/z 513 [M+H]⁺.

### Example 47

### 6-[1-[1-(1-But-2-ynoyl-4-piperidyl)azetidin-3-yl]-5-methyl-pyrazol-4-yl]-4-methoxy-pyrazolo[1,5-a]pyridine-3-carbonitrile

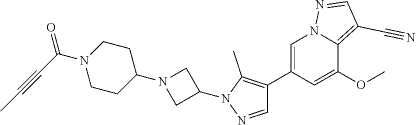 [SMILES: CC#CC(=O)N1CCC(N2CC(n3ncc(-c4cc(OC)c5c(C#N)cnn5c4)c3C)C2)CC1]

To a mixture of 4-methoxy-6-[5-methyl-1-[1-(4-piperidyl)azetidin-3-yl]pyrazol-4-yl]pyrazolo[1,5-a]pyridine-3-carbonitrile; 2,2,2-trifluoroacetic acid (80 mg, 0.16 mmol), 2-butynoic acid (17 mg, 0.20 mmol) and DIPEA (61 mg, 0.47 mmol) in DCM (2 mL) was added T₃P (50% in EA, 298 mg, 0.47 mmol) at RT under N₂. The mixture was stirred for 2 hr at RT. The mixture was concentrated. The residue was purified by reverse phase flash chromatography and eluted with 10% to 40% ACN in H₂O (0.1% NH₃H₂O) to afford the title compound (33 mg, 45%) as a light-green solid. ES/MS m/z 458 [M+H]⁺.

The following compound was prepared essentially as described in Example 47 using the appropriate reagents, adjusting the temperature, adjusting the reaction times to determine completion of the reactions, and adjusting the purification system as appropriate.

TABLE 25

| | | | ES/MS |
|---|---|---|---|
| Example | | | m/z |
| # | Chemical Name | Structure | [M + H]+ |
| 48¹ | N-[1-[[4-[4-(3-Cyano-4- methoxy-pyrazolo[1,5- a]pyridin-6-yl)-5-methyl- pyrazol-1-yl]-1- piperidyl]methyl]cyclopro- pyl]but-2-ynamide | 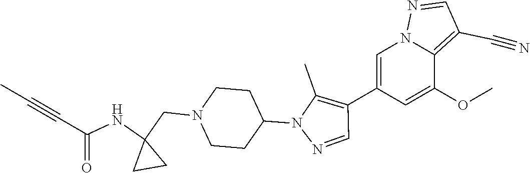 [SMILES: CC#CC(=O)NC1(CN2CCC(n3ncc(-c4cc(OC)c5c(C#N)cnn5c4)c3C)CC2)CC1] | 472 |

¹Purified by reverse phase flash chromatography eluted with 0% to 70% ACN in H₂O (0.1% FA).

### Example 49

### 6-[1-[1-[2-Cyano-4-methyl-pent-2-enoyl]-4-piperidyl]-5-methyl-pyrazol-4-yl]-4-methoxy-pyrazolo[1,5-a]pyridine-3-carbonitrile

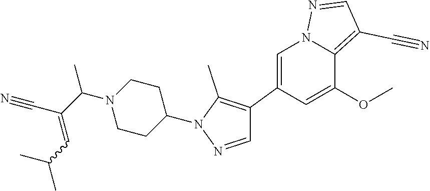 [SMILES: COc1cc(-c2cnn(C3CCN(C(C)C(C#N)=CC(C)C)CC3)c2C)cn2ncc(C#N)c12]

To a soln of 6-[1-[1-(2-cyanoacetyl)-4-piperidyl]-5-methyl-pyrazol-4-yl]-4-methoxy-pyrazolo[1,5-a]pyridine-3-carbonitrile (80 mg, 0.2 mmol) and piperidine acetic acid salt (29 mg, 0.2 mmol) in IPA (3 mL) was added isobutyraldehyde (14 mg, 0.20 mmol). The soln was stirred for 2 hr at 80° C. under N₂. Upon cooling to RT, the reaction was concentrated. The residue was purified by reverse phase flash chromatography: Column, C18; eluted with 20% to 40% ACN in H₂O (0.1% FA) to afford the title compound (9.8 mg, 11%) as a red solid. ES/MS m/z 458 [M+H]⁺. ¹H NMR (400 MHz, DMSO-d₆) δ 8.58 (s, 1H), 8.54 (s, 1H), 7.83 (s, 1H), 7.09 (s, 1H), 6.95 (d, 1H), 4.68-4.58 (m, 1H), 4.52-4.30 (m, 1H), 4.14-3.93 (m, 4H), 3.58-3.43 (m, 1H), 3.11-2.92 (m, 1H), 2.89-2.75 (m, 1H), 2.49 (s, 3H), 2.09-1.82 (m, 4H), 1.12 (d, 6H).

The following compound was prepared essentially as described in Example 49 using the appropriate reagents, adjusting the temperature, adjusting the reaction times to determine completion of the reactions, and adjusting the purification system as appropriate.

TABLE 26

| | | | ES/MS |
|---|---|---|---|
| Example | | | m/z |
| # | Chemical Name | Structure | [M + H]+ |
| 50^{1,2,3} | 6-[1-[1-[2- Cyanobut-2- enoyl]-4- piperidyl]-5- methyl-pyrazol-4- yl]-4-methoxy- pyrazolo[1,5- a]pyridine-3- carbonitrile | 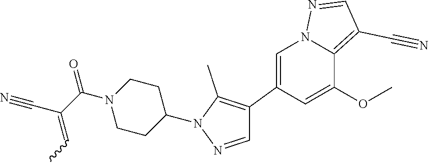 | 430 |

¹NEt₃ used in place of piperidine acetic acid salt.
²Purified by C18 reverse phase flash chromatography: Column, C18; eluted with 25% to 35% ACN in H₂O (0.1%FA).
³¹H NMR (300 MHz, CDCl₃) δ 8.19 (s, 1H), 8.15 (d, 1H), 7.64 (s, 1H), 7.26 (q, 1H), 6.68 (s, 1H), 4.80-4.30 (m, 3H), 4.08 (s, 3H), 3.43-3.02 (m, 2H), 2.46 (s, 3H), 2.39-2.26 (m, 2H), 2.22 (d, 3H), 2.14-2.03 (m, 2H).

### Example 51

### 6-[1-[1-[2-Cyano-3-(1-methylpyrazol-4-yl)prop-2-enoyl]-4-piperidyl]-5-methyl-pyrazol-4-yl]-4-methoxy-pyrazolo[1,5-a]pyridine-3-carbonitrile

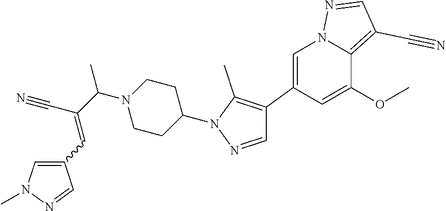 [SMILES: COc1cc(-c2cnn(C3CCN(C(C)C(C#N)=Cc4cnn(C)c4)CC3)c2C)cn2ncc(C#N)c12]

A soln of 6-[1-[1-(2-cyanoacetyl)-4-piperidyl]-5-methyl-pyrazol-4-yl]-4-methoxy-pyrazolo[1,5-a]pyridine-3-carbonitrile (50 mg, 0.10 mmol) in MeOH (6.2 mL) was treated with 1-methylpyrazole-4-carbaldehyde (23 mg, 0.21 mmol) followed by piperidine (0.02 mL, 0.21 mmol). The reaction was heated in a sealed tube at 50° C. overnight. Upon cooling to RT, the reaction was diluted with DCM/IPA (4:1) and the layers were separated. The organic layer was washed with H₂O, brine, dried over Na₂SO₄, filtered, and concentrated to a yellow oil. The oil was dissolved into DCM. The soln was purified by silica gel chromatography and eluted with 0% to 20% MeOH in DCM. Fractions containing the title compound were combined and concentrated. The solid was dissolved into DMSO and H₂O was added dropwise, with stirring, until cloudiness persisted. The mixture was stirred for 30 minutes, then filtered to collect the title compound (23 mg, 43% yield) as an off-white solid ES/MS m/z 496 [M+H]⁺.

The following compound was prepared essentially as described in Example 51 using the appropriate reagents, adjusting the temperature, adjusting the reaction times to determine completion of the reactions, and adjusting the purification system as

TABLE 27

| | | | ES/MS |
|---|---|---|---|
| Example | | | m/z |
| # | Chemical Name | Structure | [M + H]+ |
| 52¹ | 6-[1-[1-[2-Cyano-3- tetrahydropyran-4- yl-prop-2-enoyl]-4- piperidyl]-5-methyl- pyrazol-4-yl]-4- methoxy- pyrazolo[1,5- a]pyridine-3- carbonitrile | 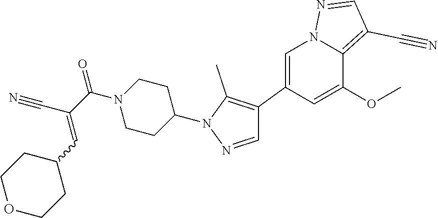 [SMILES: COc1cc(-c2cnn(C3CCN(C(=O)C(C#N)=CC4CCOCC4)CC3)c2C)cn2ncc(C#N)c12] | 500 |

¹Workup: Concentrated. Residue purified by reverse phase chromatography, eluted with 5% to 95% ACN in H₂O (1% TFA).

### Example 53

### 6-[1-[1-[2-Cyano-4-(2,2-difluoroethylamino)-4-methyl-pent-2-enoyl]-4-piperidyl]-5-methyl-pyrazol-4-yl]-4-methoxy-pyrazolo[1,5-a]pyridine-3-carbonitrile

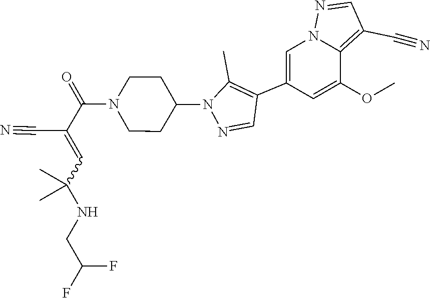 [SMILES: COc1cc(-c2cnn(C3CCN(C(=O)C(C#N)=CC(C)(C)NCC(F)F)CC3)c2C)cn2ncc(C#N)c12]

A soln of 2-(2,2-difluoroethylamino)-2-methyl-propanal (0.10 g, 0.66 mmol) and 6-[1-[1-(2-cyanoacetyl)-4-piperidyl]-5-methyl-pyrazol-4-yl]-4-methoxy-pyrazolo[1,5-a]pyridine-3-carbonitrile (0.13 g, 0.33 mmol) in iPrOH (15 mL) was treated with piperidine (0.17 g, 2 mmol) and stirred at 80° C. for 2 hr. After cooling to RT, the reaction was concentrated, the residue dissolved in ACN (5 mL). The soln was loaded onto a C18 column and eluted with 10% to 50% ACN in H₂O (0.1% NH₄HCO₃) to afford the title compound (28.4 mg, 10%) as an off-white solid. ES/MS m/z 537 [M+H]⁺.

### Example 54

### 6-[1-[1-[2-Cyano-3-isothiazol-4-yl-prop-2-enoyl]-4-piperidyl]-5-methyl-pyrazol-4-yl]-4-methoxy-pyrazolo[1,5-a]pyridine-3-carbonitrile

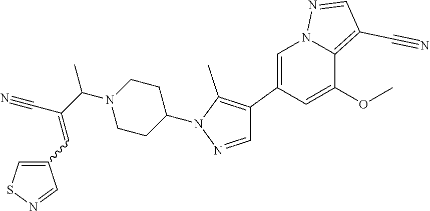 [SMILES: COc1cc(-c2cnn(C3CCN(C(C)C(C#N)=Cc4cnsc4)CC3)c2C)cn2ncc(C#N)c12]

A soln of 6-[1-[1-(2-cyanoacetyl)-4-piperidyl]-5-methyl-pyrazol-4-yl]-4-methoxy-pyrazolo[1,5-a]pyridine-3-carbonitrile (50 mg, 0.1 mmol) in MeOH (6.3 mL) was treated with isothiazole-4-carbaldehyde (23 mg, 0.2 mmol) followed by piperidine (0.02 mL, 0.2 mmol). The reaction was heated at 50° C. overnight in a sealed tube. Upon cooling to RT, the reaction was diluted with DCM/IPA (4:1) and the layers were separated. The organic layer was washed with H₂O, brine, dried over Na₂SO₄, filtered, and concentrated to a yellow oil. The oil was dissolved into in ACN/H₂O (2% TFA) (1:1) and loaded onto a C18 column. Material eluted with 10% to 95% ACN in H₂O (0.1% FA). Fractions containing the title compound were combined and extracted with DCM/IPA (4:1). Organic layers were combined and washed with H₂O, brine, dried over Na₂SO₄, filtered, and concentrated to a yellow oil. Oil taken up in DCM and purified by silica gel chromatography and eluted with 20% to 100% EA in DCM to afford the title compound (25 mg, 49%). ES/MS m/z 499 [M+H]⁺.

### Example 55

### 6-[1-[1-[2-Cyano-4,4-dimethyl-5-morpholino-pent-2-enoyl]-4-piperidyl]-5-methyl-pyrazol-4-yl]-4-methoxy-pyrazolo[1,5-a]pyridine-3-carbonitrile

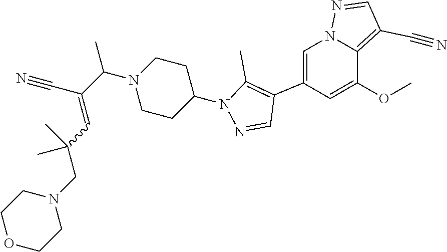 [SMILES: COc1cc(-c2cnn(C3CCN(C(C)C(C#N)=CC(C)(C)CN4CCOCC4)CC3)c2C)cn2ncc(C#N)c12]

A soln of 6-[1-[1-(2-cyanoacetyl)-4-piperidyl]-5-methyl-pyrazol-4-yl]-4-methoxy-pyrazolo[1,5-a]pyridine-3-carbonitrile (100 mg, 0.24 mmol), 2,2-dimethyl-3-(morpholin-4-yl)propanal (127 mg, 0.74 mmol), TMSCl (135 mg, 1.24 mmol) and pyrrolidine (88 mg, 1.24 mmol) in DCM (5 mL) was stirred for 1 hr at 50° C. under N₂. The resulting mixture was cooled to RT and diluted with H₂O (50 mL). The mixture was extracted with DCM (2×50 mL). The combined organic layers were washed with brine (2×50 mL), dried over Na₂SO₄, filtered, and the filtrate was concentrated. The residue was purified by reverse phase flash chromatography: Column, C18; eluted with 30% to 50% ACN in H₂O (0.05% NH₃·H₂O) to afford the title compound (17 mg, 13%) as a white solid. ES/MS m/z 557 [M+H]⁺. ¹H NMR (400 MHz, CDCl₃) δ 8.19 (s, 1H), 8.15 (s, 1H), 7.64 (s, 1H), 7.03 (s, 1H), 6.68 (s, 1H), 4.72-4.49 (m, 1H), 4.40-4.32 (m, 2H), 4.08 (s, 3H), 3.72 (t, 4H), 3.47-2.99 (m, 2H), 2.57 (t, 4H), 2.48-2.24 (m, 5H), 2.39-2.24 (m, 2H), 2.12-2.05 (m, 2H), 1.34 (s, 6H).

The following compound was prepared essentially as described in Example 55 using the appropriate reagents, adjusting the temperature, adjusting the reaction times to determine completion of the reactions, and adjusting the purification system as appropriate.

TABLE 28

| | | | ES/MS |
|---|---|---|---|
| Example | | | m/z |
| # | Chemical Name | Structure | [M + H]⁺ |
| 56^{1,2} | 6-[1-[1-[2-Cyano- 4-methyl-4- [methyl(oxetan-3- yl)amino]pent-2- enoyl]-4-piperidyl]- 5-methyl-pyrazol- 4-yl]-4-methoxy- pyrazolo[1,5- a]pyridine-3- carbonitrile | 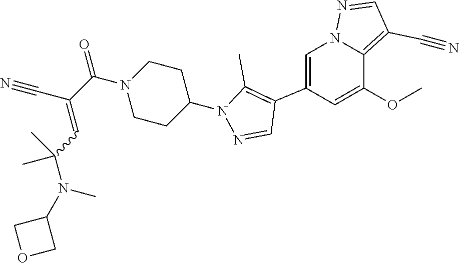 [SMILES: COc1cc(-c2cnn(C3CCN(C(=O)C(C#N)=CC(C)(C)N(C)C4COC4)CC3)c2C)cn2ncc(C#N)c12] | 543 |

¹Purified by reverse phase flash chromatography: Column, C18; eluted with 30% to 50% ACN in H₂O (0.1% NH₄HCO₃).
²¹H NMR (300 MHz, DMSO-d₆) δ 8.58 (s, 1H), 8.54 (d, 1H), 7.83 (s, 1H), 7.09 (d, 1H), 6.84 (d, 1H), 4.66-4.30 (m, 6H), 4.15-4.03 (m, 4H), 4.00-3.86 (m, 1H), 3.50-3.39 (m, 1H), 3.10-2.92 (m, 1H), 2.49 (s, 3H), 2.33 (s, 3H), 2.08-1.90 (m, 4H), 1.18 (s, 6H).

### Example 57

### 6-[1-[1-[2-Cyano-4-(2,7-diazaspiro[3.5]nonan-2-yl)-4-methyl-pent-2-enoyl]-4-piperidyl]-5-methyl-pyrazol-4-yl]-4-methoxy-pyrazolo[1,5-a]pyridine-3-carbonitrile; 2,2,2-trifluoroacetic acid

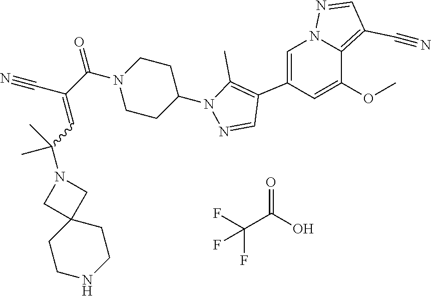 [SMILES: COc1cc(-c2cnn(C3CCN(C(=O)C(C#N)=CC(C)(C)N4CC5(CCNCC5)C4)CC3)c2C)cn2ncc(C#N)c12 ; O=C(O)C(F)(F)F]

To a stirred soln of tert-butyl 2-[(E)-3-cyano-4-[4-[4-(3-cyano-4-methoxy-pyrazolo[1,5-a]pyridin-6-yl)-5-methyl-pyrazol-1-yl]-1-piperidyl]-1,1-dimethyl-4-oxo-but-2-enyl]-2,7-diazaspiro[3.5]nonane-7-carboxylate (100 mg, 0.17 mmol) in DCM (5 mL) was added TFA (1 mL) dropwise at RT under N₂. The mixture was stirred for 0.5 hr at RT then concentrated. Et₂O (5 mL) was added, and the organic phase was separated. ACN (3 mL) and H₂O (5 mL) were added, and the mixture was lyophilized to afford the title compound (56 mg, 53%) as a white solid. ES/MS m/z 582 [M+H]⁺. ¹H NMR (300 MHz, DMSO-d₆) δ 8.49 (s, 1H), 8.44 (s, 1H), 7.80 (s, 1H), 7.01 (s, 1H), 6.96 (s, 1H), 4.66-4.52 (m, 1H), 4.47-4.31 (m, 1H), 4.12-3.95 (m, 8H), 3.45-3.30 (m, 1H), 3.11-2.91 (m, 5H), 2.44 (s, 3H), 2.11-1.85 (m, 8H), 1.59 (s, 6H).

The following compound was prepared essentially as described in Example 57 using the appropriate reagents, adjusting the temperature, adjusting the reaction times to determine completion of the reactions, and adjusting the purification system as appropriate.

TABLE 29

| | | | ES/MS |
|---|---|---|---|
| | | | m/z |
| Example # | Chemical Name | Structure | [M + H]⁺ |
| 58¹ | 6-[1-[1-[2-Cyano- 4-hydroxy-4- methyl-pent-2- enoyl]-4- piperidyl]-5- methyl-pyrazol-4- yl]-4-methoxy- pyrazolo[1,5- a]pyridine-3- carbonitrile; 2,2,2- trifluoroacetic acid | 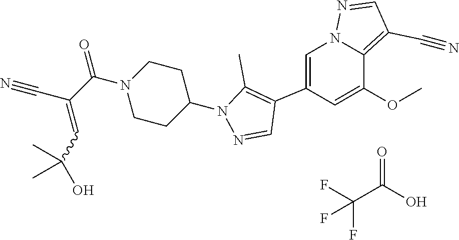 [SMILES: COc1cc(-c2cnn(C3CCN(C(=O)C(C#N)=CC(C)(C)O)CC3)c2C)cn2ncc(C#N)c12 ; O=C(O)C(F)(F)F] | 474 |

¹¹H NMR (300 MHz, DMSO-d₆) δ 11.11 (brs, 1H), 8.59 (s, 1H), 8.55 (s, 1H), 8.24 (s, 1H), 7.84 (s, 1H), 7.09 (s, 1H), 4.73-4.51 (m, 2H), 4.08 (s, 3H), 4.07-3.93 (m, 1H), 3.31-3.18 (m, 1H), 3.11-2.97 (m, 1H), 2.50 (s, 3H), 2.25-2.11 (m, 1H), 2.05-1.88 (m, 3H), 1.65 (s, 6H).

### Biological Assays

The following assays demonstrate that compounds provided herein are FGFR3 inhibitors The following assays demonstrate that certain compounds provided herein selectively target FGFR3.

### FGFR3 and FGFR1 Enzyme Assay 1

FGFR3 protein was purchased from Reaction Biology (Cat. No. 1068), and FGFR1 protein was purchased from ThermoFisher Scientific (Cat. No. PV4105). Enzyme activity was monitored using the KinEASE™-TK Assay Kit (CisBio, Cat. No. 62TK0PEC) according to the manufacturer's instructions. All assays were performed at the respective KmATP for each kinase in KinEASE™ Kinase Buffer. Reactions were performed in a white, small volume polystyrene 384 well plate (Greiner, Cat. No. 784075-25).

An incubation was conducted with FGFR3 protein or FGFR1 protein, 125.0 nM TK-Biotin Substrate (CisBio), 7.81 nM Streptavidin-XL665 (CisBio), 0.25×Anti-Phosphorylate TK-Biotin-Cryptate (CisBio). Final enzyme concentrations were 0.25 nM in 10 uL reactions. Titration of compounds were performed in a half-log manner in 100% dimethyl sulfoxide (DMSO) starting at 1 uM. Prior to the initiation of the reaction by adenosine triphosphate (ATP), FGFR1 protein and compounds were pre-incubated for 15 minutes at room temperature, and FGFR3 protein and compounds were pre-incubated on ice for 15 minutes. Reactions proceeded for 30 min at 30° C. Plates were quenched by the addition of the Anti-TK cryptate antibody/Streptavidin-XL665 mixture. After 1 hour. in the stopping solution, the plates were read on the Envision plate reader ((Perkin Elmer) (Ex. Filter. 320 nm and Em1 665 nm/Em2 615 nm)).

### FGFR3 and FGFR1 Enzyme Assay 2

FGFR3 protein was purchased from Reaction Biology (Cat. No. 1068), and FGFR1 protein was purchased from ThermoFisher Scientific (Cat. No. PR4660A). Enzyme activity was monitored using the KinEASE™-TK Assay Kit (CisBio, Cat. No. 62TK0PEC) according to the manufacturer's instructions. All assays were performed at the respective KmATP for each kinase in KinEASE™ Kinase Buffer. Reactions were performed in a white, small volume polystyrene 384 well plate (Corning, Cat. No. 3825).

An incubation was conducted with FGFR3 protein or FGFR1 protein, 125.0 nM TK-Biotin Substrate (CisBio), 15.62 nM Streptavidin-XL665 (CisBio), 0.25×Anti-Phosphorylate TK-Biotin-Cryptate (CisBio). Final enzyme concentrations varied by construct and lot, ranging from 0.07 to 0.5 nM in 10 uL reactions (FGFR1 0.5 nM, FGFR3 0.07 nM. Titration of compounds were performed in a 2.5 fold dilution manner in 100% dimethyl sulfoxide (DMSO) starting at 20 uM. Prior to the initiation of the reaction by adenosine triphosphate (ATP), FGFR1 protein and compounds were pre-incubated for 15 minutes at room temperature, and FGFR3 protein and compounds were pre-incubated on ice for 15 minutes. Reactions proceeded for 30 min at 25° C. Plates were quenched by the addition of the Anti-TK cryptate antibody/Streptavidin-XL665 mixture. After 1 hour. in the stopping solution, the plates were read on the Pherastar plate reader ((BMG) (Ex. Filter. 320 nm and Em1 665 nm/Em2 615 nm)).

For enzyme assays 1 and 2, ratios were converted to a percent of control (POC) using a ratiometric emission factor. One hundred POC was determined using no test compound, and 0 POC was determined in the presence of 1 uM of an appropriate control inhibitor. A 4-parameter logistic curve was fit to the POC values as a function of the concentration of compound, and the IC₅₀ value was the point where the best fit curve crossed 50 POC.

In enzyme assay 1 the compounds of Examples 1, 3-24, 29-34 and 45-58 exhibited IC₅₀ values of less than 350 nM for FGFR3.

In enzyme assay 1 the compounds of Examples 1, 3-13, 15-17, 19-21, 24, 29-34, 45, 46 and 48-58 exhibited IC₅₀ values of less than 100 nM for FGFR3 and are at least 3 fold more selective for FGFR3 than for FGFR1.

In enzyme assay 1 the compounds of Examples 1, 6-8, 10, 11, 24, 29, 32, 46, 49, 50-52, 54-56 and 58 exhibited IC₅₀ values of less than 50 nM for FGFR3 and are at least 10 fold more selective for FGFR3 than for FGFR1.

In enzyme assay 2 the compounds of Examples 36, 37, 39-41, 43 and 44 exhibited IC₅₀ values of less than 350 nM for FGFR3.

In enzyme assay 2 the compounds of Examples 36, 37, 39-41, 43 and 44 exhibited IC₅₀ values of less than 100 nM for FGFR3 and are at least 3 fold more selective for FGFR3 than for FGFR1.

In enzyme assay 2 the compounds of Examples 36, 39-41, 43 and 44 exhibited IC₅₀ values of less than 50 nM for FGFR3 and are at least 10 fold more selective for FGFR3 than for FGFR1.

1-55. (canceled)

56. A compound of the formula: 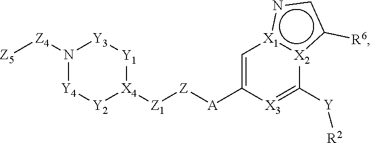
wherein
Z₅ is 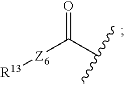
A is pyrazole, triazole, thiadiazole, or oxadiazole, substituted with R¹ and R^{1A};
R¹ is hydrogen or C₁-C₃ alkyl;
R^{1A} is hydrogen, halo, CN, or C₁-C₃ alkyl optionally substituted with one or more substituents independently selected from halo, OH, and OCH₃;
X₁ and X₂ are independently selected from N and C, wherein when one of X₁ or X₂ is N the other is C;
X₃ is N or CH;
X₄ is N or C—R⁹;
Y is NH, O, S, or a bond;
Y₁ is a bond, CHR⁷, CH₂—CHR⁷ or CHR⁷—CH₂, CF₂, CH₂—CF₂, or CF₂—CH₂;
Y₂ is a bond, CHR³, CH₂—CHR³ or CHR³—CH₂, CF₂, CH₂—CF₂, or CF₂—CH₂;
Y₃ is CR⁴R⁵ or CF₂;
Y₄ is CR³R⁴ or CF₂;
Y₅ is CR^{5A}R^{6A} or 3-6 membered cycloalkyl;
Z is a bond, CHR^{9A}, CR⁴R^{4A}, CR⁴R^{4A}—CH₂, CH₂—CR⁴R^{4A}, cyclobutyl, cyclopentyl, cyclohexyl, bicyclo(1.1.1)pentane, bicyclo(2.1.1)hexane, azetidine, pyrrolidine, or piperidine;
Z₁ is a bond when Z is a bond, CR⁴R^{4A}, CR⁴R^{4A}—CH₂, CH₂—CR⁴R^{4A}, cyclobutyl, cyclopentyl, cyclohexyl, bicyclo(1.1.1)pentane, bicyclo(2.1.1)hexane, azetidine, pyrrolidine, or piperidine, or Z₁ is CH₂ or CH₂—CH₂ when Z is CHR^{9A};
Z₂ is a bond, C(O), SO₂, or —NR⁴C(O);
Z₃ is a bond, C(O), SO₂, or —NR⁴C(O);
Z₄ is a bond, Y₅—NR¹⁵, or CH₂—Y₅—NR¹⁵, wherein the N of NR¹⁵ is connected to Z₅;
Z₆ is C═C or C≡C, wherein C═C is optionally substituted with R¹⁴;
R² is C₁-C₅ alkyl or R⁸, wherein C₁-C₅ alkyl is optionally substituted with one or more substituents independently selected from halo, OH, CN, oxo, —OC₁-C₄ alkyl, —OC₃-C₅ cycloalkyl, —Z₂—R¹¹, and R¹⁰, wherein C₁-C₄ alkyl and C₃-C₅ cycloalkyl are optionally substituted with one or more substituents independently selected from halo, OH, OCH₃, methylamine, N,N-dimethylamine, and CN;
R³ is hydrogen, F, OH, OCH₃, C₁-C₃ alkyl, cyclopropyl, or one R³ is fused with R⁵ or R⁷ to form CH₂, CH₂—CH₂, or CH₂OCH₂;
R⁴ is hydrogen or C₁-C₃ alkyl;
R^{4A} is hydrogen, halo, OH, or C₁-C₃ alkyl;
R⁵ is hydrogen, F, OH, OCH₃, C₁-C₃ alkyl, cyclopropyl, or is fused with one R³ to form CH₂, CH₂—CH₂, or CH₂OCH₂;
R^{5A} is hydrogen or C₁-C₃ alkyl;
R⁶ is hydrogen, halo, C₁-C₅ alkyl, CN, 3-6 membered cycloalkyl, 4-6 membered heterocycloalkyl, 5-6 membered aryl, or 5-6 membered heteroaryl, wherein 3-6 membered cycloalkyl, 4-6 membered heterocycloalkyl, 5-6 membered aryl, and 5-6 membered heteroaryl are optionally substituted with one or more substituents independently selected from halo, methyl, halomethyl, OH, or OCH₃ and wherein C₁-C₅ alkyl is optionally substituted with one or more substituents independently selected from halo, OH, and OCH₃;
R^{6A} is hydrogen or C₁-C₃ alkyl;
R⁷ is hydrogen, F, OH, OCH₃, C₁-C₃ alkyl, or is fused with one R³ to form CH₂, CH₂—CH₂, or CH₂OCH₂;
R⁸ is 3-6 membered cycloalkyl, 4-6 membered heterocycloalkyl, 5-6 membered aryl, or 5-6 membered heteroaryl, optionally fused or substituted with R^{8A};
R^{8A} is 3-6 membered cycloalkyl, 4-6 membered heterocycloalkyl, 5-6 membered aryl, or 5-6 membered heteroaryl;
R⁹ is hydrogen, C₁-C₃ alkyl, or is fused with R^{9A} to form CH₂ or CH₂—CH₂;
R¹⁰ is 3-6 membered cycloalkyl, 4-6 membered heterocycloalkyl, 5-6 membered aryl, or 5-6 membered heteroaryl, optionally fused or substituted with RSA;
R₁₁ is C₁-C₄ alkyl, NH₂, NHC₁-C₃ alkyl, NHC₃-C₅ cycloalkyl, or N(C₁-C₃ alkyl)₂, wherein C₁-C₄ alkyl, C₁-C₃ alkyl, and C₃-C₅ cycloalkyl are optionally substituted with one or more substituents independently selected from halo, OH, OCH₃, methylamine, N,N-dimethylamine, and CN;
R¹² is C₁-C₄ alkyl, C₃-C₅ cycloalkyl, NH₂, NHC₁-C₃ alkyl, NHC₃-C₅ cycloalkyl, or N(C₁-C₃ alkyl)₂, wherein C₁-C₄ alky, C₁-C₃ alkyl, and C₃-C₅ cycloalkyl are optionally substituted with one or more substituents independently selected from halo, OH, OCH₃, methylamine, N,N-dimethylamine, and CN;
R¹³ is hydrogen, C₁-C₅ alkyl, or R¹⁷, wherein C₁-C₅ alkyl is optionally substituted with one or more substituents independently selected from halo, OH, CN, —OC₁-C₃ alkyl, NH₂, NHC₁-C₃ alkyl or N(C₁-C₃ alkyl)₂, R¹⁷, NR¹⁶R¹⁷, and —OR¹⁷, wherein C₁-C₃ alkyl is optionally substituted with one or more substituents independently selected from halo, OH, OCH₃, and CN;
R¹⁴ is F, CF₃, or CN;
R¹⁵ is hydrogen or C₁-C₃ alkyl;
R¹⁶ is hydrogen or C₁-C₃ alkyl
R¹⁷ is 3-6 membered cycloalkyl, 4-6 membered heterocycloalkyl, 5-6 membered aryl, 5-6 membered heteroaryl, or 7-12 membered spiroheteroalkyl having 1-2 ring nitrogen atoms, wherein 3-6 membered cycloalkyl, 4-6 membered heterocycloalkyl, 5-6 membered aryl, and 5-6 membered heteroaryl are optionally fused or substituted with R^{17A};
R^{17A} is 3-6 membered cycloalkyl, 4-6 membered heterocycloalkyl, 5-6 membered aryl, or 5-6 membered heteroaryl; and
R⁸, R¹⁰, R¹⁷, R^{8A}, and R^{17A} are optionally substituted with one or more substituents independently selected from halo, OH, CN, —OC₁-C₄ alkyl, —OC₃-C₅ cycloalkyl, and —Z₃—R¹² wherein C₁-C₄ alkyl and C₃-C₅ cycloalkyl are optionally substituted with one or more substituents independently selected from halo, OH, OCH₃, methylamine, N,N-dimethylamine, and CN;
or a pharmaceutically acceptable salt thereof.

57. The compound according to claim 56 wherein Z₆ is C═C, or a pharmaceutically acceptable salt thereof.

58. The compound according to claim 56, wherein R¹³ is hydrogen or C₁-C₃ alkyl, wherein C₁-C₃ alkyl is optionally substituted with one or more substituents independently selected from halo, OH, and CN, or a pharmaceutically acceptable salt thereof.

59. The compound according to claim 56, wherein Z₄ is a bond, or a pharmaceutically acceptable salt thereof.

60. The compound according to claim 56, wherein X₁ is N, and X₂ is C, or a pharmaceutically acceptable salt thereof.

61. The compound according to claim 56, wherein X₃ is CH, or a pharmaceutically acceptable salt thereof.

62. The compound according to claim 56 wherein A is pyrazole or triazole, substituted with R¹ and R^{1A}, or a pharmaceutically acceptable salt thereof.

63. The compound according to claim 56, wherein R^{1A} is hydrogen, or a pharmaceutically acceptable salt thereof.

64. The compound according to claim 56, wherein R¹ is CH₃, or a pharmaceutically acceptable salt thereof.

65. The compound according to claim 56, wherein Y is O, or a pharmaceutically acceptable salt thereof.

66. The compound according to claim 56, wherein R⁶ is CN, F, Cl, or CF₃, or a pharmaceutically acceptable salt thereof.

67. The compound according to claim 56, wherein Z is a bond, cyclobutyl, azetidine, or piperidine, or a pharmaceutically acceptable salt thereof.

68. The compound according to claim 56, wherein Z₁ is a bond, or a pharmaceutically acceptable salt thereof.

69. The compound according to claim 56, wherein X₄ is C—R⁹, wherein R⁹ is hydrogen or CH₃, or a pharmaceutically acceptable salt thereof.

70. The compound according to claim 56, wherein Y₁ is a bond, CHR⁷, CH₂—CHR⁷, or CHR⁷—CH₂, wherein R⁷ is selected from hydrogen, F, OH, and CH₃; Y₂ is a bond, CHR³, CH₂—CHR³, or CHR³—CH₂, wherein R³ is selected from hydrogen, F, OH, and CH₃; Y₃ is CR⁴R⁵ or CF₂, wherein R⁴ is hydrogen or CH₃ and R⁵ is hydrogen, F, OH, or CH₃; and Y₄ is CR³R⁴ or CF₂ wherein R⁴ is hydrogen or CH₃ and R³ is hydrogen, F, OH, or CH₃, or a pharmaceutically acceptable salt thereof.

71. The compound according to claim 56, wherein R² is C₁-C₃ alkyl optionally substituted with one, two, three, or four substituents independently selected from halo, OH, CN, oxo, —OC₁-C₄ alkyl, —OC₃-C₅ cycloalkyl, —Z₃—R¹¹, and R¹⁰, wherein C₁-C₄ alkyl and C₃-C₅ cycloalkyl are optionally substituted with one or more substituents independently selected from halo, OH, OCH₃, methylamine, N,N-dimethylamine, and CN, or a pharmaceutically acceptable salt thereof.

72. The compound according to claim 56, wherein R² is selected from: 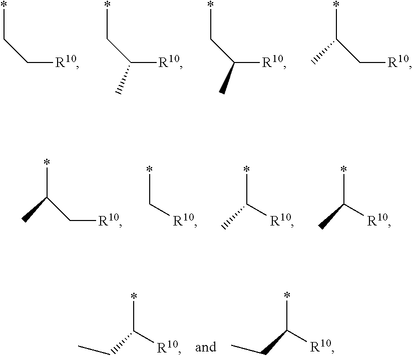
optionally substituted with one, two, three or four substituents independently selected from halo, OH, CN, oxo, —OC₁-C₄ alkyl, —OC₃-C₅ cycloalkyl, —Z₃—R¹¹, and R¹⁰, wherein C₁-C₄ alkyl and C₃-C₅ cycloalkyl are optionally substituted with one or more substituents independently selected from halo, OH, OCH₃, methylamine, N,N-dimethylamine, and CN, wherein * indicates the connection point to Y, or a pharmaceutically acceptable salt thereof.

73. The compound according to claim 56, wherein R² is selected from: 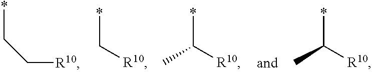
optionally substituted with one, two, three or four substituents independently selected from halo, OH, CN, oxo, —OC₁-C₄ alkyl, —OC₃-C₅ cycloalkyl, —Z₃—R¹¹, and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y, or a pharmaceutically acceptable salt thereof.

74. The compound according to claim 56, wherein R¹⁰ is 5-6 membered heteroaryl, optionally substituted or fused with R^{8A}, or a pharmaceutically acceptable salt thereof.

75. The compound according to claim 56, wherein R¹⁰ is independently selected from cyclopropane, cyclobutane, pyrrolidine, thiazole, pyrazole, triazole, phenyl, pyridine, pyrazine, and pyridazine, optionally substituted or fused with R^{8A}, or a pharmaceutically acceptable salt thereof.

76. The compound according to claim 56, wherein R¹⁰ and R^{8A} are optionally substituted with one or two substituents independently selected from F, Cl, C₁-C₃ alkyl, CH₂F, CHF₂, CF₃, and —OCH₃, or a pharmaceutically acceptable salt thereof.

77. The compound according to claim 56, selected from: 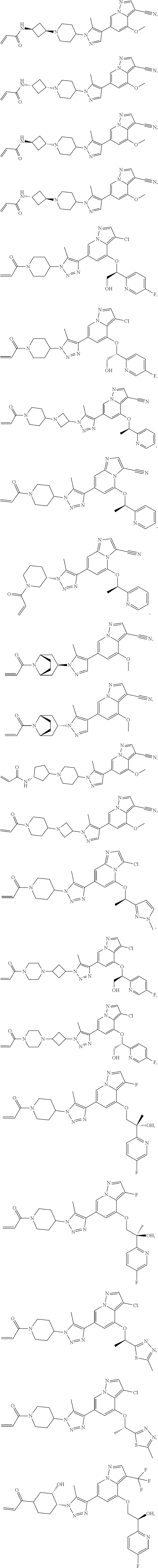 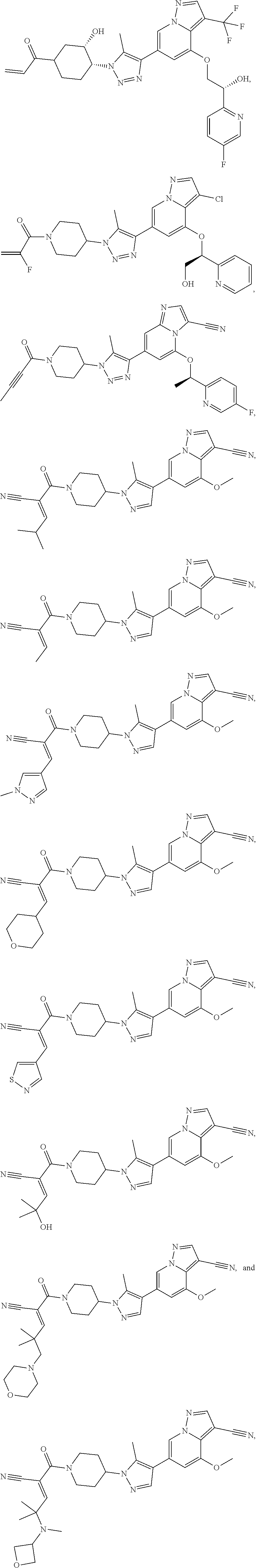
or a pharmaceutically acceptable salt thereof.

78. A pharmaceutical composition comprising a compound, or a pharmaceutically acceptable salt thereof, according to claim 56, and a pharmaceutically acceptable carrier, diluent, or excipient.

79. A method of treating systemic sclerosis, fibrosis, pulmonary fibrosis, achondroplasia, thanatophoric dysplasia, severe achondroplasia with developmental delay and acanthosis nigricans (SADDAN), muenke syndrome or cancer, comprising administering to a patient in need of such treatment an effective amount of a compound of claim 56, or a pharmaceutically acceptable salt thereof.

80. The method of claim 79 wherein the treatment is cancer and the cancer is selected from the group consisting of breast cancer, invasive ductal breast cancer, invasive lobular breast cancer, lung cancer, non-small-cell lung cancer, lung adenocarcinoma, squamous cell lung cancer, small-cell lung cancer, urothelial cancer, bladder cancer, urothelial bladder cancer, non-muscle invasive bladder cancer, high risk non-muscle invasive bladder cancer, intermediate risk non-muscle invasive bladder cancer, Bacillus Calmette-Guerin (BCG)-unresponsive non-muscle invasive bladder cancer, Bacillus Calmette-Guerin (BCG) recurrent non-muscle invasive bladder cancer, muscle invasive bladder cancer, upper tract cancer, urothelial upper tract cancer, urethral cancer, gastric cancer, pancreatic cancer, prostate cancer, colorectal cancer, multiple myeloma, liver cancer, melanoma, cutaneous melanoma, head and neck cancer, oral cancer, thyroid cancer, renal cancer, renal pelvis cancer, glioblastoma, endometrial cancer, cervical cancer, ovarian cancer, and testicular cancer.

81. The method of claim 79, wherein the cancer is selected from the group consisting of bladder cancer, urothelial bladder cancer, non-muscle invasive bladder cancer, and muscle invasive bladder cancer.
